(19) United States
(12) Patent Application Publication
(10) Pub. No.: US 2012/0252782 A1
(43) Pub. Date: Oct. 4, 2012

(54) Xanthine Derivatives, the Preparation Thereof and Their Use as Pharmaceutical Compositions

(75) Inventors: Frank HIMMELSBACH, Mittelbiberach (DE); Michael MARK, Biberach an der Riss (DE); Matthias ECKHARDT, Biberach an der Riss (DE); Elke LANGKOPF, Biberach an der Riss (DE); Roland MAIER, Biberach an der Riss (DE); Ralf R.H. LOTZ, Schemmerhofen (DE)

(73) Assignee: BOEHRINGER INGELHEIM PHARMA GMBH AND CO. KG, Ingelheim am Rhein (DE)

(21) Appl. No.: 13/523,938

(22) Filed: Jun. 15, 2012

Related U.S. Application Data
(63) Continuation of application No. 13/280,394, filed on Oct. 25, 2011.
Application No. 13/280,394 is a continuation of application No. 13/032,686, filed on Feb. 23, 2011, now abandoned.
Application No. 13/032,686 is a continuation of application No. 12/724,653, filed on Mar. 16, 2010, now abandoned.
Application No. 12/724,653 is a continuation of application No. 11/419,756, filed on May 22, 2006, now abandoned.
Application No. 11/419,756 is a continuation of application No. 10/467,961, filed on Dec. 5, 2003, now abandoned.

(30) Foreign Application Priority Data
Jan. 30, 2001 (DE) 10203486
Feb. 24, 2001 (DE) 10109021
Apr. 10, 2001 (DE) 10117803
Aug. 17, 2001 (DE) 10140345

(51) Int. Cl. A61K 31/522; A61K 31/55; C07D 473/06; A61K 31/5377; A61P 3/10; A61P 3/00; A61P 3/08; A61P 3/06; A61P 19/02; A61P 9/10; A61P 3/04; A61P 37/06; A61P 19/10; A61K 31/538; C07D 473/08

(52) U.S. Cl. 514/210.21; 544/272; 514/263.2; 514/263.22; 514/263.34; 540/600; 514/217.06; 544/271; 544/269; 544/118; 514/234.2; 514/252.16; 544/270; 544/105; 514/230.5

(57) ABSTRACT

The present invention relates to substituted xanthines of general formula

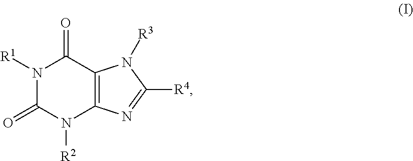

wherein R¹ to R⁴ are defined as in claim 1, the tautomers and the stereoisomers thereof, mixtures thereof, the prodrugs and the salts thereof which have valuable pharmacological properties, particularly an inhibiting effect on the activity of the enzyme dipeptidylpeptidase-IV (DPP-IV).

This application is a continuation of U.S. patent application Ser. No. 13/032,686 filed on Feb. 23, 2011, which is a continuation of U.S. patent application Ser. No. 12/724,653 filed on Mar. 16, 2010, now abandoned, which is a continuation of U.S. patent application Ser. No. 11/419,756 filed on May 22, 2006, now abandoned, which is a continuation of U.S. patent application Ser. No. 10/467,961 filed on Dec. 5, 2003, now abandoned, which is a national stage application filed under 35 USC 371 based on PCT/EP02/01820 filed on Feb. 21, 2002, which international application claims priority of German Application Nos. 101 09 021 filed on Feb. 24, 2001, 101 17 803 filed on Apr. 10, 2001, 101 40 345 filed on Aug. 17, 2001, and 102 03 486 filed on Jan. 30, 2002, all of which applications are incorporated herein by reference in their entireties.

The present invention relates to substituted xanthines of general formula

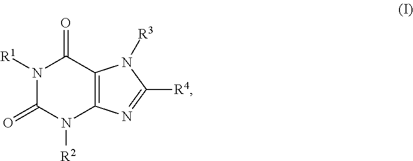

the tautomers, the stereoisomers, the mixtures thereof and the salts thereof, particularly the physiologically acceptable salts thereof with inorganic or organic acids or bases which have valuable pharmacological properties, particularly an inhibiting effect on the activity of the enzyme dipeptidylpeptidase-IV (DPP-IV), the preparation thereof, the use thereof for preventing or treating illnesses or conditions connected with an increased DPP-IV activity or capable of being prevented or alleviated by reducing the DPP-IV activity, particularly type I or type II diabetes mellitus, the pharmaceutical compositions containing a compound of general formula (I) or a physiologically acceptable salt thereof and processes for the preparation thereof.
In the above formula I
R¹ denotes a hydrogen atom,
a C₁₋₈-alkyl group,
a C₁₋₈-alkenyl group,
a C₃₋₄-alkenyl group which is substituted by a C₁₋₂-alkyloxy-carbonyl, aminocarbonyl, C₁₋₃-alkylamino-carbonyl, di-(C₁₋₃-alkyl)-amino-carbonyl, pyrrolidin-1-ylcarbonyl, piperidin-1-ylcarbonyl or morpholin-4-ylcarbonyl-group,
a C₃₋₈-alkynyl group,
a C₁₋₆-alkyl group substituted by a group Rₐ, wherein
Rₐ denotes a C₃₋₇-cycloalkyl, heteroaryl, cyano, carboxy, C₁₋₃-alkyloxy-carbonyl, aminocarbonyl, C₁₋₃-alkylamino-carbonyl, di-(C₁₋₃-alkyl)-amino-carbonyl, pyrrolidin-1-ylcarbonyl, piperidin-1-ylcarbonyl, morpholin-4-ylcarbonyl, piperazin-1-ylcarbonyl, 4-methylpiperazin-1-ylcarbonyl or 4-ethylpiperazin-1-ylcarbonyl group,
   a C₁₋₆-alkyl group substituted by a phenyl group, wherein the phenyl ring is substituted by the groups R¹⁰ to R¹⁴ and
R¹⁰ denotes a hydrogen atom,
a fluorine, chlorine, bromine or iodine atom,
a C₁₋₄-alkyl, hydroxy, or C₁₋₄-alkyloxy group,
   a nitro, amino, C₁₋₃-alkylamino, di-(C₁₋₃-alkyl)amino, cyano-C₁₋₃-alkylamino, [N-(cyano-C₁₋₃-alkyl)-N—C₁₋₃-alkyl-amino], C₁₋₃-alkyloxy-carbonyl-C₁₋₃-alkylamino, pyrrolidin-1-yl, piperidin-1-yl, morpholin-4-yl, piperazin-1-yl or 4-(C₁₋₃-alkyl)-piperazin-1-yl group,
a C₁₋₃-alkyl-carbonylamino, arylcarbonylamino, aryl-C₁₋₃-alkyl-carbonylamino, C₁₋₃-alkyloxy-carbonylamino, aminocarbonylamino, C₁₋₃-alkyl-aminocarbonyl-amino, di-(C₁₋₃-alkyl)aminocarbonylamino, pyrrolidin-1-yl-carbonylamino, piperidin-1-yl-carbonylamino, morpholin-4-yl-carbonylamino, piperazin-1-yl-carbonylamino or 4-(C₁₋₃-alkyl)-piperazin-1-yl-carbonylamino, C₁₋₃-alkyl-sulphonylamino, bis-(C₁₋₃-alkylsulphonyl)-amino, aminosulphonylamino, C₁₋₃-alkylamino-sulphonylamino, di-(C₁₋₃-alkyl)amino-sulphonylamino, pyrrolidin-1-yl-sulphonylamino, piperidin-1-yl-sulphonylamino, morpholin-4-yl-sulphonylamino, piperazin-1-yl-sulphonylamino or 4-(C₁₋₃-alkyl)-piperazin-1-yl-sulphonylamino, (C₁₋₃-alkylamino)thiocarbonylamino, (C₁₋₃-alkyloxy-carbonylamino)carbonylamino, arylsulphonylamino or aryl-C₁₋₃-alkyl-sulphonylamino group,
an N—(C₁₋₃-alkyl)-C₁₋₃-alkyl-carbonylamino, N—(C₁₋₃-alkyl)-arylcarbonylamino, N—(C₁₋₃-alkyl)-aryl-C₁₋₃-alkyl-carbonylamino, N—(C₁₋₃-alkyl)-C₁₋₃-alkyloxy-carbonyl-amino, N-(aminocarbonyl)-C₁₋₃-alkylamino, N—(C₁₋₃-alkyl-aminocarbonyl)-C₁₋₃-alkylamino, N-[di-(C₁₋₃-alkyl)aminocarbonyl]-C₁₋₃-alkylamino, N—(C₁₋₃-alkyl)-C₁₋₃-alkyl-sulphonylamino, N—(C₁₋₃-alkyl)-arylsulphonylamino or N—(C₁₋₃-alkyl)-aryl-C₁₋₃-alkyl-sulphonylamino group,
a 2-oxo-imidazolidin-1-yl, 2,4-dioxo-imidazolidin-1-yl, 2,5-dioxo-imidazolidin-1-yl or 2-oxo-hexahydropyrimidin-1-yl group wherein the nitrogen atom in the 3 position in each case may be substituted by a methyl or ethyl group,
a cyano, carboxy, C₁₋₃-alkyloxy-carbonyl, aminocarbonyl, C₁₋₃-alkyl-aminocarbonyl, di-(C₁₋₃-alkyl)-aminocarbonyl, pyrrolidin-1-yl-carbonyl, piperidin-1-yl-carbonyl, morpholin-4-yl-carbonyl, piperazin-1-yl-carbonyl or 4-(C₁₋₃-alkyl)-piperazin-1-yl-carbonyl group,
a C₁₋₃-alkyl-carbonyl or an arylcarbonyl group,
a carboxy-C₁₋₃-alkyl, C₁₋₃-alkyloxy-carbonyl-C₁₋₃-alkyl, cyano-C₁₋₃-alkyl, aminocarbonyl-C₁₋₃-alkyl, C₁₋₃-alkyl-aminocarbonyl-C₁₋₃-alkyl, di-(C₁₋₃-alkyl)-aminocarbonyl-C₁₋₃-alkyl, pyrrolidin-1-yl-carbonyl-C₁₋₃-alkyl, piperidin-1-yl-carbonyl-C₁₋₃-alkyl, morpholin-4-yl-carbonyl-C₁₋₃-alkyl, piperazin-1-yl-carbonyl-C₁₋₃-alkyl or 4-(C₁₋₃-alkyl)-piperazin-1-yl-carbonyl-C₁₋₃-alkyl group,
a carboxy-C₁₋₃-alkyloxy, C₁₋₃-alkyloxy-carbonyl-C₁₋₃-alkyloxy, cyano-C₁₋₃-alkyloxy, aminocarbonyl-C₁₋₃-alkyloxy, C₁₋₃-alkyl-aminocarbonyl-C₁₋₃-alkyloxy, di-(C₁₋₃-alkyl)-aminocarbonyl-C₁₋₃-alkyloxy, pyrrolidin-1-yl-carbonyl-C₁₋₃-alkyl-oxy, piperidin-1-yl-carbonyl-C₁₋₃-alkyloxy, morpholin-4-yl-carbonyl-C₁₋₃-alkyl-oxy, piperazin-1-yl-carbonyl-C₁₋₃-alkyloxy or 4-(C₁₋₃-alkyl)-piperazin-1-yl-carbonyl-C₁₋₃-alkyloxy group,
a hydroxy-C₁₋₃-alkyl, C₁₋₃-alkyloxy-C₁₋₃-alkyl, amino-C₁₋₃-alkyl, C₁₋₃-alkylamino-C₁₋₃-alkyl, di-(C₁₋₃-alkyl)-amino-C₁₋₃-alkyl, pyrrolidin-1-yl-C₁₋₃-alkyl, piperidin-1-yl-C₁₋₃-alkyl, morpholin-4-yl-C₁₋₃-alkyl, piperazin-1-yl-C₁₋₃-alkyl, 4-(C₁₋₃-alkyl)-piperazin-1-yl-C₁₋₃-alkyl group,
a hydroxy-C₁₋₃-alkyloxy, C₁₋₃-alkyloxy-C₁₋₃-alkyloxy, C₁₋₃-alkylsulphanyl-C₁₋₃-alkyloxy, C₁₋₃-alkylsulphinyl-C₁₋₃-alkyloxy, C₁₋₃-alkylsulphonyl-C₁₋₃-alkyloxy, amino-C₁₋₃-alkyloxy, C₁₋₃-alkylamino-C₁₋₃-alkyloxy, di-(C₁₋₃-alkyl)-amino-C₁₋₃-alkyloxy, pyrrolidin-1-yl-C₁₋₃-alkyloxy, piperidin-1-yl-C₁₋₃-alkyloxy, morpholin-4-yl-C₁₋₃-alkyloxy, piperazin-1-yl-C₁₋₃-alkyloxy, 4-(C₁₋₃-alkyl)-piperazin-1-yl-C₁₋₃-alkyloxy group,
a mercapto, C₁₋₃-alkylsulphanyl, C₁₋₃-alkysulphinyl, C₁₋₃-alkylsulphonyl, C₁₋₃-alkylsulphonyloxy, arylsulphonyloxy, trifluoromethylsulphanyl, trifluoromethylsulphinyl or trifluoromethylsulphonyl group,
a sulpho, aminosulphonyl, C₁₋₃-alkyl-aminosulphonyl, di-(C₁₋₃-alkyl)-amino-sulphonyl, pyrrolidin-1-yl-sulphonyl, piperidin-1-yl-sulphonyl, morpholin-4-yl-sulphonyl, piperazin-1-yl-sulphonyl or 4-(C₁₋₃-alkyl)-piperazin-1-yl-sulphonyl group,
a methyl or methoxy group substituted by 1 to 3 fluorine atoms,
an ethyl or ethoxy group substituted by 1 to 5 fluorine atoms,
a C₂₋₄-alkenyl or C₂₋₄-alkynyl group,
a C₃₋₄-alkenyloxy or C₃₋₄-alkynyloxy group,
a C₃₋₆-cycloalkyl or C₃₋₆-cycloalkyloxy group,
a C₃₋₆-cycloalkyl-C₁₋₃-alkyl or C₃₋₆-cycloalkyl-C₁₋₃-alkyloxy group or
an aryl, aryloxy, aryl-C₁₋₃-alkyl or aryl-C₁₋₃-alkyloxy group,
   R¹¹ and R¹², which may be identical or different, each denote a hydrogen atom, a fluorine, chlorine, bromine or iodine atom, a C₁₋₃-alkyl, trifluoromethyl, hydroxy or C₁₋₃-alkyloxy group or a cyano group, or
   R¹¹ together with R¹², if they are bound to adjacent carbon atoms, also denote a methylenedioxy, difluoromethylenedioxy or a straight-chain C₃₋₅-alkylene group,
   and
   R¹³ and R¹⁴, which may be identical or different, each denote a hydrogen atom, a fluorine, chlorine or bromine atom, a trifluoromethyl, C₁₋₃-alkyl or C₁₋₃-alkyloxy group,
a phenyl-C₁₋₄-alkyl group wherein the alkyl moiety is substituted by a cyano, carboxy, C₁₋₃-alkyloxy-carbonyl, aminocarbonyl, C₁₋₃-alkyl-aminocarbonyl, di-(C₁₋₃-alkyl)-aminocarbonyl, pyrrolidin-1-yl-carbonyl, piperidin-1-yl-carbonyl, morpholin-4-yl-carbonyl group and the phenyl moiety is substituted by the groups R¹⁰ to R¹⁴, wherein R¹⁰ to R¹⁴ are as hereinbefore defined,
   a phenyl group substituted by the groups R¹⁰ to R¹⁴, wherein R¹⁰ to R¹⁴ are as hereinbefore defined,
   a phenyl-C₂₋₃-alkenyl group wherein the phenyl moiety is substituted by the groups R¹⁰ to R¹⁴, wherein R¹⁰ to R¹⁴ are as hereinbefore defined,
   a phenyl-(CH₂)ₘ-A-(CH₂)ₙ-group wherein the phenyl moiety is substituted by R¹⁰ to R¹⁴, wherein R¹⁰ to R¹⁴ are as hereinbefore defined and
A denotes a carbonyl, cyanoiminomethylene, hydroxyiminomethylene or C₁₋₃-alkyloxyiminomethylene group, m denotes the number 0, 1 or 2 and n denotes the number 1, 2 or 3,
   a phenylcarbonylmethyl group wherein the phenyl moiety is substituted by R¹⁰ to R¹⁴, wherein R¹⁰ to R¹⁴ are as hereinbefore defined and the methyl moiety is substituted by a C₁₋₃-alkyl group,
   a phenyl-(CH₂)ₘ-B-(CH₂)ₙ group wherein the phenyl moiety is substituted by R¹⁰ to R¹⁴, wherein R¹⁰ to R¹⁴, m and n are as hereinbefore defined and
B denotes a methylene group which is substituted by a hydroxy, C₁₋₃-alkyloxy, amino, C₁₋₃-alkylamino, di-(C₁₋₃-alkyl)-amino, mercapto, C₁₋₃-alkylsulphanyl, C₁₋₃-alkylsulphinyl or C₁₋₃-alkylsulphonyl group and is optionally additionally substituted by a methyl or ethyl group,
   a naphthyl-C₁₋₃-alkyl group wherein the naphthyl moiety is substituted by the groups R¹⁰ to R¹⁴, wherein R¹⁰ to R¹⁴ are as hereinbefore defined,
   a naphthyl-(CH₂)ₘ-A-(CH₂)ₙ group wherein the naphthyl moiety is substituted by R¹⁰ to R¹⁴, wherein R¹⁰ to R¹⁴, A, m and n are as hereinbefore defined,
   a naphthyl-(CH₂)ₘ-B-(CH₂)ₙ group wherein the naphthyl moiety is substituted by R¹⁰ to R¹⁴, wherein R¹⁰ to R¹⁴, B, m and n are as hereinbefore defined,
   a [1,4]naphthoquinon-2-yl, chromen-4-on-3-yl, 1-oxoindan-2-yl, 1,3-dioxoindan-2-yl- or 2,3-dihydro-3-oxo-benzofuran-2-yl group
   a heteroaryl-(CH₂)ₘ-A-(CH₂)ₙ group, wherein A, m and n are as hereinbefore defined,
   a heteroaryl-(CH₂)ₘ-B-(CH₂)ₙ group, wherein B, m and n are as hereinbefore defined,
   a C₁₋₆-alkyl-A-(CH₂)ₙ group, wherein A and n are as hereinbefore defined,
   a C₃₋₇-cycloalkyl-(CH₂)ₘ-A-(CH₂)ₙ group, wherein A, m and n are as hereinbefore defined,
   a C₃₋₇-cycloalkyl-(CH₂)ₘ-B-(CH₂)ₙ group, wherein B, m and n are as hereinbefore defined,
   an R²¹-A-(CH₂)ₙ group wherein R²¹ denotes a C₁₋₃-alkyloxycarbonyl, aminocarbonyl, C₁₋₃-alkylaminocarbonyl, di-(C₁₋₃-alkyl)aminocarbonyl, pyrrolidin-1-yl-carbonyl, piperidin-1-yl-carbonyl or morpholin-4-yl-carbonyl, piperazin-1-yl-carbonyl, 4-methylpiperazin-1-yl-carbonyl or 4-ethylpiperazin-1-yl-carbonyl group and A and n are as hereinbefore defined,
   a phenyl-(CH₂)ₘ-D-C₁₋₃-alkyl group wherein the phenyl moiety is substituted by the groups R¹⁰ to R¹⁴, wherein R¹⁰ to R¹⁴ and m are as hereinbefore defined and D denotes an oxygen or sulphur atom, an imino, C₁₋₃-alkylimino, sulphinyl or sulphonyl group,
   a naphthyl-(CH₂)ₘ-D-C₁₋₃-alkyl group wherein the naphthyl moiety is substituted by the groups R¹⁰ to R¹⁴, wherein R¹⁰ to R¹⁴, D and m are as hereinbefore defined,
   a C₂₋₆-alkyl group substituted by a group R_{b}, wherein
R_{b} is isolated by at least two carbon atoms from the cyclic nitrogen atom in the 1 position of the xanthine skeleton and
R_{b} denotes a hydroxy, C₁₋₃-alkyloxy, mercapto, C₁₋₃-alkylsulphanyl, C₁₋₃-alkylsulphinyl, C₁₋₃-alkylsulphonyl, amino, C₁₋₃-alkylamino, di-(C₁₋₃-alkyl)-amino, pyrrolidin-1-yl, piperidin-1-yl, morpholin-4-yl, piperazin-1-yl or 4-(C₁₋₃-alkyl)-piperazin-1-yl group,
   a C₃₋₆-cycloalkyl group,
   or an amino or arylcarbonylamino group,
   R² denotes a hydrogen atom,
   a C₁₋₈-alkyl group,
   a C₂₋₆-alkenyl group,
   a C₃₋₆-alkynyl group,
   a C₁₋₆-alkyl group substituted by a group Rₐ, wherein Rₐ is as hereinbefore defined,
   a tetrahydrofuran-3-yl, tetrahydropyran-3-yl, tetrahydropyran-4-yl, tetrahydrofuranyl-C₁₋₃-alkyl or tetrahydropyranyl-C₁₋₃-alkyl group,
   a C₁₋₆-alkyl group substituted by a phenyl group, wherein the phenyl ring is substituted by the groups R¹⁰ to R¹⁴ and R¹⁰ to R¹⁴ are as hereinbefore defined,
   a phenyl group substituted by the groups R¹⁰ to R¹⁴, wherein R¹⁰ to R¹⁴ are as hereinbefore defined,
   a phenyl-C₂₋₃-alkenyl group wherein the phenyl moiety is substituted by the groups R¹⁰ to R¹⁴, wherein R¹⁰ to R¹⁴ are as hereinbefore defined,
   a phenyl-(CH₂)ₘ-A-(CH₂)ₙ group wherein the phenyl moiety is substituted by R¹⁰ to R¹⁴, wherein R¹⁰ to R¹⁴, A, m and n are as hereinbefore defined,
   a phenyl-(CH₂)ₘ-B-(CH₂)ₙ group wherein the phenyl moiety is substituted by R¹⁰ to R¹⁴, wherein R¹⁰ to R¹⁴, B, m and n are as hereinbefore defined,
   a heteroaryl-(CH₂)ₘ-A-(CH₂)ₙ group, wherein A, m and n are as hereinbefore defined,
   a heteroaryl-(CH₂)ₘ-B-(CH₂)ₙ group, wherein B, m and n are as hereinbefore defined,
   a C₁₋₆-alkyl-A-(CH₂)ₙ group, wherein A and n are as hereinbefore defined,
   a C₃₋₇-cycloalkyl-(CH₂)ₘ-A-(CH₂)ₙ group, wherein A, m and n are as hereinbefore defined,
   a C₃₋₇-cycloalkyl-(CH₂)ₘ-B-(CH₂)ₙ group, wherein B, m and n are as hereinbefore defined,
   an R²¹-A-(CH₂)ₙ group wherein R²¹, A and n are as hereinbefore defined,
   a phenyl-(CH₂)ₘ-D-C₁₋₃-alkyl group wherein the phenyl moiety is substituted by the groups R¹⁰ to R¹⁴, wherein R¹⁰ to R¹⁴, m and D are as hereinbefore defined,
   a C₂₋₆-alkyl group substituted by a group R_{b}, wherein
R_{b} is isolated by at least two carbon atoms from the cyclic nitrogen atom in the 3 position of the xanthine skeleton and is as hereinbefore defined,
   or a C₃₋₆-cycloalkyl group,
   R³ denotes a C₁₋₈-alkyl group,
   a C₁₋₄-alkyl group substituted by the group R_{c}, wherein
R_{c} denotes a C₃₋₇-cycloalkyl group optionally substituted by one or two C₁₋₃-alkyl groups,
a C₅₋₇-cycloalkenyl group optionally substituted by one or two C₁₋₃-alkyl groups,
an aryl group, or
a furanyl, thienyl, oxazolyl, isoxazolyl, thiazolyl, isothiazolyl, pyridyl, pyridazinyl, pyrimidyl or pyrazinyl group, wherein the abovementioned heterocyclic groups may each be substituted by one or two C₁₋₃-alkyl groups or by a fluorine, chlorine, bromine or iodine atom or by a trifluoromethyl, cyano or C₁₋₃-alkyloxy group,
   a Cₘ-alkenyl group,
   a C₃₋₆-alkenyl group substituted by a fluorine, chlorine or bromine atom or a trifluoromethyl group,
   a C₃₋₈-alkynyl group,
   an aryl group or
   an aryl-C₂₋₄-alkenyl group,
   and
   R⁴ denotes an azetidin-1-yl or pyrrolidin-1-yl group which is substituted in the 3 position by an RₑNR_{d} group and may additionally be substituted by one or two C₁₋₃-alkyl groups, wherein
Rₑ denotes a hydrogen atom or a C₁₋₃-alkyl group and
R_{d} denotes a hydrogen atom, a C₁₋₃-alkyl group, an R_{f}—C₁₋₃-alkyl group or an R_{g}—C₂₋₃-alkyl group, wherein
   R_{f} denotes a carboxy, C₁₋₃-alkyloxy-carbonyl, aminocarbonyl, C₁₋₃-alkyl-amino-carbonyl, di-(C₁₋₃-alkyl)-aminocarbonyl, pyrrolidin-1-yl-carbonyl, 2-cyanopyrrolidin-1-yl-carbonyl, 2-carboxypyrrolidin-1-yl-carbonyl, 2-methoxycarbonylpyrrolidin-1-yl-carbonyl, 2-ethoxycarbonylpyrrolidin-1-yl-carbonyl, 2-aminocarbonylpyrrolidin-1-yl-carbonyl, 4-cyanothiazolidin-3-yl-carbonyl, 4-carboxythiazolidin-3-yl-carbonyl, 4-methoxycarbonylthiazolidin-3-yl-carbonyl, 4-ethoxy-carbonylthiazolidin-3-yl-carbonyl, 4-aminocarbonylthiazolidin-3-yl-carbonyl, piperidin-1-yl-carbonyl, morpholin-4-yl-carbonyl, piperazin-1-yl-carbonyl, 4-methyl-piperazin-1-yl-carbonyl or 4-ethyl-piperazin-1-yl-carbonyl group and
   R_{g}, which is separated by two carbon atoms from the nitrogen atom of the RₑNR_{d} group, denotes a hydroxy, methoxy or ethoxy group,
      a piperidin-1-yl or hexahydroazepin-1-yl group which is substituted in the 3 position or in the 4 position by an RₑNR_{d} group and may additionally be substituted by one or two C₁₋₃-alkyl groups, wherein Rₑ and R_{d} are as hereinbefore defined,
      a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
      a 3-amino-piperidin-1-yl group wherein the piperidin-1-yl moiety in the 4 position or in the 5 position is additionally substituted by a hydroxy or methoxy group,
      a 3-amino-piperidin-1-yl group wherein the methylene group in the 2 position or in the 6 position is replaced by a carbonyl group,
      a piperidin-1-yl or hexahydroazepin-1-yl-group substituted in the 3 position by an amino, C₁₋₃-alkylamino or di-(C₁₋₃-alkyl)-amino group, wherein in each case two hydrogen atoms at the carbon skeleton of the piperidin-1-yl or hexahydroazepin-1-yl-group are replaced by a straight-chain alkylene bridge, this bridge containing 2 to 5 carbon atoms if the two hydrogen atoms are located on the same carbon atom, or 1 to 4 carbon atoms if the hydrogen atoms are located on adjacent carbon atoms, or 1 to 4 carbon atoms, if the hydrogen atoms are located at carbon atoms separated by one atom, or 1 to 3 carbon atoms if the two hydrogen atoms are located at carbon atoms separated by two atoms,
      an azetidin-1-yl, pyrrolidin-1-yl, piperidin-1-yl or hexahydroazepin-1-yl group which is substituted by an amino-C₁₋₃-alkyl, C₁₋₃-alkylamino-C₁₋₃-alkyl or a —(C₁₋₃-alkyl)amino-C₁₋₃-alkyl group,
      a piperazin-1-yl or [1,4]diazepan-1-yl group optionally substituted at the carbon skeleton by one or two C₁₋₃-alkyl groups,
      a 3-imino-piperazin-1-yl, 3-imino-[1,4]diazepan-1-yl or 5-imino-[1,4]diazepan-1-yl group optionally substituted at the carbon skeleton by one or two C₁₋₃-alkyl groups,
      a [1,4]diazepan-1-yl group optionally substituted by one or two C₁₋₃-alkyl groups, which is substituted in the 6 position by an amino group,
      a C₃₋₇-cycloalkyl group which is substituted by an amino, C₁₋₃-alkylamino or di-(C₁₋₃-alkyl)-amino group,
      a C₃₋₇-cycloalkyl group which is substituted by an amino-C₁₋₃-alkyl, C₁₋₃-alkylamino-C₁₋₃-alkyl or a di-(C₁₋₃-alkyl)amino-C₁₋₃-alkyl group,
      a C₃₋₇-cycloalkyl-C₁₋₂-alkyl group wherein the cycloalkyl moiety is substituted by an amino, C₁₋₃-alkylamino or di-(C₁₋₃-alkyl)-amino group,
      a C₃₋₇-cycloalkyl-C₁₋₂-alkyl group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
      a C₃₋₇-cycloalkylamino group wherein the cycloalkyl moiety is substituted by an amino, C₁₋₃-alkylamino or di-(C₁₋₃-alkyl)-amino group, wherein the two nitrogen atoms on the cycloalkyl moiety are separated from one another by at least two carbon atoms,
      an N—(C₃₋₇-cycloalkyl)-N—(C₁₋₃-alkyl)-amino group wherein the cycloalkyl moiety is substituted by an amino, C₁₋₃-alkylamino or di-(C₁₋₃-alkyl)-amino group, wherein the two nitrogen atoms on the cycloalkyl moiety are separated from one another by at least two carbon atoms,
      a C₃₋₇-cycloalkylamino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
      an N—(C₃₋₇-cycloalkyl)-N—(C₁₋₃-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
      a C₃₋₇-cycloalkyl-C₁₋₂-alkyl-amino group wherein the cycloalkyl moiety is substituted by an amino, C₁₋₃-alkylamino or di-(C₁₋₃-alkyl)-amino group,
      an N—(C₃₋₇-cycloalkyl-C₁₋₂-alkyl)-N—(C₁₋₂-alkyl)-amino group wherein the cycloalkyl moiety is substituted by an amino, C₁₋₃-alkylamino or di-(C₁₋₃-alkyl)-amino group,
      a C₃₋₇-cycloalkyl-C₁₋₂-alkyl-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
      an N—(C₃₋₇-cycloalkyl-C₁₋₂-alkyl)-N—(C₁₋₂-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
      an amino group substituted by the groups R¹⁵ and R¹⁶ wherein
R¹⁵ denotes a C₁₋₆-alkyl group, a C₃₋₆-cycloalkyl, C₃₋₆-cycloalkyl-C₁₋₃-alkyl, aryl or aryl-C₁₋₃-alkyl group and
R¹⁶ denotes an R¹⁷—C₂₋₃-alkyl group, wherein the C₂₋₃-alkyl moiety is straight-chained and may be substituted by one to four C₁₋₃-alkyl groups, which may be identical or different, or by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl)carbonyl, thiazolidin-3-yl-carbonyl, (4-cyano-thiazolidin-3-yl)carbonyl, piperidin-1-ylcarbonyl or morpholin-4-ylcarbonyl group and
R¹⁷ denotes an amino, C₁₋₃-alkylamino or di-(C₁₋₃-alkyl)-amino group, wherein, if R³ denotes a methyl group, R¹⁷ cannot represent a di-(C₁₋₃-alkyl)-amino group,
   an amino group substituted by R²⁰, wherein
R²⁰ denotes an azetidin-3-yl, azetidin-2-ylmethyl, azetidin-3-ylmethyl, pyrrolidin-3-yl, pyrrolidin-2-ylmethyl, pyrrolidin-3-ylmethyl, piperidin-3-yl, piperidin-4-yl, piperidin-2-ylmethyl, piperidin-3-ylmethyl or piperidin-4-ylmethyl group, while the groups mentioned for R²⁰ may each be substituted by one or two C₁₋₃-alkyl groups,
   an amino group substituted by the groups R¹⁵ and R²⁰, wherein
R¹⁵ and R²⁰ are as hereinbefore defined, while the groups mentioned for R²⁰ may each be substituted by one or two C₁₋₃-alkyl groups,
   an R¹⁹—C₃₋₄-alkyl group wherein the C₃₋₄-alkyl moiety is straight-chained and may be substituted by the group R¹⁵ and may additionally be substituted by one or two C₁₋₃-alkyl groups, wherein R¹⁵ is as hereinbefore defined and R¹⁹ denotes an amino, C₁₋₃-alkylamino or di-(C₁₋₃-alkyl)-amino group,
   a 3-amino-2-oxo-piperidin-5-yl or 3-amino-2-oxo-1-methyl-piperidin-5-yl group,
   a pyrrolidin-3-yl, piperidin-3-yl, piperidin-4-yl, hexahydroazepin-3-yl or hexahydroazepin-4-yl group which is substituted in the 1 position by an amino, C₁₋₃-alkylamino or di-(C₁₋₃-alkyl)amino group,
   or an azetidin-2-yl-C₁₋₂-alkyl, azetidin-3-yl-C₁₋₂-alkyl, pyrrolidin-2-yl-C₁₋₂-alkyl, pyrrolidin-3-yl, pyrrolidin-3-yl-C₁₋₂-alkyl, piperidin-2-yl-C₁₋₂-alkyl, piperidin-3-yl, piperidin-3-yl-C₁₋₂-alkyl, piperidin-4-yl or piperidin-4-yl-C₁₋₂-alkyl group, wherein the abovementioned groups may each be substituted by one or two C₁₋₃-alkyl groups,
   while by the aryl groups mentioned in the definition of the groups mentioned above are meant phenyl or naphthyl groups which may be mono- or disubstituted by Rₕ independently of one another, while the substituents may be identical or different and Rₕ denotes a fluorine, chlorine, bromine or iodine atom, a trifluoromethyl, cyano, nitro, amino, aminocarbonyl, aminosulphonyl, methylsulphonyl, acetylamino, methylsulphonylamino, C₁₋₃-alkyl, cyclopropyl, ethenyl, ethynyl, hydroxy, C₁₋₃-alkyloxy, difluoromethoxy or trifluoromethoxy group,
   by the heteroaryl groups mentioned in the definition of the groups mentioned above is meant a pyrrolyl, furanyl, thienyl, pyridyl, indolyl, benzofuranyl, benzothiophenyl, quinolinyl or isoquinolinyl group,
   or a pyrrolyl, furanyl, thienyl or pyridyl group wherein one or two methyne groups are replaced by nitrogen atoms,
   or an indolyl, benzofuranyl, benzothiophenyl, quinolinyl or isoquinolinyl group wherein one to three methyne groups are replaced by nitrogen atoms,
   or a 1,2-dihydro-2-oxo-pyridinyl, 1,4-dihydro-4-oxo-pyridinyl, 2,3-dihydro-3-oxo-pyridazinyl, 1,2,3,6-tetrahydro-3,6-dioxo-pyridazinyl, 1,2-dihydro-2-oxo-pyrimidinyl, 3,4-dihydro-4-oxo-pyrimidinyl, 1,2,3,4-tetrahydro-2,4-dioxo-pyrimidinyl, 1,2-dihydro-2-oxo-pyrazinyl, 1,2,3,4-tetrahydro-2,3-dioxo-pyrazinyl, 2,3-dihydro-2-oxo-indolyl, 2,3-dihydrobenzofuranyl, 2,3-dihydro-2-oxo-1H-benzimidazolyl, 2,3-dihydro-2-oxo-benzoxazolyl, 1,2-dihydro-2-oxo-quinolinyl, 1,4-dihydro-4-oxo-quinolinyl, 1,2-dihydro-1-oxo-isoquinolinyl, 1,4-dihydro-4-oxo-cinnolinyl, 1,2-dihydro-2-oxo-quinazolinyl, 1,4-dihydro-4-oxo-quinazolinyl, 1,2,3,4-tetrahydro-2,4-dioxo-quinazolinyl, 1,2-dihydro-2-oxoquinoxalinyl, 1,2,3,4-tetrahydro-2,3-dioxo-quinoxalinyl, 1,2-dihydro-1-oxo-phthalazinyl, 1,2,3,4-tetrahydro-1,4-dioxo-phthalazinyl, chromanyl, cumarinyl, 2,3-dihydro-benzo[1,4]dioxinyl or 3,4-dihydro-3-oxo-2H-benzo[1,4]oxazinyl group,
wherein the abovementioned heteroaryl groups may be substituted by R¹⁰ to R¹⁴, wherein R¹⁰ to R¹⁴ are as hereinbefore defined,
   while, unless otherwise stated, the abovementioned alkyl, alkenyl and alkynyl groups may be straight-chain or branched,
   as well as the derivatives which are N-oxidised or methylated or ethylated at the cyclic nitrogen atom in the 9 position of the xanthine skeleton,
   as well as the derivatives wherein the 2-oxo, the 6-oxo- or the 2-oxo- and the 6-oxo group of the xanthine skeleton are replaced by thioxo groups,
   with the proviso that the compounds wherein
   R¹ denotes a hydrogen atom, a methyl, propyl, 2-hydroxypropyl, aminocarbonyl-methyl or benzyl group,
   R² denotes a methyl group,
   R³ denotes a C₁₋₈-alkyl group, a benzyl group optionally substituted by a fluorine, chlorine or bromine atom or by a methyl group, a 1-phenylethyl or 2-phenylethyl group, a 2-propen-1-yl, 2-buten-1-yl, 3-chloro-2-buten-1-yl or 2-methyl-2-propen-1-yl group
   and
   R⁴ denotes a piperazin-1-yl group, are excluded,
   and with the proviso that the compounds wherein
   R¹ denotes a hydrogen atom or a methyl group,
   R² denotes a hydrogen atom or a methyl group,
   R³ denotes a methyl group
   and
   R⁴ denotes a 3-aminopropyl, 3-[di-(C₁₋₃-alkyl)amino]-propyl, 1-phenyl-3-[di-(C₁₋₃-alkyl)amino]-propyl, 1-phenyl-3-methyl-3-(dimethylamino)-propyl, 1-(4-chlorophenyl)-3-(dimethylamino)-propyl, 1-phenyl-2-methyl-3-(dimethylamino)-propyl, 1-(3-methoxyphenyl)-3-(dimethylamino)-propyl or a 4-aminobutyl group, are excluded,
   and with the proviso that the compound
   1,3,7-trimethyl-8-(1-aminocyclohexyl)-xanthine
   is excluded,
   the tautomers, enantiomers, diastereomers, mixtures thereof and the salts thereof.

The carboxy groups mentioned in the definition of the abovementioned groups may be replaced by a group which can be converted into a carboxy group in vivo or by a group which is negatively charged under physiological conditions,

and furthermore the amino and imino groups mentioned in the definition of the abovementioned groups may be substituted by a group which can be cleaved in vivo. Such groups are described for example in WO 98/46576 and by N. M. Nielsen et al. in International Journal of Pharmaceutics 39, 75-85 (1987).

By a group which can be converted in vivo into a carboxy group is meant, for example, a hydroxymethyl group, a carboxy group esterified with an alcohol wherein the alcohol moiety is preferably a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxycarbonyl or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol or phenyl-C₃₋₅-alkynol with the proviso that no bonds to the oxygen atom start from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol or an alcohol of formula

Rₚ—CO—O—(R_{q}CRᵣ)—OH,

wherein
Rₚ denotes a C₁₋₈-alkyl, C₅₋₇-cycloalkyl, phenyl or phenyl-C₁₋₃-alkyl group,
R_{q} denotes a hydrogen atom, a C₁₋₃-alkyl, C₅₋₇-cycloalkyl or phenyl group and
Rᵣ denotes a hydrogen atom or a C₁₋₃-alkyl group,
by a group which is negatively charged under physiological conditions is meant, for example, a tetrazol-5-yl, phenylcarbonylaminocarbonyl, trifluoromethylcarbonylaminocarbonyl, C₁₋₆-alkylsulphonylamino, phenylsulphonylamino, benzylsulphonylamino, trifluoromethylsulphonylamino, C₁₋₆-alkylsulphonylaminocarbonyl, phenylsulphonylaminocarbonyl, benzylsulphonylaminocarbonyl or perfluoro-C₁₋₆-alkylsulphonylaminocarbonyl group
and by a group which can be cleaved in vivo from an imino or amino group is meant, for example, a hydroxy group, an acyl group such as a phenylcarbonyl group optionally mono- or disubstituted by fluorine, chlorine, bromine or iodine atoms, by C₁₋₃-alkyl or C₁₋₃-alkoxy groups, while the substituents may be identical or different, a pyridinoyl group or a C₁₋₁₆-alkanoyl group such as the formyl, acetyl, propionyl, butanoyl, pentanoyl or hexanoyl group, a 3,3,3-trichloropropionyl or allyloxycarbonyl group, a C₁₋₁₆-alkoxycarbonyl or C₁₋₁₆-alkylcarbonyloxy group, wherein hydrogen atoms may be wholly or partially replaced by fluorine or chlorine atoms such as the methoxycarbonyl, ethoxycarbonyl, propoxycarbonyl, isopropoxycarbonyl, butoxycarbonyl, tert.butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl, hexadecyloxycarbonyl, methylcarbonyloxy, ethylcarbonyloxy, 2,2,2-trichloroethylcarbonyloxy, propylcarbonyloxy, isopropylcarbonyloxy, butylcarbonyloxy, tert.butylcarbonyloxy, pentylcarbonyloxy, hexylcarbonyloxy, octylcarbonyloxy, nonylcarbonyloxy, decylcarbonyloxy, undecylcarbonyloxy, dodecylcarbonyloxy or hexadecylcarbonyloxy group, a phenyl-C₁₋₆-alkoxycarbonyl group such as the benzyloxycarbonyl, phenylethoxycarbonyl or phenylpropoxycarbonyl group, a 3-amino-propionyl group wherein the amino group may be mono- or disubstituted by C₁₋₆-alkyl or C₃₋₇-cycloalkyl groups and the substituents may be identical or different, a C₁₋₃-alkylsulphonyl-C₂₋₄-alkoxycarbonyl, C₁₋₃-alkoxy-C₂₋₄-alkoxy-C₂₋₄-alkoxycarbonyl, Rₚ—CO—O—(R_{q}CRᵣ)—O—CO, C₁₋₆-alkyl-CO—NH—(RₛCRₜ)—O—CO— or C₁₋₆-alkyl-CO—O—(RₛCRₜ)—(RₛCRₜ)—O—CO— group, wherein Rₚ to Rᵣ are as hereinbefore defined,
Rₛ and Rₜ, which may be identical or different, denote hydrogen atoms or C₁₋₃-alkyl groups.

Moreover, unless otherwise stated, the saturated alkyl and alkoxy moieties containing more than 2 carbon atoms mentioned in the definitions above also include the branched isomers thereof such as the isopropyl, tert.butyl, isobutyl group, etc.

R¹ and R² may denote, for example a hydrogen atom, a methyl, ethyl, propyl, 2-propyl, butyl, 2-butyl, 2-methylpropyl, 2-propen-1-yl, 2-propyn-1-yl, cyclopropylmethyl, benzyl, 2-phenylethyl, phenylcarbonylmethyl, 3-phenylpropyl, 2-hydroxyethyl, 2-methoxyethyl, 2-ethoxyethyl, 2-(dimethylamino)ethyl, 2-(di-ethylamino)ethyl, 2-(pyrrolidino)ethyl, 2-(piperidino)ethyl, 2-(morpholino)ethyl, 2-(piperazino)ethyl, 2-(4-methylpiperazino)ethyl, 3-hydroxypropyl, 3-methoxypropyl, 3-ethoxypropyl, 3-(dimethylamino)propyl, 3-(diethylamino)propyl, 3-(pyrrolidino)propyl, 3-(piperidino)propyl, 3-(morpholino)propyl, 3-(piperazino)-propyl, 3-(4-methylpiperazino)propyl, carboxymethyl, (methoxycarbonyl)methyl, (ethoxycarbonyl)methyl, 2-carboxyethyl, 2-(methoxycarbonyl)ethyl, 2-(ethoxy-carbonyl)ethyl, 3-carboxypropyl, 3-(methoxycarbonyl)propyl, 3-(ethoxycarbonyl)-propyl, (aminocarbonyl)methyl, (methylaminocarbonyl)methyl, (dimethylamino-carbonyl)methyl, (pyrrolidinocarbonyl)methyl, (piperidinocarbonyl)methyl, (morpholinocarbonyl)methyl, 2-(aminocarbonyl)ethyl, 2-(methylaminocarbonyl)ethyl, 2-(dimethylaminocarbonyl)ethyl, 2-(pyrrolidinocarbonyl)ethyl, 2-(piperidinocarbonyl)-ethyl, 2-(morpholinocarbonyl)ethyl, cyanomethyl or 2-cyanoethyl group.

R³ may denote, for example, a methyl, ethyl, propyl, 2-propyl, butyl, 2-butyl, 2-methylpropyl, pentyl, 2-methylbutyl, 3-methylbutyl, 2,2-dimethylpropyl, cyclopropylmethyl, (1-methylcyclopropyl)methyl, (2-methylcyclopropyl)methyl, cyclobutylmethyl, cyclopentylmethyl, cyclohexylmethyl, 2-(cyclopropyl)ethyl, 2-propen-1-yl, 2-methyl-2-propen-1-yl, 3-phenyl-2-propen-1-yl, 2-buten-1-yl, 4,4,4-trifluoro-2-buten-1-yl, 3-buten-1-yl, 2-chloro-2-buten-1-yl, 2-bromo-2-buten-1-yl, 3-chloro-2-buten-1-yl, 3-bromo-2-buten-1-yl, 2-methyl-2-buten-1-yl, 3-methyl-2-buten-1-yl, 2,3-dimethyl-2-buten-1-yl, 3-trifluoromethyl-2-buten-1-yl, 3-methyl-3-buten-1-yl, 1-cyclopenten-1-ylmethyl, (2-methyl-1-cyclopenten-1-yl)methyl, 1-cyclohexen-1-ylmethyl, 2-(1-cyclopenten-1-yl)ethyl, 2-propyn-1-yl, 2-butyn-1-yl, 3-butyn-1-yl, phenyl, methylphenyl, benzyl, a fluorobenzyl, chlorobenzyl, bromobenzyl, methylbenzyl, methoxybenzyl, 1-phenylethyl, 2-phenylethyl, 3-phenylpropyl, 2-furanylmethyl, 3-furanylmethyl, 2-thienylmethyl- or 3-thienylmethyl group.

R⁴ may denote, for example, a 3-aminopyrrolidin-1-yl, 3-aminopiperidin-1-yl, 3-(methylamino)-piperidin-1-yl, 3-(ethylamino)-piperidin-1-yl, 3-(dimethylamino)-piperidin-1-yl, 3-(diethylamino)-piperidin-1-yl, 3-[(2-hydroxyethyl)amino]-piperidin-1-yl, 3-[N-methyl-N-(2-hydroxyethyl)-amino]-piperidin-1-yl, 3-[(3-hydroxypropyl)amino]-piperidin-1-yl, 3-[N-methyl-N-(3-hydroxypropyl)-amino]-piperidin-1-yl, 3-[(carboxy-methyl)amino]-piperidin-1-yl, 3-[(methoxycarbonylmethyl)amino]-piperidin-1-yl, 3-[(ethoxycarbonylmethyl)amino]-piperidin-1-yl, 3-[N-methyl-N-(methoxycarbonyl-methyl)-amino]-piperidin-1-yl, 3-[N-methyl-N-(ethoxycarbonylmethyl)-amino]-piperidin-1-yl, 3-[(2-carboxyethyl)amino]-piperidin-1-yl, 3-{[2-(methoxycarbonyl)ethyl]amino}-piperidin-1-yl, 3-{([2-(ethoxycarbonyl)ethyl]amino}-piperidin-1-yl, 3-{N-methyl-N[2-(methoxycarbonyl)ethyl]-amino}-piperidin-1-yl, 3-{N-methyl-N-[2-(ethoxycarbonyl)ethyl]-amino}-piperidin-1-yl, 3-[(aminocarbonylmethyl)-amino]-piperidin-1-yl, 3-[(methylaminocarbonylmethyl)amino]-piperidin-1-yl, 3-[(dimethylaminocarbonylmethyl)amino]-piperidin-1-yl, 3-[(ethylaminocarbonylmethyl)amino]-piperidin-1-yl, 3-[(diethylaminocarbonylmethyl)amino]-piperidin-1-yl, 3-[(pyrrolidin-1-ylcarbonyl-methyl)amino]-piperidin-1-yl, 3-[(2-cyanopyrrolidin-1-ylcarbonylmethyl)amino]-piperidin-1-yl, 3-[(4-cyanothiazolidin-3-ylcarbonylmethyl)amino]-piperidin-1-yl, 3-[(2-aminocarbonylpyrrolidin-1-ylcarbonylmethyl)amino]-piperidin-1-yl, 3-[(2-carboxypyrrolidin-1-ylcarbonylmethyl)amino]-piperidin-1-yl, 3-[(2-methoxycarbonylpyrrolidin-1-ylcarbonylmethyl)amino]-piperidin-1-yl, 3-[(2-ethoxycarbonylpyrrolidin-1-ylcarbonylmethyl)amino]-piperidin-1-yl, 3-[(piperidin-1-ylcarbonylmethyl)amino]-piperidin-1-yl, 3-[(morpholin-4-ylcarbonylmethyl)amino]-piperidin-1-yl, 3-amino-2-methyl-piperidin-1-yl, 3-amino-3-methyl-piperidin-1-yl, 3-amino-4-methyl-piperidin-1-yl, 3-amino-5-methyl-piperidin-1-yl, 3-amino-6-methyl-piperidin-1-yl, 2-amino-8-aza-bicyclo[3.2.1]oct-8-yl, 6-amino-2-aza-bicyclo[2.2.2]oct-2-yl, 4-aminopiperidin-1-yl, 3-amino-hexahydroazepin-1-yl, 4-amino-hexahydroazepin-1-yl, piperazin-1-yl, [1,4]diazepan-1-yl, 3-aminocyclopentyl, 3-aminocyclohexyl, 3-(methylamino)-cyclohexyl, 3-(ethylamino)-cyclohexyl, 3-(dimethylamino)-cyclohexyl, 3-(diethylamino)-cyclohexyl, 4-aminocyclohexyl, (2-aminocyclopropyl)amino, (2-aminocyclobutyl)amino, (3-aminocyclobutyl)amino, (2-aminocyclopentyl)amino, (3-aminocyclopentyl)amino, (2-aminocyclohexyl)amino or (3-aminocyclohexyl)amino group.

A sub-group deserving special mention relates to those compounds of general formula I wherein R¹ to R⁴ are as hereinbefore defined, with the extra proviso that the compounds wherein R⁴ denotes an optionally substituted piperazin-1-yl or [1,4]diazepan-1-yl group are excluded, the tautomers, enantiomers, diastereomers, mixtures thereof and the salts thereof.

A second sub-group deserving special mention relates to those compounds of general formula I wherein

R¹ denotes a hydrogen atom,
a C₁₋₆-alkyl group,
a C₃₋₆-alkenyl group,
a C₃₋₄-alkenyl group which is substituted by a C₁₋₂-alkyloxy-carbonyl group,
a C₃₋₆-alkynyl group,
a C₃₋₆-cycloalkyl-C₁₋₃-alkyl group,
a phenyl group which may be substituted by a fluorine, chlorine or bromine atom or by a methyl, trifluoromethyl, hydroxy or methoxy group,
a phenyl-C₁₋₄-alkyl group wherein the phenyl moiety is substituted by R¹⁰ to R¹², wherein
R¹⁰ denotes a hydrogen atom, a fluorine, chlorine or bromine atom,
a C₁₋₄-alkyl, trifluoromethyl, hydroxymethyl, C₃₋₆-cycloalkyl, ethynyl or phenyl group,
a hydroxy, C₁₋₄-alkyloxy, difluoromethoxy, trifluoromethoxy, 2,2,2-trifluoroethoxy, phenoxy, benzyloxy, 2-propen-1-yloxy, 2-propyn-1-yloxy, cyano-C₁₋₂-alkyloxy, C₁₋₂-alkylsulphonyloxy, phenylsulphonyloxy, carboxy-C₁₋₃-alkyloxy, C₁₋₃-alkyloxy-carbonyl-C₁₋₃-alkyloxy, aminocarbonyl-C₁₋₃-alkyloxy, C₁₋₂-alkyl-aminocarbonyl-C₁₋₃-alkyloxy, di-(C₁₋₂-alkyl)aminocarbonyl-C₁₋₃-alkyloxy, pyrrolidin-1-yl-carbonyl-C₁₋₃-alkyloxy, piperidin-1-ylcarbonyl-C₁₋₃-alkyloxy, morpholin-4-ylcarbonyl-C₁₋₃-alkyloxy, methylsulphanylmethoxy, methylsulphinylmethoxy, methylsulphonylmethoxy, C₃₋₆-cycloalkyloxy or C₃₋₆-cycloalkyl-C₁₋₂-alkyloxy group,
a carboxy, C₁₋₃-alkyloxycarbonyl, carboxy-C₁₋₃-alkyl, C₁₋₃-alkyloxy-carbonyl-C₁₋₃-alkyl, aminocarbonyl, C₁₋₂-alkylaminocarbonyl, di-(C₁₋₂-alkyl)aminocarbonyl, morpholin-4-ylcarbonyl or cyano group,
a nitro, amino, C₁₋₂-alkylamino, di-(C₁₋₂-alkyl)amino, cyano-C₁₋₂-alkylamino, [N-(cyano-C₁₋₂-alkyl)-N—C₁₋₂-alkyl-amino], C₁₋₂-alkyloxy-carbonyl-C₁₋₂-alkylamino, C₁₋₂-alkyl-carbonylamino, C₁₋₂-alkyloxy-carbonylamino, C₁₋₃-alkylsulphonylamino, bis-(C₁₋₂-alkylsulphonyl)-amino, aminosulphonylamino, C₁₋₂-alkylamino-sulphonylamino, di-(C₁₋₂-alkyl)amino-sulphonylamino, morpholin-4-yl-sulphonylamino, (C₁₋₂-alkylamino)thiocarbonylamino, (C₁₋₂-alkyloxy-carbonylamino)carbonylamino, aminocarbonylamino, C₁₋₂-alkyl-aminocarbonylamino, di-(C₁₋₂-alkyl)aminocarbonylamino or morpholin-4-ylcarbonylamino group,
a 2-oxo-imidazolidin-1-yl, 3-methyl-2-oxo-imidazolidin-1-yl, 2,4-dioxo-imidazolidin-1-yl, 3-methyl-2,4-dioxo-imidazolidin-1-yl, 2,5-dioxo-imidazolidin-1-yl, 3-methyl-2,5-dioxo-imidazolidin-1-yl, 2-oxo-hexahydropyrimidin-1-yl or 3-methyl-2-oxo-hexahydropyrimidin-1-yl group,
or
a C₁₋₂-alkylsulphanyl, C₁₋₂-alkylsulphinyl, C₁₋₂-alkylsulphonyl, aminosulphonyl, C₁₋₂-alkylaminosulphonyl or di-(C₁₋₂-alkyl)aminosulphonyl group,
and R¹¹ and R¹², which may be identical or different, denote a hydrogen, fluorine, chlorine or bromine atom or
a methyl, cyano, trifluoromethyl or methoxy group,
or, R¹¹ together with R¹², if they are bound to adjacent carbon atoms, also denote a methylenedioxy, difluoromethylenedioxy, 1,3-propylene or 1,4-butylene group,
   a phenyl-C₁₋₃-alkyl group wherein the alkyl moiety is substituted by a carboxy, C₁₋₂-alkyloxy-carbonyl, aminocarbonyl, C₁₋₂-alkylaminocarbonyl or di-(C₁₋₂-alkyl)amino-carbonyl group,
   a phenyl-C₂₋₃-alkenyl group, wherein the phenyl moiety may be substituted by a fluorine, chlorine or bromine atom or by a methyl, trifluoromethyl or methoxy group, a phenyl-(CH₂)ₘ-A-(CH₂)ₙ group wherein the phenyl moiety is substituted by R¹⁰ to R¹², wherein R¹⁰ to R¹² are as hereinbefore defined and
A denotes a carbonyl, hydroxyiminomethylene or C₁₋₂-alkyloxyiminomethylene group, m denotes the number 0 or 1 and n denotes the number 1 or 2,
   a phenylcarbonylmethyl group wherein the phenyl moiety is substituted by R¹⁰ to R¹², wherein R¹⁰ to R¹² are as hereinbefore defined and the methyl moiety is substituted by a methyl or ethyl group,
   a phenylcarbonylmethyl group wherein two adjacent hydrogen atoms of the phenyl moiety are replaced by a —O—CO—NH, —NH—CO—NH, —N═CH—NH, —N═CH—O or —O—CH₂—CO—NH— bridge, wherein the abovementioned bridges may be substituted by one or two methyl groups,
   a phenyl-(CH₂)ₘ-B-(CH₂)ₙ group wherein the phenyl moiety is substituted by R¹⁰ to R¹², wherein R¹⁰ to R¹², m and n are as hereinbefore defined and
B denotes a methylene group which is substituted by a hydroxy or C₁₋₂-alkyloxy group and is optionally additionally substituted by a methyl group,
   a naphthylmethyl or naphthylethyl group, wherein the naphthyl moiety is substituted in each case by R¹⁰ to R¹², wherein R¹⁰ to R¹² are as hereinbefore defined,
   a [1,4]naphthoquinon-2-yl, chromen-4-on-3-yl or 1-oxoindan-2-yl group,
   a heteroaryl-C₁₋₃-alkyl group, wherein the term heteroaryl denotes a pyrrolyl, imidazolyl, triazolyl, furanyl, thienyl, oxazolyl, isoxazolyl, thiazolyl, isothiazolyl, pyridyl, pyridazinyl, pyrimidinyl, pyrazinyl, indolyl, benzimidazolyl, 2,3-dihydro-2-oxo-1H-benzimidazolyl, indazolyl, benzofuranyl, 2,3-dihydrobenzofuranyl, benzoxazolyl, dihydro-2-oxo-benzoxazolyl, benzoisoxazolyl, benzothiophenyl, benzothiazolyl, benzoisothiazolyl, quinolinyl, 1,2-dihydro-2-oxo-quinolinyl, isoquinolinyl, 1,2-dihydro-1-oxo-isoquinolinyl, cinnolinyl, quinazolinyl, 1,2-dihydro-2-oxo-quinazolinyl, 1,2-dihydro-1-oxo-phthalazin-4-yl, cumarinyl or 3,4-dihydro-3-oxo-2H-benzo[1,4]oxazinyl group,
wherein the abovementioned heteroaryl groups may be substituted at carbon atoms by a fluorine, chlorine or bromine atom, by a methyl, trifluoromethyl, cyano, aminocarbonyl, aminosulphonyl, methylsulphonyl, nitro, amino, acetylamino, methylsulphonylamino, methoxy, difluoromethoxy or trifluoromethoxy group and the imino groups of the abovementioned heteroaryl groups may be substituted by methyl or ethyl groups, a furanyl-A-CH₂, thienyl-A-CH₂, thiazolyl-A-CH₂ or pyridyl-A-CH₂ group, wherein A is as hereinbefore defined,
   a furanyl-B-CH₂, thienyl-B-CH₂, thiazolyl-B-CH₂ or pyridyl-B-CH₂ group, wherein B is as hereinbefore defined,
   a C₁₋₄-alkyl-A-(CH₂)ₙ group, wherein A and n are as hereinbefore defined,
   a C₃₋₆-cycloalkyl-(CH₂)ₘ-A-(CH₂)ₙ group, wherein A, m and n are as hereinbefore defined,
   a C₃₋₆-cycloalkyl-(CH₂)ₘ-B-(CH₂)ₙ group, wherein B, m and n are as hereinbefore defined,
   a R²¹-A-(CH₂)ₙ group wherein R²¹ denotes a C₁₋₂-alkyloxycarbonyl, aminocarbonyl, C₁₋₂-alkylaminocarbonyl, di-(C₁₋₂-alkyl)aminocarbonyl, pyrrolidin-1-yl-carbonyl, piperidin-1-yl-carbonyl or morpholin-4-yl-carbonyl group and A and n are as hereinbefore defined,
   a phenyl-D-C₁₋₃-alkyl group wherein the phenyl moiety is optionally substituted by a fluorine, chlorine or bromine atom, a methyl, trifluoromethyl or methoxy group and D denotes an oxygen or sulphur atom, a sulphinyl or sulphonyl group,
   a C₁₋₄-alkyl group substituted by a group Rₐ, wherein
Rₐ denotes a cyano, carboxy, C₁₋₃-alkyloxy-carbonyl, aminocarbonyl, C₁₋₂-alkyl-aminocarbonyl, di-(C₁₋₂-alkyl)aminocarbonyl, pyrrolidin-1-yl-carbonyl, piperidin-1-ylcarbonyl or morpholin-4-ylcarbonyl group,
   a C₂₋₄-alkyl group substituted by a group R_{b}, wherein
R_{b} denotes a hydroxy, C₁₋₃-alkyloxy, amino, C₁₋₃-alkylamino, di-(C₁₋₃-alkyl)-amino, pyrrolidin-1-yl, piperidin-1-yl, morpholin-4-yl, piperazin-1-yl, 4-methyl-piperazin-1-yl or 4-ethyl-piperazin-1-yl group and is isolated from the cyclic nitrogen atom in the 1 position of the xanthine skeleton by at least two carbon atoms,
   or an amino or benzoylamino group,
   R² denotes a hydrogen atom,
   a C₁₋₆-alkyl group,
   a C₂₋₄-alkenyl group,
   a C₃₋₄-alkynyl group,
   a C₃₋₆-cycloalkyl group,
   a C₃₋₆-cycloalkyl-C₁₋₃-alkyl group,
   a tetrahydrofuran-3-yl, tetrahydropyran-3-yl, tetrahydropyran-4-yl, tetrahydrofuranylmethyl or tetrahydropyranylmethyl group,
   a phenyl group which is optionally substituted by a fluorine, chlorine or bromine atom or by a methyl, trifluoromethyl, hydroxy, methoxy, difluoromethoxy or trifluoromethoxy group,
   a phenyl-C₁₋₄-alkyl group wherein the phenyl moiety is optionally substituted by a fluorine, chlorine or bromine atom, a methyl, trifluoromethyl, dimethylamino, hydroxy, methoxy, difluoromethoxy or trifluoromethoxy group,
   a phenyl-C₂₋₃-alkenyl group, wherein the phenyl moiety may be substituted by a fluorine, chlorine or bromine atom or by a methyl, trifluoromethyl or methoxy group,
   a phenylcarbonyl-C₁₋₂-alkyl group wherein the phenyl moiety is optionally substituted by a fluorine, chlorine or bromine atom, a methyl, trifluoromethyl, hydroxy, methoxy, difluoromethoxy or trifluoromethoxy group,
   a heteroaryl-C₁₋₃-alkyl group, wherein the term heteroaryl is as hereinbefore defined,
   a furanylcarbonylmethyl, thienylcarbonylmethyl, thiazolylcarbonylmethyl or pyridylcarbonylmethyl group,
   a C₁₋₄-alkyl-carbonyl-C₁₋₂-alkyl group,
   a C₃₋₆-cycloalkyl-carbonyl-C₁₋₂-alkyl group,
   a phenyl-D-C₁₋₃-alkyl group wherein the phenyl moiety is optionally substituted by a fluorine, chlorine or bromine atom, a methyl, trifluoromethyl, hydroxy, methoxy, difluoromethoxy or trifluoromethoxy group, and D is as hereinbefore defined, or
   a C₁₋₄-alkyl group substituted by a group Rₐ, wherein Rₐ is as hereinbefore defined, or
   a C₂₋₄-alkyl group substituted by a group R_{b}, wherein R_{b} is as hereinbefore defined and is isolated from the cyclic nitrogen atom in the 3 position of the xanthine skeleton by at least two carbon atoms,
   R³ denotes a C₁₋₃-alkyl group substituted by the group R_{c}, wherein
R_{c} denotes a C₃₋₇-cycloalkyl group optionally substituted by one or two C₁₋₃-alkyl groups,
a C₅₋₇-cycloalkenyl group optionally substituted by one or two C₁₋₃-alkyl groups or
an aryl group or
a furanyl, thienyl, oxazolyl, isoxazolyl, thiazolyl, isothiazolyl, pyridyl, pyridazinyl, pyrimidyl or pyrazinyl group, wherein the abovementioned heterocyclic groups may each be substituted by one or two C₁₋₃-alkyl groups or by a fluorine, chlorine, bromine or iodine atom or by a trifluoromethyl, cyano or C₁₋₃-alkyloxy group,
   a C₃₋₈-alkenyl group,
   a C₃₋₆-alkenyl group substituted by a fluorine, chlorine or bromine atom, or a trifluoromethyl group,
   a C₃₋₈-alkynyl group,
   an aryl group or
   an aryl-C₂₋₄-alkenyl group,
   and
   R⁴ denotes an azetidin-1-yl or pyrrolidin-1-yl group which is substituted in the 3 position by an RₑNR_{d} group and may additionally be substituted by one or two C₁₋₃-alkyl groups, wherein
Rₑ denotes a hydrogen atom or a C₁₋₃-alkyl group and
R_{d} denotes a hydrogen atom or a C₁₋₃-alkyl group,
   a piperidin-1-yl or hexahydroazepin-1-yl group which is substituted in the 3 position or in the 4 position by an RₑNR_{d} group and may additionally be substituted by one or two C₁₋₃-alkyl groups, wherein Rₑ and R_{d} are as hereinbefore defined,
   a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
   a 3-amino-piperidin-1-yl group wherein the piperidin-1-yl moiety is additionally substituted in the 4 position or in the 5 position by a hydroxy or methoxy group,
   a 3-amino-piperidin-1-yl group wherein the methylene group is replaced in the 2 position or in the 6 position by a carbonyl group,
   a piperidin-1-yl or hexahydroazepin-1-yl group substituted in the 3 position by an amino, C₁₋₃-alkylamino or di-(C₁₋₃-alkyl)-amino group, wherein in each case two hydrogen atoms on the carbon skeleton of the piperidin-1-yl or hexahydroazepin-1-yl group are replaced by a straight-chain alkylene bridge, this bridge containing 2 to 5 carbon atoms if the two hydrogen atoms are located on the same carbon atom, or 1 to 4 carbon atoms if the hydrogen atoms are located on adjacent carbon atoms, or 1 to 4 carbon atoms if the hydrogen atoms are located on carbon atoms which are separated by one atom, or 1 to 3 carbon atoms if the two hydrogen atoms are located on carbon atoms separated by two atoms,
   an azetidin-1-yl, pyrrolidin-1-yl, piperidin-1-yl or hexahydroazepin-1-yl group which is substituted by an amino-C₁₋₃-alkyl, C₁₋₃-alkylamino-C₁₋₃-alkyl or a di-(C₁₋₃-alkyl)amino-C₁₋₃-alkyl group,
   a 3-imino-piperazin-1-yl, 3-imino-[1,4]diazepan-1-yl or 5-imino-[1,4]diazepan-1-yl group optionally substituted by one or two C₁₋₃-alkyl groups on the carbon skeleton,
   a [1,4]diazepan-1-yl group optionally substituted by one or two C₁₋₃-alkyl groups, which is substituted in the 6 position by an amino group,
   a C₃₋₇-cycloalkyl group which is substituted by an amino, C₁₋₃-alkylamino or di-(C₁₋₃-alkyl)-amino group,
   a C₃₋₇-cycloalkyl group which is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkyl-C₁₋₂-alkyl group wherein the cycloalkyl moiety is substituted by an amino, C₁₋₃-alkylamino or di-(C₁₋₃-alkyl)-amino group,
   a C₃₋₇-cycloalkyl-C₁₋₂-alkyl group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkylamino group wherein the cycloalkyl moiety is substituted by an amino, C₁₋₃-alkylamino or di-(C₁₋₃-alkyl)-amino group, wherein the two nitrogen atoms are separated from one another at the cycloalkyl moiety by at least two carbon atoms,
   a N—(C₃₋₇-cycloalkyl)-N—(C₁₋₃-alkyl)-amino group wherein the cycloalkyl moiety is substituted by an amino, C₁₋₃-alkylamino or di-(C₁₋₃-alkyl)-amino group, wherein the two nitrogen atoms are separated from one another at the cycloalkyl moiety by at least two carbon atoms,
   a C₃₋₇-cycloalkylamino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a N—(C₃₋₇-cycloalkyl)-N—(C₁₋₃-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkyl-C₁₋₂-alkyl-amino group wherein the cycloalkyl moiety is substituted by an amino, C₁₋₃-alkylamino or di-(C₁₋₃-alkyl)-amino group,
   a N—(C₃₋₇-cycloalkyl-C₁₋₂-alkyl)-N—(C₁₋₂-alkyl)-amino group wherein the cycloalkyl moiety is substituted by an amino, C₁₋₃-alkylamino or di-(C₁₋₃-alkyl)-amino group,
   a C₃₋₇-cycloalkyl-C₁₋₂-alkyl-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   an N—(C₃₋₇-cycloalkyl-C₁₋₂-alkyl)-N—(C₁₋₂-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   an amino group substituted by the groups R¹⁵ and R¹⁶ wherein
R¹⁵ denotes a C₁₋₃-alkyl group and
R¹⁶ denotes a R¹⁷—C₂₋₃-alkyl group, wherein the C₂₋₃-alkyl moiety is straight-chained and may be substituted by one to four C₁₋₃-alkyl groups, which may be identical or different, or by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl)carbonyl, thiazolidin-3-yl-carbonyl, (4-cyano-thiazolidin-3-yl)carbonyl, piperidin-1-ylcarbonyl or morpholin-4-ylcarbonyl group and
R¹⁷ denotes an amino, C₁₋₃-alkylamino or di-(C₁₋₃-alkyl)-amino group,
   an amino group substituted by the group R²⁰, wherein
R²⁰ denotes an azetidin-3-yl, azetidin-2-ylmethyl, azetidin-3-ylmethyl, pyrrolidin-3-yl, pyrrolidin-2-ylmethyl, pyrrolidin-3-ylmethyl, piperidin-3-yl, piperidin-4-yl, piperidin-2-ylmethyl, piperidin-3-ylmethyl or piperidin-4-ylmethyl group, wherein the groups mentioned for R²⁰ may each be substituted by one or two C₁₋₃-alkyl groups,
   an amino group substituted by the groups R¹⁵ and R²⁰, wherein
R¹⁵ and R²⁰ are as hereinbefore defined, wherein the groups mentioned for R²⁰ may each be substituted by one or two C₁₋₃-alkyl groups,
   a R¹⁹—C₃₋₄-alkyl group wherein the C₃₋₄-alkyl moiety is straight-chained and may be substituted by the group R¹⁵ and may additionally be substituted by one or two C₁₋₃-alkyl groups, wherein R¹⁵ is as hereinbefore defined and R¹⁹ denotes an amino, C₁₋₃-alkylamino or di-(C₁₋₃-alkyl)-amino group,
   a 3-amino-2-oxo-piperidin-5-yl or 3-amino-2-oxo-1-methyl-piperidin-5-yl group,
   a pyrrolidin-3-yl, piperidin-3-yl, piperidin-4-yl, hexahydroazepin-3-yl or hexahydro-azepin-4-yl group, which is substituted in the 1 position by an amino, C₁₋₃-alkylamino or di-(C₁₋₃-alkyl)amino group,
   or an azetidin-2-yl-C₁₋₂-alkyl, azetidin-3-yl-C₁₋₂-alkyl, pyrrolidin-2-yl-C₁₋₂-alkyl, pyrrolidin-3-yl, pyrrolidin-3-yl-C₁₋₂-alkyl, piperidin-2-yl-C₁₋₂-alkyl, piperidin-3-yl, piperidin-3-yl-C₁₋₂-alkyl, piperidin-4-yl or piperidin-4-yl-C₁₋₂-alkyl group, wherein the abovementioned groups may each be substituted by one or two C₁₋₃-alkyl groups,
   while by the aryl groups mentioned in the definition of the groups mentioned above are meant phenyl or naphthyl groups which may be mono- or disubstituted independently of one another by Rₕ, while the substituents may be identical or different and Rₕ denotes a fluorine, chlorine, bromine or iodine atom, a trifluoromethyl, cyano, nitro, amino, C₁₋₃-alkyl, cyclopropyl, ethenyl, ethynyl, hydroxy, C₁₋₃-alkyloxy, difluoromethoxy or trifluoromethoxy group and
   unless otherwise stated, the abovementioned alkyl and alkenyl groups may be straight-chained or branched,
   the tautomers, enantiomers, diastereomers, mixtures thereof and the salts thereof.

A third sub-group deserving special mention relates to those compounds of general formula I wherein

R¹, R² and R³ are as hereinbefore defined and
R⁴ denotes an azetidin-1-yl or pyrrolidin-1-yl group which is substituted in the 3 position by a RₑNR_{d} group and may additionally be substituted by one or two C₁₋₃-alkyl groups, wherein
Rₑ denotes a hydrogen atom or a C₁₋₃-alkyl group and
R_{d} denotes a hydrogen atom or a C₁₋₃-alkyl group,
   a piperidin-1-yl or hexahydroazepin-1-yl group which is substituted in the 3 position or in the 4 position by a RₑNR_{d} group and may additionally be substituted by one or two C₁₋₃-alkyl groups, wherein Rₑ and R_{d} are as hereinbefore defined,
   a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
   a 3-amino-piperidin-1-yl group wherein the piperidin-1-yl moiety is additionally substituted in the 4 position or in the 5 position by a hydroxy or methoxy group,
   a 3-amino-piperidin-1-yl group wherein the methylene group in the 2 position or in the 6 position is replaced by a carbonyl group,
   a piperidin-1-yl or hexahydroazepin-1-yl group substituted in the 3 position by an amino, C₁₋₃-alkylamino or di-(C₁₋₃-alkyl)-amino group, wherein in each case two hydrogen atoms on the carbon skeleton of the piperidin-1-yl or hexahydroazepin-1-yl group are replaced by a straight-chain alkylene bridge, this bridge containing 2 to 5 carbon atoms if the two hydrogen atoms are located on the same carbon atom, or 1 to 4 carbon atoms, if the hydrogen atoms are located on adjacent carbon atoms, or 1 to 4 carbon atoms, if the hydrogen atoms are located on carbon atoms separated by one atom, or 1 to 3 carbon atoms if the two hydrogen atoms are located on carbon atoms separated by two atoms,
   an azetidin-1-yl, pyrrolidin-1-yl, piperidin-1-yl or hexahydroazepin-1-yl group which is substituted by an amino-C₁₋₃-alkyl, C₁₋₃-alkylamino-C₁₋₃-alkyl or a di-(C₁₋₃-alkyl)-amino-C₁₋₃-alkyl group,
   a C₃₋₇-cycloalkyl group which is substituted by an amino, C₁₋₃-alkylamino or di-(C₁₋₃-alkyl)-amino group,
   a C₃₋₇-cycloalkyl group which is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkyl-C₁₋₂-alkyl group wherein the cycloalkyl moiety is substituted by an amino, C₁₋₃-alkylamino or di-(C₁₋₃-alkyl)-amino group,
   a C₃₋₇-cycloalkyl-C₁₋₂-alkyl group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkylamino group wherein the cycloalkyl moiety is substituted by an amino, C₁₋₃-alkylamino or di-(C₁₋₃-alkyl)-amino group, wherein the two nitrogen atoms at the cycloalkyl moiety are separated from one another by at least two carbon atoms,
   a N—(C₃₋₇-cycloalkyl)-N—(C₁₋₃-alkyl)-amino group wherein the cycloalkyl moiety is substituted by an amino, C₁₋₃-alkylamino or di-(C₁₋₃-alkyl)-amino group, wherein the two nitrogen atoms at the cycloalkyl moiety are separated from one another by at least two carbon atoms,
   a C₃₋₇-cycloalkylamino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a N—(C₃₋₇-cycloalkyl)-N—(C₁₋₃-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkyl-C₁₋₂-alkyl-amino group wherein the cycloalkyl moiety is substituted by an amino, C₁₋₃-alkylamino or di-(C₁₋₃-alkyl)-amino group,
   a N—(C₃₋₇-cycloalkyl-C₁₋₂-alkyl)-N—(C₁₋₂-alkyl)-amino group wherein the cycloalkyl moiety is substituted by an amino, C₁₋₃-alkylamino or di-(C₁₋₃-alkyl)-amino group,
   a C₃₋₇-cycloalkyl-C₁₋₂-alkyl-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a N—(C₃₋₇-cycloalkyl-C₁₋₂-alkyl)-N—(C₁₋₂-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   an amino group substituted by the groups R¹⁵ and R¹⁶ wherein
R¹⁵ denotes a C₁₋₄-alkyl group and
R¹⁶ denotes a R¹⁷—C₂₋₃-alkyl group, wherein the C₂₋₃-alkyl moiety is straight-chained and may be substituted by one to four C₁₋₃-alkyl groups, which may be identical or different, or may be substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group and
R¹⁷ denotes an amino, C₁₋₃-alkylamino or di-(C₁₋₃-alkyl)-amino group,
   an amino group substituted by the group R²⁰, wherein
R²⁰ denotes an azetidin-3-yl, azetidin-2-ylmethyl, azetidin-3-ylmethyl, pyrrolidin-3-yl, pyrrolidin-2-ylmethyl, pyrrolidin-3-ylmethyl, piperidin-3-yl, piperidin-4-yl, piperidin-2-ylmethyl, piperidin-3-ylmethyl or piperidin-4-ylmethyl group, wherein the groups mentioned for R²⁰ may each be substituted by one or two C₁₋₃-alkyl groups,
   an amino group substituted by the groups R¹⁵ and R²⁰ wherein
R¹⁵ and R²⁰ are as hereinbefore defined, wherein the groups mentioned for R²⁰ may each be substituted by one or two C₁₋₃-alkyl groups,
   an R¹⁹—C₃₋₄-alkyl group wherein the C₃₋₄-alkyl moiety is straight-chained and may be substituted by the group R¹⁵ and may additionally be substituted by one or two C₁₋₃-alkyl groups, wherein R¹⁵ is as hereinbefore defined and R¹⁹ denotes an amino, C₁₋₃-alkylamino or di-(C₁₋₃-alkyl)-amino group,
   a 3-amino-2-oxo-piperidin-5-yl or 3-amino-2-oxo-1-methyl-piperidin-5-yl group,
   a pyrrolidin-3-yl, piperidin-3-yl, piperidin-4-yl, hexahydroazepin-3-yl or hexahydroazepin-4-yl group, which is substituted in the 1 position by an amino, C₁₋₃-alkylamino or di-(C₁₋₃-alkyl)amino group,
   or an azetidin-2-yl-C₁₋₂-alkyl, azetidin-3-yl-C₁₋₂-alkyl, pyrrolidin-2-yl-C₁₋₂-alkyl, pyrrolidin-3-yl, pyrrolidin-3-yl-C₁₋₂-alkyl, piperidin-2-yl-C₁₋₂-alkyl, piperidin-3-yl, piperidin-3-yl-C₁₋₂-alkyl, piperidin-4-yl or piperidin-4-yl-C₁₋₂-alkyl group, wherein the abovementioned groups may each be substituted by one or two C₁₋₃-alkyl groups, the tautomers, enantiomers, diastereomers, mixtures thereof and the salts thereof.

Preferred compounds of the above general formula I are those wherein

R¹ denotes a hydrogen atom,
a C₁₋₆-alkyl group,
a C₃₋₆-alkenyl group,
a C₃₋₄-alkenyl group which is substituted by a C₁₋₂-alkyloxy-carbonyl group,
a C₃₋₆-alkynyl group,
a C₃₋₆-cycloalkyl-C₁₋₃-alkyl group,
a phenyl group which may be substituted by a fluorine, chlorine or bromine atom or by a methyl, trifluoromethyl, hydroxy or methoxy group,
a phenyl-C₁₋₄-alkyl group wherein the phenyl moiety is substituted by R¹⁰ to R¹², wherein
R¹⁰ denotes a hydrogen atom, a fluorine, chlorine or bromine atom,
a C₁₋₄-alkyl, trifluoromethyl, hydroxymethyl, C₃₋₆-cycloalkyl, ethynyl or phenyl group,
a hydroxy, C₁₋₄-alkyloxy, difluoromethoxy, trifluoromethoxy, 2,2,2-trifluoroethoxy, phenoxy, benzyloxy, 2-propen-1-yloxy, 2-propyn-1-yloxy, cyano-C₁₋₂-alkyloxy, C₁₋₂-alkylsulphonyloxy, phenylsulphonyloxy, carboxy-C₁₋₃-alkyloxy, C₁₋₃-alkyloxy-carbonyl-C₁₋₃-alkyloxy, aminocarbonyl-C₁₋₃-alkyloxy, C₁₋₂-alkyl-aminocarbonyl-C₁₋₃-alkyloxy, di-(C₁₋₂-alkyl)aminocarbonyl-C₁₋₃-alkyloxy, pyrrolidin-1-yl-carbonyl-C₁₋₃-alkyloxy, piperidin-1-ylcarbonyl-C₁₋₃-alkyloxy, morpholin-4-ylcarbonyl-C₁₋₃-alkyloxy, methylsulphanylmethoxy, methylsulphinylmethoxy, methylsulphonylmethoxy, C₃₋₆-cycloalkyloxy or C₃₋₆-cycloalkyl-C₁₋₂-alkyloxy group,
a carboxy, C₁₋₃-alkyloxycarbonyl, carboxy-C₁₋₃-alkyl, C₁₋₃-alkyloxy-carbonyl-C₁₋₃-alkyl, aminocarbonyl, C₁₋₂-alkylaminocarbonyl, di-(C₁₋₂-alkyl)aminocarbonyl, morpholin-4-ylcarbonyl or cyano group,
a nitro, amino, C₁₋₂-alkylamino, di-(C₁₋₂-alkyl)amino, cyano-C₁₋₂-alkylamino, [N-(cyano-C₁₋₂-alkyl)-N—C₁₋₂-alkyl-amino], C₁₋₂-alkyloxy-carbonyl-C₁₋₂-alkylamino, C₁₋₂-alkylcarbonylamino, C₁₋₂-alkyloxy-carbonylamino, C₁₋₃-alkylsulphonylamino, bis-(C₁₋₂-alkylsulphonyl)-amino, aminosulphonylamino, C₁₋₂-alkylamino-sulphonylamino, di-(C₁₋₂-alkyl)amino-sulphonylamino, morpholin-4-yl-sulphonylamino, (C₁₋₂-alkylamino)thiocarbonylamino, (C₁₋₂-alkyloxy-carbonylamino)carbonylamino, aminocarbonylamino, C₁₋₂-alkylaminocarbonylamino, di-(C₁₋₂-alkyl)aminocarbonylamino or morpholin-4-ylcarbonylamino group,
a 2-oxo-imidazolidin-1-yl, 3-methyl-2-oxo-imidazolidin-1-yl, 2,4-dioxo-imidazolidin-1-yl, 3-methyl-2,4-dioxo-imidazolidin-1-yl, 2,5-dioxo-imidazolidin-1-yl, 3-methyl-2,5-dioxo-imidazolidin-1-yl, 2-oxo-hexahydropyrimidin-1-yl or 3-methyl-2-oxo-hexahydropyrimidin-1-yl group,
or
a C₁₋₂-alkylsulphanyl, C₁₋₂-alkylsulphinyl, C₁₋₂-alkylsulphonyl, aminosulphonyl, C₁₋₂-alkylaminosulphonyl or di-(C₁₋₂-alkyl)aminosulphonyl group,
and R¹¹ and R¹², which may be identical or different, denote a hydrogen, fluorine, chlorine or bromine atom or
a methyl, cyano, trifluoromethyl or methoxy group,
or, R¹¹ together with R¹², if they are bound to adjacent carbon atoms, also denote a methylenedioxy, difluoromethylenedioxy, 1,3-propylene or 1,4-butylene group,
   a phenyl-C₁₋₃-alkyl group wherein the alkyl moiety is substituted by a carboxy, C₁₋₂-alkyloxy-carbonyl, aminocarbonyl, C₁₋₂-alkylaminocarbonyl or di-(C₁₋₂-alkyl)amino-carbonyl group,
   a phenyl-C₂₋₃-alkenyl group, wherein the phenyl moiety may be substituted by a fluorine, chlorine or bromine atom or by a methyl, trifluoromethyl or methoxy group,
   a phenyl-(CH₂)ₘ-A-(CH₂)ₙ group wherein the phenyl moiety is substituted by R¹⁰ to R¹², wherein R¹⁰ to R¹² are as hereinbefore defined and
A denotes a carbonyl, hydroxyiminomethylene or C₁₋₂-alkyloxyiminomethylene group, m denotes the number 0 or 1 and n denotes the number 1 or 2,
   a phenylcarbonylmethyl group wherein the phenyl moiety is substituted by R¹⁰ to R¹², wherein R¹⁰ to R¹² are as hereinbefore defined and the methyl moiety is substituted by a methyl or ethyl group,
   a phenylcarbonylmethyl group wherein two adjacent hydrogen atoms of the phenyl moiety are replaced by a —O—CO—NH, —NH—CO—NH, —N═CH—NH, —N═CH—O or —O—CH₂—CO—NH— bridge, wherein the abovementioned bridges may be substituted by one or two methyl groups,
   a phenyl-(CH₂)ₘ-B-(CH₂)ₙ group wherein the phenyl moiety is substituted by R¹⁰ to R¹², wherein R¹⁰ to R¹², m and n are as hereinbefore defined and
B denotes a methylene group which is substituted by a hydroxy or C₁₋₂-alkyloxy group and is optionally additionally substituted by a methyl group,
   a naphthylmethyl or naphthylethyl group, wherein the naphthyl moiety is substituted in each case by R¹⁰ to R¹², wherein R¹⁰ to R¹² are as hereinbefore defined,
   a [1,4]naphthoquinon-2-yl, chromen-4-on-3-yl or 1-oxoindan-2-yl group,
   a heteroaryl-C₁₋₃-alkyl group, wherein by the term heteroaryl is meant a pyrrolyl, imidazolyl, triazolyl, furanyl, thienyl, oxazolyl, isoxazolyl, thiazolyl, isothiazolyl, pyridyl, pyridazinyl, pyrimidinyl, pyrazinyl, indolyl, benzimidazolyl, 2,3-dihydro-2-oxo-1H-benzimidazolyl, indazolyl, benzofuranyl, 2,3-dihydrobenzofuranyl, benzoxazolyl, dihydro-2-oxo-benzoxazolyl, benzisoxazolyl, benzothiophenyl, benzothiazolyl, benzoisothiazolyl, quinolinyl, 1,2-dihydro-2-oxo-quinolinyl, isoquinolinyl, 1,2-dihydro-1-oxo-isoquinolinyl, cinnolinyl, quinazolinyl, 1,2-dihydro-2-oxo-quinazolinyl, 1,2-dihydro-1-oxo-phthalazin-4-yl, cumarinyl or 3,4-dihydro-3-oxo-2H-benzo[1,4]oxazinyl group,
wherein the abovementioned heteroaryl groups may be substituted at carbon atoms by a fluorine, chlorine or bromine atom, by a methyl, trifluoromethyl, cyano, aminocarbonyl, aminosulphonyl, methylsulphonyl, nitro, amino, acetylamino, methylsulphonylamino, methoxy, difluoromethoxy or trifluoromethoxy group and the imino groups of the abovementioned heteroaryl groups may be substituted by methyl or ethyl groups,
   a furanyl-A-CH₂, thienyl-A-CH₂, thiazolyl-A-CH₂ or pyridyl-A-CH₂ group, wherein A is as hereinbefore defined,
   a furanyl-B-CH₂, thienyl-B-CH₂, thiazolyl-B-CH₂ or pyridyl-B-CH₂ group, wherein B is as hereinbefore defined,
   a C₁₋₄-alkyl-A-(CH₂)ₙ group, wherein A and n are as hereinbefore defined,
   a C₃₋₆-cycloalkyl-(CH₂)ₘ-A-(CH₂)ₙ group, wherein A, m and n are as hereinbefore defined,
   a C₃₋₆-cycloalkyl-(CH₂)ₘ-B-(CH₂)ₙ group, wherein B, m and n are as hereinbefore defined,
   an R²¹-A-(CH₂)ₙ group wherein R²¹ denotes a C₁₋₂-alkyloxycarbonyl, aminocarbonyl, C₁₋₂-alkylaminocarbonyl, di-(C₁₋₂-alkyl)aminocarbonyl, pyrrolidin-1-yl-carbonyl, piperidin-1-yl-carbonyl or morpholin-4-yl-carbonyl group and A and n are as hereinbefore defined,
   a phenyl-D-C₁₋₃-alkyl group wherein the phenyl moiety is optionally substituted by a fluorine, chlorine or bromine atom, a methyl, trifluoromethyl or methoxy group and D denotes an oxygen or sulphur atom, a sulphinyl or sulphonyl group,
   a C₁₋₄-alkyl group substituted by a group Rₐ, wherein
Rₐ denotes a cyano, carboxy, C₁₋₃-alkyloxy-carbonyl, aminocarbonyl, C₁₋₂-alkyl-aminocarbonyl, di-(C₁₋₂-alkyl)aminocarbonyl, pyrrolidin-1-yl-carbonyl, piperidin-1-ylcarbonyl or morpholin-4-ylcarbonyl group,
   a C₂₋₄-alkyl group substituted by a group R_{b}, wherein
R_{b} denotes a hydroxy, C₁₋₃-alkyloxy, amino, C₁₋₃-alkylamino, di-(C₁₋₃-alkyl)-amino, pyrrolidin-1-yl, piperidin-1-yl, morpholin-4-yl, piperazin-1-yl, 4-methyl-piperazin-1-yl or 4-ethyl-piperazin-1-yl group and is isolated by at least two carbon atoms from the cyclic nitrogen atom in the 1 position of the xanthine skeleton,
   or an amino or benzoylamino group,
   R² denotes a hydrogen atom,
   a C₁₋₆-alkyl group,
   a C₂₋₄-alkenyl group,
   a C₃₋₄-alkynyl group,
   a C₃₋₆-cycloalkyl group,
   a C₃₋₆-cycloalkyl-C₁₋₃-alkyl group,
   a tetrahydrofuran-3-yl, tetrahydropyran-3-yl, tetrahydropyran-4-yl, tetrahydrofuranylmethyl or tetrahydropyranylmethyl group,
   a phenyl group which is optionally substituted by a fluorine, chlorine or bromine atom or by a methyl, trifluoromethyl, hydroxy, methoxy, difluoromethoxy or trifluoromethoxy group,
   a phenyl-C₁₋₄-alkyl group wherein the phenyl moiety is optionally substituted by a fluorine, chlorine or bromine atom, a methyl, trifluoromethyl, dimethylamino, hydroxy, methoxy, difluoromethoxy or trifluoromethoxy group,
   a phenyl-C₂₋₃-alkenyl group, wherein the phenyl moiety may be substituted by a fluorine, chlorine or bromine atom or by a methyl, trifluoromethyl or methoxy group,
   a phenylcarbonyl-C₁₋₂-alkyl group wherein the phenyl moiety is optionally substituted by a fluorine, chlorine or bromine atom, a methyl, trifluoromethyl, hydroxy, methoxy, difluoromethoxy or trifluoromethoxy group,
   a heteroaryl-C₁₋₃-alkyl group, wherein the term heteroaryl is as hereinbefore defined,
   a furanylcarbonylmethyl, thienylcarbonylmethyl, thiazolylcarbonylmethyl or pyridylcarbonylmethyl group,
   a C₁₋₄-alkyl-carbonyl-C₁₋₂-alkyl group,
   a C₃₋₆-cycloalkyl-carbonyl-C₁₋₂-alkyl group,
   a phenyl-D-C₁₋₃-alkyl group wherein the phenyl moiety is optionally substituted by a fluorine, chlorine or bromine atom, a methyl, trifluoromethyl, hydroxy, methoxy, difluoromethoxy or trifluoromethoxy group, and D is as hereinbefore defined, or
   a C₁₋₄-alkyl group substituted by a group Rₐ, wherein Rₐ is as hereinbefore defined,
   a C₂₋₄-alkyl group substituted by a group R_{b}, wherein R_{b} is as hereinbefore defined and is isolated by at least two carbon atoms from the cyclic nitrogen atom in the 3 position of the xanthine skeleton,
   R³ denotes a C₂₋₆-alkyl group,
   a C₃₋₇-alkenyl group,
   a C₃₋₅-alkenyl group which is substituted by a fluorine, chlorine or bromine atom or a trifluoromethyl group,
   a C₃₋₆-alkynyl group,
   a C₁₋₃-alkyl group substituted by the group R_{c}, wherein
R_{c} denotes a C₃₋₆-cycloalkyl group optionally substituted by one or two methyl groups,
a C₅₋₆-cycloalkenyl group optionally substituted by one or two methyl groups,
a phenyl group optionally substituted by a fluorine, chlorine, bromine or iodine atom, by a methyl, trifluoromethyl, cyano, nitro, amino, hydroxy, methoxy, difluoromethoxy or trifluoromethoxy group,
a phenyl group which is substituted by two fluorine atoms,
a naphthyl group or
a furanyl, thienyl, oxazolyl, isoxazolyl, thiazolyl, isothiazolyl or pyridyl group optionally substituted by a methyl or trifluoromethyl group,
   a phenyl group optionally substituted by a fluorine, chlorine or bromine atom, by a methyl, trifluoromethyl, cyano, hydroxy, methoxy, difluoromethoxy or trifluoromethoxy group,
   a phenyl group which is substituted by two methyl groups,
   a naphthyl group
   or a phenyl-C₂₋₃-alkenyl group
   and
   R⁴ denotes a pyrrolidin-1-yl group which is substituted in the 3 position by an amino, methylamino or dimethylamino group,
   an azetidin-1-yl group which is substituted by an aminomethyl group,
   a pyrrolidin-1-yl group which is substituted by an aminomethyl group,
   a piperidin-1-yl group which is substituted in the 3 position or in the 4 position by an amino, methylamino, dimethylamino or [(2-cyano-pyrrolidin-1-yl-)carbonylmethyl]-amino group, wherein the piperidin-1-yl moiety may additionally be substituted by a methyl or ethyl group,
   a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
   a 3-amino-piperidin-1-yl group wherein the piperidin-1-yl moiety in the 4 position or in the 5 position is additionally substituted by a hydroxy or methoxy group,
   a 3-amino-piperidin-1-yl group wherein the methylene group in the 2 position or in the 6 position is replaced by a carbonyl group,
   a 3-amino-piperidin-1-yl group wherein a hydrogen atom in the 2 position together with a hydrogen atom in the 5 position is replaced by a —CH₂—CH₂— bridge,
   a 3-amino-piperidin-1-yl group wherein a hydrogen atom in the 2 position together with a hydrogen atom in the 6 position is replaced by a —CH₂—CH₂— bridge,
   a 3-amino-piperidin-1-yl group wherein a hydrogen atom in the 4 position together with a hydrogen atom in the 6 position is replaced by a —CH₂—CH₂— bridge,
   a piperidin-1-yl group which is substituted by an aminomethyl group,
   a piperidin-3-yl or piperidin-4-yl group,
   a piperidin-3-yl or piperidin-4-yl group which is substituted in the 1 position by an amino group,
   a hexahydroazepin-1-yl group which is substituted in the 3 position or in the 4 position by an amino group,
   a piperazin-1-yl or [1,4]diazepan-1-yl group optionally substituted at the carbon skeleton by one or two methyl groups,
   a 3-imino-piperazin-1-yl, 3-imino-[1,4]diazepan-1-yl or 5-imino-[1,4]diazepan-1-yl group,
   a [1,4]diazepan-1-yl group, which is substituted in the 6 position by an amino group, a C₃₋₆-cycloalkyl-amino group wherein the cycloalkyl moiety is substituted by an amino, methylamino or dimethylamino group, wherein the two nitrogen atoms are isolated from one another at the cycloalkyl moiety by at least two carbon atoms,
   an N—(C₃₋₆-cycloalkyl)-N—(C₁₋₂-alkyl)-amino group wherein the cycloalkyl moiety is substituted by an amino, methylamino or dimethylamino group, wherein the two nitrogen atoms are isolated from one another at the cycloalkyl moiety by at least two carbon atoms,
   a C₃₋₆-cycloalkyl-amino group wherein the cycloalkyl moiety is substituted by an aminomethyl or aminoethyl group,
   an N—(C₃₋₆-cycloalkyl)-N—(C₁₋₂-alkyl)-amino group wherein the cycloalkyl moiety is substituted by an aminomethyl or aminoethyl group,
   a C₃₋₆-cycloalkyl-C₁₋₂-alkyl-amino group wherein the cycloalkyl moiety is substituted by an amino, aminomethyl or aminoethyl group,
   an N—(C₃₋₆-cycloalkyl-C₁₋₂-alkyl)-N—(C₁₋₂-alkyl)-amino group wherein the cycloalkyl moiety is substituted by an amino, aminomethyl or aminoethyl group,
   an amino group substituted by the groups R¹⁵ and R¹⁶ wherein
R¹⁵ denotes a C₁₋₄-alkyl group and
R¹⁶ denotes a 2-aminoethyl, 2-(methylamino)ethyl or 2-(dimethylamino)ethyl group, wherein the ethyl moiety may in each case be substituted by one or two methyl or ethyl groups or by an aminocarbonyl, C₁₋₂-alkyl-aminocarbonyl, di-(C₁₋₂-alkyl)aminocarbonyl, pyrrolidin-1-yl-carbonyl, piperidin-1-ylcarbonyl or morpholin-4-ylcarbonyl group,
   an amino group wherein the nitrogen atom is substituted by a pyrrolidin-3-yl, piperidin-3-yl, piperidin-4-yl, pyrrolidin-2-ylmethyl, pyrrolidin-3-ylmethyl, piperidin-2-ylmethyl, piperidin-3-ylmethyl or piperidin-4-ylmethyl group,
   a C₁₋₂-alkylamino group wherein the nitrogen atom is substituted by a pyrrolidin-3-yl, piperidin-3-yl, piperidin-4-yl, pyrrolidin-2-ylmethyl, pyrrolidin-3-ylmethyl, piperidin-2-ylmethyl, piperidin-3-ylmethyl or piperidin-4-ylmethyl group,
   a 3-amino-propyl, 3-methylamino-propyl or 3-dimethylamino-propyl group wherein the propyl moiety may be substituted by one or two methyl groups,
   a 4-amino-butyl, 4-methylamino-butyl or 4-dimethylamino-butyl group wherein the butyl moiety may be substituted by one or two methyl groups,
   a C₁₋₂-alkyl group which is substituted by a 2-pyrrolidinyl, 3-pyrrolidinyl, 2-piperidinyl, 3-piperidinyl or 4-piperidinyl group,
   a 3-amino-2-oxo-piperidin-5-yl or 3-amino-2-oxo-1-methyl-piperidin-5-yl group,
   a C₃₋₆-cycloalkyl group which is substituted by an amino, aminomethyl or aminoethyl group or
   a C₃₋₆-cycloalkyl-C₁₋₂-alkyl group wherein the cycloalkyl moiety is substituted by an amino, aminomethyl or aminoethyl group,
   while, unless otherwise stated, the abovementioned alkyl, alkenyl and alkynyl groups may be straight-chain or branched,
   with the proviso that the compounds wherein
   R¹ denotes a hydrogen atom, a methyl, propyl, 2-hydroxypropyl, aminocarbonyl-methyl or benzyl group,
   R² denotes a methyl group,
   R³ denotes a C₁₋₅-alkyl group, a benzyl group optionally substituted by a fluorine, chlorine or bromine atom or by a methyl group, a 1-phenylethyl or 2-phenylethyl group, a 2-propen-1-yl, 2-buten-1-yl, 3-chloro-2-buten-1-yl or 2-methyl-2-propen-1-yl group
   and
   R⁴ denotes a piperazin-1-yl group, are excluded,
   the tautomers, enantiomers, diastereomers, mixtures thereof and the salts thereof.

A sub-group of the preferred compounds of formula I deserving special mention relates to those compounds of general formula I wherein R¹ to R⁴ are as hereinbefore defined, with the additional proviso that the compounds wherein R⁴ denotes an optionally substituted piperazin-1-yl or [1,4]diazepan-1-yl group are excluded, the tautomers, enantiomers, diastereomers, mixtures thereof and the salts thereof.

A second sub-group of the preferred compounds of formula I deserving special mention relates to those compounds of general formula I wherein

R¹ denotes a hydrogen atom,
a C₁₋₄-alkyl group,
a C₃₋₅-alkenyl group,
a 2-propen-1-yl group which is substituted by a methoxycarbonyl group,
a C₃₋₅-alkynyl group,
a phenyl-C₁₋₄-alkyl group wherein the phenyl moiety is substituted by R¹⁰ to R¹², wherein
R¹⁰ denotes a hydrogen atom, a fluorine, chlorine or bromine atom,
a methyl, ethyl, trifluoromethyl or ethynyl group,
a hydroxy, methoxy, ethoxy, difluoromethoxy, trifluoromethoxy, 2,2,2-trifluoroethoxy, phenoxy, benzyloxy, 2-propen-1-yloxy, 2-propyn-1-yloxy, cyano-C₁₋₂-alkyloxy, C₁₋₂-alkyl-sulphonyloxy, phenylsulphonyloxy, carboxy-C₁₋₂-alkyloxy, C₁₋₂-alkyloxy-carbonyl-C₁₋₂-alkyloxy, aminocarbonyl-C₁₋₂-alkyloxy, C₁₋₂-alkyl-aminocarbonyl-C₁₋₂-alkyloxy, di-(C₁₋₂-alkyl)aminocarbonyl-C₁₋₂-alkyloxy, pyrrolidin-1-ylcarbonyl-C₁₋₂-alkyloxy, piperidin-1-ylcarbonyl-C₁₋₂-alkyloxy, morpholin-4-ylcarbonyl-C₁₋₂-alkyloxy group,
a carboxy, C₁₋₂-alkyloxy-carbonyl, aminocarbonyl, C₁₋₂-alkylaminocarbonyl, di-(C₁₋₂-alkyl)aminocarbonyl, morpholin-4-ylcarbonyl or cyano group,
a nitro, amino, C₁₋₂-alkylamino, di-(C₁₋₂-alkyl)amino, cyano-C₁₋₂-alkylamino, [N-(cyano-C₁₋₂-alkyl)-N-methyl-amino], C₁₋₂-alkyloxy-carbonyl-C₁₋₂-alkylamino, C₁₋₂-alkyl-carbonylamino, C₁₋₂-alkyloxy-carbonylamino, C₁₋₂-alkylsulphonylamino, bis-(C₁₋₂-alkylsulphonyl)-amino, aminosulphonylamino, C₁₋₂-alkylamino-sulphonylamino, di-(C₁₋₂-alkyl)amino-sulphonylamino, morpholin-4-yl-sulphonylamino, (C₁₋₂-alkylamino)thiocarbonylamino, (C₁₋₂-alkyloxy-carbonylamino)carbonylamino, aminocarbonylamino, C₁₋₂-alkylaminocarbonylamino, di-(C₁₋₂-alkyl)aminocarbonylamino or morpholin-4-yl-carbonylamino group,
a 2-oxo-imidazolidin-1-yl, 3-methyl-2-oxo-imidazolidin-1-yl, 2,4-dioxo-imidazolidin-1-yl, 3-methyl-2,4-dioxo-imidazolidin-1-yl, 2,5-dioxo-imidazolidin-1-yl, 3-methyl-2,5-dioxo-imidazolidin-1-yl, 2-oxo-hexahydropyrimidin-1-yl or 3-methyl-2-oxo-hexahydropyrimidin-1-yl group,
or
a C₁₋₂-alkylsulphanyl, C₁₋₂-alkylsulphinyl, C₁₋₂-alkylsulphonyl, aminosulphonyl, C₁₋₂-alkylaminosulphonyl or di-(C₁₋₂-alkyl)aminosulphonyl group,
and R¹¹ and R¹², which may be identical or different, denote a hydrogen, fluorine, chlorine or bromine atom or
a methyl, cyano or methoxy group,
or, R¹¹ together with R¹², if they are bound to adjacent carbon atoms, also denote a methylenedioxy group,
   a phenylmethyl group wherein the methyl moiety is substituted by a carboxy, methoxycarbonyl or aminocarbonyl group,
   a 2-phenylethyl group wherein the ethyl moiety is substituted by a carboxy, methoxycarbonyl or aminocarbonyl group,
   a 2-phenylethyl group wherein the ethyl moiety is substituted in the 2 position by a hydroxy, methoxy, hydroxyimino or methoxyimino group,
   a 2-phenylethyl group wherein the ethyl moiety is substituted in the 2 position by a hydroxy group and a methyl group,
   a phenylcarbonylmethyl group wherein the phenyl moiety is substituted by R¹⁰ to R¹², wherein R¹⁰ to R¹² are as hereinbefore defined,
   a 1-(phenylcarbonyl)ethyl or 2-(phenylcarbonyl)ethyl group,
   a 2-phenylethenyl group,
   a phenylsulphanylmethyl or phenylsulphinylmethyl group,
   a 2-(phenyloxy)ethyl group,
   a naphthylmethyl or naphthylethyl group, wherein the naphthyl moiety may be substituted in each case by a methyl, nitro, amino, acetylamino, methylsulphonylamino, cyano, aminocarbonyl or aminosulphonyl group,
   a [1,4]naphthoquinon-2-yl, chromen-4-on-3-yl or 1-oxoindan-2-yl group
   an oxazolylmethyl, isoxazolylmethyl, thiazolylmethyl, pyridylmethyl, benzo-furanylmethyl, 2,3-dihydrobenzofuranylmethyl, benzo[d]isoxazolylmethyl, benzo-[d]isothiazolylmethyl, (1H-indazol-3-yl)methyl, quinolinylmethyl, (1,2-dihydro-2-oxo-quinolin-4-yl)methyl, isoquinolinylmethyl, (1,2-dihydro-1-oxo-isoquinolin-4-yl)methyl, cinnolinylmethyl, quinazolinylmethyl, (1,2-dihydro-2-oxo-quinazolin-4-yl)methyl, (1,2-dihydro-1-oxo-phthalazin-4-yl)methyl or cumarinylmethyl group, wherein the heterocyclic moiety may be substituted by a methyl group in each case,
   a quinolinylmethyl or isoquinolinylmethyl group, wherein the heterocyclic moiety is substituted in each case by a cyano, nitro, amino, acetylamino, methylsulphonylamino, aminocarbonyl or aminosulphonyl group,
   a pyrrolylethyl, triazolylethyl, thienylethyl, thiazolylethyl or pyridylethyl group, wherein the heterocyclic moiety may be substituted in each case by a methyl group,
   a furanylcarbonylmethyl, thienylcarbonylmethyl, thiazolylcarbonylmethyl or pyridylcarbonylmethyl group,
   a methyl group which is substituted by a cyclopropyl, cyano, carboxy, aminocarbonyl or methoxycarbonyl group,
   an ethyl group which is substituted in the 2 position by a hydroxy, methoxy, dimethylamino, carboxy or methoxycarbonyl group, or
   a propyl group which is substituted in the 3 position by a hydroxy, dimethylamino, carboxy or methoxycarbonyl group,
   a 2-oxopropyl group or
   an amino or benzoylamino group,
   R² denotes a hydrogen atom,
   a C₁₋₆-alkyl group,
   an ethenyl group,
   a 2-propen-1-yl or 2-propyn-1-yl group,
   a C₃₋₆-cycloalkyl group,
   a tetrahydrofuran-3-yl, tetrahydropyran-3-yl, tetrahydropyran-4-yl, tetrahydro-furanylmethyl or tetrahydropyranylmethyl group,
   a phenyl group,
   a phenyl-C₁₋₄-alkyl group, wherein the phenyl moiety may be substituted by a fluorine or chlorine atom, a methyl, dimethylamino, hydroxy, methoxy or trifluoromethoxy group,
   a phenylcarbonylmethyl group, wherein the phenyl moiety may be substituted by a fluorine or chlorine atom, a hydroxy, methoxy or trifluoromethoxy group,
   a 2-phenylethenyl group,
   a 2-(phenyloxy)ethyl group,
   a pyridylmethyl or pyridylethyl group,
   a methyl group which is substituted by a C₃₋₆-cycloalkyl, cyano, carboxy or methoxy-carbonyl group, or
   an ethyl group which is substituted in the 2 position by a C₃₋₆-cycloalkyl, cyano, carboxy, methoxycarbonyl, hydroxy, methoxy or dimethylamino group,
   or a propyl group which is substituted in the 3 position by a C₃₋₆-cycloalkyl, cyano, carboxy, methoxycarbonyl, hydroxy, methoxy or dimethylamino group,
   R³ denotes a C₄₋₆-alkenyl group,
   a 1-cyclopenten-1-ylmethyl or 1-cyclohexen-1-ylmethyl group,
   a 1-cyclopenten-1-ylmethyl group wherein the 1-cyclopenten-1-yl moiety is substituted by a methyl group,
   a 2-propyn-1-yl, 2-butyn-1-yl or 2-pentyn-1-yl group,
   a phenyl group which may be substituted by a fluorine atom or a cyano, methyl-methoxy or trifluoromethyl group,
   a phenyl group which is substituted by two methyl groups,
   a benzyl group wherein the phenyl moiety may be substituted by one or two fluorine atoms, a chlorine, bromine or iodine atom, or a methyl, methoxy, cyano, nitro or amino group,
   a furanylmethyl or thienylmethyl group,
   a cyclopropylmethyl group or
   a cyclopropylmethyl group wherein the cyclopropyl moiety is substituted by a methyl group, and
   R⁴ denotes a piperidin-1-yl group which is substituted in the 3 position by an amino group, wherein the piperidin-1-yl moiety may additionally be substituted by a methyl group,
   a 3-amino-piperidin-1-yl group wherein the piperidin-1-yl moiety is additionally substituted by an aminocarbonyl, methylaminocarbonyl, dimethylaminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
   a 3-amino-piperidin-1-yl group wherein the piperidin-1-yl moiety in the 4 position or in the 5 position is additionally substituted by a hydroxy or methoxy group,
   a 3-amino-piperidin-1-yl group wherein a hydrogen atom in the 2 position together with a hydrogen atom in the 5 position is replaced by a —CH₂—CH₂-bridge,
   a hexahydroazepin-1-yl group which is substituted in the 3 position by an amino group,
   a 3-amino-2-oxo-piperidin-5-yl or 3-amino-2-oxo-1-methyl-piperidin-5-yl group,
   a [1,4]diazepan-1-yl group, which is substituted in the 6 position by an amino group,
   a cyclohexyl group which is substituted in the 3 position by an amino group,
   a 2-amino-cyclohexylamino group,
   or an amino group substituted by the groups R¹⁵ and R¹⁶ wherein
R¹⁵ denotes a methyl or ethyl group and
R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
   unless otherwise stated, the abovementioned alkyl and alkenyl groups may be straight-chained or branched,
   the tautomers, enantiomers, diastereomers, mixtures thereof and the salts thereof.

A third sub-group of the preferred compounds of formula I deserving special mention relates to those compounds of general formula I wherein

R¹, R² and R³ are as hereinbefore defined and
R⁴ denotes a piperidin-1-yl group which is substituted in the 3 position by an amino group, wherein the piperidin-1-yl moiety may additionally be substituted by a methyl group,
a 3-amino-piperidin-1-yl group wherein the piperidin-1-yl moiety is additionally substituted by an aminocarbonyl, methylaminocarbonyl, dimethylaminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
a 3-amino-piperidin-1-yl group wherein the piperidin-1-yl moiety is additionally substituted in the 4 position or in the 5 position by a hydroxy or methoxy group,
a 3-amino-piperidin-1-yl group wherein a hydrogen atom in the 2 position together with a hydrogen atom in the 5 position is replaced by a —CH₂—CH₂-bridge,
a hexahydroazepin-1-yl group which is substituted in the 3 position by an amino group,
a 3-amino-2-oxo-piperidin-5-yl or 3-amino-2-oxo-1-methyl-piperidin-5-yl group,
a cyclohexyl group which is substituted in the 3 position by an amino group,
a 2-amino-cyclohexylamino group,
or an amino group substituted by the groups R¹⁵ and R¹⁶, wherein
R¹⁵ denotes a methyl or ethyl group and
R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
   unless otherwise stated, the abovementioned alkyl- and alkenyl groups may be straight-chained or branched,
   the tautomers, enantiomers, diastereomers, mixtures thereof and the salts thereof.

Particularly preferred compounds of the above general formula I are those wherein

R¹ denotes a hydrogen atom,
a C₁₋₄-alkyl group,
a C₃₋₅-alkenyl group,
a 2-propen-1-yl group which is substituted by a methoxycarbonyl group,
a C₃₋₅-alkynyl group,
a phenyl group,
a phenyl-C₁₋₄-alkyl group wherein the phenyl moiety may be substituted by one or two fluorine atoms, one or two chlorine atoms, a bromine atom, one to three methyl groups, a butyl, trifluoromethyl, hydroxy, methoxy, nitro, amino, carboxy or ethoxycarbonyl group,
a 2-phenylethyl group wherein the ethyl moiety is substituted in the 2 position by a hydroxy, methoxy or hydroxyimino group,
a phenylcarbonylmethyl group wherein the phenyl moiety may be substituted by a fluorine atom or by a methyl, aminocarbonyl, aminosulphonyl, cyano, hydroxy, methoxy, phenoxy, benzyloxy, 2-propen-1-yloxy, 2-propyn-1-yloxy, cyanomethoxy, (methoxycarbonyl)methoxy, (aminocarbonyl)methoxy, (methylaminocarbonyl)-methoxy, (dimethylaminocarbonyl)methoxy, methylsulphonyloxy, phenylsulphonyloxy, nitro, amino, (methoxycarbonyl)methylamino, acetylamino, methoxycarbonylamino, methylsulphonylamino, bis-(methylsulphonyl)-amino, aminocarbonylamino, dimethylaminocarbonylamino, (methylamino)thiocarbonylamino, (ethoxy carbonylamino)carbonylamino or cyanomethylamino group,
a phenylcarbonylmethyl group wherein the phenyl moiety is substituted by two methoxy groups or by a bromine atom and by a dimethylamino group,
a 2-(phenylcarbonyl)ethyl group,
a 2-phenylethenyl group,
a 2-(phenoxy)ethyl group,
a phenylsulphanylmethyl or phenylsulphinylmethyl group,
a naphthylmethyl or naphthylethyl group,
an isoxazolylmethyl, thiazolylmethyl, pyridylmethyl, benzo[d]isoxazolylmethyl, benzo[d]isothiazolylmethyl, (1H-indazol-3-yl)methyl, quinolinylmethyl or isoquinolinylmethyl group, wherein the heterocyclic moiety may in each case be substituted by a methyl group,
a isoquinolinylmethyl group wherein the isoquinolinyl moiety is substituted by a nitro or amino group,
a (1,2-dihydro-2-oxo-quinolin-4-yl)methyl group,
a chromen-4-on-3-yl group,
a pyrrolylethyl, triazolylethyl, thienylethyl, thiazolylethyl or pyridylethyl group, wherein the heterocyclic moiety may in each case be substituted by a methyl group,
a thienylcarbonylmethyl group,
a methyl group which is substituted by a cyclopropyl, cyano, carboxy, aminocarbonyl or methoxycarbonyl group,
an ethyl group which is substituted in the 2 position by a hydroxy, methoxy, dimethylamino, carboxy or methoxycarbonyl group, or
a propyl group which is substituted in the 3 position by a hydroxy, dimethylamino, carboxy or methoxycarbonyl group,
a 2-oxopropyl group or
an amino or benzoylamino group,
R² denotes a hydrogen atom,
a C₁₋₆-alkyl group,
an ethenyl group,
a 2-propen-1-yl or 2-propyn-1-yl group,
a phenyl group,
a phenyl-C₁₋₄-alkyl group, wherein the phenyl moiety may be substituted by a fluorine atom, a methyl or methoxy group,
a phenylcarbonylmethyl group,
a 2-phenylethenyl group,
a methyl group which is substituted by a cyclopropyl, cyano, carboxy or methoxy-carbonyl group, or
an ethyl group which is substituted in the 2 position by a cyano, hydroxy, methoxy or dimethylamino group,
R³ denotes a C₄₋₆-alkenyl group,
a 1-cyclopenten-1-ylmethyl or 1-cyclohexen-1-ylmethyl group,
a 2-propyn-1-yl, 2-butyn-1-yl or 2-pentyn-1-yl group,
a phenyl group which may be substituted by a fluorine atom or a cyano, methyl or trifluoromethyl group,
a phenyl group which is substituted by two methyl groups,
a naphthyl group,
a benzyl group wherein the phenyl moiety may be substituted by one or two fluorine atoms, an iodine atom or a cyano, nitro or amino group,
a naphthylmethyl group,
a 2-phenylethenyl group,
a furanylmethyl or thienylmethyl group or
a cyclopropylmethyl group and
R⁴ denotes a pyrrolidin-1-yl group which is substituted in the 3 position by an amino group,
an azetidin-1-yl group which is substituted by an aminomethyl group,
a pyrrolidin-1-yl group which is substituted by an aminomethyl group,
a piperidin-1-yl group which is substituted in the 3 position or in the 4 position by an amino, methylamino, dimethylamino or [(2-cyano-pyrrolidin-1-yl)carbonylmethyl]-amino group, wherein the piperidin-1-yl moiety may additionally be substituted by a methyl group,
a 3-amino-piperidin-1-yl group wherein the piperidin-1-yl moiety is additionally substituted by a pyrrolidin-1-yl-carbonyl group,
a 3-amino-piperidin-1-yl group wherein the piperidin-1-yl moiety in the 4 position is additionally substituted by a hydroxy group,
a 3-amino-piperidin-1-yl group wherein a hydrogen atom in the 2 position together with a hydrogen atom in the 5 position is replaced by a —CH₂—CH₂-bridge,
a piperidin-1-yl group which is substituted by an aminomethyl group,
a piperidin-3-yl or piperidin-4-yl group,
a 1-amino-piperidin-3-yl or 1-amino-piperidin-4-yl group,
a hexahydroazepin-1-yl group which is substituted in the 3 position or in the 4 position by an amino group,
a piperazin-1-yl or [1,4]diazepan-1-yl group,
a [1,4]diazepan-1-yl group, which is substituted in the 6 position by an amino group,
a 3-aminopropyl group,
a cyclohexyl group which is substituted by an amino group,
a 2-amino-cyclopropylamino group,
a 2-amino-cyclobutylamino group,
a 2-amino-cyclopentylamino or 3-amino-cyclopentylamino group,
a 2-amino-cyclohexylamino, 2-(methylamino)-cyclohexylamino or 3-amino-cyclohexylamino group,
an N-(2-aminocyclohexyl)-methylamino group,
an amino group substituted by the groups R¹⁵ and R¹⁶ wherein
R¹⁵ denotes a methyl or ethyl group and
R¹⁶ denotes a 2-aminoethyl-2-(methylamino)ethyl or 2-(dimethylamino)ethyl group, wherein the ethyl moiety may be substituted by one or two methyl groups or by an aminocarbonyl, methylaminocarbonyl, dimethylaminocarbonyl or pyrrolidin-1-ylcarbonyl group,
   or an amino or methylamino group wherein the nitrogen atom is substituted by a pyrrolidin-3-yl, piperidin-3-yl, piperidin-4-yl or piperidin-2-ylmethyl group,
   while unless otherwise stated, the abovementioned alkyl and alkenyl groups may be straight-chain or branched,
   with the proviso that the compounds
3-methyl-7-(2-buten-1-yl)-8-(piperazin-1-yl)-xanthine,
3-methyl-7-(2-methyl-2-propen-1-yl)-8-(piperazin-1-yl)-xanthine,
3-methyl-7-benzyl-8-(piperazin-1-yl)-xanthine,
1,7-dibenzyl-3-methyl-8-(piperazin-1-yl)-xanthine and
1,3-dimethyl-7-(4-fluorobenzyl)-8-(piperazin-1-yl)-xanthine
   are excluded,
   the tautomers, enantiomers, diastereomers, mixtures thereof and the salts thereof.

A sub-group of the particularly preferred compounds of formula I deserving special mention relates to those compounds of general formula I wherein R¹ to R⁴ are as hereinbefore defined, with the additional proviso that the compounds wherein R⁴ denotes an optionally substituted piperazin-1-yl or [1,4]diazepan-1-yl group are excluded, the tautomers, enantiomers, diastereomers, mixtures thereof and the salts thereof.

A second sub-group of the particularly preferred compounds of formula I deserving special mention relates to those compounds of general formula I wherein

R¹ denotes a hydrogen atom,
a C₁₋₄-alkyl group,
a C₃₋₅-alkenyl group,
a 2-propen-1-yl group which is substituted by a methoxycarbonyl group,
a C₃₋₅-alkynyl group,
a phenyl-C₁₋₄-alkyl group wherein the phenyl moiety may be substituted by one or two fluorine atoms, one or two chlorine atoms, a bromine atom, one to three methyl groups, a trifluoromethyl, hydroxy, methoxy, nitro, amino, carboxy or ethoxycarbonyl group,
a 2-phenylethyl group wherein the ethyl moiety is substituted in the 2 position by a hydroxy, methoxy or hydroxyimino group,
a phenylcarbonylmethyl group wherein the phenyl moiety may be substituted by a fluorine atom or by a methyl, aminocarbonyl, aminosulphonyl, cyano, hydroxy, methoxy, phenoxy, benzyloxy, 2-propen-1-yloxy, 2-propyn-1-yloxy, cyanomethoxy, (methoxycarbonyl)methoxy, (aminocarbonyl)methoxy, (methylaminocarbonyl)-methoxy, (dimethylaminocarbonyl)methoxy, methylsulphonyloxy, phenylsulphonyloxy, nitro, amino, (methoxycarbonyl)methylamino, acetylamino, methoxycarbonylamino, methylsulphonylamino, bis-(methylsulphonyl)-amino, aminocarbonylamino, dimethylaminocarbonylamino, (methylamino)thiocarbonylamino, (ethoxycarbonylamino)carbonylamino or cyanomethylamino group,
a phenylcarbonylmethyl group wherein the phenyl moiety is substituted by two methoxy groups or by a bromine atom and by a dimethylamino group,
a 2-(phenylcarbonyl)ethyl group,
a 2-phenylethenyl group,
a 2-(phenoxy)ethyl group,
a phenylsulphanylmethyl or phenylsulphinylmethyl group,
a naphthylmethyl or naphthylethyl group,
an isoxazolylmethyl, thiazolylmethyl, pyridylmethyl, benzo[d]isoxazolylmethyl, benzo[d]isothiazolylmethyl, (1H-indazol-3-yl)methyl, quinolinylmethyl or iso-quinolinylmethyl group, wherein the heterocyclic moiety may be substituted in each case by a methyl group,
an isoquinolinylmethyl group wherein the isoquinolinyl moiety is substituted by a nitro or amino group,
a (1,2-dihydro-2-oxo-quinolin-4-yl)methyl group,
a pyrrolylethyl, triazolylethyl, thienylethyl, thiazolylethyl or pyridylethyl group, wherein the heterocyclic moiety may be substituted in each case by a methyl group,
a thienylcarbonylmethyl group,
a methyl group which is substituted by a cyclopropyl, cyano, carboxy, aminocarbonyl or methoxycarbonyl group,
an ethyl group which is substituted in the 2 position by a hydroxy, methoxy, dimethylamino, carboxy or methoxycarbonyl group, or
a propyl group which is substituted in the 3 position by a hydroxy, dimethylamino, carboxy or methoxycarbonyl group,
a 2-oxopropyl group or
an amino or benzoylamino group,
R² denotes a hydrogen atom,
a C₁₋₆-alkyl group,
an ethenyl group,
a 2-propen-1-yl or 2-propyn-1-yl group,
a phenyl group,
a phenyl-C₁₋₄-alkyl group wherein the phenyl moiety may be substituted by a fluorine atom, a methyl or methoxy group,
a phenylcarbonylmethyl group,
a 2-phenylethenyl group,
a methyl group which is substituted by a cyclopropyl, cyano, carboxy or methoxy-carbonyl group, or
an ethyl group which is substituted in the 2 position by a cyano, hydroxy, methoxy or dimethylamino group,
R³ denotes a C₄₋₆-alkenyl group,
a 1-cyclopenten-1-ylmethyl or 1-cyclohexen-1-ylmethyl group,
a 2-propyn-1-yl, 2-butyn-1-yl or 2-pentyn-1-yl group,
a phenyl group which may be substituted by a fluorine atom or a cyano, methyl or trifluoromethyl group,
a phenyl group which is substituted by two methyl groups,
a benzyl group wherein the phenyl moiety may be substituted by one or two fluorine atoms, an iodine atom or a cyano, nitro or amino group,
a furanylmethyl or thienylmethyl group or
a cyclopropylmethyl group and
R⁴ denotes a piperidin-1-yl group which is substituted in the 3 position by an amino group, wherein the piperidin-1-yl moiety may additionally be substituted by a methyl group,
a 3-amino-piperidin-1-yl group wherein the piperidin-1-yl moiety is additionally substituted by a pyrrolidin-1-yl-carbonyl group,
a 3-amino-piperidin-1-yl group wherein the piperidin-1-yl moiety is additionally substituted in the 4 position by a hydroxy group,
a 3-amino-piperidin-1-yl group wherein a hydrogen atom in the 2 position together with a hydrogen atom in the 5 position is replaced by a —CH₂—CH₂-bridge,
a hexahydroazepin-1-yl group which is substituted in the 3 position by an amino group,
a [1,4]diazepan-1-yl group, which is substituted in the 6 position by an amino group,
a cyclohexyl group which is substituted in the 3 position by an amino group,
a 2-amino-cyclohexylamino group,
or an amino group substituted by the groups R¹⁵ and R¹⁶ wherein
R¹⁵ denotes a methyl or ethyl group and
R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
   unless otherwise stated, the abovementioned alkyl and alkenyl groups may be straight-chained or branched,
   the tautomers, enantiomers, diastereomers, mixtures thereof and the salts thereof.

A third sub-group of the particularly preferred compounds of formula I deserving special mention comprises those compounds of general formula I wherein

R¹, R² and R³ are as hereinbefore defined and
R⁴ denotes a piperidin-1-yl group which is substituted in the 3 position by an amino group, wherein the piperidin-1-yl moiety may additionally be substituted by a methyl group,
a 3-amino-piperidin-1-yl group wherein the piperidin-1-yl moiety is additionally substituted by a pyrrolidin-1-yl-carbonyl group,
a 3-amino-piperidin-1-yl group wherein the piperidin-1-yl moiety in the 4 position is additionally substituted by a hydroxy group,
a 3-amino-piperidin-1-yl group wherein a hydrogen atom in the 2 position together with a hydrogen atom in the 5 position is replaced by a —CH₂—CH₂-bridge,
a hexahydroazepin-1-yl group which is substituted in the 3 position by an amino group,
a cyclohexyl group which is substituted in the 3 position by an amino group, a 2-amino-cyclohexylamino group,
or an amino group substituted by the groups R¹⁵ and R¹⁶, wherein
R¹⁵ denotes a methyl or ethyl group and
R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
   unless otherwise stated, the abovementioned alkyl- and alkenyl groups may be straight-chained or branched,
   the tautomers, enantiomers, diastereomers, mixtures thereof and the salts thereof.

Another sub-group of compounds of general formula I which should be mentioned comprises those compounds wherein

R¹ denotes a hydrogen atom,
a C₁₋₈-alkyl group,
a C₃₋₈-alkenyl group,
a C₃₋₈-alkynyl group,
a C₁₋₆-alkyl group substituted by a group Rₐ, wherein
Rₐ denotes a C₃₋₇-cycloalkyl, heteroaryl, cyano, carboxy, C₁₋₃-alkyloxy-carbonyl, aminocarbonyl, C₁₋₃-alkylamino-carbonyl, di-(C₁₋₃-alkyl)-amino-carbonyl, pyrrolidin-1-ylcarbonyl, piperidin-1-ylcarbonyl, morpholin-4-ylcarbonyl, piperazin-1-ylcarbonyl, 4-methylpiperazin-1-ylcarbonyl or 4-ethylpiperazin-1-ylcarbonyl group,
   a C₁₋₆-alkyl group substituted by a phenyl group, wherein the phenyl ring is substituted by the groups R¹⁰ to R¹⁴ and
R¹⁰ denotes a hydrogen atom,
a fluorine, chlorine, bromine or iodine atom,
a C₁₋₄-alkyl, hydroxy, or C₁₋₄-alkyloxy group,
a nitro, amino, C₁₋₃-alkylamino, di-(C₁₋₃-alkyl)amino, pyrrolidin-1-yl, piperidin-1-yl, morpholin-4-yl, piperazin-1-yl, 4-(C₁₋₃-alkyl)-piperazin-1-yl, C₁₋₃-alkyl-carbonylamino, arylcarbonylamino, aryl-C₁₋₃-alkyl-carbonylamino, C₁₋₃-alkyloxy-carbonylamino, aminocarbonylamino, C₁₋₃-alkyl-aminocarbonylamino, di-(C₁₋₃-alkyl)aminocarbonylamino, C₁₋₃-alkyl-sulphonylamino, arylsulphonylamino or aryl-C₁₋₃-alkyl-sulphonylamino group,
an N—(C₁₋₃-alkyl)-C₁₋₃-alkyl-carbonylamino, N—(C₁₋₃-alkyl)-arylcarbonylamino, N—(C₁₋₃-alkyl)-aryl-C₁₋₃-alkyl-carbonylamino, N—(C₁₋₃-alkyl)-C₁₋₃-alkyloxy-carbonylamino, N-(aminocarbonyl)-C₁₋₃-alkylamino, N—(C₁₋₃-alkyl-aminocarbonyl)-C₁₋₃-alkylamino, N-[d-(C₁₋₃-alkyl)aminocarbonyl]-C₁₋₃-alkylamino, N—(C₁₋₃-alkyl)-C₁₋₃-alkyl-sulphonylamino, N—(C₁₋₃-alkyl)-aryl-sulphonylamino or N—(C₁₋₃-alkyl)-aryl-C₁₋₃-alkyl-sulphonylamino group,
a cyano, carboxy, C₁₋₃-alkyloxy-carbonyl, aminocarbonyl, C₁₋₃-alkyl-aminocarbonyl, di-(C₁₋₃-alkyl)-aminocarbonyl, pyrrolidin-1-yl-carbonyl, piperidin-1-yl-carbonyl, morpholin-4-yl-carbonyl, piperazin-1-yl-carbonyl or 4-(C₁₋₃-alkyl)-piperazin-1-yl-carbonyl group,
a C₁₋₃-alkyl-carbonyl or an arylcarbonyl group,
a carboxy-C₁₋₃-alkyl, C₁₋₃-alkyloxy-carbonyl-C₁₋₃-alkyl, cyano-C₁₋₃-alkyl, aminocarbonyl-C₁₋₃-alkyl, C₁₋₃-alkyl-aminocarbonyl-C₁₋₃-alkyl, di-(C₁₋₃-alkyl)-aminocarbonyl-C₁₋₃-alkyl, pyrrolidin-1-yl-carbonyl-C₁₋₃-alkyl, piperidin-1-yl-carbonyl-C₁₋₃-alkyl, morpholin-4-yl-carbonyl-C₁₋₃-alkyl, piperazin-1-yl-carbonyl-C₁₋₃-alkyl or 4-(C₁₋₃-alkyl)-piperazin-1-yl-carbonyl-C₁₋₃-alkyl group,
a carboxy-C₁₋₃-alkyloxy, C₁₋₃-alkyloxy-carbonyl-C₁₋₃-alkyloxy, cyano-C₁₋₃-alkyloxy, aminocarbonyl-C₁₋₃-alkyloxy, C₁₋₃-alkyl-aminocarbonyl-C₁₋₃-alkyloxy, di-(C₁₋₃-alkyl)-aminocarbonyl-C₁₋₃-alkyloxy, pyrrolidin-1-yl-carbonyl-C₁₋₃-alkyl-oxy, piperidin-1-yl-carbonyl-C₁₋₃-alkyloxy, morpholin-4-yl-carbonyl-C₁₋₃-alkyl-oxy, piperazin-1-yl-carbonyl-C₁₋₃-alkyloxy or 4-(C₁₋₃-alkyl)-piperazin-1-yl-carbonyl-C₁₋₃-alkyloxy group,
a hydroxy-C₁₋₃-alkyl, C₁₋₃-alkyloxy-C₁₋₃-alkyl, amino-C₁₋₃-alkyl, C₁₋₃-alkylamino-C₁₋₃-alkyl, di-(C₁₋₃-alkyl)-amino-C₁₋₃-alkyl, pyrrolidin-1-yl-C₁₋₃-alkyl, piperidin-1-yl-C₁₋₃-alkyl, morpholin-4-yl-C₁₋₃-alkyl, piperazin-1-yl-C₁₋₃-alkyl, 4-(C₁₋₃-alkyl)-piperazin-1-yl-C₁₋₃-alkyl group,
a hydroxy-C₁₋₃-alkyloxy, C₁₋₃-alkyloxy-C₁₋₃-alkyloxy, amino-C₁₋₃-alkyloxy, C₁₋₃-alkylamino-C₁₋₃-alkyloxy, di-(C₁₋₃-alkyl)-amino-C₁₋₃-alkyloxy, pyrrolidin-1-yl-C₁₋₃-alkyloxy, piperidin-1-yl-C₁₋₃-alkyloxy, morpholin-4-yl-C₁₋₃-alkyloxy, piperazin-1-yl-C₁₋₃-alkyloxy, 4-(C₁₋₃-alkyl)-piperazin-1-yl-C₁₋₃-alkyloxy group, a mercapto, C₁₋₃-alkylsulphanyl, C₁₋₃-alkysulphinyl, C₁₋₃-alkylsulphonyl, C₁₋₃-alkylsulphonyloxy, trifluoromethylsulphanyl, trifluoromethylsulphinyl or trifluoromethylsulphonyl group,
a sulpho, aminosulphonyl, C₁₋₃-alkyl-aminosulphonyl, di-(C₁₋₃-alkyl)-amino-sulphonyl, pyrrolidin-1-yl-sulphonyl, piperidin-1-yl-sulphonyl, morpholin-4-yl-sulphonyl, piperazin-1-yl-sulphonyl or 4-(C₁₋₃-alkyl)-piperazin-1-yl-sulphonyl group,
a methyl or methoxy group substituted by 1 to 3 fluorine atoms,
an ethyl or ethoxy group substituted by 1 to 5 fluorine atoms,
a C₂₋₄-alkenyl or C₂₋₄-alkynyl group,
a 2-propen-1-yloxy or 2-propyn-1-yloxy group,
a C₃₋₆-cycloalkyl or C₃₋₆-cycloalkyloxy group,
a C₃₋₆-cycloalkyl-C₁₋₃-alkyl or C₃₋₆-cycloalkyl-C₁₋₃-alkyloxy group or
an aryl, aryloxy, aryl-C₁₋₃-alkyl or aryl-C₁₋₃-alkyloxy group,
R¹¹ and R¹², which may be identical or different, in each case denote a hydrogen atom, a fluorine, chlorine, bromine or iodine atom, a C₁₋₃-alkyl, trifluoromethyl, hydroxy, or C₁₋₃-alkyloxy group or a cyano group, or
R¹¹ together with R¹², if they are bound to adjacent carbon atoms, also denote a methylenedioxy, difluoromethylenedioxy, straight-chain C₃₋₅-alkylene, —CH═CH—CH═CH, —CH═CH—CH═N or —CH═CH—N═CH group and
R¹³ and R¹⁴, which may be identical or different, in each case denote a hydrogen atom, a fluorine, chlorine or bromine atom, a trifluoromethyl, C₁₋₃-alkyl or C₁₋₃-alkyloxy group,
   a phenyl group substituted by the groups R¹⁰ to R¹⁴, wherein R¹⁰ to R¹⁴ are as hereinbefore defined,
   a phenyl-C₂₋₃-alkenyl group wherein the phenyl moiety is substituted by the groups R¹⁰ to R¹⁴, wherein R¹⁰ to R¹⁴ are as hereinbefore defined,
   a phenyl-(CH₂)ₘ-A-(CH₂)ₙ group wherein the phenyl moiety is substituted by R¹⁰ to R¹⁴, wherein R¹⁰ to R¹⁴ are as hereinbefore defined and
A denotes a carbonyl, cyanoiminomethylene, hydroxyiminomethylene or C₁₋₃-alkyloxyiminomethylene group, m denotes the number 0, 1 or 2 and n denotes the number 1, 2 or 3,
   a phenyl-(CH₂)ₘ-B-(CH₂)ₙ group wherein the phenyl moiety is substituted by R¹⁰ to R¹⁴, wherein R¹⁰ to R¹⁴, m and n are as hereinbefore defined and
B denotes a methylene group which is substituted by a hydroxy, C₁₋₃-alkyloxy, amino, C₁₋₃-alkylamino, di-(C₁₋₃-alkyl)-amino, mercapto, C₁₋₃-alkylsulphanyl, C₁₋₃-alkylsulphinyl or C₁₋₃-alkylsulphonyl group and is optionally additionally substituted by a methyl or ethyl group,
   a heteroaryl-(CH₂)ₘ-A-(CH₂)ₙ group, wherein A, m and n are as hereinbefore defined,
   a heteroaryl-(CH₂)ₘ-B-(CH₂)ₙ group, wherein B, m and n are as hereinbefore defined,
   a C₁₋₆-alkyl-A-(CH₂)ₙ group, wherein A and n are as hereinbefore defined,
   a C₃₋₇-cycloalkyl-(CH₂)ₘ-A-(CH₂)ₙ group, wherein A, m and n are as hereinbefore defined,
   a C₃₋₇-cycloalkyl-(CH₂)ₘ-B-(CH₂)ₙ group, wherein B, m and n are as hereinbefore defined,
   a R²¹-A-(CH₂)ₙ group wherein R²¹ denotes a C₁₋₃-alkyloxycarbonyl, aminocarbonyl, C₁₋₃-alkylaminocarbonyl, di-(C₁₋₃-alkyl)aminocarbonyl, pyrrolidin-1-yl-carbonyl, piperidin-1-yl-carbonyl or morpholin-4-yl-carbonyl, piperazin-1-yl-carbonyl, 4-methylpiperazin-1-yl-carbonyl or 4-ethylpiperazin-1-yl-carbonyl group and A and n are as hereinbefore defined,
   a phenyl-(CH₂)ₘ-D-C₁₋₃-alkyl group wherein the phenyl moiety is substituted by the groups R¹⁰ to R¹⁴, wherein R¹⁰ to R¹⁴ and m are as hereinbefore defined and D denotes an oxygen or sulphur atom, an imino, C₁₋₃-alkylimino, sulphinyl or sulphonyl group,
   a C₂₋₆-alkyl group substituted by a group R_{b}, wherein
R_{b} is isolated from the cyclic nitrogen atom in the 1 position of the xanthine skeleton by at least two carbon atoms and R_{b} denotes a hydroxy, C₁₋₃-alkyloxy, mercapto, C₁₋₃-alkylsulphanyl, C₁₋₃-alkylsulphinyl, C₁₋₃-alkylsulphonyl, amino, C₁₋₃-alkylamino, di-(C₁₋₃-alkyl)-amino, pyrrolidin-1-yl, piperidin-1-yl, morpholin-4-yl, piperazin-1-yl or 4-(C₁₋₃-alkyl)-piperazin-1-yl group,
   or a C₃₋₆-cycloalkyl group,
   R² denotes a hydrogen atom,
   a C₁₋₈-alkyl group,
   a C₃₋₆-alkenyl group,
   a C₃₋₆-alkynyl group,
   a C₁₋₆-alkyl group substituted by a group Rₐ, wherein Rₐ is as hereinbefore defined,
   a C₁₋₆-alkyl group substituted by a phenyl group, wherein the phenyl ring is substituted by the groups R¹⁰ to R¹⁴ and R¹⁰ to R¹⁴ are as hereinbefore defined,
   a phenyl group substituted by the groups R¹⁰ to R¹⁴, wherein R¹⁰ to R¹⁴ are as hereinbefore defined,
   a phenyl-C₂₋₃-alkenyl group wherein the phenyl moiety is substituted by the groups R¹⁰ to R¹⁴, wherein R¹⁰ to R¹⁴ are as hereinbefore defined,
   a phenyl-(CH₂)ₘ-A-(CH₂)ₙ group wherein the phenyl moiety is substituted by R¹⁰ to R¹⁴, wherein R¹⁰ to R¹⁴, A, m and n are as hereinbefore defined,
   a phenyl-(CH₂)ₘ-B-(CH₂)ₙ group wherein the phenyl moiety is substituted by R¹⁰ to R¹⁴, wherein R¹⁰ to R¹⁴, B, m and n are as hereinbefore defined,
   a heteroaryl-(CH₂)ₘ-A-(CH₂)ₙ group, wherein A, m and n are as hereinbefore defined,
   a heteroaryl-(CH₂)ₘ-B-(CH₂)ₙ group, wherein B, m and n are as hereinbefore defined,
   a C₁₋₆-alkyl-A-(CH₂)ₙ group, wherein A and n are as hereinbefore defined,
   a C₃₋₇-cycloalkyl-(CH₂)ₘ-A-(CH₂)ₙ group, wherein A, m and n are as hereinbefore defined,
   a C₃₋₇-cycloalkyl-(CH₂)ₘ-B-(CH₂)ₙ group, wherein B, m and n are as hereinbefore defined,
   a R²¹-A-(CH₂)ₙ group wherein R²¹, A and n are as hereinbefore defined, a phenyl-(CH₂)ₘ-D-C₁₋₃-alkyl group wherein the phenyl moiety is substituted by the groups R¹⁰ to R¹⁴, wherein R¹⁰ to R¹⁴, m and D are as hereinbefore defined,
   a C₂₋₆-alkyl group substituted by a group R_{b}, wherein
R_{b} is isolated from the cyclic nitrogen atom in the 3 position of the xanthine skeleton by at least two carbon atoms and is as hereinbefore defined,
   or a C₃₋₆-cycloalkyl group,
   R³ denotes a C₁₋₈-alkyl group,
   a C₁₋₄-alkyl group substituted by the group R_{c}, wherein
R_{c} denotes a C₃₋₇-cycloalkyl group optionally substituted by one or two C₁₋₃-alkyl groups,
a C₅₋₇-cycloalkenyl group optionally substituted by one or two C₁₋₃-alkyl groups or an aryl or heteroaryl group,
   a C₃₋₈-alkenyl group,
   a C₃₋₆-alkenyl group substituted by a fluorine, chlorine or bromine atom, or a trifluoromethyl group,
   a C₃₋₈-alkynyl group,
   an aryl group or
   an aryl-C₂₋₄-alkenyl group,
   and
   R⁴ denotes an azetidin-1-yl or pyrrolidin-1-yl group which is substituted in the 3 position by a RₑNR_{d} group and may additionally be substituted by one or two C₁₋₃-alkyl groups, wherein
Rₑ denotes a hydrogen atom or a C₁₋₃-alkyl group and
R_{d} denotes a hydrogen atom, a C₁₋₃-alkyl group, an R₁—C₁₋₃-alkyl group or a R_{g}—C₂₋₃-alkyl group, wherein
R_{f} denotes a carboxy, C₁₋₃-alkyloxy-carbonyl, aminocarbonyl, C₁₋₃-alkylamino-carbonyl, di-(C₁₋₃-alkyl)-aminocarbonyl, pyrrolidin-1-yl-carbonyl, 2-cyano-pyrrolidin-1-yl-carbonyl, 2-carboxypyrrolidin-1-yl-carbonyl, 2-methoxy-carbonylpyrrolidin-1-yl-carbonyl, 2-ethoxycarbonylpyrrolidin-1-yl-carbonyl, 2-aminocarbonylpyrrolidin-1-yl-carbonyl, 4-cyanothiazolidin-3-yl-carbonyl, 4-carboxythiazolidin-3-yl-carbonyl, 4-methoxycarbonylthiazolidin-3-yl-carbonyl, 4-ethoxycarbonylthiazolidin-3-yl-carbonyl, 4-aminocarbonylthiazolidin-3-yl-carbonyl, piperidin-1-yl-carbonyl, morpholin-4-yl-carbonyl, piperazin-1-yl-carbonyl, 4-methyl-piperazin-1-yl-carbonyl or 4-ethyl-piperazin-1-yl-carbonyl group and
R_{g}, which is separated from the nitrogen atom of the RₑNR_{d} group by at least two carbon atoms denotes a hydroxy, methoxy or ethoxy group,
   a piperidin-1-yl or hexahydroazepin-1-yl group which is substituted in the 3 position or in the 4 position by an RₑNR_{d} group and may additionally be substituted by one or two C₁₋₃-alkyl groups, wherein Rₑ and R_{d} are as hereinbefore defined,
   a piperidin-1-yl or hexahydroazepin-1-yl group substituted in the 3 position by an amino, C₁₋₃-alkylamino or di-(C₁₋₃-alkyl)-amino group, wherein in each case two hydrogen atoms on the carbon skeleton of the piperidin-1-yl or hexahydroazepin-1-yl group are replaced by a straight-chain alkylene bridge, this bridge containing 2 to 5 carbon atoms if the two hydrogen atoms are located on the same carbon atom, or 1 to 4 carbon atoms, if the hydrogen atoms are located on adjacent carbon atoms, or 1 to 4 carbon atoms, if the hydrogen atoms are located on carbon atoms which are separated by one atom, or 1 to 3 carbon atoms if the two hydrogen atoms are located on carbon atoms separated by two atoms,
   an azetidin-1-yl, pyrrolidin-1-yl, piperidin-1-yl or hexahydroazepin-1-yl group which is substituted by an amino-C₁₋₃-alkyl, C₁₋₃-alkylamino-C₁₋₃-alkyl or a di-(C₁₋₃-alkyl)amino-C₁₋₃-alkyl group,
   a piperazin-1-yl or [1,4]diazepan-1-yl group optionally substituted on the carbon skeleton by one or two C₁₋₃-alkyl groups,
   a 3-imino-piperazin-1-yl, 3-imino-[1,4]diazepan-1-yl or 5-imino-[1,4]diazepan-1-yl group optionally substituted on the carbon skeleton by one or two C₁₋₃-alkyl groups,
   a [1,4]diazepan-1-yl group optionally substituted by one or two C₁₋₃-alkyl groups, which is substituted in the 6 position by an amino group,
   a C₃₋₇-cycloalkyl group which is substituted by an amino, C₁₋₃-alkylamino or di-(C₁₋₃-alkyl)-amino group,
   a C₃₋₇-cycloalkyl group which is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkyl-C₁₋₂-alkyl group wherein the cycloalkyl moiety is substituted by an amino, C₁₋₃-alkylamino or di-(C₁₋₃-alkyl)-amino group,
   a C₃₋₇-cycloalkyl-C₁₋₂-alkyl group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkylamino group wherein the cycloalkyl moiety is substituted by an amino, C₁₋₃-alkylamino or di-(C₁₋₃-alkyl)-amino group, wherein the two nitrogen atoms at the cycloalkyl moiety are separated from one another by at least two carbon atoms,
   a N—(C₃₋₇-cycloalkyl)-N—(C₁₋₃-alkyl)-amino group wherein the cycloalkyl moiety is substituted by an amino, C₁₋₃-alkylamino or di-(C₁₋₃-alkyl)-amino group, wherein the two nitrogen atoms at the cycloalkyl moiety are separated from one another by at least two carbon atoms,
   a C₃₋₇-cycloalkylamino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a N—(C₃₋₇-cycloalkyl)-N—(C₁₋₃-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a C₃₋₇-cycloalkyl-C₁₋₂-alkyl-amino group wherein the cycloalkyl moiety is substituted by an amino, C₁₋₃-alkylamino or di-(C₁₋₃-alkyl)-amino group,
   a N—(C₃₋₇-cycloalkyl-C₁₋₂-alkyl)-N—(C₁₋₂-alkyl)-amino group wherein the cycloalkyl moiety is substituted by an amino, C₁₋₃-alkylamino or di-(C₁₋₃-alkyl)-amino group,
   a C₃₋₇-cycloalkyl-C₁₋₂-alkyl-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   a N—(C₃₋₇-cycloalkyl-C₁₋₂-alkyl)-N—(C₁₋₂-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
   an amino group substituted by the groups R¹⁵ and R¹⁶ wherein
R¹⁵ denotes a C₁₋₆-alkyl group, a C₃₋₆-cycloalkyl, C₃₋₆-cycloalkyl-C₁₋₃-alkyl, aryl or aryl-C₁₋₃-alkyl group and
R¹⁶ denotes a R¹⁷—C₂₋₃-alkyl group, wherein the C₂₋₃-alkyl moiety is straight-chained and may be substituted by one to four C₁₋₃-alkyl groups, which may be identical or different, and
R¹⁷ denotes an amino, C₁₋₃-alkylamino or di-(C₁₋₃-alkyl)-amino group, wherein, if R³ denotes a methyl group, R¹⁷ cannot be a di-(C₁₋₃-alkyl)-amino group,
   an amino group substituted by the group R²⁰, wherein
R²⁰ denotes an azetidin-3-yl, azetidin-2-ylmethyl, azetidin-3-ylmethyl, pyrrolidin-3-yl, pyrrolidin-2-ylmethyl, pyrrolidin-3-ylmethyl, piperidin-3-yl, piperidin-4-yl, piperidin-2-ylmethyl, piperidin-3-ylmethyl or piperidin-4-ylmethyl group, wherein the groups mentioned for R²⁰ may each be substituted by one or two C₁₋₃-alkyl groups,
   an amino group substituted by the groups R¹⁵ and R²⁰, wherein
R¹⁵ and R²⁰ are as hereinbefore defined, wherein the groups mentioned for R²⁰ may each be substituted by one or two C₁₋₃-alkyl groups,
   a R¹⁹—C₃₋₄-alkyl group wherein the C₃₋₄-alkyl moiety is straight-chained and may be substituted by the group R¹⁵ and may additionally be substituted by one or two C₁₋₃-alkyl groups, wherein R¹⁵ is as hereinbefore defined and R¹⁹ denotes an amino, C₁₋₃-alkylamino or di-(C₁₋₃-alkyl)-amino group,
   a pyrrolidin-3-yl, piperidin-3-yl, piperidin-4-yl, hexahydroazepin-3-yl or hexahydroazepin-4-yl group, which is substituted in the 1 position by an amino, C₁₋₃-alkylamino or di-(C₁₋₃-alkyl)amino group,
   or an azetidin-2-yl-C₁₋₂-alkyl, azetidin-3-yl-C₁₋₂-alkyl, pyrrolidin-2-yl-C₁₋₂-alkyl, pyrrolidin-3-yl, pyrrolidin-3-yl-C₁₋₂-alkyl, piperidin-2-yl-C₁₋₂-alkyl, piperidin-3-yl, piperidin-3-yl-C₁₋₂-alkyl, piperidin-4-yl or piperidin-4-yl-C₁₋₂-alkyl group, wherein the abovementioned groups may each be substituted by one or two C₁₋₃-alkyl groups,
   wherein by the aryl groups mentioned in the definition of the abovementioned groups are meant phenyl groups which may be mono- or disubstituted independently of one another by Rₕ, wherein the substituents may be identical or different and Rₕ denotes a fluorine, chlorine, bromine or iodine atom, a trifluoromethyl, C₁₋₃-alkyl, cyclopropyl, ethenyl, ethynyl, hydroxy, C₁₋₃-alkyloxy, difluoromethoxy or trifluoromethoxy group,
   by the heteroaryl groups mentioned in the definition of the abovementioned groups are meant a pyrrolyl, furanyl, thienyl, pyridyl, indolyl, benzofuranyl, benzothiophenyl, quinolinyl or isoquinolinyl group,
   or a pyrrolyl, furanyl, thienyl or pyridyl group wherein one or two methyne groups are replaced by nitrogen atoms,
   or an indolyl, benzofuranyl, benzothiophenyl, quinolinyl or isoquinolinyl group wherein one to three methyne groups are replaced by nitrogen atoms,
wherein the five-membered groups or parts of molecules may in each case be substituted by a C₁₋₃-alkyl or trifluoromethyl group and
the six-membered groups or parts of molecules may each be substituted by one or two C₁₋₃-alkyl groups or by a fluorine, chlorine, bromine or iodine atom, by a trifluoromethyl, hydroxy, C₁₋₃-alkyloxy, difluoromethoxy or trifluoromethoxy group,
   while unless otherwise stated the abovementioned alkyl, alkenyl and alkynyl groups may be straight-chained or branched,
   as well as the derivatives which are N-oxidised or methylated or ethylated at the cyclic nitrogen atom in the 9 position of the xanthine skeleton,
   with the proviso that the compounds wherein
   R¹ denotes a hydrogen atom, a methyl, propyl, 2-hydroxypropyl, aminocarbonyl-methyl or benzyl group,
   R² denotes a methyl group,
   R³ denotes a C₁₋₈-alkyl group, a benzyl group optionally substituted by a fluorine, chlorine or bromine atom or a methyl group, a 1-phenylethyl or 2-phenylethyl group, a 2-propen-1-yl, 2-buten-1-yl, 3-chloro-2-buten-1-yl or 2-methyl-2-propen-1-yl group
   and
   R⁴ denotes a piperazin-1-yl group, are excluded,
   and with the proviso that the compounds wherein
   R¹ denotes a hydrogen atom or a methyl group,
   R² denotes a hydrogen atom or a methyl group,
   R³ denotes a methyl group
   and
   R⁴ denotes a 3-aminopropyl, 3-[di-(C₁₋₃-alkyl)amino]-propyl, 1-phenyl-3-[di-(C₁₋₃-alkyl)amino]-propyl, 1-phenyl-3-methyl-3-(dimethylamino)-propyl, 1-(4-chlorophenyl)-3-(dimethylamino)-propyl, 1-phenyl-2-methyl-3-(dimethylamino)-propyl, 1-(3-methoxyphenyl)-3-(dimethylamino)-propyl or a 4-aminobutyl group, are excluded,
   and with the proviso that the compound
1,3,7-trimethyl-8-(1-aminocyclohexyl)-xanthine
   is excluded,
   the isomers and the salts thereof.

The following preferred compounds are mentioned by way of example:
(1) 1,3-dimethyl-7-benzyl-8-(3-amino-pyrrolidin-1-yl)-xanthine,
(2) 1,3-dimethyl-7-(3-methyl-2-buten-1-yl)-8-(3-amino-pyrrolidin-1-yl)-xanthine,
(3) 1,3-dimethyl-7-benzyl-8-(3-amino-piperidin-1-yl)-xanthine,
(4) 1,3-dimethyl-7-(3-methyl-2-buten-1-yl)-8-[(trans-2-amino-cyclohexyl)amino]-xanthine,
(5) 1,3-dimethyl-7-(3-methyl-2-buten-1-yl)-8-(3-amino-piperidin-1-yl)-xanthine,
(6) 1,3-dimethyl-7-(3-methyl-2-buten-1-yl)-8-(4-amino-piperidin-1-yl)-xanthine,
(7) 1,3-dimethyl-7-(3-methyl-2-buten-1-yl)-8-[(cis-2-amino-cyclohexyl)amino]-xanthine,
(8) 1,3-dimethyl-7-(2-butyn-1-yl)-8-(3-amino-piperidin-1-yl)-xanthine,
(9) 1,3-dimethyl-7-[(1-cyclopenten-1-yl)methyl]-8-(3-amino-piperidin-1-yl)-xanthine,
(10) 1,3-dimethyl-7-(2-thienylmethyl)-8-(3-amino-piperidin-1-yl)-xanthine,
(11) 1,3-dimethyl-7-(3-fluorobenzyl)-8-(3-amino-piperidin-1-yl)-xanthine,
(12) 1,3-dimethyl-7-(2-fluorobenzyl)-8-(3-amino-piperidin-1-yl)-xanthine,
(13) 1,3-dimethyl-7-(4-fluorobenzyl)-8-(3-amino-piperidin-1-yl)-xanthine,
(14) 1,3-dimethyl-7-(2-buten-1-yl)-8-(3-amino-piperidin-1-yl)-xanthine,
(15) 1,3-bis-(cyclopropylmethyl)-7-benzyl-8-(3-amino-piperidin-1-yl)-xanthine,
(16) (R)-1,3-dimethyl-7-(3-methyl-2-buten-1-yl)-8-(3-amino-piperidin-1-yl)-xanthine,
(17) (S)-1,3-dimethyl-7-(3-methyl-2-buten-1-yl)-8-(3-amino-piperidin-1-yl)-xanthine,
(18) 1,3-dimethyl-7-(3-methyl-2-buten-1-yl)-8-(3-amino-hexahydroazepin-1-yl)-xanthine,
(19) 1,3-dimethyl-7-(3-methyl-2-buten-1-yl)-8-(4-amino-hexahydroazepin-1-yl)-xanthine,
(20) 1,3-dimethyl-7-(3-methyl-2-buten-1-yl)-8-(cis-3-amino-cyclohexyl)-xanthine-hydrochloride,
(21) 1,3-dimethyl-7-(3-methyl-2-buten-1-yl)-8-(3-methylamino-piperidin-1-yl)-xanthine,
(22) 1-(2-phenylethyl)-3-methyl-7-(3-methyl-2-buten-1-yl)-8-(3-amino-piperidin-1-yl)-xanthine,
(23) 1,3-dimethyl-7-(3-methyl-2-buten-1-yl)-8-[N-(2-aminoethyl)-methylamino]-xanthine,
(24) 1-[2-(thiophen-2-yl)-ethyl]-3-methyl-7-(3-methyl-2-buten-1-yl)-8-(3-amino-piperidin-1-yl)-xanthine,
(25) 1-[2-(thiophen-3-yl)-ethyl]-3-methyl-7-(3-methyl-2-buten-1-yl)-8-(3-amino-piperidin-1-yl)-xanthine,
(26) 1-[2-(2-methyl-phenyl)-ethyl]-3-methyl-7-(3-methyl-2-buten-1-yl)-8-(3-amino-piperidin-1-yl)-xanthine,
(27) 1-[2-(3-methyl-phenyl)-ethyl]-3-methyl-7-(3-methyl-2-buten-1-yl)-8-(3-amino-piperidin-1-yl)-xanthine,
(28) 1-[2-(3-methoxy-phenyl)-ethyl]-3-methyl-7-(3-methyl-2-buten-1-yl)-8-(3-amino-piperidin-1-yl)-xanthine,
(29) 1-((E)-2-phenyl-vinyl)-3-methyl-7-(3-methyl-2-buten-1-yl)-8-(3-amino-piperidin-1-yl)-xanthine,
(30) 1-(2-phenyl-ethyl)-3-methyl-7-(3-methyl-2-buten-1-yl)-8-((S)-3-amino-piperidin-1-yl)-xanthine,
(31) 1-(2-phenyl-ethyl)-3-methyl-7-(3-methyl-2-buten-1-yl)-8-((R)-3-amino-piperidin-1-yl)-xanthine,
(32) 1-[2-(2-methoxy-phenyl)-2-oxo-ethyl]-3-methyl-7-(3-methyl-2-buten-1-yl)-8-(3-amino-piperidin-1-yl)-xanthine,
(33) 1-[2-(thiophen-3-yl)-2-oxo-ethyl]-3-methyl-7-(3-methyl-2-buten-1-yl)-8-(3-amino-piperidin-1-yl)-xanthine,
(34) 1-(2-phenyl-2-oxo-ethyl)-3-methyl-7-(3-methyl-2-buten-1-yl)-8-((S)-3-amino-piperidin-1-yl)-xanthine,
(35) 1-(2-phenyl-2-oxo-ethyl)-3-methyl-7-(3-methyl-2-buten-1-yl)-8-((R)-3-amino-piperidin-1-yl)-xanthine,
36) 1-[(isoquinolin-1-yl)methyl]-3-methyl-7-(3-methyl-2-buten-1-yl)-8-((R)-3-amino-piperidin-1-yl)-xanthine,
(37) 1-[(isoquinolin-1-yl)methyl]-3-methyl-7-(3-methyl-2-buten-1-yl)-8-((S)-3-amino-piperidin-1-yl)-xanthine and
(38) 1-[(1-naphthyl)methyl]-3-methyl-7-(3-methyl-2-buten-1-yl)-8-(3-amino-piperidin-1-yl)-xanthine
and the salts thereof.

According to the invention, the compounds of general formula I are obtained by methods known per se, for example by the following methods:

a) In order to prepare compounds of general formula I wherein R⁴ is one of the abovementioned groups linked to the xanthine skeleton via a nitrogen atom:
reacting a compound of general formula

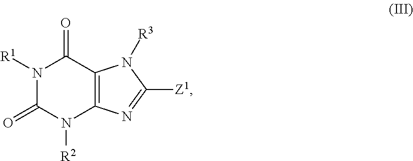

wherein
R¹ to R³ are as hereinbefore defined and
Z¹ denotes a leaving group such as a halogen atom, a substituted hydroxy, mercapto, sulphinyl, sulphonyl or sulphonyloxy group such as a chlorine or bromine atom, a methanesulphonyl or methanesulphonyloxy group, with a compound of general formula

H—R^{4′} (IV),

wherein
R^{4′} denotes one of the groups mentioned for R⁴ hereinbefore, which is linked to the xanthine skeleton of general formula I via a nitrogen atom.

The reaction is expediently carried out in a solvent such as isopropanol, butanol, tetrahydrofuran, dioxan, toluene, chlorobenzene, dimethylformamide, dimethyl-sulphoxide, methylene chloride, ethylene glycol monomethylether, ethylene glycol diethylether or sulpholane optionally in the presence of an inorganic or tertiary organic base, e.g. sodium carbonate or potassium hydroxide, a tertiary organic base, e.g. triethylamine, or in the presence of N-ethyl-diisopropylamine (Hünig base), while these organic bases may simultaneously serve as solvent, and optionally in the presence of a reaction accelerator such as an alkali metal halide or a palladium-based catalyst at temperatures between −20 and 180° C., preferably however at temperatures between −10 and 120° C. The reaction may however also be carried out without a solvent or in an excess of the compound of general formula IV used.

b) In order to prepare a compound of general formula I wherein R⁴ according to the definition given earlier contains an amino group or an alkylamino group optionally substituted in the alkyl moiety:
deprotecting a compound of general formula

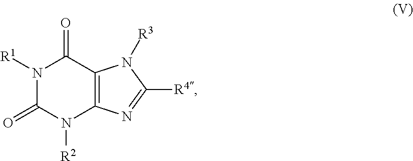

wherein R¹, R² and R³ are as hereinbefore defined and
R^{4″} contains an N-tert.-butyloxycarbonylamino group or an N-tert.-butyloxycarbonyl-N-alkylamino group, wherein the alkyl moiety of the N-tert.-butyloxycarbonyl-N-alkyl-amino group may be substituted as mentioned hereinbefore.

The tert.-butyloxycarbonyl group is preferably cleaved by treating with an acid such as trifluoroacetic acid or hydrochloric acid or by treating with bromotrimethylsilane or iodotrimethylsilane, optionally using a solvent such as methylene chloride, ethyl acetate, dioxan, methanol or diethyl ether at temperatures between 0 and 80° C.

c) In order to prepare a compound of general formula I wherein R² as hereinbefore defined denotes a hydrogen atom:
deprotecting a compound of general formula

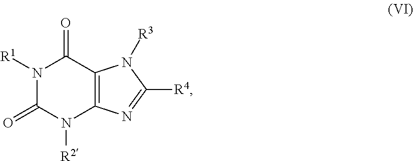

wherein R¹, R³ and R⁴ are as hereinbefore defined and R^{2′} denotes a protecting group such as a methoxymethyl, benzyloxymethyl, methoxyethoxymethyl or 2-(trimethylsilyl)ethyloxymethyl group.

The protecting group is cleaved, for example, using an acid such as acetic acid, trifluoroacetic acid, hydrochloric acid, sulphuric acid or an acid ion exchanger in a solvent such as methylene chloride, tetrahydrofuran, methanol, ethanol or isopropanol or mixtures thereof, while the 2-(trimethylsilyl)ethyloxymethyl group may also be cleaved using hydrofluoric acid or a salt of hydrofluoric acid such as tetrabutylammonium fluoride.

If according to the invention a compound of general formula I is obtained which contains an amino, alkylamino or imino group, this may be converted by acylation or sulphonylation into a corresponding acyl or sulphonyl compound of general formula I;

if a compound of general formula I is obtained which contains an amino, alkylamino or imino group, this may be converted by alkylation or reductive alkylation into a corresponding alkyl compound of general formula I;
if a compound of general formula I is obtained which contains a nitro group, this may be converted by reduction into a corresponding amino compound;
if a compound of general formula I is obtained which contains an imino group, this may be converted by nitrosation and subsequent reduction into a corresponding N-amino-imino compound;
if a compound of general formula I is obtained which contains a C₁₋₃-alkyloxy-carbonyl group, this may be converted by cleavage of the ester into the corresponding carboxy compound;
if a compound of general formula I is obtained wherein R¹ contains a carbonyl group, this may be converted by reaction with hydroxylamine into a corresponding oxime of general formula I;
if a compound of general formula I is obtained which contains a carboxy group, this may be converted by esterification into a corresponding ester of general formula I; or
if a compound of general formula I is obtained which contains a carboxy or ester group, this may be converted by reaction with an amine into a corresponding amide of general formula I.

The subsequent esterification is optionally carried out in a solvent or mixture of solvents such as methylene chloride, dimethylformamide, benzene, toluene, chlorobenzene, tetrahydrofuran, benzene/tetrahydrofuran or dioxan or particularly advantageously in a corresponding alcohol optionally in the presence of an acid such as hydrochloric acid or in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole and optionally additionally in the presence of 4-dimethylamino-pyridine, N,N′-carbonyldiimidazole or triphenylphosphine/carbon tetrachloride, conveniently at temperatures between 0 and 150° C., preferably at temperatures between 0 and 80° C.

The subsequent ester formation may also be carried out by reacting a compound which contains a carboxy group with a corresponding alkyl halide.

The subsequent acylation or sulphonylation is optionally carried out in a solvent or mixture of solvents such as methylene chloride, dimethylformamide, benzene, toluene, chlorobenzene, tetrahydrofuran, benzene/tetrahydrofuran or dioxan with a corresponding acyl or sulphonyl derivative optionally in the presence of a tertiary organic base or in the presence of an inorganic base or in the presence of a dehydrating agent, e.g. in the presence of isobutyl chloroformate, thionyl chloride, trimethylchlorosilane, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole and optionally additionally in the presence of 4-dimethylamino-pyridine, N,N′-carbonyldiimidazole or triphenylphosphine/carbon tetrachloride, conveniently at temperatures between 0 and 150° C., preferably at temperatures between 0 and 80° C.

The subsequent alkylation is optionally carried out in a solvent or mixture of solvents such as methylene chloride, dimethylformamide, benzene, toluene, chlorobenzene, tetrahydrofuran, benzene/tetrahydrofuran or dioxan with an alkylating agent such as a corresponding halide or sulphonic acid ester, e.g. with methyl iodide, ethyl bromide, dimethylsulphate or benzyl chloride, optionally in the presence of a tertiary organic base or in the presence of an inorganic base conveniently at temperatures between 0 and 150° C., preferably at temperatures between 0 and 100° C.

The subsequent reductive alkylation is carried out with a corresponding carbonyl compound such as formaldehyde, acetaldehyde, propionaldehyde, acetone or butyraldehyde in the presence of a complex metal hydride such as sodium borohydride, lithium borohydride, sodium triacetoxyborohydride or sodium cyanoborohydride conveniently at a pH of 6-7 and at ambient temperature or in the presence of a hydrogenation catalyst, e.g. with hydrogen in the presence of palladium/charcoal, at a hydrogen pressure of 1 to 5 bar. The methylation may also be carried out in the presence of formic acid as reducing agent at elevated temperature, e.g. at temperatures between 60 and 120° C.

The subsequent reduction of a nitro group is carried out for example with hydrogen and a catalyst such as palladium on activated charcoal, platinum dioxide or Raney nickel, or using other reducing agents such as iron or zinc in the presence of an acid such as acetic acid.

Subsequent nitrosation of an imino group followed by reduction to obtain the N-amino-imino compound is carried out for example so that the imino compound is nitrosated with an alkyl nitrite such as isoamyl nitrite and the N-nitroso-imino compound formed is then reduced directly to form the N-amino-imino compound; zinc, for example, in the presence of an acid such as acetic acid is suitable for this purpose.

The subsequent cleaving of a C₁₋₃-alkyloxycarbonyl group to obtain the carboxy group is carried out, for example, by hydrolysis with an acid such as hydrochloric acid or sulphuric acid or an alkali metal hydroxide such as lithium hydroxide, sodium hydroxide or potassium hydroxide.

The subsequent amide formation is carried out by reacting a corresponding reactive carboxylic acid derivative with a corresponding amine optionally in a solvent or mixture of solvents such as methylene chloride, dimethylformamide, benzene, toluene, chlorobenzene, tetrahydrofuran, benzene/tetrahydrofuran or dioxan, while the amine used may simultaneously serve as solvent, optionally in the presence of a tertiary organic base or in the presence of an inorganic base or with a corresponding carboxylic acid in the presence of a dehydrating agent, e.g. in the presence of isobutyl chloroformate, thionyl chloride, trimethylchlorosilane, phosphorus trichloride, phosphorus pentoxide, N,N′-dicyclohexylcarbodiimide, N,N′-dicyclohexylcarbodiimide/N-hydroxysuccinimide or 1-hydroxy-benzotriazole and optionally additionally in the presence of 4-dimethylamino-pyridine, N,N′-carbonyldiimidazole or triphenylphosphine/carbon tetrachloride, conveniently at temperatures between 0 and 150° C., preferably at temperatures between 0 and 80° C.

In the reactions described hereinbefore, any reactive groups present such as hydroxy, carboxy, amino, alkylamino or imino groups may be protected during the reaction by conventional protecting groups which are cleaved again after the reaction.

For example, a protecting group for a hydroxy group may be a trimethylsilyl, acetyl, benzoyl, methyl, ethyl, tert-butyl, trityl, benzyl or tetrahydropyranyl group,

protecting groups for a carboxy group may be a trimethylsilyl, methyl, ethyl, tert.butyl, benzyl or tetrahydropyranyl group and
protecting groups for an amino, alkylamino or imino group may be a formyl, acetyl, trifluoroacetyl, ethoxycarbonyl, tert-butoxycarbonyl, benzyloxycarbonyl, benzyl, methoxybenzyl or 2,4-dimethoxybenzyl group and additionally, for the amino group, a phthalyl group.

Any protecting group used is optionally subsequently cleaved for example by hydrolysis in an aqueous solvent, e.g. in water, isopropanol/water, acetic acid/water, tetrahydrofuran/water or dioxan/water, in the presence of an acid such as trifluoroacetic acid, hydrochloric acid or sulphuric acid or in the presence of an alkali metal base such as sodium hydroxide or potassium hydroxide or aprotically, e.g. in the presence of iodotrimethylsilane, at temperatures between 0 and 120° C., preferably at temperatures between 10 and 100° C.

However, a benzyl, methoxybenzyl or benzyloxycarbonyl group is cleaved, for example, hydrogenolytically, e.g. with hydrogen in the presence of a catalyst such as palladium/charcoal in a suitable solvent such as methanol, ethanol, ethyl acetate or glacial acetic acid optionally with the addition of an acid such as hydrochloric acid at temperatures between 0 and 100° C., but preferably at ambient temperatures between 20 and 60° C., and at a hydrogen pressure of 1 to 7 bar, but preferably from 3 to 5 bar. However, a 2,4-dimethoxybenzyl group is preferably cleaved in trifluoroacetic acid in the presence of anisole.

A tert.-butyl or tert.-butyloxycarbonyl group is preferably cleaved by treating with an acid such as trifluoroacetic acid or hydrochloric acid or by treating with iodotrimethylsilane optionally using a solvent such as methylene chloride, dioxan, methanol or diethyl ether.

A trifluoroacetyl group is preferably cleaved by treating with an acid such as hydrochloric acid optionally in the presence of a solvent such as acetic acid at temperatures between 50 and 120° C. or by treating with sodium hydroxide solution optionally in the presence of a solvent such as tetrahydrofuran at temperatures between 0 and 50° C.

A phthalyl group is preferably cleaved in the presence of hydrazine or a primary amine such as methylamine, ethylamine or n-butylamine in a solvent such as methanol, ethanol, isopropanol, toluene/water or dioxan at temperatures between 20 and 50° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)- or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I thus obtained contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, ethanolamine, diethanolamine and triethanolamine.

The compounds of general formulae III to VI used as starting materials are either known from the literature or may be obtained by methods known from the literature (cf. Examples I to XXXI).

For example, a starting compound of general formula III may be obtained by reacting a theophylline derivative halogenated in the 8 position with a correspondingly substituted alkyl halide.

As already mentioned hereinbefore, the compounds of general formula I according to the invention and the physiologically acceptable salts thereof have valuable pharmacological properties, particularly an inhibiting effect on the enzyme DPP-IV.

The biological properties of the new compounds were investigated as follows:

The ability of the substances and their corresponding salts to inhibit the DPP-IV activity can be demonstrated in an experiment in which an extract of the human colon carcinoma cell line Caco-2 is used as the DPP IV source. This cell line was obtained from the American Type Culture Collection (ATCC HTB 37). The differentiation of the cells in order to induce the DPP-IV expression was carried out in accordance with the description by Reiher et al. in an article entitled “Increased expression of intestinal cell line Caco-2”, which appeared in Proc. Natl. Acad. Sci. Vol. 90, pp. 5757-5761 (1993). The cell extract was obtained from cells solubilised in a buffer (10 mM Tris HCl, 0.15 M NaCl, 0.04 t.i.u. aprotinin, 0.5% Nonidet-P40, pH 8.0) by centrifugation at 35,000 g for 30 minutes at 4° C. (to remove cell debris).

The DPP-IV assay was carried out as follows:

50 μl of substrate solution (AFC; AFC is amido-4-trifluoromethylcoumarin), final concentration 100 μM, were placed in black microtitre plates. 20 μl of assay buffer (final concentrations 50 mM Tris HCl pH 7.8, 50 mM NaCl, 1% DMSO) was pipetted in. The reaction was started by the addition of 30 μl of solubilised Caco-2 protein (final concentration 0.14 μg of protein per well). The test substances under investigation were typically added prediluted to 20 μl, while the volume of assay buffer was then reduced accordingly. The reaction was carried out at ambient temperature, the incubation period was 60 minutes. Then the fluorescence was measured in a Victor 1420 Multilabel Counter, with the excitation wavelength at 405 nm and the emission wavelength at 535 nm. Dummy values (corresponding to 0% activity) were obtained in mixtures with no Caco-2 protein (volume replaced by assay buffer), control values (corresponding to 100% activity) were obtained in mixtures without any added substance. The potency of the test substances in question, expressed as IC₅₀ values, were calculated from dosage/activity curves consisting of 11 measured points in each case. The following results were obtained:

| Compound | DPP IV inhibition |
|---|---|
| (Example No.) | IC50 [nM] |
| 1(2) | 82 |
| 1(6) | 230 |
| 1(15) | 624 |
| 1(16) | 78 |
| 1(19) | 2770 |
| 1(21) | 124 |
| 1(25) | 56 |
| 1(27) | 125 |
| 1(28) | 166 |
| 1(30) | 2050 |
| 1(34) | 205 |
| 1(35) | 95 |
| 1(55) | 142 |
| 1(60) | 57 |
| 1(62) | 167 |
| 1(70) | 32 |
| 1(97) | 212 |
| 1(121) | 10 |
| 2(1) | 22 |
| 2(22) | 66 |
| 2(28) | 5 |
| 2(56) | 64 |
| 2(77) | 22 |
| 2(85) | 17 |
| 2(88) | 6 |
| 2(113) | 20 |
| 2(119) | 2 |
| 2(127) | 22 |
| 2(131) | 127 |
| 2(136) | 3 |
| 6 | 55 |

The compounds prepared according to the invention are well tolerated as no toxic side effects could be detected in rats after the oral administration of 30 mg/kg of the compound of Example 1(2), for example.

In view of their ability to inhibit DPP-IV activity, the compounds of general formula I according to the invention and the corresponding pharmaceutically acceptable salts thereof are suitable for influencing any conditions or diseases which can be affected by the inhibition of the DPP-IV activity. It is therefore to be expected that the compounds according to the invention will be suitable for the prevention or treatment of diseases or conditions such as type I and type II diabetes mellitus, diabetic complications, metabolic acidosis or ketosis, insulin resistance, dyslipidaemias of various origins, arthritis, atherosclerosis and related diseases, obesity, allograft transplantation and osteoporosis caused by calcitonin. In addition, these substances are suitable for preventing B-cell degeneration such as e.g. apoptosis or necrosis of pancreatic B-cells. The substances are also suitable for improving or restoring the function of pancreatic cells and additionally increasing the size and number of pancreatic B-cells. Additionally, on the basis of the role of the glucagon-like peptides such as e.g. GLP-1 and GLP-2 and their link with DPP-IV inhibition, it is expected that the compounds according to the invention will be suitable for achieving, inter alia, a sedative or tranquillising effect, as well as having a favourable effect on catabolic states after operations or hormonal stress responses or possibly reducing mortality and morbidity after myocardial infarct. Moreover, they are suitable for treating any conditions connected with the effects mentioned above and mediated by GLP-1 or GLP-2. The compounds according to the invention may also be used as diuretics or antihypertensives and are suitable for preventing and treating acute kidney failure. They are also suitable for preventing and treating chronic inflammatory bowel diseases. It is also expected that DPP-IV inhibitors and hence the compounds according to the invention can be used to treat infertility or to improve fertility in humans or mammals, particularly if the infertility is connected with insulin resistance or with polycystic ovary syndrome. In addition, the substances are suitable for treating growth hormone deficiencies connected with restricted growth.

The compounds according to the invention may also be used in conjunction with other active substances. Suitable therapeutic agents for such combinations include for example antidiabetic agents such metformin, sulphonylureas (e.g. glibenclamid, tolbutamide, glimepiride), nateglinide, repaglinide, thiazolidinediones (e.g. rosiglitazone, pioglitazone), PPAR-gamma-agonists (e.g. GI 262570), alpha-glucosidase inhibitors (e.g. acarbose, voglibose), alpha2-antagonists, insulin and insulin analogues, GLP-1 and GLP-1 analogues (e.g. exendin-4) or amylin. The list also includes inhibitors of protein tyrosinephosphatase 1, substances that affect deregulated glucose production in the liver, such as e.g. inhibitors of glucose-6-phosphatase, or fructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents such as for example HMG-CoA-reductase inhibitors (e.g. simvastatin, atorvastatin), fibrates (e.g. bezafibrat, fenofibrat), nicotinic acid and the derivatives thereof, cholesterol absorption inhibitors such as, for example, ezetimibe, bile acid-binding substances such as, for example, cholestyramine, HDL-increasing compounds such as CETP inhibitors or ABC1 regulators or active substances for treating obesity, such as sibutramin or tetrahydrolipstatin or B3-agonists such as SB-418790 or AD-9677. Moreover, combinations with drugs for influencing high blood pressure such as e.g. All antagonists or ACE inhibitors, diuretics, B-blockers and others or combinations thereof are suitable.

The dosage required to achieve such an effect is appropriately 1 to 100 mg, preferably 1 to 30 mg, by intravenous route, and 1 to 1000 mg, preferably 1 to 100 mg, by oral route, in each case administered 1 to 4 times a day. For this purpose, the compounds of formula I prepared according to the invention may be formulated, optionally together with other active substances, together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples which follow are intended to illustrate the invention

### Preparation of the Starting Compounds:

### EXAMPLE I

### 1,3-dimethyl-7-benzyl-8-chloro-xanthine

A mixture of 20 g of 8-chlorotheophylline, 150 ml of dimethylformamide, 10.2 ml of benzyl bromide and 15.5 ml of N-ethyl-diisopropylamine is stirred overnight at ambient temperature. The reaction mixture is poured onto 600 ml of water. The solid is suction filtered, washed with water and diethylether and dried.

Yield: 14.6 g (51% of theory)

Melting point: 155° C.

R_{f} value: 0.84 (silica gel, ethyl acetate/methanol=9:1)

The following compounds are obtained analogously to Example I:

### (1) 1,3-dimethyl-7-(3-methyl-2-buten-1-yl)-8-chloro-xanthine

Melting point: 104° C.

Mass spectrum (EI): m/z=282, 284 [M]⁺

### (2) 1,3-dimethyl-7-(2-butyn-1-yl)-8-chloro-xanthine

Melting point: 105-108° C.

R_{f} value: 0.55 (silica gel, methylene chloride/methanol=20:1)

### (3) 1,3-dimethyl-7-[(1-cyclopenten-1-yl)methyl]-8-chloro-xanthine

R_{f} value: 0.50 (silica gel, methylene chloride/methanol=20:1)

### (4) 1,3-dimethyl-7-(2-thienylmethyl)-8-chloro-xanthine

R_{f} value: 0.35 (silica gel, methylene chloride/methanol=50:1)

Mass spectrum (EI): m/z=310, 312 [M]⁺

### (5) 1,3-dimethyl-7-(3-fluorobenzyl)-8-chloro-xanthine

R_{f} value: 0.60 (silica gel, methylene chloride/methanol=20:1)

### (6) 1,3-dimethyl-7-(2-fluorobenzyl)-8-chloro-xanthine

Mass spectrum (EI): m/z=322, 324 [M]⁺

### (7) 1,3-dimethyl-7-(3-methyl-2-buten-1-yl)-8-(cis-3-tert.-butyloxycarbonylamino-cyclohexyl)-xanthine

Mass spectrum (ESI⁺): m/z=446 [M+H]⁺

### (8) 1,3-dimethyl-7-(4-fluorobenzyl)-8-chloro-xanthine

R_{f} value: 0.60 (silica gel, methylene chloride/methanol=20:1)

### (9) 1,3-dimethyl-7-(2-buten-1-yl)-8-chloro-xanthine

R_{f} value: 0.70 (silica gel, methylene chloride/methanol=10:1)

### (10) 3-methyl-7-(3-methyl-2-buten-1-yl)-8-chloro-xanthine

Melting point: 226-228° C.

R_{f} value: 0.66 (silica gel, methylene chloride/methanol=9:1)

Mass spectrum (ESI⁺): m/z=269, 271 [M+H]⁺

### (11) 3-methyl-7-(3-methyl-2-buten-1-yl)-8-bromo-xanthine

Mass spectrum (ESI⁺): m/z=313, 315 [M+H]⁺

R_{f} value: 0.48 (silica gel, methylene chloride/methanol=10:1)

### (12) 1,3-dimethyl-7-(3-methyl-2-buten-1-yl)-8-[3-(tert.-butyloxycarbonylamino)propyl]-xanthine

Mass spectrum (ESI⁺): m/z=406 [M+H]⁺

### (13) 1,3-dimethyl-7-(3-methyl-2-buten-1-yl)-8-[1-(tert.-butyloxycarbonyl)-piperidin-4-yl]-xanthine

Carried out in the presence of potassium carbonate in dimethylformamide at 60° C.

Mass spectrum (ESI⁺): m/z=432 [M+H]⁺

### (14) 1,3-dimethyl-7-(3-methyl-2-buten-1-yl)-8-[trans-2-(tert.-butyloxycarbonylamino)-cyclohexyl]-xanthine

Mass spectrum (ESI⁺): m/z=446 [M+H]⁺

### (15) 1,3-dimethyl-7-(2-pentyn-1-yl)-8-chloro-xanthine

Mass spectrum (ESI⁺): m/z=281, 283 [M+H]⁺

### (16) 3-methyl-7-benzyl-8-chloro-xanthine

Mass spectrum (ESI⁺): m/z=291, 293 [M+H]⁺

### (17) 3-methyl-7-cyclopropylmethyl-8-chloro-xanthine

Mass spectrum (EI): m/z=254, 256 [M]⁺

### (18) 3-methyl-7-(2-butyn-1-yl)-8-chloro-xanthine

Mass spectrum (ESI⁺): m/z=253, 255 [M+H]⁺

### (19) 1,3-dimethyl-7-(3-methyl-2-buten-1-yl)-8-bromo-xanthine

Mass spectrum (ESI⁺): m/z=327, 329 [M+H]⁺

### (20) 1,3-dimethyl-7-(3-methyl-2-buten-1-yl)-8-[3-(tert.-butyloxycarbonylamino)-cyclohexyl]-xanthine (cis/trans mixture)

Mass spectrum (ESI⁺): m/z=446 [M+H]⁺

### (21) 1,3-dimethyl-7-[(thiophen-3-yl)-methyl]-8-chloro-xanthine

R_{f} value: 0.42 (silica gel, cyclohexane/ethyl acetate=1:1)

### (22) 1,3-dimethyl-7-[(thiophen-2-yl)-methyl]-8-chloro-xanthine

¹H-NMR (300 MHz, CDCl₃): characteristic signals at 3.40 and 3.52 ppm (in each case s, in each case 3H), 5.70 ppm (s, 2H), 6.95 ppm (m, 1H) and 7.25 ppm (m, 2H)

### (23) 1,3-dimethyl-7-[(furan-3-yl)-methyl]-8-chloro-xanthine

R_{f} value: 0.44 (silica gel, ethyl acetate/hexane=1:1)

### (24) 1,3-dimethyl-7-[(furan-2-yl)-methyl]-8-chloro-xanthine

R_{f} value: 0.50 (silica gel, ethyl acetate/hexane=1:1)

### (25) 1,3-dimethyl-7-(2-propyn-1-yl)-8-chloro-xanthine

R_{f} value: 0.33 (silica gel, ethyl acetate/hexane=1:1)

### (26) 1,3-dimethyl-7-(2,3-dimethyl-2-buten-1-yl)-8-chloro-xanthine

R_{f} value: 0.51 (silica gel, ethyl acetate/hexane=1:1)

### (27) 1,3-dimethyl-7-((E)-2-methyl-2-buten-1-yl)-8-chloro-xanthine

R_{f} value: 0.57 (silica gel, ethyl acetate/hexane=1:1)

### (28) 1,3-dimethyl-7-[(cyclohexen-1-yl)-methyl]-8-chloro-xanthine

R_{f} value: 0.62 (silica gel, ethyl acetate/hexane=1:1)

### (29) 1,3-dimethyl-7-[(cyclopenten-1-yl)-methyl]-8-chloro-xanthine

R_{f} value: 0.54 (silica gel, ethyl acetate/hexane=1:1)

### (30) 1,3-dimethyl-7-((Z)-2-methyl-2-buten-1-yl)-8-(piperazin-1-yl)-xanthine

R_{f} value: 0.51 (silica gel, ethyl acetate=1:1)

### (31) 1,3-dimethyl-7-(3-methyl-2-buten-1-yl)-8-[1-(tert.-butyloxycarbonyl)-piperidin-3-yl]-xanthine

Carried out in the presence of potassium carbonate

Mass spectrum (ESI⁺): m/z=432 [M+H]⁺

### (32) 1,3-dimethyl-7-[(2-naphthyl)methyl]-8-chloro-xanthine

Carried out in the presence of potassium carbonate

R_{f} value: 0.60 (silica gel, cyclohexane/ethyl acetate=1:1)

Mass spectrum (ESI⁺): m/z=377, 379 [M+Na]⁺

### (33) 1,3-dimethyl-7-[(1-naphthyl)methyl]-8-chloro-xanthine

Carried out in the presence of potassium carbonate

R_{f} value: 0.60 (silica gel, cyclohexane/ethyl acetate=1:1)

Mass spectrum (ESI⁺): m/z=355, 357 [M+H]⁺

### (34) 1,3-dimethyl-7-(2-cyano-benzyl)-8-chloro-xanthine

Carried out in the presence of potassium carbonate

R_{f} value: 0.60 (silica gel, cyclohexane/ethyl acetate=1:1)

Mass spectrum (ESI⁺): m/z=330, 332 [M+H]⁺

### (35) 1,3-dimethyl-7-(3-cyano-benzyl)-8-chloro-xanthine

Carried out in the presence of potassium carbonate

R_{f} value: 0.60 (silica gel, cyclohexane/ethyl acetate=1:1)

Mass spectrum (ESI⁺): m/z=330, 332 [M+H]⁺

### (36) 1,3-dimethyl-7-(3,5-difluoro-benzyl)-8-chloro-xanthine

Carried out in the presence of potassium carbonate

R_{f} value: 0.60 (silica gel, cyclohexane/ethyl acetate=1:1)

Mass spectrum (EI): m/z=340, 342 [M]⁺

### (37) 1,3-dimethyl-7-(4-cyano-benzyl)-8-chloro-xanthine

Carried out in the presence of potassium carbonate

R_{f} value: 0.60 (silica gel, cyclohexane/ethyl acetate=1:1)

Mass spectrum (EI): m/z=329, 331 [M]⁺

### (38) 1,3-dimethyl-7-(3-nitro-benzyl)-8-chloro-xanthine

Carried out in the presence of potassium carbonate

R_{f} value: 0.60 (silica gel, cyclohexane/ethyl acetate=1:1)

Mass spectrum (ESI⁺): m/z=350, 352 [M+H]⁺

### (39) 1,3-dimethyl-7-(4-nitro-benzyl)-8-chloro-xanthine

Carried out in the presence of potassium carbonate

R_{f} value: 0.60 (silica gel, cyclohexane/ethyl acetate=1:1)

### (40) 3-methyl-7-(2-cyano-benzyl)-8-chloro-xanthine

R_{f} value: 0.60 (silica gel, cyclohexane/ethyl acetate=1:1)

Mass spectrum (ESI⁺): m/z=316, 318 [M+H]⁺

### (41) 1,3-dimethyl-7-(2-nitro-benzyl)-8-chloro-xanthine

Carried out in the presence of potassium carbonate

R_{f} value: 0.60 (silica gel, cyclohexane/ethyl acetate=1:1)

### (42) 1,3-dimethyl-7-(2-iod-benzyl)-8-chloro-xanthine

Carried out in the presence of potassium carbonate.

Mass spectrum (ESI⁺): m/z=431, 433 [M+H]⁺

### EXAMPLE II

### (R)-1,3-dimethyl-7-(3-methyl-2-buten-1-yl)-8-[3-(tert.-butyloxycarbonylamino)-piperidin-1-yl]-xanthine

A mixture of 1 g of 1,3-dimethyl-7-(3-methyl-2-buten-1-yl)-8-chloro-xanthine, 1.32 g of (R)-3-tert.-butyloxycarbonylamino-piperidine, 1 ml of triethylamine and 10 ml of dimethylformamide is stirred at 50° C. for two and a half days. The reaction mixture is diluted with 100 ml of water and then extracted with ethyl acetate. The organic phase is dried, evaporated down and the residue is stirred with diethylether.

The solid is suction filtered and dried.

Yield: 1.0 g (63% of theory)

Melting point: 164° C.

R_{f} value: 0.36 (aluminium oxide, cyclohexane/ethyl acetate=1:1)

The following compounds are obtained analogously to Example II:

### (1) (S)-1,3-dimethyl-7-(3-methyl-2-buten-1-yl)-8-[3-(tert.-butyloxycarbonylamino)-piperidin-1-yl]-xanthine

Melting point: 164° C.

Mass spectrum (ESI⁻): m/z=445 [M−H]⁻

### (2) 1,3-dimethyl-7-(3-methyl-2-buten-1-yl)-8-[3-(tert.-butyloxycarbonylamino)-hexahydroazepin-1-yl]-xanthine

Melting point: 154° C.

Mass spectrum (ESI⁻): m/z=459 [M−H]⁻

### (3) 1,3-dimethyl-7-(3-methyl-2-buten-1-yl)-8-[4-(tert.-butyloxycarbonylamino)-hexahydroazepin-1-yl]-xanthine

Mass spectrum (ESI⁻): m/z=459 [M−H]⁻

R_{f} value: 0.67 (silica gel, ethyl acetate)

### (4) 1,3-dimethyl-7-(3-methyl-2-buten-1-yl)-8-[3-(tert.-butyloxycarbonylamino)-4-methyl-piperidin-1-yl]-xanthine

Mass spectrum (ESI⁺): m/z=461 [M+H]⁺

R_{f} value: 0.88 (silica gel, ethyl acetate/methanol=5:1)

### (5) 1-methyl-3-(4-methoxy-benzyl)-7-benzyl-8-[(S)-3-(tert.-butyloxycarbonylamino)-piperidin-1-yl]-xanthine

Mass spectrum (ESI⁺): m/z=575 [M+H]⁺

R_{f} value: 0.74 (silica gel, methylene chloride/methanol=95:5)

### (6) 1,3-dimethyl-7-(3-methyl-2-buten-1-yl)-8-{N-[2-(tert.-butyloxycarbonylamino)-ethyl]-N-ethyl-amino}-xanthine

Mass spectrum (ESI⁺): m/z=435 [M+H]⁺

### (7) 1-methyl-3-hexyl-7-benzyl-8-[(S)-3-(tert.-butyloxycarbonylamino)-piperidin-1-yl]-xanthine

Melting point: 152-159° C.

Mass spectrum (ESI⁺): m/z=539 [M+H]⁺

### (8) 1-methyl-3-(2-trimethylsilanyl-ethoxymethyl)-7-benzyl-8-(3-amino-piperidin-1-yl)-xanthine

Carried out with potassium carbonate at 120° C.

Mass spectrum (ESI⁺): m/z=485 [M+H]⁺

### (9) 1-methyl-3-(2-hydroxy-ethyl)-7-benzyl-8-[(S)-3-(tert.-butyloxycarbonylamino)-piperidin-1-yl]-xanthine

Carried out with potassium carbonate at 110° C.

R_{f} value: 0.41 (silica gel, methylene chloride/methanol/conc. aqueous ammonia=9:1:0.1)

Mass spectrum (ESI⁺): m/z=499 [M+H]⁺

### (10) 1-(2-phenyl-ethyl)-3-methyl-7-(3-methyl-2-buten-1-yl)-8-[(S)-3-(tert.-butyloxycarbonylamino)-piperidin-1-yl]-xanthine

Carried out with Hünig base at 100° C.

Mass spectrum (ESI⁺): m/z=537 [M+H]⁺

### (11) 1-(2-phenyl-ethyl)-3-methyl-7-(3-methyl-2-buten-1-yl)-8-[(R)-3-(tert.-butyloxycarbonylamino)-piperidin-1-yl]-xanthine

Mass spectrum (ESI⁺): m/z=537 [M+H]⁺

### (12) 1,3-dimethyl-7-(3-methyl-2-buten-1-yl)-8-{2-[(tert.-butyloxycarbonylamino)methyl]-piperidin-1-yl}-xanthine

Carried out with potassium carbonate and sodium iodide in dimethylsulphoxide at 120° C.

R_{f} value: 0.73 (silica gel, ethyl acetate)

Mass spectrum (ESI⁺): m/z=461 [M+H]⁺

### (13) 1,3-dimethyl-7-(3-methyl-2-buten-1-yl)-8-{[1-(tert.-butyloxycarbonyl)-pyrrolidin-3-yl]amino}-xanthine

Carried out with sodium carbonate in dimethylsulphoxide at 130° C.

R_{f} value: 0.50 (silica gel, ethyl acetate)

Mass spectrum (ESI⁺): m/z=433 [M+H]⁺

### (14) 1,3-dimethyl-7-(3-methyl-2-buten-1-yl)-8-{N-[1-(tert.-butyloxycarbonyl)-piperidin-3-yl]-N-methyl-amino}-xanthine

Carried out with Hünig base, 4-dimethylaminopyridine and sodium carbonate in dimethylsulphoxide at 150° C.

R_{f} value: 0.62 (silica gel, ethyl acetate)

Mass spectrum (ESI⁺): m/z=461 [M+H]⁺

### (15) 3-methyl-7-(3-methyl-2-buten-1-yl)-8-[(S)-3-(tert.-butyloxycarbonylamino)-piperidin-1-yl]-xanthine

R_{f} value: 0.30 (silica gel, methylene chloride/methanol=9:1)

Mass spectrum (ESI⁺): m/z=433 [M+H]⁺

### (16) 1,3-dimethyl-7-(3-methyl-2-buten-1-yl)-8-{[1-(tert.-butyloxycarbonyl)-piperidin-4-yl]amino}-xanthine

Carried out with Hünig base and 4-dimethylaminopyridine in dimethylsulphoxide at 100° C.

R_{f} value: 0.81 (silica gel, methylene chloride/methanol/conc. aqueous ammonia=9:1:0.1)

### (17) 1,3-dimethyl-7-(3-methyl-2-buten-1-yl)-8-{[1-(tert.-butyloxycarbonyl)-piperidin-3-yl]amino}-xanthine

Carried out with Hünig base and 4-dimethylaminopyridine in dimethylsulphoxide at 100° C.

R_{f} value: 0.37 (silica gel, ethyl acetate/hexane=7:3)

### (18) 3-methyl-7-(3-methyl-2-buten-1-yl)-8-[3-(tert.-butyloxycarbonylamino)-piperidin-1-yl]-xanthine

R_{f} value: 0.49 (silica gel, petroleum ether/ethyl acetate/methanol=5:4:1)

Mass spectrum (ESI⁺): m/z=433 [M+H]⁺

### (19) 1,3-dimethyl-7-(3-methyl-2-buten-1-yl)-8-{N-[1-(tert.-butyloxycarbonyl)-pyrrolidin-3-yl]-N-methyl-amino}-xanthine

Carried out with sodium carbonate in dimethylsulphoxide at 160° C.

R_{f} value: 0.68 (silica gel, methylene chloride/methanol/conc. aqueous ammonia=90:10:1)

Mass spectrum (ESI⁺): m/z=447 [M+H]⁺

### (20) 1-[2-(2-nitro-phenyl)-ethyl]-3-methyl-7-(3-methyl-2-buten-1-yl)-8-[3-(tert.-butyloxycarbonylamino)-piperidin-1-yl]-xanthine

R_{f} value: 0.34 (silica gel, petroleum ether/ethyl acetate/methanol=7:2:1)

Mass spectrum (ESI⁺): m/z=582 [M+H]⁺

### (21) 1-[2-(3,5-difluoro-phenyl)-ethyl]-3-methyl-7-(3-methyl-2-buten-1-yl)-8-[3-(tert.-butyloxycarbonylamino)-piperidin-1-yl]-xanthine

R_{f} value: 0.38 (silica gel, petroleum ether/ethyl acetate/methanol=7:2:1)

Mass spectrum (ESI⁺): m/z=573 [M+H]⁺

### (22) 1-[2-(2,6-difluoro-phenyl)-ethyl]-3-methyl-7-(3-methyl-2-buten-1-yl)-8-[3-(tert.-butyloxycarbonylamino)-piperidin-1-yl]-xanthine

R_{f} value: 0.38 (silica gel, petroleum ether/ethyl acetate/methanol=7:2:1)

Mass spectrum (ESI⁺): m/z=573 [M+H]⁺

### (23) 3-methyl-7-(3-methyl-2-buten-1-yl)-8-[(R)-3-(tert.-butyloxycarbonylamino)-piperidin-1-yl]-xanthine

Mass spectrum (ESI⁺): m/z=433 [M+H]⁺

### (24) 1-[2-(3,5-dimethyl-phenyl)-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=565 [M+H]⁺

### (25) 1,3-dimethyl-7-(3-methyl-2-buten-1-yl)-8-[cis-2-(tert.-butyloxycarbonylamino)-cyclopropylamino]-xanthine

R_{f} value: 0.41 (silica gel, ethyl acetate)

Mass spectrum (ESI⁺): m/z=419 [M+H]⁺

### (26) 3-methyl-7-(2-cyano-benzyl)-8-[3-(tert.-butyloxycarbonylamino)-piperidin-1-yl]-xanthine

Carried out with sodium carbonate in dimethylsulphoxide

Mass spectrum (ESI⁻): m/z=478 [M−H]⁻

### (27) 1-(2-phenyl-2-oxo-ethyl)-3-methyl-7-(3-methyl-2-buten-1-yl)-8-[4-(tert.-butyloxycarbonyl)-piperazin-1-yl]-xanthine

Carried out with potassium carbonate at 100° C.

R_{f} value: 0.70 (silica gel, cyclohexane/ethyl acetate=1:1)

Mass spectrum (ESI⁺): m/z=537 [M+H]⁺

### (28) 1-[2-(3-nitro-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=596 [M+H]⁺

### (29) 1-(2-phenyl-2-oxo-ethyl)-3-methyl-7-(3-methyl-2-buten-1-yl)-8-[4-(tert.-butyloxycarbonyl)-homopiperazin-1-yl]-xanthine

R_{f} value: 0.70 (silica gel, cyclohexane/ethyl acetate=1:1)

### (30) 1,3-dimethyl-7-(3-methyl-2-buten-1-yl)-8-{4-[(tert.-butyloxycarbonylamino)-methyl]-piperidin-1-yl}-xanthine

Carried out in 1-methyl-2-pyrrolidone at 135° C.

R_{f} value: 0.69 (silica gel, ethyl acetate)

Mass spectrum (ESI⁺): m/z=461 [M+H]⁺

### (31) 1,3-dimethyl-7-(3-methyl-2-buten-1-yl)-8-{3-[(tert.-butyloxycarbonylamino)-methyl)-piperidin-1-yl}-xanthine

Carried out in 1-methyl-2-pyrrolidone at 135° C.

R_{f} value: 0.74 (silica gel, ethyl acetate)

Mass spectrum (ESI⁺): m/z=461 [M+H]⁺

### (32) 1,3-dimethyl-7-(3-methyl-2-buten-1-yl)-8-[trans-2-(tert.-butyloxycarbonylamino)-cyclobutylamino]-xanthine

Carried out in the presence of Hünig base in 1-methyl-2-pyrrolidone at 135° C.

R_{f} value: 0.65 (silica gel, ethyl acetate/petroleum ether=8:2)

Mass spectrum (ESI⁺): m/z=433 [M+H]⁺

### (33) 1,3-dimethyl-7-(3-methyl-2-buten-1-yl)-8-{N—[(S)-2-(tert.-butyloxycarbonylamino)-1-methyl-ethyl]-N-methyl-amino}-xanthine

Carried out with sodium carbonate in dimethylsulphoxide

R_{f} value: 0.69 (silica gel, ethyl acetate)

Mass spectrum (ESI⁺): m/z=435 [M+H]⁺

### (34) 1,3-dimethyl-7-(3-methyl-2-buten-1-yl)-8-{N—[(R)-2-(tert.-butyloxycarbonylamino)-1-methyl-ethyl]-N-methyl-amino}-xanthine

Carried out with sodium carbonate in dimethylsulphoxide

R_{f} value: 0.32 (silica gel, cyclohexane/ethyl acetate=1:1)

Mass spectrum (ESI⁺): m/z=435 [M+H]⁺

### (35) 1,3-dimethyl-7-(3-methyl-2-buten-1-yl)-8-[cis-2-(tert.-butyloxycarbonylamino)-cyclohexylamino]-xanthine

Carried out with sodium carbonate in dimethylsulphoxide

R_{f} value: 0.35 (silica gel, cyclohexane/ethyl acetate=1:1)

Mass spectrum (ESI⁺): m/z=461 [M+H]⁺

### (36) 1,3-dimethyl-7-(3-methyl-2-buten-1-yl)-8-[6-(tert.-butyloxycarbonylamino)-[1,4]diazepan-1-yl]-xanthine

Carried out with sodium carbonate in dimethylsulphoxide

R_{f} value: 0.08 (silica gel, methylene chloride/methanol=95:5)

### (37) 1-[(pyridin-2-yl)methyl]-3-methyl-7-(3-methyl-2-buten-1-yl)-8-[3-(tert.-butyloxycarbonylamino)-piperidin-1-yl]-xanthine

Carried out with sodium carbonate in dimethylsulphoxide

R_{f} value: 0.43 (silica gel, ethyl acetate)

Mass spectrum (ESI⁺): m/z=524 [M+H]⁺

### (38) 1,3-dimethyl-7-(3-methyl-2-buten-1-yl)-8-[trans-2-(tert.-butyloxycarbonylamino)-cyclopentylamino]-xanthine

Carried out in the presence of Hünig base in 1-methyl-2-pyrrolidone at 135° C.

Melting point: 177-179° C.

Mass spectrum (ESI⁺): m/z=447 [M+H]⁺

### (39) 1,3-dimethyl-7-(3-methyl-2-buten-1-yl)-8-[3-(tert.-butyloxycarbonylamino)-cyclohexylamino]-xanthine (cis/trans mixture)

Carried out in the presence of Hünig base in 1-methyl-2-pyrrolidone at 135° C.

R_{f} value: 0.36 (silica gel, ethyl acetate/petroleum ether=1:1)

Mass spectrum (ESI⁻): m/z=459 [M−H]⁻

### (40) 1,3-dimethyl-7-(3-methyl-2-buten-1-yl)-8-[cis-2-(tert.-butyloxycarbonylamino)-cyclopentylamino]-xanthine

Melting point: 175-178° C.

Mass spectrum (ESI⁻): m/z=445 [M−H]⁻

### (41) 1-[(isoquinolin-1-yl)methyl]-3-methyl-7-(3-methyl-2-buten-1-yl)-8-[3-(tert.-butyloxycarbonylamino)-piperidin-1-yl]-xanthine

Carried out with sodium carbonate in dimethylsulphoxide

R_{f} value: 0.51 (silica gel, methylene chloride/methanol=95:5)

### (42) 1,3-dimethyl-7-(3-methyl-2-buten-1-yl)-8-[cis-3-(tert.-butyloxycarbonylamino)-cyclopentylamino]-xanthine

Carried out in the presence of Hünig base in 1-methyl-2-pyrrolidone at 135° C.

R_{f} value: 0.23 (silica gel, ethyl acetate/petroleum ether=1:1)

Mass spectrum (ESI⁺): m/z=447 [M+H]⁺

### (43) 1-[(pyridin-3-yl)methyl]-3-methyl-7-(3-methyl-2-buten-1-yl)-8-[3-(tert.-butyloxycarbonylamino)-piperidin-1-yl]-xanthine Carried out with sodium carbonate in dimethylsulphoxide

R_{f} value: 0.44 (silica gel, methylene chloride/methanol=95:5)

Mass spectrum (ESI⁺): m/z=524 [M+H]⁺

### (44) 1-[(pyridin-4-yl)methyl]-3-methyl-7-(3-methyl-2-buten-1-yl)-8-[3-(tert.-butyloxycarbonylamino)-piperidin-1-yl]-xanthine

Carried out with sodium carbonate in dimethylsulphoxide

R_{f} value: 0.28 (silica gel, ethyl acetate)

Mass spectrum (ESI⁺): m/z=524 [M+H]⁺

### (45) 1-[(isoquinolin-1-yl)methyl]-3-methyl-7-(3-methyl-2-buten-1-yl)-8-[(R)-3-(tert.-butyloxycarbonylamino)-piperidin-1-yl]-xanthine

Carried out with potassium carbonate in dimethylsulphoxide

R_{f} value: 0.37 (silica gel, ethyl acetate)

Mass spectrum (ESI⁺): m/z=574 [M+H]⁺

### (46) 1-[(isoquinolin-1-yl)methyl]-3-methyl-7-(3-methyl-2-buten-1-yl)-8-[(S)-3-(tert.-butyloxycarbonylamino)-piperidin-1-yl]-xanthine

Carried out with potassium carbonate in dimethylsulphoxide

R_{f} value: 0.37 (silica gel, ethyl acetate)

Mass spectrum (ESI⁺): m/z=574 [M+H]⁺

### (47) 1-(2-phenyl-2-oxo-ethyl)-3-methyl-7-(3-methyl-2-buten-1-yl)-8-[3-(tert.-butyloxycarbonylamino)-3-methyl-piperidin-1-yl]-xanthine

R_{f} value: 0.51 (silica gel, cyclohexane/ethyl acetate/methanol=6:3:1)

Mass spectrum (ESI⁺): m/z=565 [M+H]⁺

### (48) 1,3-dimethyl-7-(3-methyl-2-buten-1-yl)-8-[3-(tert.-butyloxycarbonylamino)-3-methyl-piperidin-1-yl]-xanthine

R_{f} value: 0.48 (silica gel, cyclohexane/ethyl acetate/methanol=6:3:1)

Mass spectrum (EI): m/z=460 [M]⁺

### (49) 1,3-dimethyl-7-(3-methyl-2-buten-1-yl)-8-{N-[2-(tert.-butyloxycarbonylamino)-3-dimethylamino-3-oxo-propyl]-N-methyl-amino}-xanthine

R_{f} value: 0.48 (silica gel, methylene chloride/methanol=9:1)

Mass spectrum (ESI⁺): m/z=492 [M+H]⁺

### (50) 1,3-dimethyl-7-(3-methyl-2-buten-1-yl)-8-{N-[2-(tert.-butyloxycarbonylamino)-3-amino-3-oxo-propyl]-N-methyl-amino}-xanthine

R_{f} value: 0.40 (silica gel, methylene chloride/methanol=9:1)

Mass spectrum (EI): m/z=463 [M]⁺

### (51) 1-[2-(2-nitro-phenyl)-2-oxo-ethyl]-3-methyl-7-(3-methyl-2-buten-1-yl)-8-[3-(tert.-butyloxycarbonylamino)-piperidin-1-yl]-xanthine

Carried out with sodium carbonate in dimethylsulphoxide.

Mass spectrum (ESI⁺): m/z=596 [M+H]⁺

### (52) 1-[(isoquinolin-4-yl)methyl]-3-methyl-7-(3-methyl-2-buten-1-yl)-8-[3-(tert.-butyloxycarbonylamino)-piperidin-1-yl]-xanthine

Carried out with sodium carbonate in dimethylsulphoxide.

R_{f} value: 0.48 (silica gel, ethyl acetate)

Mass spectrum (ESI⁺): m/z=574 [M+H]⁺

### (53) 1-[(1-methyl-1H-indazol-3-yl)methyl]-3-methyl-7-(3-methyl-2-buten-1-yl)-8-[3-(tert.-butyloxycarbonylamino)-piperidin-1-yl]-xanthine

Carried out with sodium carbonate in dimethylsulphoxide.

Mass spectrum (ESI⁺): m/z=577 [M+H]⁺

### (54) 1,3-dimethyl-7-(3-methyl-2-buten-1-yl)-8-{N-[2-(tert.-butyloxycarbonylamino)-3-oxo-3-(pyrrolidin-1-yl)-propyl]-N-methyl-amino}-xanthine

Carried out with Hünig base in N-methylpyrrolidinone.

Melting point: 173-175° C.

Mass spectrum (ESI⁺): m/z=518 [M+H]⁺

### (55) 1,3-dimethyl-7-(3-methyl-2-buten-1-yl)-8-{N-[2-(tert.-butyloxycarbonylamino)-3-methylamino-3-oxo-propyl]-N-methyl-amino}-xanthine

Carried out with Hünig base in N-methylpyrrolidinone.

Mass spectrum (ESI⁺): m/z=478 [M+H]⁺

### (56) 1-[2-(2-hydroxy-phenyl)-2-oxo-ethyl]-3-methyl-7-(3-methyl-2-buten-1-yl)-8-[3-(tert.-butyloxycarbonylamino)-piperidin-1-yl]-xanthin

Mass spectrum (ESI⁺): m/z=567 [M+H]⁺

### (57) 1-methyl-3-[2-(4-methoxy-phenyl)-ethyl]-7-(2-cyano-benzyl)-8-[3-(tert.-butyloxy-carbonylamino)-piperidin-1-yl]-xanthin

Carried out in the presence of sodium carbonate in dimethylsulphoxide.

Rf value: 0.50 (silica gel, methylene chloride/methanol=9:1)

Mass spectrum (ESI⁺): m/z=614 [M+H]⁺

### (58) 1-methyl-3-(2-phenyl-ethyl)-7-(2-cyano-benzyl)-8-[3-(tert.-butyloxycarbonyl-amino)-piperidin-1-yl]-xanthine

Carried out in the presence of sodium carbonate in dimethylsulphoxide.

Mass spectrum (ESI⁺): m/z=584 [M+H]⁺

### (59) 1-[(quinolin-411)methyl]-3-methyl-7-(3-methyl-2-buten-1-yl)-8-[3-(tert.-butyloxy-carbonylamino)-piperidin-1-yl]-xanthine

Carried out in the presence of sodium carbonate in dimethylsulphoxide.

Rf value: 0.50 (silica gel, ethyl acetate)

Mass spectrum (ESI⁺): m/z=574 [M+H]⁺

### (60) 1,3-dimethyl-7-(3-methyl-2-buten-1-yl)-8-[endo-6-(tert.-butyloxycarbonylamino)-2-aza-bicyclo[2.2.2]oct-2-yl]-xanthine Carried out in the presence of potassium carbonate and Hünig base in dimethylsulphoxide.

Rf value: 0.52 (silica gel, cyclohexane/ethyl acetate=1:1)

Mass spectrum (ESI⁺): m/z=473 [M+H]⁺

### (61) 1-[(quinolin-8-yl)methyl]-3-methyl-7-(3-methyl-2-buten-1-yl)-8-[3-(tert.-butyloxy-carbonylamino)-piperidin-1-yl]-xanthine

Carried out in the presence of sodium carbonate in dimethylsulphoxide.

Rf value: 0.73 (silica gel, ethyl acetate)

Mass spectrum (ESI⁺): m/z=574 [M+H]⁺

### (62) 1,3-dimethyl-7-(3-methyl-2-buten-1-yl)-8-[exo-6-(tert.-butyloxycarbonylamino)-2-aza-bicyclo[2.2.2]oct-2-yl]-xanthine

Carried out in the presence of potassium carbonate and Hünig base in dimethylsulphoxide.

Rf value: 0.45 (silica gel, cyclohexane/ethyl acetate=1:1)

Mass spectrum (ESI⁺): m/z=473 [M+H]⁺

### (63) 1-[2-(3-cyano-phenyl)-2-oxo-ethyl]-3-methyl-7-(3-methyl-2-buten-1-yl)-8-[3-(tert.-butyloxycarbonylamino)-piperidin-1-yl]-xanthine

Carried out in the presence of sodium carbonate in dimethylsulphoxide.

Rf value: 0.33 (silica gel, cyclohexane/ethyl acetate=1:1)

Mass spectrum (ESI⁺): m/z=576 [M+H]⁺

### (64) 1-[2-(3-aminosulphonyl-phenyl)-2-oxo-ethyl]-3-methyl-7-(3-methyl-2-buten-1-yl)-8-[3-(tert.-butyloxycarbonylamino)-piperidin-1-yl]-xanthine

Carried out in the presence of sodium carbonate in dimethylsulphoxide.

Rf value: 0.15 (silica gel, cyclohexane/ethyl acetate=1:1)

Mass spectrum (ESI⁻): m/z=628 [M−H]⁻

### (65) 1-[2-(3-aminocarbonyl-phenyl)-2-oxo-ethyl]-3-methyl-7-(3-methyl-2-buten-1-yl)-8-[3-(tert.-butyloxycarbonylamino)-piperidin-1-yl]-xanthine

Carried out in the presence of sodium carbonate in dimethylsulphoxide.

Rf value: 0.36 (silica gel, methylene chloride/methanol=9:1)

Mass spectrum (ESI⁺): m/z=594 [M+H]⁺

### EXAMPLE III

### 3-(tert.-butyloxycarbonylamino)-hexahydroazepine

2 g of 1-benzyl-3-(tert.-butyloxycarbonylamino)-hexahydroazepine in 20 ml of methanol are hydrogenated for 24 hours at ambient temperature under a hydrogen pressure of 3 bar in the presence of 200 mg palladium on activated charcoal (10% Pd). Then the catalyst is removed by suction filtering and the filtrate is evaporated to dryness.

Yield: 1.3 g (90% of theory)

Melting point: 78° C.

Mass spectrum (ESI⁺): m/z=215 [M+H]⁺

The following compounds are obtained analogously to Example III:

### (1) (S)-3-(tert.-butyloxycarbonylamino)-piperidine

Melting point: 122° C.

Mass spectrum (ESI⁺): m/z=201 [M+H]⁺

### (2) (R)-3-(tert.-butyloxycarbonylamino)-piperidine

The starting material, (R)-1-benzyl-3-(tert.-butyloxycarbonylamino)-piperidine, was prepared analogously to the (S)-enantiomer known from the literature (Moon, Sung-Hwan; Lee, Sujin; Synth. Commun.; 28; 21; 1998; 3919-3926)

Melting point: 119° C.

Mass spectrum (ESI⁺): m/z=201 [M+H]⁺

### (3) 4-(tert.-butyloxycarbonylamino)-hexahydroazepine

Mass spectrum (ESI⁺): m/z=215 [M+H]⁺

R_{f} value: 0.02 (aluminium oxide, cyclohexane/ethyl acetate=1:1)

### (4) 3-(tert.-butyloxycarbonylamino)-4-methyl-piperidine

The crude product is further reacted directly to form the compound of Example II (4).

### (5) 6-(tert.-butyloxycarbonylamino)-[1,4]diazepan

The starting material 1,4-dibenzyl-6-(tert.-butyloxycarbonylamino)-[1,4]diazepan was prepared analogously to J. heterocycl. Chem. 1995, 32, 637-642.

The crude product is further reacted directly to form the compound of Example II (36).

### (6) 2-(tert.-butyloxycarbonylamino)-3-methylamino-propionic acid-dimethylamide

R_{f} value: 0.40 (silica gel, methylene chloride/methanol/conc. aqueous ammonia=40:10:1)

Mass spectrum (ESI⁺): m/z=246 [M+H]⁺

### (7) 2-(tert.-butyloxycarbonylamino)-3-methylamino-propionic acid-amide

R_{f} value: 0.20 (silica gel, methylene chloride/methanol/conc. aqueous ammonia=40:10:1)

Mass spectrum (ESI⁺): m/z=218 [M+H]⁺

### (8) 2-(tert.-butyloxycarbonylamino)-3-methylamino-1-(pyrrolidin-1-yl)-propan-1-one

Palladium(II)hydroxide is used as catalyst.

Mass spectrum (ESI⁺): m/z=272 [M+H]⁺

### (9) 2-(tert.-butyloxycarbonylamino)-1,3-bis(methylamino)-propan-1-one

Palladium(II)hydroxide is used as catalyst.

Mass spectrum (ESI⁺): m/z=232 [M+H]⁺

### (10) endo-6-(tert.-Butyloxycarbonylamino)-2-aza-bicyclo[2.2.2]octan

Rf value: 0.25 (silica gel, methylene chloride/methanol/conc. aqueous ammonia=90:10:0.1)

Mass spectrum (ESI⁺): m/z=227 [M+H]⁺

### (11) exo-6-(tert.-butyloxycarbonylamino)-2-aza-bicyclo[2.2.2]octane

Rf value: 0.27 (silica gel, methylene chloride/methanol/conc. aqueous ammonia=90:10:1)

### (12) 1-(tert.-butyloxycarbonyl)-3-amino-4-hydroxy-piperidin

Rf value: 0.17 (silica gel, methylene chloride/methanol/conc. aqueous ammonia=90:10:1)

Mass spectrum (ESI⁺): m/z=217 [M+H]⁺

### EXAMPLE IV

### 1-benzyl-3-(tert.-butyloxycarbonylamino)-hexahydroazepine

Prepared by reacting 1-benzyl-3-amino-hexahydroazepine with di-tert.butyl pyrocarbonate

Melting point: 48-50° C.

Mass spectrum (ESI⁺): m/z=305 [M+H]⁺

The following compounds are obtained analogously to Example IV:

### (1) 1-benzyl-4-(tert.-butyloxycarbonylamino)-hexahydroazepine

Mass spectrum (ESI⁺): m/z=305 [M+H]⁺

R_{f} value: 0.79 (aluminium oxide, cyclohexane/ethyl acetate=1:1)

### (2) 3-(tert.-butyloxycarbonylamino)-4-methyl-pyridine

Carried out with sodium-bis-(trimethylsilyl)-amide/di-tert.butyl pyrocarbonate in tetrahydrofuran at 0° C.

R_{f} value: 0.45 (silica gel, ethyl acetate)

### (3) 1-(tert.-butyloxycarbonyl)-3-[(2,2,2-trifluoro-acetyl)amino]-pyrrolidine

Carried out with triethylamine in tetrahydrofuran

R_{f} value: 0.77 (silica gel, methylene chloride/methanol/conc. aqueous ammonia=90:10:1)

Mass spectrum (ESI⁺): m/z=281 [M+H]⁺

### (4) trans-2-amino-1-(tert.-butyloxycarbonylamino)-cyclobutane

Carried out with di-tert.butyl pyrocarbonate in the presence of 1N sodium hydroxide solution in methanol at 0° C.

R_{f} value: 0.60 (silica gel, methylene chloride/methanol/conc. aqueous ammonia=90:10:0.1)

Mass spectrum (ESI⁺): m/z=187 [M+H]⁺

### (5) (S)-1-(tert.-butyloxycarbonylamino)-2-methylamino-propane

Carried out with di-tert.butyl pyrocarbonate in the presence of Hünig base in methanol.

Mass spectrum (ESI⁺): m/z=189 [M+H]⁺

R_{f} value: 0.30 (silica gel, methylene chloride/methanol/conc. aqueous ammonia=90:10:1)

### (6) (R)-1-(tert.-butyloxycarbonylamino)-2-methylamino-propane

Carried out with di-tert.butyl pyrocarbonate in the presence of Hünig base in methanol.

Mass spectrum (ESI⁺): m/z=189 [M+H]⁺

### (7) 1,3-dimethyl-7-(3-methyl-2-buten-1-yl)-8-[2-(tert.-butyloxycarbonylamino)-2-methyl-propylamino]-xanthine

Carried out with di-tert.butyl pyrocarbonate in the presence of Hünig base in methanol.

R_{f} value: 0.82 (silica gel, methylene chloride/methanol/conc. aqueous ammonia=90:10:1)

### (8) cis-3-amino-1-(tert.-butyloxycarbonylamino)-cyclopentane

Carried out with di-tert.butyl pyrocarbonate in the presence of 1N sodium hydroxide solution in methanol.

R_{f} value: 0.63 (silica gel, methylene chloride/methanol/conc. aqueous ammonia=40:10:1)

Mass spectrum (ESI⁺): m/z=201 [M+H]⁺

### (9) endo-6-(tert.-butyloxycarbonylamino)-2-benzyl-2-aza-bicyclo[2.2.2]octane

Rf value: 0.53 (aluminium oxide, cyclohexane/ethyl acetate=9:1)

Mass spectrum (ESI⁺): m/z=317 [M+H]⁺

### (10) exo-6-(tert.-butyloxycarbonylamino)-2-benzyl-2-aza-bicyclo[2.2.2]octane

Rf value: 0.37 (aluminium oxide, cyclohexane/ethyl acetate=9:1)

Mass spectrum (ESI⁺): m/z=317 [M+H]⁺

### EXAMPLE V

1,3-dimethyl-8-(cis-3-tert.-butyloxycarbonylamino-cyclohexyl)-xanthine

Prepared from the compound of Example VI by treating with 4N sodium hydroxide solution in methanol at 100° C. in a bomb tube

Mass spectrum (ESI⁺): m/z=378 [M+H]⁺

The following compound is obtained analogously to Example V:

### (1) 1,3-dimethyl-8-[3-(tert.-butyloxycarbonylamino)propyl]-xanthine

Mass spectrum (ESI⁺): m/z=338 [M+H]⁺

### (2) 1,3-dimethyl-8-[1-(tert.-butyloxycarbonyl)-piperidin-4-yl]-xanthine

### (3) 1,3-dimethyl-8-[trans-2-(tert.-butyloxycarbonylamino)-cyclohexyl]-xanthine

Mass spectrum (ESI⁺): m/z=378 [M+H]⁺

### (4) 1,3-dimethyl-8-[3-(tert.-butyloxycarbonylamino)-cyclohexyl]-xanthine (cis/trans mixture)

Mass spectrum (ESI⁺): m/z=378 [M+H]⁺

### (5) 1,3-dimethyl-8-[1-(tert.-butyloxycarbonyl)-piperidin-3-yl]-xanthine

Mass spectrum (ESI⁺): m/z=364 [M+H]⁺

### EXAMPLE VI

### 1,3-dimethyl-5-[(cis-3-tert.-butyloxycarbonylamino-cyclohexyl)-carbonylamino]-6-amino-uracil

Prepared from 5,6-diamino-1,3-dimethyluracil and cis-3-tert.-butyloxycarbonylamino-cyclohexanecarboxylic acid in the presence of O-(benzotriazol-1-yl)-N,N,N′,N′-tetramethyluronium hexafluorophosphate and N-ethyl-diisopropylamine in dimethylformamide at ambient temperature

Mass spectrum (ESI⁺): m/z=396 [M+H]⁺

The following compound is obtained analogously to Example VI:

### (1) 1,3-dimethyl-5-{[3-(tert.-butyloxycarbonylamino)propyl]-carbonylamino}-6-amino-uracil

### (2) 1,3-dimethyl-5-{[1-(tert.-butyloxycarbonyl)-piperidin-4-yl]-carbonylamino}-6-amino-uracil

Carried out with O-(benzotriazol-1-yl)-N,N,N′,N′-tetramethyluronium tetrafluoroborate and N-hydroxybenzotriazole

Mass spectrum (ESI⁺): m/z=382 [M+H]⁺

### (3) 1,3-dimethyl-5-({trans-2-[(fluoren-9-ylmethoxycarbonyl)amino]-cyclohexyl}-carbonylamino)-6-amino-uracil

Carried out with O-(benzotriazol-1-yl)-N,N,N′,N′-tetramethyluronium tetrafluoroborate

Mass spectrum (ESI⁺): m/z=518 [M+H]⁺

### (4) 1,3-dimethyl-5-{[3-(tert.-butyloxycarbonylamino)-cyclohexyl]-carbonylamino}-6-amino-uracil (cis/trans mixture)

Carried out with O-(benzotriazol-1-yl)-N,N,N′,N′-tetramethyluronium tetrafluoroborate

Mass spectrum (ESI⁺): m/z=396 [M+H]⁺

### (5) 1,3-dimethyl-5-{[1-(tert.-butyloxycarbonyl)-piperidin-3-yl]-carbonylamino}-6-amino-uracil

Carried out with O-(benzotriazol-1-yl)-N,N,N′,N′-tetramethyluronium tetrafluoroborate

Mass spectrum (ESI⁺): m/z=382 [M+H]⁺

### (6) 2-(tert.-butyloxycarbonylamino)-3-(N-benzyl-N-methyl-amino)-propionic acid-dimethylamide

Carried out with dimethylamine in the presence of O-(benzotriazol-1-yl)-N,N,N′,N′-tetramethyluronium tetrafluoroborate and hydroxybenzotriazole in tetrahydrofuran.

R_{f} value: 0.80 (silica gel, methylene chloride/methanol/conc. aqueous ammonia=40:10:1)

Mass spectrum (ESI⁺): m/z=336 [M+H]⁺

### (7) 2-(tert.-butyloxycarbonylamino)-3-(N-benzyl-N-methyl-amino)-propionic acid-amide

Carried out with ammonium carbonate in the presence of O-(benzotriazol-1-yl)-N,N,N′,N′-tetramethyluronium tetrafluoroborate and hydroxybenzotriazole in tetrahydrofuran.

R_{f} value: 0.75 (silica gel, methylene chloride/methanol/conc. aqueous ammonia=40:10:1)

Mass spectrum (ESI⁺): m/z=308 [M+H]⁺

### (8) 2-(tert.-butyloxycarbonylamino)-3-(N-benzyl-N-methyl-amino)-1-(pyrrolidin-1-yl)-propane-1-one

Carried out with pyrrolidine in the presence of O-(benzotriazol-1-yl)-N,N,N′,N′-tetramethyluronium tetrafluoroborate and hydroxybenzotriazole in tetrahydrofuran.

R_{f} value: 0.40 (silica gel, methylene chloride/methanol=9:1)

Mass spectrum (ESI⁺): m/z=362 [M+H]⁺

### (9) 2-(tert.-butyloxycarbonylamino)-3-(N-benzyl-N-methyl-amino)-1-dimethylamino-propane-1-one

Carried out with methylamine (40% aqueous solution) in the presence of O-(benzotriazol-1-yl)-N,N,N′,N′-tetramethyluronium tetrafluoroborate and hydroxybenzotriazole in tetrahydrofuran.

R_{f} value: 0.40 (silica gel, methylene chloride/methanol=9:1)

Mass spectrum (ESI⁺): m/z=322 [M+H]⁺

### (10) 1-(tert.-butyloxycarbonyl)-3-{[(9H-fluoren-9-ylmethoxy)carbonyl]amino}-3-(pyrrolidin-1-ylcarbonyl)-piperidine

Carried out with pyrrolidine in the presence of 0-(benzotriazol-1-yl)-N,N,N′,N′-tetra-methyluronium tetrafluoroborate, hydroxybenzotriazole and Hünig base in dimethyl-formamide. The starting material 1-(tert.-butyloxycarbonyl)-3-{[(9H-fluoren-9-ylmethoxy)carbonyl]amino}-piperidin-3-yl-carboxylic acid is obtainable from Pharmacore, Inc. (USA).

Rf value: 0.52 (aluminium oxide, methylene chloride/methanol=9:1)

Mass spectrum (ESI⁺): m/z=520 [M+H]⁺

### EXAMPLE VII

1,3-bis-(cyclopropylmethyl)-7-benzyl-8-chloro-xanthine

Prepared from the compound of Example VIII by refluxing with N-chlorosuccinimide in 1,2-dichloroethane.

Mass spectrum (ESI⁺): m/z=407, 409 [M+Na]⁺

The following compounds are obtained analogously to Example VII:

### (1) 1-methyl-3-(cyclopropylmethyl)-7-benzyl-8-chloro-xanthine

Mass spectrum (ESI⁺): m/z=345, 347 [M+H]⁺

### (2) 1,3-diethyl-7-benzyl-8-chloro-xanthine

Mass spectrum (ESI⁺): m/z=355, 357 [M+Na]⁺

### (3) 1-methyl-3-ethyl-7-benzyl-8-chloro-xanthine

Mass spectrum (ESI⁺): m/z=341, 343 [M+Na]⁺

### (4) 1-methyl-3-(4-methoxy-benzyl)-7-benzyl-8-chloro-xanthine

Melting point: 172-175° C.

Mass spectrum (ESI⁺): m/z=411, 413 [M+H]⁺

### (5) 1-methyl-3,7-dibenzyl-8-chloro-xanthine

R_{f} value: 0.72 (silica gel, methylene chloride/methanol/conc. aqueous ammonia=98:2:1)

Mass spectrum (ESI⁺): m/z=381, 383 [M+H]⁺

### (6) 1-methyl-3-[(methoxycarbonyl)-methyl]-7-benzyl-8-chloro-xanthine

R_{f} value: 0.83 (silica gel, methylene chloride/methanol/conc. aqueous ammonia=95:5:1)

Mass spectrum (ESI⁺): m/z=363, 365 [M+H]⁺

### (7) 1-methyl-3-isopropyl-7-benzyl-8-chloro-xanthine

R_{f} value: 0.69 (silica gel, methylene chloride/methanol/conc. aqueous ammonia=98:2:1)

Mass spectrum (EI): m/z=332, 334 [M]⁺

### (8) 1-methyl-3-hexyl-7-benzyl-8-chloro-xanthine

R_{f} value: 0.68 (silica gel, methylene chloride/methanol/conc. aqueous ammonia=98:2:1)

Mass spectrum (ESI⁺): m/z=375, 377 [M+H]⁺

### (9) 1-methyl-3-(2-trimethylsilanyl-ethoxymethyl)-7-benzyl-8-chloro-xanthine

Mass spectrum (ESI⁺): m/z=421, 423 [M+H]⁺

### (10) 1-methyl-3-(2-methoxy-ethyl)-7-benzyl-8-chloro-xanthine

R_{f} value: 0.84 (silica gel, methylene chloride/methanol/conc. aqueous ammonia=9:1:0.1)

Mass spectrum (ESI⁺): m/z=349, 351 [M+H]⁺

### (11) 1-methyl-3-cyanomethyl-7-benzyl-8-chloro-xanthine

R_{f} value: 0.90 (silica gel, methylene chloride/methanol/conc. aqueous ammonia=95:5:1)

Mass spectrum (ESI⁺): m/z=352 [M+Na]⁺

### (12) 1-methyl-3-(2-hydroxy-ethyl)-7-benzyl-8-chloro-xanthine

R_{f} value: 0.48 (silica gel, methylene chloride/methanol/conc. aqueous ammonia=9:1:0.1)

Mass spectrum (ESI⁺): m/z=335, 337 [M+H]⁺

### (13) 1-methyl-3-(2-trimethylsilanyl-ethoxymethyl)-7-benzyl-8-chloro-xanthine

Mass spectrum (ESI⁺): m/z=421, 423 [M+H]⁺

### (14) 1-methyl-3-(2-trimethylsilanyl-ethoxymethyl)-7-(2-cyano-benzyl)-8-chloro-xanthine

Mass spectrum (ESI⁺): m/z=468, 470 [M+Na]⁺

### EXAMPLE VIII

### 1,3-bis-(cyclopropylmethyl)-7-benzyl-xanthine

Prepared from 7-benzyl-xanthine by reacting with cyclopropylmethylbromide in dimethylformamide in the presence of caesium carbonate

Mass spectrum (ESI⁺): m/z=351 [M+H]⁺

The following compounds are obtained analogously to Example VIII:

### (1) 3-(cyclopropylmethyl)-7-benzyl-xanthine

Mass spectrum (ESI⁺): m/z=297 [M+H]⁺

### (2) 1,3-diethyl-7-benzyl-xanthine

Carried out with potassium carbonate

Mass spectrum (ESI⁺): m/z=321 [M+Na]⁺

### (3) 3-ethyl-7-benzyl-xanthine

Carried out with potassium carbonate

Mass spectrum (ESI⁺): m/z=293 [M+Na]⁺

### (4) 3-(4-methoxy-benzyl)-7-benzyl-xanthine

Carried out with 1,8-diazabicyclo[5.4.0]undec-7-ene

Mass spectrum (ESI⁺): m/z=363 [M+H]⁺

### (5) 3,7-dibenzyl-xanthine

Carried out with 1,8-diazabicyclo[5.4.0]undec-7-ene

Melting point: 184-187° C.

Mass spectrum (ESI⁺): m/z=333 [M+H]⁺

### (6) 3-[(methoxycarbonyl)-methyl]-7-benzyl-xanthine

Carried out with 1,8-diazabicyclo[5.4.0]undec-7-ene

R_{f} value: 0.21 (silica gel, methylene chloride/methanol/conc. aqueous ammonia=95:5:1)

Mass spectrum (ESI⁺): m/z=315 [M+H]⁺

### (7) 3-isopropyl-7-benzyl-xanthine

Carried out with 1,8-diazabicyclo[5.4.0]undec-7-ene

Melting point: 215-218° C.

Mass spectrum (ESI⁺): m/z=285 [M+H]⁺

### (8) 3-hexyl-7-benzyl-xanthine

Carried out with 1,8-diazabicyclo[5.4.0]undec-7-ene

R_{f} value: 0.52 (silica gel, methylene chloride/methanol/conc. aqueous ammonia=9:1:0.1)

Mass spectrum (ESI⁺): m/z=327 [M+H]⁺

### (9) 3-(2-trimethylsilanyl-ethoxymethyl)-7-benzyl-xanthine

Carried out with 1,8-diazabicyclo[5.4.0]undec-7-ene

Mass spectrum (ESI⁺): m/z=373 [M+H]⁺

### (10) 3-(2-methoxy-ethyl)-7-benzyl-xanthine

Carried out with 1,8-diazabicyclo[5.4.0]undec-7-ene

R_{f} value: 0.45 (silica gel, methylene chloride/methanol/conc. aqueous ammonia=9:1:0.1)

Mass spectrum (ESI⁺): m/z=301 [M+H]⁺

### (11) 3-cyanomethyl-7-benzyl-xanthine

Carried out with 1,8-diazabicyclo[5.4.0]undec-7-ene

R_{f} value: 0.41 (silica gel, methylene chloride/methanol/conc. aqueous ammonia=9:1:0.1)

Mass spectrum (ESI⁻): m/z=280 [M−H]⁻

### (12) 3-(2-hydroxy-ethyl)-7-benzyl-xanthine

Carried out with 1,8-diazabicyclo[5.4.0]undec-7-ene

R_{f} value: 0.28 (silica gel, methylene chloride/methanol/conc. aqueous ammonia=9:1:0.1)

Mass spectrum (ESI⁺): m/z=287 [M+H]⁺

### (13) 3-(2-trimethylsilanyl-ethoxymethyl)-7-benzyl-xanthine

Carried out with 1,8-diazabicyclo[5.4.0]undec-7-ene

R_{f} value: 0.30 (silica gel, methylene chloride/methanol=98:2)

Mass spectrum (ESI⁺): m/z=373 [M+H]⁺

### (14) 3-[(methoxycarbonyl)methyl]-7-(3-methyl-2-buten-1-yl)-8-[3-(tert.-butyloxycarbonylamino)-piperidin-1-yl]-xanthine

Carried out with 1,8-diazabicyclo[5.4.0]undec-7-ene

R_{f} value: 0.31 (silica gel, methylene chloride/methanol/conc. aqueous ammonia=90:10:1)

Mass spectrum (ESI⁺): m/z=491 [M+H]⁺

### (15) 3-(2-trimethylsilanyl-ethoxymethyl)-7-(2-cyano-benzyl)-xanthine

Carried out in the presence of 1,8-diazabicyclo[5.4.0]undec-7-ene.

Mass spectrum (ESI⁺): m/z=420 [M+Na]⁺

### EXAMPLE IX

### 1-ethyl-3-methyl-7-(3-methyl-2-buten-1-yl)-8-bromo-xanthine

Prepared from 3-methyl-7-(3-methyl-2-buten-1-yl)-8-bromo-xanthine by reacting with ethyl bromide in the presence of potassium carbonate in dimethylformamide at 70° C.

Mass spectrum (ESI⁺): m/z=341, 343 [M+H]⁺

Retention time: 1.48 min (HPLC, Multosphere 100FBS, 50 mm, 50% acetonitrile)

The following compounds are obtained analogously to Example IX:

### (1) 1-propyl-3-methyl-7-(3-methyl-2-buten-1-yl)-8-bromo-xanthine

Mass spectrum (ESI⁺): m/z=355, 357 [M+H]⁺

### (2) 1-butyl-3-methyl-7-(3-methyl-2-buten-1-yl)-8-bromo-xanthine

Mass spectrum (ESI⁺): m/z=369, 371 [M+H]⁺

### (3) 1-(2-propyl)-3-methyl-7-(3-methyl-2-buten-1-yl)-8-bromo-xanthine

Retention time: 2.11 min (HPLC, Multosphere 100FBS, 50 mm, 50% acetonitrile)

### (4) 1-(2-methylpropyl)-3-methyl-7-(3-methyl-2-buten-1-yl)-8-bromo-xanthine

Retention time: 2.46 min (HPLC, Multosphere 100FBS, 50 mm, 50% acetonitrile)

### (5) 1-(2-propen-1-yl)-3-methyl-7-(3-methyl-2-buten-1-yl)-8-bromo-xanthine

Retention time: 1.55 min (HPLC, Multosphere 100FBS, 50 mm, 50% acetonitrile)

Mass spectrum (ESI⁺): m/z=353, 355 [M+H]⁺

### (6) 1-(2-propyn-1-yl)-3-methyl-7-(3-methyl-2-buten-1-yl)-8-bromo-xanthine

Retention time: 1.20 min (HPLC, Multosphere 100FBS, 50 mm, 50% acetonitrile)

Mass spectrum (ESI⁺): m/z=351, 353 [M+H]⁺

### (7) 1-(cyclopropylmethyl)-3-methyl-7-(3-methyl-2-buten-1-yl)-8-bromo-xanthine

Retention time: 2.19 min (HPLC, Multosphere 100FBS, 50 mm, 50% acetonitrile)

Mass spectrum (ESI⁺): m/z=367, 369 [M+H]⁺

### (8) 1-benzyl-3-methyl-7-(3-methyl-2-buten-1-yl)-8-bromo-xanthine

Retention time: 2.40 min (HPLC, Multosphere 100FBS, 50 mm, 50% acetonitrile)

Mass spectrum (ESI⁺): m/z=403, 405 [M+H]⁺

### (9) 1-(2-phenylethyl)-3-methyl-7-(3-methyl-2-buten-1-yl)-8-bromo-xanthine

Retention time: 3.29 min (HPLC, Multosphere 100FBS, 50 mm, 50% acetonitrile)

### (10) 1-(3-phenylpropyl)-3-methyl-7-(3-methyl-2-buten-1-yl)-8-bromo-xanthine

Retention time: 2.95 min (HPLC, Multosphere 100FBS, 50 mm, 50% acetonitrile)

### (11) 1-(2-hydroxyethyl)-3-methyl-7-(3-methyl-2-buten-1-yl)-8-bromo-xanthine

Retention time: 2.35 min (HPLC, Multosphere 100FBS, 50 mm, 20% acetonitrile)

### (12) 1-(2-methoxyethyl)-3-methyl-7-(3-methyl-2-buten-1-yl)-8-bromo-xanthine

Retention time: 2.54 min (HPLC, Multosphere 100FBS, 50 mm, 30% acetonitrile)

### (13) 1-(3-hydroxypropyl)-3-methyl-7-(3-methyl-2-buten-1-yl)-8-bromo-xanthine

Retention time: 2.52 min (HPLC, Multosphere 100FBS, 50 mm, 20% acetonitrile)

### (14) 1-[2-(dimethylamino)ethyl]-3-methyl-7-(3-methyl-2-buten-1-yl)-8-bromo-xanthine

Retention time: 2.73 min (HPLC, Multosphere 100FBS, 50 mm, 5% acetonitrile)

### (15) 1-[3-(dimethylamino)propyl]-3-methyl-7-(3-methyl-2-buten-1-yl)-8-bromo-xanthine

Retention time: 2.79 min (HPLC, Multosphere 100FBS, 50 mm, 5% acetonitrile)

### (16) 1-methyl-3-(cyclopropylmethyl)-7-benzyl-xanthine

Carried out with methyl iodide at ambient temperature

Mass spectrum (ESI⁺): m/z=311 [M+H]⁺

### (17) 1-methyl-3-ethyl-7-benzyl-xanthine

Carried out with methyl iodide at ambient temperature

### (18) 1-methyl-3-(4-methoxy-benzyl)-7-benzyl-xanthine

Carried out with methyl iodide at ambient temperature

Mass spectrum (ESI⁺): m/z=377 [M+H]⁺

### (19) 1-methyl-3,7-dibenzyl-xanthine

Carried out with methyl iodide at ambient temperature

R_{f} value: 0.51 (silica gel, methylene chloride/methanol/conc. aqueous ammonia=95:5:1)

Mass spectrum (ESI⁺): m/z=347 [M+H]⁺

### (20) 1-methyl-3-[(methoxycarbonyl)-methyl]-7-benzyl-xanthine

Carried out with methyl iodide at ambient temperature

Melting point: 182° C.

Mass spectrum (ESI⁺): m/z=329 [M+H]⁺

### (21) 1-methyl-3-isopropyl-7-benzyl-xanthine Carried out with methyl iodide at ambient temperature

R_{f} value: 0.66 (silica gel, methylene chloride/methanol/conc. aqueous ammonia=9:1:0.1)

Mass spectrum (ESI⁺): m/z=299 [M+H]⁺

### (22) 1-methyl-3-hexyl-7-benzyl-xanthine

Carried out with methyl iodide at ambient temperature

R_{f} value: 0.77 (silica gel, methylene chloride/methanol/conc. aqueous ammonia=95:5:1)

Mass spectrum (ESI⁺): m/z=341 [M+H]⁺

### (23) 1-methyl-3-(2-trimethylsilanyl-ethoxymethyl)-7-benzyl-xanthine

Carried out with methyl iodide at ambient temperature

### (24) 1-methyl-3-(2-methoxy-ethyl)-7-benzyl-xanthine

Carried out with methyl iodide at ambient temperature

R_{f} value: 0.70 (silica gel, methylene chloride/methanol/conc. aqueous ammonia=9:1:0.1)

Mass spectrum (ESI⁺): m/z=315 [M+H]⁺

### (25) 1-methyl-3-cyanomethyl-7-benzyl-xanthine

Carried out with methyl iodide at ambient temperature

R_{f} value: 0.74 (silica gel, methylene chloride/methanol/conc. aqueous ammonia=9:1:0.1)

Mass spectrum (ESI⁺): m/z=296 [M+H]⁺

### (26) 1-methyl-3-(2-hydroxy-ethyl)-7-benzyl-xanthine

Carried out with methyl iodide at ambient temperature

R_{f} value: 0.44 (silica gel, methylene chloride/methanol/conc. aqueous ammonia=9:1:0.1)

Mass spectrum (ESI⁺): m/z=301 [M+H]⁺

### (27) 1-methyl-3-(2-trimethylsilanyl-ethoxymethyl)-7-benzyl-xanthine

Carried out with methyl iodide at ambient temperature

R_{f} value: 0.44 (silica gel, methylene chloride/methanol=95:5)

Mass spectrum (ESI⁺): m/z=387 [M+H]⁺

### (28) 1-(2-phenyl-ethyl)-3-methyl-7-benzyl-8-chloro-xanthine

Carried out with 2-phenyl-ethyl bromide at 60° C.

Mass spectrum (ESI⁺): m/z=395, 397 [M+H]⁺

### (29) 1-(2-phenyl-ethyl)-3-methyl-7-cyclopropylmethyl-8-chloro-xanthine

Carried out with 2-phenyl-ethyl bromide at 60° C.

Mass spectrum (ESI⁺): m/z=359, 361 [M+H]⁺

### (30) 1-(2-phenyl-ethyl)-3-methyl-7-(2-butyn-1-yl)-8-chloro-xanthine

Mass spectrum (ESI⁺): m/z=357, 359 [M+H]⁺

### (31) 1-(2-phenyl-ethyl)-3-methyl-7-(3-methyl-2-buten-1-yl)-8-chloro-xanthine

Mass spectrum (ESI⁺): m/z=395, 397 [M+Na]⁺

### (32) 1-[(methoxycarbonyl)-methyl]-3-methyl-7-(3-methyl-2-buten-1-yl)-8-[(S)-3-(tert.-butyloxycarbonylamino)-piperidin-1-yl]-xanthine

Carried out with methyl bromoacetate at 50° C.

Melting point: 143-145° C.

Mass spectrum (ESI⁺): m/z=505 [M+H]⁺

### (33) 1-[3-(methoxycarbonyl)-propyl]-3-methyl-7-(3-methyl-2-buten-1-yl)-8-[(S)-3-(tert.-butyloxycarbonylamino)-piperidin-1-yl]-xanthine

Carried out with methyl 4-bromobutyrate at 50° C.

Melting point: 130-131° C.

Mass spectrum (ESI⁺): m/z=533 [M+H]⁺

### (34) 1-{2-[4-(ethoxycarbonyl)-phenyl]-ethyl}-3-methyl-7-(3-methyl-2-buten-1-yl)-8-[(S)-3-(tert.-butyloxycarbonylamino)-piperidin-1-yl]-xanthine

Carried out with ethyl 4-(2-bromo-ethyl)-benzoate at 50° C.

R_{f} value: 0.40 (silica gel, cyclohexane/ethyl acetate=1:1)

Mass spectrum (ESI⁺): m/z=609 [M+H]⁺

### (35) 1-[2-(methoxycarbonyl)-ethyl]-3-methyl-7-(3-methyl-2-buten-1-yl)-8-[(S)-3-(tert.-butyloxycarbonylamino)-piperidin-1-yl]-xanthine

Carried out with methyl 3-bromopropionate at 50° C.

R_{f} value: 0.35 (silica gel, cyclohexane/ethyl acetate=1:1)

Mass spectrum (ESI⁺): m/z=519 [M+H]⁺

### (36) 1-cyanomethyl-3-methyl-7-(3-methyl-2-buten-1-yl)-8-bromo-xanthine

R_{f} value: 0.58 (silica gel, petroleum ether/ethyl acetate/methanol=6:3.5:0.5)

Mass spectrum (ESI⁺): m/z=352, 354 [M+H]⁺

### (37) 1-(2-phenyl-2-oxo-ethyl)-3-methyl-7-(3-methyl-2-buten-1-yl)-8-[3-(tert.-butyloxycarbonylamino)-piperidin-1-yl]-xanthine

R_{f} value: 0.30 (silica gel, petroleum ether/ethyl acetate/methanol=7:2.5:0.5)

Mass spectrum (ESI⁺): m/z=551 [M+H]⁺

### (38) 1-[2-(2-methoxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=581 [M+H]⁺

### (39) 1-[2-(thiophen-3-yl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=557 [M+H]⁺

### (40) 1-[2-(4-methoxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=581 [M+H]⁺

### (41) 1-(2-phenyl-2-oxo-ethyl)-3-methyl-7-(3-methyl-2-buten-1-yl)-8-[(S)-3-(tert.-butyloxycarbonylamino)-piperidin-1-yl]-xanthine

### (42) 1-(2-phenyl-2-oxo-ethyl)-3-methyl-7-(3-methyl-2-buten-1-yl)-8-[(R)-3-(tert.-butyloxycarbonylamino)-piperidin-1-yl]-xanthine

Mass spectrum (ESI⁺): m/z=551 [M+H]⁺

### (43) 1-(phenylsulphanylmethyl)-3-methyl-7-(3-methyl-2-buten-1-yl)-8-[3-(tert.-butyloxycarbonylamino)-piperidin-1-yl]-xanthine

R_{f} value: 0.30 (silica gel, petroleum ether/ethyl acetate/methanol=7:2:1)

Mass spectrum (ESI⁺): m/z=555 [M+H]⁺

### (44) 1-[2-(3-methoxy-phenyl)-2-oxo-ethyl]-3-methyl-7-(3-methyl-2-buten-1-yl)-8-[3-(tert.-butyloxycarbonylamino)-piperidin-1-yl]-xanthine

R_{f} value: 0.30 (silica gel, petroleum ether/ethyl acetate/methanol=7:2:1)

### (45) 1-[2-(4-methyl-phenyl)-2-oxo-ethyl]-3-methyl-7-(3-methyl-2-buten-1-yl)-8-[3-(tert.-butyloxycarbonylamino)-piperidin-1-yl]-xanthine

R_{f} value: 0.20 (silica gel, petroleum ether/ethyl acetate/methanol=7:2:1)

Mass spectrum (ESI⁺): m/z=565 [M+H]⁺

### (46) 1-(2-methoxycarbonyl-2-propen-1-yl)-3-methyl-7-(3-methyl-2-buten-1-yl)-8-[3-(tert.-butyloxycarbonylamino)-piperidin-1-yl]-xanthine

R_{f} value: 0.15 (silica gel, petroleum ether/ethyl acetate/methanol=75:20:5)

Mass spectrum (ESI⁺): m/z=531 [M+H]⁺

### (47) 1-(3-oxo-3-phenyl-propyl)-3-methyl-7-(3-methyl-2-buten-1-yl)-8-[3-(tert.-butyloxycarbonylamino)-piperidin-1-yl]-xanthine

Mass spectrum (ESI⁺): m/z=565 [M+H]⁺

### (49) 1-(2-oxo-propyl)-3-methyl-7-(3-methyl-2-buten-1-yl)-8-[3-(tert.-butyloxycarbonylamino)-piperidin-1-yl]-xanthine

R_{f} value: 0.10 (silica gel, petroleum ether/ethyl acetate/methanol=6:3:1)

Mass spectrum (ESI⁺): m/z=489 [M+H]⁺

### (50) 1-(2-phenyl-2-oxo-ethyl)-3-methyl-7-(2-cyano-benzyl)-8-[3-(tert.-butyloxycarbonylamino)-piperidin-1-yl]-xanthine

Mass spectrum (ESI⁺): m/z=598 [M+H]⁺

### (51) 1-(2-phenyl-ethyl)-3-methyl-7-(2-cyano-benzyl)-8-[3-(tert.-butyloxycarbonylamino)-piperidin-1-yl]-xanthine

R_{f} value: 0.50 (silica gel, cyclohexane/ethyl acetate=1:1)

Mass spectrum (ESI⁺): m/z=584 [M+H]⁺

### (52) 1-(3-methoxycarbonyl-2-propen-1-yl)-3-methyl-7-(3-methyl-2-buten-1-yl)-8-[3-(tert.-butyloxycarbonylamino)-piperidin-1-yl]-xanthine

Mass spectrum (ESI⁺): m/z=531 [M+H]⁺

### (53) 1-[2-(2,5-dimethoxy-phenyl)-2-oxo-ethyl]-3-methyl-7-(3-methyl-2-buten-1-yl)-8-[3-(tert.-butyloxycarbonylamino)-piperidin-1-yl]-xanthine

R_{f} value: 0.31 (silica gel, cyclohexane/ethyl acetate/methanol=6:3:1)

### (54) 1-[2-(4-fluoro-phenyl)-2-oxo-ethyl]-3-methyl-7-(3-methyl-2-buten-1-yl)-8-[3-(tert.-butyloxycarbonylamino)-piperidin-1-yl]-xanthine

R_{f} value: 0.40 (silica gel, petroleum ether/ethyl acetate/methanol=6:3:1)

### (55) 1-[2-(3-hydroxy-phenyl)-2-oxo-ethyl]-3-methyl-7-(3-methyl-2-buten-1-yl)-8-[3-(tert.-butyloxycarbonylamino)-piperidin-1-yl]-xanthine

(By reacting Example II(18) with 2-bromo-1-[3-(tert.-butyl-dimethyl-silanyloxy)-phenyl]-ethanone in the presence of potassium tert. butoxide in dimethylformamide at ambient temperature)

Mass spectrum (ESI⁺): m/z=567 [M+H]⁺

### (56) 1-(3-methoxycarbonyl-2-propen-1-yl)-3-methyl-7-(2-cyano-benzyl)-8-[3-(tert.-butyloxycarbonylamino)-piperidin-1-yl]-xanthine

R_{f} value: 0.50 (silica gel, cyclohexane/ethyl acetate=1:1)

Mass spectrum (ESI⁺): m/z=600 [M+Na]⁺

### (57) 1-[(pyridin-2-yl)methyl]-3-methyl-7-(2-cyano-benzyl)-8-[3-(tert.-butyloxycarbonylamino)-piperidin-1-yl]-xanthine

Mass spectrum (ESI⁺): m/z=571 [M+H]⁺

### (58) 1-(2-phenyl-2-oxo-ethyl)-3-[(methoxycarbonyl)methyl]-7-(3-methyl-2-buten-1-yl)-8-[3-(tert.-butyloxycarbonylamino)-piperidin-1-yl]-xanthine

R_{f} value: 0.68 (silica gel, methylene chloride/methanol/conc. aqueous ammonia=90:10:1)

Mass spectrum (ESI⁺): m/z=609 [M+H]⁺

### (59) 1-(2-phenyl-2-oxo-ethyl)-3-methyl-7-(3-methyl-2-buten-1-yl)-8-chloro-xanthine

R_{f} value: 0.55 (silica gel, cyclohexane/ethyl acetate/methanol=6:3:1)

Mass spectrum (ESI⁺): m/z=387, 389 [M+H]⁺

### (60) 1-[2-(3-allyloxycarbonylamino-phenyl)-2-oxo-ethyl]-3-methyl-7-(3-methyl-2-buten-1-yl)-8-[3-(tert.-butyloxycarbonylamino)-piperidin-1-yl]-xanthine

R_{f} value: 0.40 (silica gel, cyclohexane/ethyl acetate/methanol=6:3:1)

Mass spectrum (ESI⁺): m/z=650 [M+H]⁺

### (61) 1-[2-(3-nitro-phenyl)-2-oxo-ethyl]-3-methyl-7-(3-methyl-2-buten-1-yl)-8-chloro-xanthine

Mass spectrum (ESI⁺): m/z=432, 434 [M+H]⁺

### (62) 1-[2-(2-bromo-5-dimethylamino-phenyl)-2-oxo-ethyl]-3-methyl-7-(3-methyl-2-buten-1-yl)-8-[3-(tert.-butyloxycarbonylamino)-piperidin-1-yl]-xanthine

### (63) 1-[(thiazol-2-yl)methyl]-3-methyl-7-(3-methyl-2-buten-1-yl)-8-[3-(tert.-butyloxycarbonylamino)-piperidin-1-yl]-xanthine

R_{f} value: 0.34 (silica gel, methylene chloride/methanol=95:5)

Mass spectrum (ESI⁺): m/z=530 [M+H]⁺

### (64) 1-[(benzo[d]isothiazol-3-yl)methyl]-3-methyl-7-(3-methyl-2-buten-1-yl)-8-[3-(tert.-butyloxycarbonylamino)-piperidin-1-yl]-xanthine

R_{f} value: 0.40 (silica gel, cyclohexane/ethyl acetate=1:1)

Mass spectrum (ESI⁺): m/z=580 [M+H]⁺

### (65) 1-[(isoxazol-3-yl)methyl]-3-methyl-7-(3-methyl-2-buten-1-yl)-8-[3-(tert.-butyloxycarbonylamino)-piperidin-1-yl]-xanthine

R_{f} value: 0.20 (silica gel, ethyl acetate)

Mass spectrum (ESI⁺): m/z=514 [M+H]⁺

### (66) 1-[(1-naphthyl)methyl]-3-methyl-7-(3-methyl-2-buten-1-yl)-8-[3-(tert.-butyloxycarbonylamino)-piperidin-1-yl]-xanthine

R_{f} value: 0.41 (silica gel, cyclohexane/ethyl acetate=1:1)

Mass spectrum (ESI⁺): m/z=595 [M+Na]⁺

### (67) 1-[(benzo[d]isoxazol-3-yl)methyl]-3-methyl-7-(3-methyl-2-buten-1-yl)-8-[3-(tert.-butyloxycarbonylamino)-piperidin-1-yl]-xanthine

R_{f} value: 0.60 (silica gel, methylene chloride/methanol=95:5)

Mass spectrum (ESI⁺): m/z=564 [M+H]⁺

### (68) 1-cyanomethyl-3-methyl-7-(2-cyano-benzyl)-8-[3-(tert.-butyloxycarbonylamino)-piperidin-1-yl]-xanthine

R_{f} value: 0.40 (silica gel, cyclohexane/ethyl acetate=1:1)

Mass spectrum (ESI⁺): m/z=541 [M+Na]⁺

### (69) 1-[2-(2-nitro-phenyl)-2-oxo-ethyl]-3-methyl-7-(3-methyl-2-buten-1-yl)-8-chloro-xanthine

R_{f} value: 0.25 (silica gel, cyclohexane/ethyl acetate/methanol=7:2:1)

Mass spectrum (ESI⁺): m/z=432, 434 [M+H]⁺

### (70) 1-[(6-methyl-pyridin-2-yl)methyl]-3-methyl-7-(3-methyl-2-buten-1-yl)-8-[3-(tert.-butyloxycarbonylamino)-piperidin-1-yl]-xanthine

Carried out in the presence of sodium iodide.

R_{f} value: 0.47 (silica gel, ethyl acetate)

Mass spectrum (ESI⁺): m/z=538 [M+H]⁺

### (71) 1-cyanomethyl-3-methyl-7-(2-cyano-benzyl)-8-[3-(tert.-butyloxycarbonylamino)-piperidin-1-yl]-xanthine

Rf value: 0.40 (silica gel, cyclohexane/ethyl acetate=1:1)

### (72) 1-[2-(2-methoxy-phenyl)-2-oxo-ethyl]-3-methyl-7-(3-methyl-2-buten-1-yl)-8-chloro-xanthine

Mass spectrum (ESI⁺): m/z=417, 419 [M+H]⁺

### (73) 1-methyl-3-(2-trimethylsilanyl-ethoxymethyl)-7-(2-cyano-benzyl)-xanthine

Mass spectrum (ESI⁺): m/z=412 [M+H]⁺

### (74) 1-[(3-methyl-pyridin-2-yl)methyl]-3-methyl-7-(3-methyl-2-buten-1-yl)-8-[3-(tert.-butyloxycarbonylamino)-piperidin-1-yl]-xanthine

Rf value: 0.27 (silica gel, ethyl acetate)

Mass spectrum (ESI⁺): m/z=538 [M+H]⁺

### (75) 1-[(5-methyl-pyridin-2-yl)methyl]-3-methyl-7-(3-methyl-2-buten-1-yl)-8-[3-(tert.-butyloxycarbonylamino)-piperidin-1-yl]-xanthine

Rf value: 0.45 (silica gel, ethyl acetate)

Mass spectrum (ESI⁺): m/z=538 [M+H]⁺

### (76) 1-[(4-methyl-pyridin-2-yl)methyl]-3-methyl-7-(3-methyl-2-buten-1-yl)-8-[3-(tert.-butyloxycarbonylamino)-piperidin-1-yl]xanthine

Rf value: 0.26 (silica gel, ethyl acetate)

Mass spectrum (ESI⁺): m/z=538 [M+H]⁺

### (77) 1-[(5-nitro-isoquinolin-1-yl)methyl]-3-methyl-7-(3-methyl-2-buten-1-yl)-8-[3-(tert.-butyloxycarbonylamino)-piperidin-1-yl]-xanthine

Rf value: 0.54 (silica gel, methylene chloride/methanol=95:5)

### (78) 1-[(2-oxo-1,2-dihydro-quinolin-4-yl)methyl]-3-methyl-7-(3-methyl-2-buten-1-yl)-8-[3-(tert.-butyloxycarbonylamino)-piperidin-1-yl]-xanthine

Rf value: 0.38 (silica gel, methylene chloride/methanol=95:5)

Mass spectrum (ESI⁺): m/z=590 [M+H]⁺

### (79) 1-[2-(3-cyano-phenyl)-2-oxo-ethyl]-3-methyl-7-(3-methyl-2-buten-1-yl)-8-chloro-xanthine

Rf value: 0.52 (silica gel, cyclohexane/ethyl acetate=1:1)

Mass spectrum (ESI⁺): m/z=434, 436 [M+Na]⁺

### (80) 1-[2-(3-aminosulphonyl-phenyl)-2-oxo-ethyl]-3-methyl-7-(3-methyl-2-buten-1-yl)-8-chloro-xanthine

Rf value: 0.25 (silica gel, cyclohexane/ethyl acetate=1:1)

Mass spectrum (ESI⁺): m/z=466, 468 [M+H]⁺

### (81) 1-[2-(3-aminocarbonyl-phenyl)-2-oxo-ethyl]-3-methyl-7-(3-methyl-2-buten-1-yl)-8-chloro-xanthine

Rf value: 0.10 (silica gel, cyclohexane/ethyl acetate=1:1)

Mass spectrum (ESI⁺): m/z=430, 432 [M+H]⁺

### (82) 1-(2-phenoxy-ethyl)-3-methyl-7-(3-methyl-2-buten-1-yl)-8-[3-(tert.-butyloxy-carbonylamino)-piperidin-1-yl]-xanthine

Rf value: 0.75 (silica gel, cyclohexane/ethyl acetate=1:4)

Mass spectrum (ESI⁺): m/z=553 [M+H]⁺

### EXAMPLE X

### 1-benzyl-3-(tert.-butyloxycarbonylamino)-4-methyl-piperidine

Prepared by catalytic hydrogenation of 1-benzyl-3-(tert.-butyloxycarbonylamino)-4-methyl-pyridinium-bromide in methanol in the presence of platinum dioxide under a hydrogen pressure of 4 bar.

Mass spectrum (EI): m/z=304 [M]⁺

### EXAMPLE XI

### 1-benzyl-3-(tert.-butyloxycarbonylamino)-4-methyl-pyridinium-bromide

Prepared by reacting 3-(tert.-butyloxycarbonylamino)-4-methyl-pyridine with benzyl bromide in toluene

Melting point: 200-201° C.

### EXAMPLE XII

### 1-[2-(2,4,6-trimethyl-phenyl)-ethyl]-3-methyl-7-(3-methyl-2-buten-1-yl)-8-bromo-xanthine

Prepared by reacting 3-methyl-7-(3-methyl-2-buten-1-yl)-8-bromo-xanthine with 2-(2,4,6-trimethyl-phenyl)-ethanol in the presence of triphenylphosphine and diisopropylazodicarboxylate in tetrahydrofuran at ambient temperature

R_{f} value: 0.40 (silica gel, methylene chloride/ethyl acetate=15:1)

Mass spectrum (ESI⁺): m/z=459, 461 [M+H]⁺

The following compounds are obtained analogously to Example XII:

### (1) 1-[2-(2,4-dichloro-phenyl)-ethyl]-3-methyl-7-(3-methyl-2-buten-1-yl)-8-bromo-xanthine

R_{f} value: 0.40 (silica gel, methylene chloride/ethyl acetate=15:1)

Mass spectrum (EI): m/z=484, 486, 488 [M]⁺

### (2) 1-[2-(thiophen-2-yl)-ethyl]-3-methyl-7-(3-methyl-2-buten-1-yl)-8-bromo-xanthine

R_{f} value: 0.50 (silica gel, methylene chloride/ethyl acetate=15:1)

Mass spectrum (EI): m/z=422, 424 [M]⁺

### (3) 1-[2-(thiophen-3-yl)-ethyl]-3-methyl-7-(3-methyl-2-buten-1-yl)-8-bromo-xanthine

Melting point: 173.8-174.5° C.

Mass spectrum (ESI⁺): m/z=445, 447 [M+Na]⁺

### (4) 1-[2-(4-tert.-butyl-phenyl)-ethyl]-3-methyl-7-(3-methyl-2-buten-1-yl)-8-bromo-xanthine

R_{f} value: 0.85 (silica gel, methylene chloride/methanol=30:1)

Mass spectrum (ESI⁺): m/z=473, 475 [M+H]⁺

### (5) 1-[2-(4-fluoro-phenyl)-ethyl]-3-methyl-7-(3-methyl-2-buten-1-yl)-8-bromo-xanthine

R_{f} value: 0.70 (silica gel, methylene chloride/ethyl acetate=15:1)

### (6) 1-[2-(4-methoxy-phenyl)-ethyl]-3-methyl-7-(3-methyl-2-buten-1-yl)-8-bromo-xanthine

R_{f} value: 0.70 (silica gel, methylene chloride/ethyl acetate=15:1)

### (7) 1-[2-(2-fluoro-phenyl)-ethyl]-3-methyl-7-(3-methyl-2-buten-1-yl)-8-chloro-xanthine

R_{f} value: 0.75 (silica gel, methylene chloride/ethyl acetate=20:1)

Mass spectrum (ESI⁺): m/z=391, 393 [M+H]⁺

### (8) 1-[2-(2-methyl-phenyl)-ethyl]-3-methyl-7-(3-methyl-2-buten-1-yl)-8-chloro-xanthine

R_{f} value: 0.60 (silica gel, methylene chloride/ethyl acetate=20:1)

Mass spectrum (ESI⁺): m/z=387, 389 [M+H]⁺

### (9) 1-[2-(3-methyl-phenyl)-ethyl]-3-methyl-7-(3-methyl-2-buten-1-yl)-8-chloro-xanthine

R_{f} value: 0.80 (silica gel, methylene chloride/ethyl acetate=20:1)

Mass spectrum (EI): m/z=386, 388 [M]⁺

### (10) 1-[2-(1-naphthyl)-ethyl]-3-methyl-7-(3-methyl-2-buten-1-yl)-8-chloro-xanthine

R_{f} value: 0.70 (silica gel, methylene chloride/ethyl acetate=20:1)

Mass spectrum (ESI⁺): m/z=423, 425 [M+H]⁺

### (11) 1-[2-(2-naphthyl)-ethyl]-3-methyl-7-(3-methyl-2-buten-1-yl)-8-chloro-xanthine

R_{f} value: 0.72 (silica gel, methylene chloride/ethyl acetate=20:1)

Mass spectrum (ESI⁺): m/z=423, 425 [M+H]⁺

### (12) 1-(4-phenyl-butyl]-3-methyl-7-(3-methyl-2-buten-1-yl)-8-chloro-xanthine

Mass spectrum (ESI⁺): m/z=401, 403 [M+H]⁺

### (13) 1-[2-(3-trifluoromethyl-phenyl)-ethyl]-3-methyl-7-(3-methyl-2-buten-1-yl)-8-chloro-xanthine

R_{f} value: 0.55 (silica gel, petroleum ether/ethyl acetate/methanol=75:20:5)

Mass spectrum (ESI⁺): m/z=463, 465 [M+Na]⁺

### (14) 1-[2-(pyridin-2-yl)-ethyl]-3-methyl-7-(3-methyl-2-buten-1-yl)-8-bromo-xanthine

Mass spectrum (ESI⁺): m/z=417, 419 [M+H]⁺

### (15) 1-[2-(pyrrol-1-yl)-ethyl]-3-methyl-7-(3-methyl-2-buten-1-yl)-8-chloro-xanthine

R_{f} value: 0.40 (silica gel, petroleum ether/ethyl acetate/methanol=75:20:5)

Mass spectrum (ESI⁺): m/z=384, 386 [M+Na]⁺

### (16) 1-[2-([1,2,3]triazol-1-yl)-ethyl]-3-methyl-7-(3-methyl-2-buten-1-yl)-8-chloro-xanthine

R_{f} value: 0.22 (silica gel, petroleum ether/ethyl acetate/methanol=7:2:1)

Mass spectrum (ESI⁺): m/z=364, 366 [M+H]⁺

### (17) 1-[2-(pyridin-4-yl)-ethyl]-3-methyl-7-(3-methyl-2-buten-1-yl)-8-chloro-xanthine

R_{f} value: 0.15 (silica gel, petroleum ether/ethyl acetate/methanol=7:2:1)

Mass spectrum (ESI⁺): m/z=374, 376 [M+H]⁺

### (18) 1-(3-butyn-1-yl)-3-methyl-7-(3-methyl-2-buten-1-yl)-8-bromo-xanthine

R_{f} value: 0.45 (silica gel, petroleum ether/ethyl acetate=7:3)

Mass spectrum (ESI⁺): m/z=387, 389 [M+Na]⁺

### (19) 1-(3-butene-1-yl)-3-methyl-7-(3-methyl-2-buten-1-yl)-8-bromo-xanthine

R_{f} value: 0.45 (silica gel, petroleum ether/ethyl acetate=7:3)

Mass spectrum (ESI⁺): m/z=389, 391 [M+Na]⁺

### (20) 1-(4-pentyn-1-yl)-3-methyl-7-(3-methyl-2-buten-1-yl)-8-chloro-xanthine

R_{f} value: 0.37 (silica gel, petroleum ether/ethyl acetate/methanol=80:15:5)

Mass spectrum (EI): m/z=378, 380 [M]⁺

### (21) 1-(4-penten-1-yl)-3-methyl-7-(3-methyl-2-buten-1-yl)-8-bromo-xanthine

R_{f} value: 0.30 (silica gel, petroleum ether/ethyl acetate=8:2)

Mass spectrum (ESI⁺): m/z=381, 383 [M+H]⁺

### (22) 1-{2-[4-(tert.-butyl-dimethyl-silanyloxy)-phenyl]-ethyl}-3-methyl-7-(3-methyl-2-buten-1-yl)-8-[(S)-3-(tert.-butyloxycarbonylamino)-piperidin-1-yl]-xanthine

R_{f} value: 0.68 (silica gel, cyclohexane/ethyl acetate=3:1)

Mass spectrum (ESI⁺): m/z=667 [M+H]⁺

### (23) 1-{2-[3-(tert.-butyl-dimethyl-silanyloxy)-phenyl]-ethyl}-3-methyl-7-(3-methyl-2-buten-1-yl)-8-[(S)-3-(tert.-butyloxycarbonylamino)-piperidin-1-yl]-xanthine

R_{f} value: 0.60 (silica gel, cyclohexane/ethyl acetate=1:1)

Mass spectrum (ESI⁺): m/z=667 [M+H]⁺

### (24) 1-[2-(pyridin-3-yl)-ethyl]-3-methyl-7-(3-methyl-2-buten-1-yl)-8-bromo-xanthine

R_{f} value: 0.17 (silica gel, petroleum ether/ethyl acetate/methanol/conc. aqueous ammonia=7:2:1:0.1)

Mass spectrum (ESI⁺): m/z=418, 420 [M+H]⁺

### (25) 1-[2-(4-methyl-thiazol-5-yl)-ethyl]-3-methyl-7-(3-methyl-2-buten-1-yl)-8-bromo-xanthine

R_{f} value: 0.55 (silica gel, petroleum ether/ethyl acetate/methanol=5:4:1)

Mass spectrum (ESI⁺): m/z=438, 440 [M+H]⁺

### (26) 1-[2-(3-methoxy-phenyl)-ethyl]-3-methyl-7-(3-methyl-2-buten-1-yl)-8-bromo-xanthine

R_{f} value: 0.60 (silica gel, petroleum ether/ethyl acetate/methanol=7:2.5:0.5)

Mass spectrum (ESI⁺): m/z=447, 449 [M+H]⁺

### (27) 1-[2-(3-bromo-phenyl)-ethyl]-3-methyl-7-(3-methyl-2-buten-1-yl)-8-bromo-xanthine

R_{f} value: 0.60 (silica gel, petroleum ether/ethyl acetate/methanol=7:2.5:0.5)

Mass spectrum (EI): m/z=494, 496, 498 [M]⁺

### (28) 1-[2-(3-chloro-phenyl)-ethyl]-3-methyl-7-(3-methyl-2-buten-1-yl)-8-bromo-xanthine

R_{f} value: 0.60 (silica gel, petroleum ether/ethyl acetate/methanol=7:2.5:0.5)

Mass spectrum (EI): m/z=450, 452, 454 [M]⁺

### (29) 1-[2-(2-chloro-phenyl)-ethyl]-3-methyl-7-(3-methyl-2-buten-1-yl)-8-chloro-xanthine

R_{f} value: 0.65 (silica gel, petroleum ether/ethyl acetate/methanol=7:2.5:0.5)

Mass spectrum (ESI⁺): m/z=407, 409, 411 [M+H]⁺

### (30) 1-[2-(2-methoxy-phenyl)-ethyl]-3-methyl-7-(3-methyl-2-buten-1-yl)-8-chloro-xanthine

R_{f} value: 0.65 (silica gel, petroleum ether/ethyl acetate/methanol=7:2.5:0.5)

Mass spectrum (ESI⁺): m/z=403, 405 [M+H]⁺

### (31) 1-[2-(2-trifluoromethyl-phenyl)-ethyl]-3-methyl-7-(3-methyl-2-buten-1-yl)-8-bromo-xanthine

R_{f} value: 0.55 (silica gel, petroleum ether/ethyl acetate=8:2)

Mass spectrum (ESI⁺): m/z=485, 487 [M+H]⁺

### (32) 1-[2-(2-bromo-phenyl)-ethyl]-3-methyl-7-(3-methyl-2-buten-1-yl)-8-chloro-xanthine

R_{f} value: 0.55 (silica gel, petroleum ether/ethyl acetate=8:2)

Mass spectrum (ESI⁺): m/z=451, 453, 455 [M+H]⁺

### (33) 1-[2-(3-fluoro-phenyl)-ethyl]-3-methyl-7-(3-methyl-2-buten-1-yl)-8-chloro-xanthine

R_{f} value: 0.60 (silica gel, petroleum ether/ethyl acetate=8:2)

Mass spectrum (ESI⁺): m/z=391, 393 [M+H]⁺

### (34) 1-[2-(3-nitro-phenyl)-ethyl]-3-methyl-7-(3-methyl-2-buten-1-yl)-8-chloro-xanthine

R_{f} value: 0.45 (silica gel, petroleum ether/ethyl acetate/methanol=7:2:1)

Mass spectrum (ESI⁺): m/z=440, 442 [M+Na]⁺

### (35) 1-[2-(4-methyl-phenyl)-ethyl]-3-methyl-7-(3-methyl-2-buten-1-yl)-8-chloro-xanthine

R_{f} value: 0.50 (silica gel, petroleum ether/ethyl acetate/methanol=7:2:1)

Mass spectrum (ESI⁺): m/z=387, 389 [M+H]⁺

### (36) 1-[2-(2-nitro-phenyl)-ethyl]-3-methyl-7-(3-methyl-2-buten-1-yl)-8-chloro-xanthine

R_{f} value: 0.85 (silica gel, petroleum ether/ethyl acetate/methanol=6:3:1)

Mass spectrum (ESI⁺): m/z=418, 420 [M+H]⁺

### (37) 1-[2-(3,5-difluoro-phenyl)-ethyl]-3-methyl-7-(3-methyl-2-buten-1-yl)-8-chloro-xanthine

R_{f} value: 0.50 (silica gel, petroleum ether/ethyl acetate=7:3)

Mass spectrum (EI): m/z=408, 410 [M]⁺

### (38) 1-[2-(2,6-difluoro-phenyl)-ethyl]-3-methyl-7-(3-methyl-2-buten-1-yl)-8-chloro-xanthine

R_{f} value: 0.50 (silica gel, petroleum ether/ethyl acetate=7:3)

Mass spectrum (ESI⁺): m/z=409, 411 [M+H]⁺

### (39) 1-[2-(3,5-dimethyl-phenyl)-ethyl]-3-methyl-7-(3-methyl-2-buten-1-yl)-8-chloro-xanthine

R_{f} value: 0.58 (silica gel, petroleum ether/ethyl acetate=7:3)

Mass spectrum (ESI⁺): m/z=401, 403 [M+H]⁺

### (40) 1-(2-phenyl-propyl)-3-methyl-7-(3-methyl-2-buten-1-yl)-8-chloro-xanthine

R_{f} value: 0.60 (silica gel, petroleum ether/ethyl acetate/methanol=7:2:1)

Mass spectrum (ESI⁺): m/z=387, 389 [M+H]⁺

### (41) 1-(2-methoxy-2-phenyl-ethyl)-3-methyl-7-(3-methyl-2-buten-1-yl)-8-chloro-xanthine

R_{f} value: 0.70 (silica gel, petroleum ether/ethyl acetate/methanol=7:2:1)

Mass spectrum (ESI⁺): m/z=425, 427 [M+Na]+

### (42) 1-[(pyridin-2-yl)methyl]-3-methyl-7-(3-methyl-2-buten-1-yl)-8-chloro-xanthine

R_{f} value: 0.14 (silica gel, petroleum ether/ethyl acetate=1:1)

Mass spectrum (ESI⁺): m/z=360, 362 [M+H]⁺

### (43) 1-[(isoquinolin-1-yl)methyl]-3-methyl-7-(3-methyl-2-buten-1-yl)-8-chloro-xanthine

R_{f} value: 0.31 (silica gel, cyclohexane/ethyl acetate=1:1)

Mass spectrum (ESI⁺): m/z=410, 412 [M+H]⁺

### (44) 1-[(pyridin-3-yl)methyl]-3-methyl-7-(3-methyl-2-buten-1-yl)-8-chloro-xanthine

R_{f} value: 0.10 (silica gel, methylene chloride/methanol=98:2)

Mass spectrum (ESI⁺): m/z=360, 362 [M+H]⁺

### (45) 1-[(pyridin-4-yl)methyl]-3-methyl-7-(3-methyl-2-buten-1-yl)-8-chloro-xanthine

R_{f} value: 0.24 (silica gel, methylene chloride/methanol=95:2)

Mass spectrum (ESI⁺): m/z=360, 362 [M+H]⁺

### (46) 1-[(isoquinolin-4-yl)methyl]-3-methyl-7-(3-methyl-2-buten-1-yl)-8-chloro-xanthine

R_{f} value: 0.28 (silica gel, ethyl acetate/petroleum ether=2:1)

Mass spectrum (ESI⁺): m/z=410, 412 [M+H]⁺

### (47) 1-[(1-methyl-1H-indazol-3-yl)methyl]-3-methyl-7-(3-methyl-2-buten-1-yl)-8-chloro-xanthine

Mass spectrum (ESI⁺): m/z=413, 415 [M+H]⁺

### (48) 1-[(quinolin-4-yl)methyl]-3-methyl-7-(3-methyl-2-buten-1-yl)-8-chloro-xanthine

Rf value: 0.39 (silica gel, ethyl acetate)

Mass spectrum (ESI⁺): m/z=410, 412 [M+H]⁺

### (49) 1-[(quinolin-8-yl)methyl]-3-methyl-7-(3-methyl-2-buten-1-yl)-8-chloro-xanthine

Rf value: 0.74 (silica gel, ethyl acetate)

Mass spectrum (ESI⁺): m/z=410, 412 [M+H]⁺

### EXAMPLE XIII

### 1,3-dimethyl-5-[trans-2-(tert.-butyloxycarbonylamino)-cyclohexyl]-carbonylamino}-6-amino-uracil

Prepared by treating 1,3-dimethyl-5-({trans-2-[(fluoren-9-ylmethoxycarbonyl)amino]-cyclohexyl}-carbonylamino)-6-amino-uracil with piperidine in dimethylformamide and subsequently reacting with di-tert.butyl pyrocarbonate

Mass spectrum (ESI⁺): m/z=396 [M+H]⁺

### EXAMPLE XIV

### 1-methyl-3-(2-propyn-1-yl)-7-benzyl-8-chloro-xanthine

Prepared by reacting 1-methyl-7-benzyl-8-chloro-xanthine with propargyl bromide in the presence of potassium carbonate in dimethylformamide at ambient temperature

Melting point: 169-172° C.

Mass spectrum (EI): m/z=328, 330 [M]⁺

The following compounds are obtained analogously to Example XIV:

### (1) 1-methyl-3-(2-propen-1-yl)-7-benzyl-8-chloro-xanthine

R_{f} value: 0.83 (silica gel, methylene chloride/methanol=95:5)

Mass spectrum (EI): m/z=330, 332 [M]⁺

### (2) 1-methyl-3-(2-phenyl-ethyl)-7-benzyl-8-chloro-xanthine

Melting point: 174-179° C.

Mass spectrum (ESI⁺): m/z=395, 397 [M+H]⁺

### (3) 1-phenyl-3-methyl-7-(3-methyl-2-buten-1-yl)-8-[(3-(tert.-butyloxycarbonylamino)-piperidin-1-yl]-xanthine

R_{f} value: 0.66 (aluminium oxide, ethyl acetate/petroleum ether=8:2)

Mass spectrum (ESI⁺): m/z=509 [M+H]⁺

### (4) 1-methyl-3-(2-dimethylamino-ethyl)-7-benzyl-8-chloro-xanthine

R_{f} value: 0.30 (silica gel, methylene chloride/methanol/conc. aqueous ammonia=9:1:0.1)

Mass spectrum (ESI⁺): m/z=362, 364 [M+H]⁺

### (5) 1,3-bis(2-phenyl-ethyl)-7-(3-methyl-2-buten-1-yl)-8-[3-(tert.-butyloxycarbonylamino)-piperidin-1-yl]-xanthine

R_{f} value: 0.79 (silica gel, petroleum ether/ethyl acetate=4:6)

Mass spectrum (ESI⁺): m/z=627 [M+H]⁺

### (6) 1-(2-phenyl-ethyl)-3-cyanomethyl-7-(3-methyl-2-buten-1-yl)-8-[3-(tert.-butyloxycarbonylamino)-piperidin-1-yl]-xanthine

R_{f} value: 0.74 (silica gel, ethyl acetate/petroleum ether=6:4)

Mass spectrum (ESI⁺): m/z=562 [M+H]⁺

### (7) 1-(2-phenyl-ethyl)-3-[(methoxycarbonyl)-methyl]-7-(3-methyl-2-buten-1-yl)-8-[3-(tert.-butyloxycarbonylamino)-piperidin-1-yl]-xanthine

R_{f} value: 0.65 (silica gel, ethyl acetate/petroleum ether=6:4)

Mass spectrum (ESI⁺): m/z=595 [M+H]⁺

### (8) 1-(2-phenyl-ethyl)-3-(2-dimethylamino-ethyl)-7-(3-methyl-2-buten-1-yl)-8-[3-(tert.-butyloxycarbonylamino)-piperidin-1-yl]-xanthine

R_{f} value: 0.39 (silica gel, methylene chloride/methanol/conc. aqueous ammonia=90:10:1)

Mass spectrum (ESI⁺): m/z=594 [M+H]⁺

### (9) 1-(2-phenyl-ethyl)-3-(2-propyn-1-yl)-7-(3-methyl-2-buten-1-yl)-8-[3-(tert.-butyloxycarbonylamino)-piperidin-1-yl]-xanthine

R_{f} value: 0.77 (silica gel, ethyl acetate/petroleum ether=6:4)

Mass spectrum (ESI⁺): m/z=561 [M+H]⁺

### (10) 1-methyl-3-(2-phenyl-2-oxo-ethyl)-7-(3-methyl-2-buten-1-yl)-8-[3-(tert.-butyloxycarbonylamino)-piperidin-1-yl]-xanthine

R_{f} value: 0.69 (silica gel, methylene chloride/methanol/conc. aqueous ammonia=95:5:1)

Mass spectrum (ESI⁺): m/z=551 [M+H]⁺

### (11) 1-methyl-3-cyanomethyl-7-(3-methyl-2-buten-1-yl)-8-[3-(tert.-butyloxycarbonylamino)-piperidin-1-yl]-xanthine

R_{f} value: 0.80 (silica gel, methylene chloride/methanol/conc. aqueous ammonia=90:10:1)

Mass spectrum (ESI⁺): m/z=472 [M+H]⁺

### (12) 1-methyl-3-(2-phenyl-ethyl)-7-(3-methyl-2-buten-1-yl)-8-[3-(tert.-butyloxycarbonylamino)-piperidin-1-yl]-xanthine

R_{f} value: 0.88 (silica gel, methylene chloride/methanol/conc. aqueous ammonia=90:10:1)

Mass spectrum (ESI⁺): m/z=537 [M+H]⁺

### (13) 1-methyl-3-(2-dimethylamino-ethyl)-7-(3-methyl-2-buten-1-yl)-8-[3-(tert.-butyloxycarbonylamino)-piperidin-1-yl]-xanthine

R_{f} value: 0.21 (silica gel, methylene chloride/methanol/conc. aqueous ammonia=90:10:1)

Mass spectrum (ESI⁺): m/z=504 [M+H]⁺

### (14) 1-methyl-3-isopropyl-7-(3-methyl-2-buten-1-yl)-8-[3-(tert.-butyloxycarbonylamino)-piperidin-1-yl]-xanthine

R_{f} value: 0.54 (silica gel, methylene chloride/methanol/conc. aqueous ammonia=95:5:1)

### (15) 1-methyl-3-(2-cyano-ethyl)-7-(3-methyl-2-buten-1-yl)-8-[3-(tert.-butyloxycarbonylamino)-piperidin-1-yl]-xanthine

R_{f} value: 0.59 (silica gel, methylene chloride/methanol/conc. aqueous ammonia=90:10:1)

### (16) 1-methyl-3-[2-(4-methoxy-phenyl)-ethyl]-7-(3-methyl-2-buten-1-yl)-8-[3-(tert.-butyloxycarbonylamino)-piperidin-1-yl]-xanthine

R_{f} value: 0.88 (silica gel, methylene chloride/methanol/conc. aqueous ammonia=90:10:1)

Mass spectrum (ESI⁺): m/z=567 [M+H]⁺

### (17) 1-methyl-3-[2-(3-methoxy-phenyl)-ethyl]-7-(3-methyl-2-buten-1-yl)-8-[3-(tert.-butyloxycarbonylamino)-piperidin-1-yl]-xanthine

R_{f} value: 0.76 (silica gel, methylene chloride/methanol/conc. aqueous ammonia=90:10:1)

Mass spectrum (ESI⁺): m/z=567 [M+H]⁺

### (18) 1-methyl-3-[2-(2-methoxy-phenyl)-ethyl]-7-(3-methyl-2-buten-1-yl)-8-[3-(tert.-butyloxycarbonylamino)-piperidin-1-yl]-xanthine

R_{f} value: 0.68 (silica gel, methylene chloride/methanol/conc. aqueous ammonia=90:10:1)

### (19) 1-methyl-3-[2-(3-methyl-phenyl)-ethyl]-7-(3-methyl-2-buten-1-yl)-8-[3-(tert.-butyloxycarbonylamino)-piperidin-1-yl]-xanthine

R_{f} value: 0.81 (silica gel, methylene chloride/methanol/conc. aqueous ammonia=90:10:1)

Mass spectrum (ESI⁺): m/z=551 [M+H]⁺

### (20) 1-methyl-3-[2-(4-methyl-phenyl)-ethyl]-7-(3-methyl-2-buten-1-yl)-8-[3-(tert.-butyloxycarbonylamino)-piperidin-1-yl]-xanthine

R_{f} value: 0.81 (silica gel, methylene chloride/methanol/conc. aqueous ammonia=90:10:1)

Mass spectrum (ESI⁺): m/z=551 [M+H]⁺

### (21) 1-methyl-3-[2-(2-methyl-phenyl)-ethyl]-7-(3-methyl-2-buten-1-yl)-8-[3-(tert.-butyloxycarbonylamino)-piperidin-1-yl]-xanthine

R_{f} value: 0.72 (silica gel, methylene chloride/methanol/conc. aqueous ammonia=90:10:1)

(22) 1-methyl-3-[2-(2-fluoro-phenyl)-ethyl]-7-(3-methyl-2-buten-1-yl)-8-[3-(tert.-butyloxycarbonylamino)-piperidin-1-yl]-xanthine

R_{f} value: 0.89 (silica gel, methylene chloride/methanol/conc. aqueous ammonia=90:10:1)

Mass spectrum (ESI⁺): m/z=555 [M+H]⁺

### (23) 1-methyl-3-(4-phenyl-butyl)-7-(3-methyl-2-buten-1-yl)-8-[3-(tert.-butyloxycarbonylamino)-piperidin-1-yl]-xanthine

R_{f} value: 0.65 (silica gel, methylene chloride/methanol/conc. aqueous ammonia=90:10:1)

Mass spectrum (ESI⁺): m/z=565 [M+H]⁺

### (24) 1-methyl-3-(3-phenyl-propyl)-7-(3-methyl-2-buten-1-yl)-8-[3-(tert.-butyloxycarbonylamino)-piperidin-1-yl]-xanthine

R_{f} value: 0.84 (silica gel, methylene chloride/methanol/conc. aqueous ammonia=90:10:1)

Mass spectrum (ESI⁺): m/z=551 [M+H]⁺

### (25) 1-methyl-3-[2-(4-fluoro-phenyl)-ethyl]-7-(3-methyl-2-buten-1-yl)-8-[3-(tert.-butyloxycarbonylamino)-piperidin-1-yl]-xanthine

R_{f} value: 0.80 (silica gel, methylene chloride/methanol/conc. aqueous ammonia=98:2:1)

Mass spectrum (ESI⁺): m/z=555 [M+H]⁺

### (26) 1-methyl-3-[2-(3-fluoro-phenyl)-ethyl]-7-(3-methyl-2-buten-1-yl)-8-[3-(tert.-butyloxycarbonylamino)-piperidin-1-yl]-xanthine

R_{f} value: 0.82 (silica gel, methylene chloride/methanol/conc. aqueous ammonia=90:10:1)

Mass spectrum (ESI⁺): m/z=555 [M+H]⁺

### (27) 1-methyl-3-(2-phenyl-ethyl)-7-(2-cyano-benzyl)-8-chloro-xanthine

Mass spectrum (ESI⁺): m/z=420, 422 [M+H]⁺

### EXAMPLE XV

### 1-methyl-7-benzyl-8-chloro-xanthine

Prepared by treating 1-methyl-3-(2-trimethylsilanyl-ethoxymethyl)-7-benzyl-8-chloro-xanthine with trifluoroacetic acid in methylene chloride at ambient temperature

R_{f} value: 0.10 (silica gel, methylene chloride/methanol=98:2)

The following compound is obtained analogously to Example XV:

### 1) 1-methyl-7-(2-cyano-benzyl)-8-chloro-xanthine

Mass spectrum (ESI⁺): m/z=338, 340 [M+Na]⁺

### EXAMPLE XVI

### 1,3-dimethyl-7-(3-methyl-phenyl)-8-chloro-xanthine

Prepared by reacting 8-chloro-theophylline with 3-methylphenylboric acid in the presence of anhydrous copper(II)acetate, pyridine and molecular sieve 4 Å in methylene chloride at ambient temperature

Mass spectrum (ESI⁺): m/z=305, 307 [M+H]⁺

The following compounds are obtained analogously to Example XVI:

### (1) 1,3-dimethyl-7-((E)-1-hexen-1-yl)-8-chloro-xanthine

Mass spectrum (ESI⁺): m/z=297, 299 [M+H]⁺

### (2) 1,3-dimethyl-7-((E)-2-phenyl-vinyl)-8-chloro-xanthine

Mass spectrum (ESI⁺): m/z=317, 319 [M+H]⁺

### (3) 1,3-dimethyl-7-(2-naphthyl)-8-chloro-xanthine

R_{f} value: 0.60 (silica gel, cyclohexane/ethyl acetate=1:1)

Mass spectrum (ESI⁺): m/z=341, 343 [M+H]⁺

### (4) 1,3-dimethyl-7-phenyl-8-chloro-xanthine

R_{f} value: 0.60 (silica gel, cyclohexane/ethyl acetate=1:1)

Mass spectrum (ESI⁺): m/z=291, 293 [M+H]⁺

### (5) 1,3-dimethyl-7-(3,5-dimethyl-phenyl)-8-chloro-xanthine

R_{f} value: 0.60 (silica gel, cyclohexane/ethyl acetate=1:1)

Mass spectrum (ESI⁺): m/z=319, 321 [M+H]⁺

### (6) 1,3-dimethyl-7-(4-methyl-phenyl)-8-chloro-xanthine

R_{f} value: 0.60 (silica gel, cyclohexane/ethyl acetate=1:1)

Mass spectrum (ESI⁺): m/z=305, 307 [M+H]⁺

### (7) 1,3-dimethyl-7-(3-trifluoromethyl-phenyl)-8-chloro-xanthine

R_{f} value: 0.60 (silica gel, cyclohexane/ethyl acetate=1:1)

Mass spectrum (ESI⁺): m/z=381, 383 [M+Na]⁺

### (8) 1,3-dimethyl-7-(3-cyano-phenyl)-8-chloro-xanthine

R_{f} value: 0.50 (silica gel, cyclohexane/ethyl acetate=1:1)

Mass spectrum (ESI⁺): m/z=338, 340 [M+Na]⁺

### (9) 1,3-dimethyl-7-(3-fluoro-phenyl)-8-chloro-xanthine

R_{f} value: 0.50 (silica gel, cyclohexane/ethyl acetate=1:1)

Mass spectrum (EI): m/z=308, 310 [M]⁺

### EXAMPLE XVII

### cis-N-methyl-cyclohexane-1,2-diamine

Prepared by treating cis-N-(tert.-butyloxycarbonyl)-cyclohexane-1,2-diamine with lithium aluminium hydride in tetrahydrofuran by refluxing

R_{f} value: 0.10 (silica gel, methylene chloride/methanol/conc. aqueous ammonia=90:10:1)

Mass spectrum (ESI⁺): m/z=129 [M+H]⁺

### EXAMPLE XVIII

### 1-(tert.-butyloxycarbonyl)-3-methylamino-piperidine

Prepared by treating 1-(tert.-butyloxycarbonyl)-3-[N-(2,2,2-trifluoro-acetyl)-N-methyl-amino]-piperidine with 2N sodium hydroxide solution in methanol at ambient temperature

R_{f} value: 0.40 (silica gel, methylene chloride/methanol/conc. aqueous ammonia=90:10:1)

Mass spectrum (ESI⁺): m/z=215 [M+H]⁺

The following compounds are obtained analogously to Example XVIII:

### (1) 1-(tert.-butyloxycarbonyl)-3-methylamino-pyrrolidine

R_{f} value: 0.42 (silica gel, methylene chloride/methanol/conc. aqueous ammonia=90:10:1)

Mass spectrum (ESI⁺): m/z=201 [M+H]⁺

### (2) 2-[3-(tert.-butyloxycarbonylamino)-piperidin-1-yl]-3-benzyl-4-ethoxycarbonyl-5-methylamino-3H-imidazole

Carried out with sodium ethoxide in ethanol.

Rf value: 0.60 (silica gel, petroleum ether/ethyl acetate=1:1)

### EXAMPLE XIX

### 1-(tert.-butyloxycarbonyl)-3-[N-(2,2,2-trifluoro-acetyl)-N-methyl-amino]-piperidine

Prepared by reacting 1-(tert.-butyloxycarbonyl)-3-[(2,2,2-trifluoro-acetyl)amino]-piperidine with sodium hydride and methyl iodide in tetrahydrofuran at ambient temperature

R_{f} value: 0.78 (silica gel, methylene chloride/methanol=95:5)

The following compounds are obtained analogously to Example XIX:

### (1) 1-(tert.-butyloxycarbonyl)-3-[N-(2,2,2-trifluoro-acetyl)-N-methyl-amino]-pyrrolidine

### (2) 2-[3-(tert.-Butyloxycarbonylamino)-piperidin-1-yl]-3-benzyl-4-ethoxycarbonyl-5-[N-(2,2,2-trifluoro-acetyl)-N-methyl-amino]-3H-imidazole

Carried out with potassium carbonate in dimethylformamide.

Rf value: 0.60 (silica gel, petroleum ether/ethyl acetate=1:1)

### EXAMPLE XX

### 1-(tert.-butyloxycarbonyl)-3-[(2,2,2-trifluoro-acetyl)amino]-piperidine

Prepared by reacting 3-amino-1-(tert.-butyloxycarbonyl)-piperidine with methyl trifluoroacetate in methanol at ambient temperature

R_{f} value: 0.73 (silica gel, methylene chloride/methanol/conc. aqueous ammonia=90:10:1)

Mass spectrum (ESI⁻): m/z=295 [M−H]⁻

The following compound is obtained analogously to Example XX:

### (1) 2-[3-(tert.-butyloxycarbonylamino)-piperidin-1-yl]-3-benzyl-4-ethoxycarbonyl-5-[(2,2,2-trifluoro-acetyl)amino]-3H-imidazole

Carried out with trifluoroacetic anhydride in the presence of 4-dimethylamino-pyridine in methylene chloride at ambient temperature.

Rf value: 0.70 (silica gel, petroleum ether/ethyl acetate=1:1)

### EXAMPLE XXI

### (S)-2-amino-1-methylamino-propane-dihydrochloride

Prepared by refluxing (S)-alanine-methylamide-hydrochloride with lithium aluminium hydride in tetrahydrofuran and precipitating the product obtained after working up in the form of the dihydrochloride

R_{f} value: 0.08 (silica gel, methylene chloride/methanol/conc. aqueous ammonia=90:10:1)

Mass spectrum (ESI⁻): m/z=159, 161, 163 [M+HCl+Cl]⁻

The following compound is obtained analogously to Example XXI:

### (1) (R)-2-amino-1-methylamino-propane-dihydrochloride

Mass spectrum (EI): m/z=88 [M]⁺

### EXAMPLE XXII

### 1-phenyl-7-(3-methyl-2-buten-1-yl)-8-[3-(tert.-butyloxycarbonylamino)-piperidin-1-yl]-xanthine

Prepared by refluxing 2-[3-(tert.-butyloxycarbonylamino)-piperidin-1-yl]-3-(3-methyl-2-buten-1-yl)-4-ethoxycarbonyl-5-[(phenylaminocarbonyl)amino]-3H-imidazole with potassium tert. butoxide in ethanol

R_{f} value: 0.75 (aluminium oxide, methylene chloride/methanol/conc. aqueous ammonia=9:1:0.1)

Mass spectrum (ESI⁺): m/z=495 [M+H]⁺

The following compounds are obtained analogously to Example XXII:

### (1) 1-(2-phenyl-ethyl)-7-(3-methyl-2-buten-1-yl)-8-[3-(tert.-butyloxycarbonylamino)-piperidin-1-yl]-xanthine

R_{f} value: 0.71 (silica gel, ethyl acetate)

Mass spectrum (ESI⁺): m/z=523 [M+H]⁺

### (2) 1-methyl-7-(3-methyl-2-buten-1-yl)-8-[3-(tert.-butyloxycarbonylamino)-piperidin-1-yl]-xanthine

Carried out with sodium ethoxide in ethanol at ambient temperature

Melting point: 182-185° C.

Mass spectrum (ESI⁺): m/z=433 [M+H]⁺

### (3) 1-amino-7-(3-methyl-2-buten-1-yl)-8-[3-(tert.-butyloxycarbonylamino)-piperidin-1-yl]-xanthine

(Contaminated with 1-amino-7-(3-methyl-butyl)-8[3-(tert.-butyloxycarbonylamino)-piperidin-1-yl]-xanthine)

Carried out with sodium ethoxide in ethanol at ambient temperature

R_{f} value: 0.26 (silica gel, methylene chloride/methanol/conc. aqueous ammonia=90:10:1)

Mass spectrum (ESI⁺): m/z=434 [M+H]⁺

### (4) 7-(3-methyl-2-buten-1-yl)-8-[3-(tert.-butyloxycarbonylamino)-piperidin-1-yl]-xanthine

R_{f} value: 0.24 (silica gel, methylene chloride/methanol/conc. aqueous ammonia=90:10:1)

Mass spectrum (ESI⁺): m/z=419 [M+H]⁺

### (5) potassium-{3-methyl-7-benzyl-8-[3-(tert.-butyloxycarbonylamino)-piperidin-1-yl]-xanthine}-2-thiolate

Carried out in n-butanol at 105° C.

Rf value: 0.90 (aluminium oxide, methylene chloride/methanol=10:1)

### EXAMPLE XXIII

### 2-[3-(tert.-butyloxycarbonylamino)-piperidin-1-yl]-3-(3-methyl-2-buten-1-yl)-4-ethoxycarbonyl-5-[(phenyl-aminocarbonyl)amino]-3H-imidazol

Prepared by refluxing 2-[3-(tert.-butyloxycarbonylamino)-piperidin-1-yl]-3-(3-methyl-2-buten-1-yl)-4-ethoxycarbonyl-5-amino-3H-imidazole with phenylisocyanate in 1,2-dimethoxyethane

Mass spectrum (ESI⁺): m/z=541 [M+H]⁺

The following compounds are obtained analogously to Example XXIII:

### (1) 2-[3-(tert.-butyloxycarbonylamino)-piperidin-1-yl]-3-(3-methyl-2-buten-1-yl)-4-ethoxycarbonyl-5-{[(2-phenyl-ethyl)-aminocarbonyl]amino}-3H-imidazole

R_{f} value: 0.70 (silica gel, ethyl acetate)

Mass spectrum (ESI⁺): m/z=569 [M+H]⁺

### (2) 2-[3-(tert.-butyloxycarbonylamino)-piperidin-1-yl]-3-(3-methyl-2-buten-1-yl)-4-ethoxycarbonyl-5-[(methyl-aminocarbonyl)amino]-3H-imidazole

Carried out at 130° C. in a Roth bomb

Mass spectrum (ESI⁺): m/z=479 [M+H]⁺

### (3) 2-[3-(tert.-butyloxycarbonylamino)-piperidin-1-yl]-3-(3-methyl-2-buten-1-yl)-4-ethoxycarbonyl-5-{[(ethoxycarbonylamino)carbonyl]amino}-3H-imidazole

R_{f} value: 0.29 (silica gel, methylene chloride/methanol/conc. aqueous ammonia=90:10:1)

Mass spectrum (ESI⁺): m/z=537 [M+H]⁺

### (4) 1-[2-(3-{[(ethoxycarbonylamino)carbonyl]amino}-phenyl)-2-oxo-ethyl]-3-methyl-7-(3-methyl-2-buten-1-yl)-8-[3-(tert.-butyloxycarbonylamino)-piperidin-1-yl]-xanthine

Carried out in the presence of triethylamine in a mixture of methylene chloride and dimethylformamide at ambient temperature.

R_{f} value: 0.41 (silica gel, cyclohexane/ethyl acetate=1:2)

### (5) 2-[3-(tert.-butyloxycarbonylamino)-piperidin-1-yl]-3-benzyl-4-ethoxycarbonyl-5-{N-[(ethoxycarbonylamino)thiocarbonyl]-N-methyl-amino}-3H-imidazole

Carried out by refluxing with ethoxycarbonylisothiocyanate in tetrahydrofuran.

Rf value: 0.35 (silica gel, petroleum ether/ethyl acetate=1:1)

### EXAMPLE XXIV

### 2-[3-(tert.-butyloxycarbonylamino)-piperidin-1-yl]-3-(3-methyl-2-buten-1-yl)-4-ethoxycarbonyl-5-amino-3H-imidazole

Prepared by reacting cyanimino-[N-(3-methyl-2-buten-1-yl)-N-(ethoxycarbonylmethyl)-amino]-[3-(tert.-butyloxycarbonylamino)-piperidin-1-yl]-methane with sodium in ethanol by refluxing

R_{f} value: 0.26 (aluminium oxide, ethyl acetate/petroleum ether=8:2)

Mass spectrum (ESI⁺): m/z=422 [M+H]⁺

The following compound is obtained analogously to Example XXIV:

### (1) 2-[3-(tert.-butyloxycarbonylamino)-piperidin-1-yl]-3-benzyl-4-ethoxycarbonyl-5-amino-3H-imidazole

Rf value: 0.40 (silica gel, ethyl acetate/petroleum ether=4:1)

### EXAMPLE XXV

### Cyanoimino-[N-(3-methyl-2-buten-1-yl)-N-(ethoxycarbonylmethyl)-amino]-[3-(tert.-butyloxycarbonylamino)-piperidin-1-yl]methane

Prepared by reacting cyanoimino-[(ethoxycarbonylmethyl)amino]-[3-(tert.-butyloxycarbonylamino)-piperidin-1-yl]-methane with 1-bromo-3-methyl-2-butene in the presence of potassium carbonate in acetone at ambient temperature

Mass spectrum (ESI⁺): m/z=422 [M+H]⁺

The following compound is obtained analogously to Example XXV:

### (1) cyanoimino-[N-benzyl-N-(ethoxycarbonylmethyl)-amino]-[3-(tert.-butyloxy-carbonylamino)-piperidin-1-yl]-methane

Carried out with ethyl bromoacetate in the presence of potassium carbonate in dimethylformamide.

Rf value: 0.70 (silica gel, ethyl acetate/petroleum ether=4:1)

### EXAMPLE XXVI

Cyanoimino-[(ethoxycarbonylmethyl)amino]-[3-(tert.-butyloxycarbonylamino)-piperidin-1-yl]-methane

Prepared by reacting cyanoimino-[(ethoxycarbonylmethyl)amino]-phenyloxy-methane with 3-(tert.-butyloxycarbonylamino)-piperidine in isopropanol at 70° C.

R_{f} value: 0.45 (aluminium oxide, ethyl acetate)

Mass spectrum (ESI⁺): m/z=354 [M+H]⁺

The following compound is obtained analogously to Example XXVI:

### (1) cyanoimino-benzylamino-[3-(tert.-butyloxycarbonylamino)-piperidin-1-yl]-methane

Carried out in dimethylformamide at 80° C.

Rf value: 0.56 (aluminium oxide, methylene chloride/methanol=40:1)

### EXAMPLE XXVII

### Cyanoimino-[(ethoxycarbonylmethyl)amino]-phenyloxy-methane

Prepared by reacting diphenylcyanocarbonimidate with ethyl aminoacetate-hydrochloride in the presence of triethylamine in isopropanol at ambient temperature (analogously to R. Besse et al., Tetrahedron 1990, 46, 7803-7812)

Mass spectrum (ESI⁺): m/z=248 [M+H]⁺

The following compound is obtained analogously to Example XXVII:

### (1) cyanoimino-benzylamino-phenyloxy-methane

Rf value: 0.20 (silica gel, petroleum ether/ethyl acetate=3:1)

Mass spectrum (ESI⁺): m/z=252 [M+H]⁺

### EXAMPLE XXVIII

### 1-((E)-2-phenyl-vinyl)-3-methyl-7-(3-methyl-2-buten-1-yl)-8-bromo-xanthine

Prepared by reacting 3-methyl-7-(3-methyl-2-buten-1-yl)-8-bromo-xanthine with (E)-2-phenyl-vinyl-boric acid in the presence of anhydrous copper(II)acetate and pyridine in methylene chloride at ambient temperature.

R_{f} value: 0.70 (silica gel, petroleum ether/ethyl acetate/methanol=6:3:1)

Mass spectrum (ESI⁺): m/z=415, 417 [M+H]⁺

### EXAMPLE XXIX

### 1,3-dimethyl-7-((E)-2-hexen-1-yl)-8-chloro-xanthine

Prepared by reacting 8-chloro-theophylline with (E)-2-hexen-1-ol in the presence of triphenylphosphine and diisopropyl azodicarboxylate in tetrahydrofuran at ambient temperature.

Mass spectrum (EI): m/z=296, 298 [M]⁺

### EXAMPLE XXX

### 1-(phenylsulphinylmethyl)-3-methyl-7-(3-methyl-2-buten-1-yl)-8-[3-(tert.-butyloxycarbonylamino)-piperidin-1-yl]-xanthine

Prepared by oxidation of 1-(phenylsulphanylmethyl)-3-methyl-7-(3-methyl-2-buten-1-yl)-8-[3-(tert.-butyloxycarbonylamino)-piperidin-1-yl]-xanthine with hydrogen peroxide in hexafluoroisopropanol

R_{f} value: 0.40 (silica gel, petroleum ether/ethyl acetate/methanol=6.5:2:1.5)

Mass spectrum (ESI⁺): m/z=571 [M+H]⁺

### EXAMPLE XXXI

### 1,3-dimethyl-7-(3-methyl-2-buten-1-yl)-8-(1-nitroso-piperidin-4-yl)-xanthine

Prepared by treating 1,3-dimethyl-7-(3-methyl-2-buten-1-yl)-8-(piperidin-4-yl)-xanthine with isoamyl nitrite in tetrahydrofuran at 60° C.

The crude product is immediately reacted further (see Example 8).

### (1) 1,3-dimethyl-7-(3-methyl-2-buten-1-yl)-8-(1-nitroso-piperidin-3-yl)-xanthine

Mass spectrum (ESI⁺): m/z=361 [M+H]⁺

### EXAMPLE XXXII

### 1,3-dimethyl-7-((E)-1-buten-1-yl)-8-chloro-xanthine

Prepared by refluxing 1,3-dimethyl-7-(2-methanesulphonyloxy-butyl)-8-chloro-xanthine with 1,8-diazabicyclo[5.4.0]undec-7-ene in dioxan.

Mass spectrum (ESI⁺): m/z=269, 271 [M+H]⁺

### EXAMPLE XXXIII

### 1,3-dimethyl-7-(2-methanesulphonyloxy-butyl)-8-chloro-xanthine

Prepared by reacting 1,3-dimethyl-7-(2-hydroxy-butyl)-8-chloro-xanthine with methanesulphonic acid chloride in methylene chloride in the presence of triethylamine.

Mass spectrum (ESI⁺): m/z=365, 367 [M+H]⁺

The following compounds are obtained analogously to Example XXXIII:

### (1) 1-[2-(3-methanesulphonyloxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=645 [M+H]⁺

### (2) 1-(2-{3-[bis(methanesulphonyl)-amino]-phenyl}-2-oxo-ethyl)-3-methyl-7-(3-methyl-2-buten-1-yl)-8-[3-(tert.-butyloxycarbonylamino)-piperidin-1-yl]-xanthine

### (3) 1-[2-(3-methanesulphonylamino-phenyl)-2-oxo-ethyl]-3-methyl-7-(3-methyl-2-buten-1-yl)-8-[3-(tert.-butyloxycarbonylamino)-piperidin-1-yl]-xanthine

Carried out with pyridine as an auxiliary base.

Mass spectrum (ESI⁺): m/z=644 [M+H]⁺

### (4) 1-[2-(2-methanesulphonyloxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=645 [M+H]⁺

### (5) 1-(2-{2-[bis(methanesulphonyl)-amino]-phenyl}-2-oxo-ethyl)-3-methyl-7-(3-methyl-2-buten-1-yl)-8-[3-(tert.-butyloxycarbonylamino)-piperidin-1-yl]-xanthine

Carried out in dichloroethane with two equivalents of methanesulphonic acid chloride.

Mass spectrum (ESI⁺): m/z=722 [M+H]⁺

### EXAMPLE XXXIV

### 1,3-dimethyl-7-(2-hydroxy-butyl)-8-chloro-xanthine

Prepared by reacting 8-chloro-theophylline with 2-ethyl-oxirane in dimethylformamide in the presence of Hünig base at 65° C.

Mass spectrum (ESI⁺): m/z=287, 289 [M+H]⁺

### EXAMPLE XXXV

### 1-(2-phenyl-ethyl)-3-vinyl-7-(3-methyl-2-buten-1-yl)-8-[3-(tert.-butyloxycarbonylamino)-piperidin-1-yl]-xanthine

135 mg 1-(2-phenyl-ethyl)-7-(3-methyl-2-buten-1-yl)-8-[3-(tert.-butyloxycarbonylamino)-piperidin-1-yl]-xanthine, 84 μl of vinyltrimethoxysilane, 53 mg of anhydrous copper (II)acetate and 0.53 ml of a 1M solution of tetrabutyl-ammonium fluoride in tetrahydrofuran are suspended in 5 ml of methylene chloride and combined with 200 mg of molecular sieve 4 Å. Then 43 μl of pyridine are added and the turquoise reaction mixture is stirred for three days at ambient temperature. It is then diluted with methylene chloride and suction filtered through talc. The filtrate is evaporated down in vacuo and the crude product is purified by chromatography through a silica gel column with cyclohexane/ethyl acetate (8:2 to 1:1) as eluant.

Yield: 32 mg (23% of theory)

R_{f} value: 0.50 (silica gel, cyclohexane/ethyl acetate=2:1)

Mass spectrum (EI): m/z=548 [M]⁺

### EXAMPLE XXXVI

### 1-(2-phenyl-ethyl)-3-((E)-2-phenyl-vinyl)-7-(3-methyl-2-buten-1-yl)-8-[3-(tert.-butyloxycarbonylamino)-piperidin-1-yl]-xanthine

Prepared by reacting 1-(2-phenyl-ethyl)-7-(3-methyl-2-buten-1-yl)-8-[3-(tert.-butyloxycarbonylamino)-piperidin-1-yl]-xanthine with (E)-2-phenylvinyl-boric acid in methylene chloride in the presence of anhydrous copper(II)acetate, pyridine and molecular sieve 4 Å at ambient temperature.

R_{f} value: 0.71 (silica gel, petroleum ether/ethyl acetate=6:4)

Mass spectrum (ESI⁺): m/z=625 [M+H]⁺

The following compounds are obtained analogously to Example XXXVI:

### (1) 1-methyl-3-phenyl-7-(3-methyl-2-buten-1-yl)-8-[3-(tert.-butyloxycarbonylamino)-piperidin-1-yl]-xanthine

R_{f} value: 0.86 (silica gel, methylene chloride/methanol/conc. aqueous ammonia=95:5:1)

Mass spectrum (ESI⁺): m/z=509 [M+H]⁺

### (2) 1-methyl-3-((E)-2-phenyl-vinyl)-7-(3-methyl-2-buten-1-yl)-8-[3-(tert.-butyloxy-carbonylamino)-piperidin-1-yl]-xanthine

Melting point: 201-202.5° C.

Mass spectrum (ESI⁺): m/z=535 [M+H]⁺

### EXAMPLE XXXVII

### 1-(2-hydroxy-2-phenyl-ethyl)-3-methyl-7-(3-methyl-2-buten-1-yl)-8-[3-(tert.-butyloxycarbonylamino)-piperidin-1-yl]-xanthine

Prepared by treating 1-(2-phenyl-2-oxo-ethyl)-3-methyl-7-(3-methyl-2-buten-1-yl)-8-[3-(tert.-butyloxycarbonylamino)-piperidin-1-yl]-xanthine with sodium borohydride in methanol at ambient temperature.

R_{f} value: 0.30 (silica gel, petroleum ether/ethyl acetate/methanol=60:35: 5)

### EXAMPLE XXXVIII

### 1-phenylcarbonylamino-7-(3-methyl-2-buten-1-yl)-8-[3-(tert.-butyloxycarbonylamino)-piperidin-1-yl]-xanthine

Prepared by reacting 1-amino-7-(3-methyl-2-buten-1-yl)-8-[3-(tert.-butyloxycarbonylamino)-piperidin-1-yl]-xanthine (contaminated with 1-amino-7-(3-methyl-butyl)-8-[3-(tert.-butyloxycarbonylamino)-piperidin-1-yl]-xanthine) with benzoyl chloride in the presence of pyridine in methylene chloride at ambient temperature. The product obtained is contaminated with 1-phenylcarbonylamino-7-(3-methyl-butyl)-8-[3-(tert.-butyloxycarbonylamino)-piperidin-1-yl]-xanthine.

R_{f} value: 0.16 (silica gel, methylene chloride/methanol/conc. aqueous ammonia=90:10:1)

Mass spectrum (ESI⁺): m/z=538 [M+H]⁺

### EXAMPLE XXXIX

### 2-[3-(tert.-butyloxycarbonylamino)-piperidin-1-yl]-3-(3-methyl-2-buten-1-yl)-4-ethoxycarbonyl-5-hydrazinocarbonylamino-3H-imidazole

Prepared by reacting 2-[3-(tert.-butyloxycarbonylamino)-piperidin-1-yl]-3-(3-methyl-2-buten-1-yl)-4-ethoxycarbonyl-5-ethoxycarbonylamino-3H-imidazole with hydrazin-hydrate in xylene at 150° C. The product obtained is contaminated with 2-[3-(tert.-butyloxycarbonylamino)-piperidin-1-yl]-3-(3-methyl-butyl)-4-ethoxycarbonyl-5-hydrazinocarbonylamino-3H-imidazole.

R_{f} value: 0.10 (silica gel, methylene chloride/methanol/conc. aqueous ammonia=90:10:1)

### EXAMPLE XL

### 2-[3-(tert.-butyloxycarbonylamino)-piperidin-1-yl]-3-(3-methyl-2-buten-1-yl)-4-ethoxycarbonyl-5-ethoxycarbonylamino-3H-imidazole

Prepared by reacting 2-[3-(tert.-butyloxycarbonylamino)-piperidin-1-yl]-3-(3-methyl-2-buten-1-yl)-4-ethoxycarbonyl-5-amino-3H-imidazole with ethyl chloroformate in the presence of 0.5 N sodium hydroxide solution in methylene chloride at 50° C.

Melting point: 129-131° C.

Mass spectrum (ESI⁺): m/z=494 [M+H]⁺

### EXAMPLE XLI

1-[2-(3-allyloxy-phenyl)-2-oxo-ethyl]-3-methyl-7-(3-methyl-2-buten-1-yl)-8-[3-(tert.-butyloxycarbonylamino)-piperidin-1-yl]-xanthine

Prepared by reacting 1-[2-(3-hydroxy-phenyl)-2-oxo-ethyl]-3-methyl-7-(3-methyl-2-buten-1-yl)-8-[3-(tert.-butyloxycarbonylamino)-piperidin-1-yl]-xanthine with allyl bromide in the presence of potassium carbonate in dimethylformamide at ambient temperature.

Mass spectrum (ESI⁺): m/z=607 [M+H]⁺

The following compounds are obtained analogously to Example XLI:

### (1) 1-{2-oxo-2-[3-(2-propyn-1-yloxy)-phenyl]-ethyl}-3-methyl-7-(3-methyl-2-buten-1-yl)-8-[3-(tert.-butyloxycarbonylamino)-piperidin-1-yl]-xanthine

Mass spectrum (ESI⁺): m/z=627 [M+Na]⁺

### (2) 1-(2-{3-[(methoxycarbonyl)methoxy]-phenyl}-2-oxo-ethyl)-3-methyl-7-(3-methyl-2-buten-1-yl)-8-[3-(tert.-butyloxycarbonylamino)-piperidin-1-yl]-xanthine

Mass spectrum (ESI⁺): m/z=639 [M+H]⁺

### (3) 1-[2-(3-cyanomethoxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=606 [M+H]⁺

### (4) 1-[2-(3-benzyloxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=657 [M+H]⁺

### (5) 1-[2-(3-phenylsulphonyloxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=707 [M+H]⁺

### (6) 1-(2-{2-[(methoxycarbonyl)methoxy]-phenyl}-2-oxo-ethyl)-3-methyl-7-(3-methyl-2-buten-1-yl)-8-[3-(tert.-butyloxycarbonylamino)-piperidin-1-yl]-xanthine

Mass spectrum (ESI⁺): m/z=639 [M+H]⁺

### (7) 1-[2-(2-cyanomethoxy-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=606 [M+H]⁺

### (8) 1-(2-{3-[(dimethylaminocarbonyl)methoxy]-phenyl}-2-oxo-ethyl)-3-methyl-7-(3-methyl-2-buten-1-yl)-8-[3-(tert.-butyloxycarbonylamino)-piperidin-1-yl]-xanthine

Rf value: 0.25 (silica gel, cyclohexane/ethyl acetate/methanol=5:4:1)

Mass spectrum (ESI⁺): m/z=652 [M+H]⁺

### (9) 1-(2-{3-[(methylaminocarbonyl)methoxy]-phenyl}-2-oxo-ethyl)-3-methyl-7-(3-methyl-2-buten-1-yl)-8-[3-(tert.-butyloxycarbonylamino)-piperidin-1-yl]-xanthine

Rf value: 0.24 (silica gel, cyclohexane/ethyl acetate/methanol=5:4:1)

Mass spectrum (ESI⁺): m/z=638 [M+H]⁺

### (10) 1-(2-{3-[(aminocarbonyl)methoxy]-phenyl}-2-oxo-ethyl)-3-methyl-7-(3-methyl-2-buten-1-yl)-8-[3-(tert.-butyloxycarbonylamino)-piperidin-1-yl]-xanthine

Rf value: 0.30 (silica gel, cyclohexane/ethyl acetate/methanol=5:4:1)

Mass spectrum (ESI⁺): m/z=624 [M+H]⁺

### EXAMPLE XLII

### 1-[2-(3-phenyloxy-phenyl)-2-oxo-ethyl]-3-methyl-7-(3-methyl-2-buten-1-yl)-8-[3-(tert.-butyloxycarbonylamino)-piperidin-1-yl]-xanthine

Prepared by reacting 1-[2-(3-hydroxy-phenyl)-2-oxo-ethyl]-3-methyl-7-(3-methyl-2-buten-1-yl)-8-[3-(tert.-butyloxycarbonylamino)-piperidin-1-yl]-xanthine with phenylboric acid in methylene chloride in the presence of anhydrous copper(II)acetate, pyridine and molecular sieve 4 Å at ambient temperature.

Mass spectrum (ESI⁺): m/z=643 [M+H]⁺

### EXAMPLE XLIII

### 1-[2-(3-amino-phenyl)-2-oxo-ethyl]-3-methyl-7-(3-methyl-2-buten-1-yl)-8-[3-(tert-butyloxycarbonylamino)-piperidin-1-yl]-xanthine

Prepared by treating 1-[2-(3-allyloxycarbonylamino-phenyl)-2-oxo-ethyl]-3-methyl-7-(3-methyl-2-buten-1-yl)-8-[3-(tert.-butyloxycarbonylamino)-piperidin-1-yl]-xanthine with tetrakis(triphenylphosphine)palladium(0) and 5,5-dimethyl-1,3-cyclohexanedione in tetrahydrofuran at ambient temperature.

R_{f} value: 0.22 (silica gel, cyclohexane/ethyl acetate/methanol/conc. aqueous ammonia=60:30:10:1)

### EXAMPLE XLIV

### 1-(3-allyloxycarbonylamino-phenyl)-2-bromo-ethan-1-on and 1-(3-allyloxycarbonylamino-phenyl)-2-chloro-ethan-1-one

Prepared by reacting 1-(3-amino-phenyl)-2-bromo-ethan-1-one-hydrobromide with allyl chloroformate in methylene chloride in the presence of Hünig base. A mixture of the chlorine and bromine compounds is obtained.

R_{f} value: 0.50 (silica gel, cyclohexane/ethyl acetate/methanol=6:3:1)

Mass spectrum (ESI⁻): m/z=252, 254 [M1−H]⁻; 296, 298 [M2−H]⁻

### EXAMPLE XLV

### 1-[2-(3-amino-phenyl)-2-oxo-ethyl]-3-methyl-7-(3-methyl-2-buten-1-yl)-8-[3-(tert-butyloxycarbonylamino)-piperidin-1-yl]-xanthine

Prepared by treating 1-[2-(3-nitro-phenyl)-2-oxo-ethyl]-3-methyl-7-(3-methyl-2-buten-1-yl)-8-[3-(tert.-butyloxycarbonylamino)-piperidin-1-yl]-xanthine with iron filings in a mixture of ethanol, water and glacial acetic acid (80:25:10) at 100° C.

R_{f} value: 0.55 (silica gel, cyclohexane/ethyl acetate/methanol/conc. aqueous ammonia=50:30:20:1)

Mass spectrum (ESI⁺): m/z=566 [M+H]⁺

The following compounds are obtained analogously to Example XLV:

### (1) 1-[2-(2-amino-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=566 [M+H]⁺

### (2) 1-[(5-amino-isoquinolin-1-yl)methyl]-3-methyl-7-(3-methyl-2-buten-1-yl)-8-[3-(tert.-butyloxycarbonylamino)-piperidin-1-yl]-xanthine

Rf value: 0.53 (silica gel, methylene chloride/methanol/conc. aqueous ammonia=90:10:1)

Mass spectrum (ESI⁺): m/z=589 [M+H]⁺

### EXAMPLE XLVI

### 2-bromo-1-(3-dimethylamino-phenyl)-ethan-1-one and 2-bromo-1-(2-bromo-5-dimethylamino-phenyl)-ethan-1-one

Prepared by refluxing 1-(3-dimethylamino-phenyl)-ethan-1-one with bromine in the presence of acetic acid in ethyl acetate. A mixture of the mono- and dibromo compounds is obtained.

Mass spectrum (ESI⁺): m/z=242, 244 [M1+H]⁺; 320, 322, 324 [M2+H]⁺

### EXAMPLE XLVII

### 1-[2-(3-methoxycarbonylamino-phenyl)-2-oxo-ethyl]-3-methyl-7-(3-methyl-2-buten-1-yl)-8-[3-(tert-butyloxycarbonylamino)-piperidin-1-yl]-xanthine

Prepared by reacting 1-[2-(3-amino-phenyl)-2-oxo-ethyl]-3-methyl-7-(3-methyl-2-buten-1-yl)-8-[3-(tert.-butyloxycarbonylamino)-piperidin-1-yl]-xanthine with methyl chloroformate in the presence of triethylamine in a mixture of methylene chloride and dimethylformamide (3:1) at ambient temperature.

Mass spectrum (ESI⁺): m/z=624 [M+H]⁺

The following compound is obtained analogously to Example XLVII:

### (1) 1-(2-{3-[(dimethylaminocarbonyl)amino]-phenyl}-2-oxo-ethyl)-3-methyl-7-(3-methyl-2-buten-1-yl)-8-[3-(tert.-butyloxycarbonylamino)-piperidin-1-yl]-xanthine

Reaction carried out with dimethylcarbamoylchloride in the presence of potassium carbonate in dimethylformamide at 75° C.

Rf value: 0.30 (silica gel, cyclohexane/ethyl acetate/methanol=6:4:1)

Mass spectrum (EI): m/z=636 [M]⁺

### EXAMPLE XLVIII

### 1-[2-(3-acetylamino-phenyl)-2-oxo-ethyl]-3-methyl-7-(3-methyl-2-buten-1-yl)-8-[3-(tert.-butyloxycarbonylamino)-piperidin-1-yl]-xanthine

Prepared by reacting 1-[2-(3-amino-phenyl)-2-oxo-ethyl]-3-methyl-7-(3-methyl-2-buten-1-yl)-8-[3-(tert.-butyloxycarbonylamino)-piperidin-1-yl]-xanthine with acetyl chloride in the presence of pyridine in a mixture of methylene chloride and dimethylformamide (3:1) at ambient temperature.

Mass spectrum (ESI⁺): m/z=608 [M+H]⁺

The following compound is obtained analogously to Example XLVIII:

### (1) 1-[2-(2-acetylamino-phenyl)-2-oxo-ethyl]-3-methyl-7-(3-methyl-2-buten-1-yl)-8-[3-(tert.-butyloxycarbonylamino)-piperidin-1-yl]-xanthine

Mass spectrum (ESI⁺): m/z=608 [M+H]⁺

### EXAMPLE XLIX

### 1-[2-(3-cyanomethylamino-phenyl)-2-oxo-ethyl]-3-methyl-7-(3-methyl-2-buten-1-yl)-8-[3-(tert.-butyloxycarbonylamino)-piperidin-1-yl]-xanthine

Prepared by reacting 1-[2-(3-amino-phenyl)-2-oxo-ethyl]-3-methyl-7-(3-methyl-2-buten-1-yl)-8-[3-(tert.-butyloxycarbonylamino)-piperidin-1-yl]-xanthine with bromoacetonitrile in the presence of Hünig base in dimethylformamide at 70° C.

R_{f} value: 0.18 (silica gel, cyclohexane/ethyl acetate=1:2)

### EXAMPLE L

### 1,3-dimethyl-7-(3-methyl-2-buten-1-yl)-8-{cis-N-[2-(tert.-butyloxycarbonylamino)-cyclohexyl]-N-methyl-amino}-xanthine

Prepared by treating 1,3-dimethyl-7-(3-methyl-2-buten-1-yl)-8-[cis-2-(tert.-butyloxycarbonylamino)-cyclohexylamino]-xanthine with sodium hydride in dimethylformamide at 0° C. and subsequently reacting with methyliodide at 0° C. to ambient temperature.

R_{f} value: 0.42 (silica gel, cyclohexane/ethyl acetate=1:1)

The following compound is obtained analogously to Example L:

### (1) 1,3-dimethyl-7-(3-methyl-2-buten-1-yl)-8-{N-[2-(tert.-butyloxycarbonylamino)-2-methyl-propyl]-N-methyl-amino}-xanthine

R_{f} value: 0.62 (silica gel, methylene chloride/methanol=95:5)

Mass spectrum (ESI⁺): m/z=449 [M+H]⁺

### EXAMPLE LI

### 2-(tert.-butyloxycarbonylamino)-3-(N-benzyl-N-methyl-amino)-propionic acid

Prepared by reacting 3-(tert.-butyloxycarbonylamino)-oxetan-2-one with N-benzyl-N-methyl-amine in acetonitrile at ambient temperature.

R_{f} value: 0.40 (silica gel, methylene chloride/methanol=9:1)

Mass spectrum (ESI⁺): m/z=309 [M+H]⁺

### EXAMPLE LII

### 1-(2-{3-[(methylamino)thiocarbonylamino]-phenyl}-2-oxo-ethyl)-3-methyl-7-(3-methyl-2-buten-1-yl)-8-[3-(tert.-butyloxycarbonylamino)-piperidin-1-yl]-xanthine

Prepared by reacting 1-[2-(3-amino-phenyl)-2-oxo-ethyl]-3-methyl-7-(3-methyl-2-buten-1-yl)-8-[3-(tert.-butyloxycarbonylamino)-piperidin-1-yl]-xanthine with methylisothiocyanate in dimethylformamide at 90° C.

R_{f} value: 0.34 (silica gel, cyclohexane/ethyl acetate/methanol=7:2:1)

Mass spectrum (ESI⁺): m/z=639 [M+H]⁺

The following compound is obtained analogously to Example LII:

### (1) 1-(2-{3-[(aminocarbonyl)amino]-phenyl}-2-oxo-ethyl)-3-methyl-7-(3-methyl-2-buten-1-yl)-8-[3-(tert.-butyloxycarbonylamino)-piperidin-1-yl]-xanthine

Reaction carried out with trimethylsilyl isocyanate.

Mass spectrum (ESI⁺): m/z=609 [M+H]⁺

### EXAMPLE LIII

### 1-(2-{3-[(methoxycarbonyl)methylamino]-phenyl}-2-oxo-ethyl)-3-methyl-7-(3-methyl-2-buten-1-yl)-8-[(3-(tert.-butyloxycarbonylamino)-piperidin-1-yl]-xanthine

Prepared by reacting 1-[2-(3-amino-phenyl)-2-oxo-ethyl]-3-methyl-7-(3-methyl-2-buten-1-yl)-8-[3-(tert.-butyloxycarbonylamino)-piperidin-1-yl]-xanthine with methyl bromoacetate in the presence of potassium carbonate in dimethylformamide at 80° C.

Rf value: 0.38 (silica gel, cyclohexane/ethyl acetate=3:7)

Mass spectrum (ESI⁺): m/z=638 [M+H]⁺

### EXAMPLE LIV

### 1-[2-(2-hydroxy-phenyl)-2-oxo-ethyl]-3-methyl-7-(3-methyl-2-buten-1-yl)-8-chloro-xanthine

Prepared by treating 1-[2-(2-methoxy-phenyl)-2-oxo-ethyl]-3-methyl-7-(3-methyl-2-buten-1-yl)-8-chloro-xanthine with boron tribromide in methylene chloride. The desired product is contaminated with about 20% of 1-[2-(2-hydroxy-phenyl)-2-oxo-ethyl]-3-methyl-7-(3-brom-3-methyl-butyl)-8-chloro-xanthine.

Mass spectrum (ESI⁺): m/z=403, 405 [M+H]⁺

### EXAMPLE LV

### 1-methyl-3-[2-(4-methoxy-phenyl)-ethyl]-7-(2-cyano-benzyl)-8-chloro-xanthine

Prepared by reacting 1-methyl-7-(2-cyano-benzyl)-8-chloro-xanthine with 2-(4-methoxy-phenyl)-ethanol in the presence of triphenylphosphine and diethyl azodicarboxylate in tetrahydrofuran at ambient temperature.

Mass spectrum (ESI⁺): m/z=450 [M+H]⁺

### EXAMPLE LVI

### 7-(2-cyano-benzyl)-xanthine

Prepared by treating 16.68 g of 2-amino-7-(2-cyano-benzyl)-1,7-dihydro-purin-6-one with 17.00 g of sodium nitrite in a mixture of 375 ml of conc. acetic acid, 84 ml of water and 5.2 ml of conc. hydrochloric acid at 50° C.

Yield: 8.46 g (50% of theory)

Mass spectrum (ESI⁺): m/z=268 [M+H]⁺

### EXAMPLE LVII

### 2-amino-7-(2-cyano-benzyl)-1,7-dihydro-purin-6-one

Prepared by reacting 20.00 g of guanosine-hydrate with 22.54 g of 2-cyano-benzylbromide in dimethylsulphoxide at 60° C. and subsequently treating with 57 ml of conc. hydrochloric acid.

Yield: 18.00 g (97% of theory)

Mass spectrum (ESI⁺): m/z=267 [M+H]⁺

### EXAMPLE LVIII

### 1-(4-oxo-4H-chromen-3-yl)-3-methyl-7-(3-methyl-2-buten-1-yl)-8-[(3-(tert.-butyloxycarbonylamino)-piperidin-1-yl]-xanthine

Prepared by reacting 1-[2-(2-hydroxy-phenyl)-2-oxo-ethyl]-3-methyl-7-(3-methyl-2-buten-1-yl)-8-[3-(tert.-butyloxycarbonylamino)-piperidin-1-yl]-xanthine with dimethylformamide-dimethylacetal in the presence of pyridine in toluene by refluxing.

Mass spectrum (ESI⁺): m/z=577 [M+H]⁺

### EXAMPLE LIX

### Endo-6-amino-2-benzyl-2-aza-bicyclo[2.2.2]octane and exo-6-amino-2-benzyl-2-aza-bicyclo[2.2.2]octane

Prepared by reacting 2-benzyl-2-aza-bicyclo[2.2.2]octan-6-one (R. F. Borne et al., J. Het. Chem. 1973, 10, 241) with ammonium acetate in the presence of glacial acetic acid and molecular sieve 4 Å in methanol and subsequently treating with sodium cyanoborohydride at ambient temperature. A mixture of endo- and exo-compound is obtained which is separated by chromatography after reaction with di-tert. butyl pyrocarbonate (cf Example IV(9)).

Mass spectrum (ESI⁺): m/z=217 [M+H]⁺

### EXAMPLE LX

### 3-Amino-3-(pyrrolidin-1-ylcarbonyl)-piperidine x trifluoroacetic acid

Prepared by treating 1-(tert.-butyloxycarbonyl)-3-amino-3-(pyrrolidin-1-ylcarbonyl)-piperidine with trifluoroacetic acid in methylene chloride at ambient temperature.

The following compound is obtained analogously to Example LX:

### (1) 3-amino-4-hydroxy-piperidin x trifluoroacetic acid

Mass spectrum (EI): m/z=116 [M]⁺

### EXAMPLE LXI

### 1-(tert.-butyloxycarbonyl)-3-amino-3-(pyrrolidin-1-ylcarbonyl)-piperidine

Prepared by treating 1-(tert.-butyloxycarbonyl)-3-{[(9H-fluoren-9-ylmethoxy)carbonyl]amino}-3-(pyrrolidin-1-ylcarbonyl)-piperidine with diethylamine in tetrahydrofuran at ambient temperature.

Melting point: 108.5° C.

### EXAMPLE LXII

### 1-(tert.-butyloxycarbonyl)-3-benzylamino-4-hydroxy-piperidine and 1-(tert.-butyloxycarbonyl)-4-benzylamino-3-hydroxy-piperidine

Prepared by refluxing 3.10 g of 3-(tert.-butyloxycarbonyl)-7-oxa-3-aza-bicyclo[4.1.0]heptane with 1.7 ml of benzylamine in 30 ml of ethanol. The regio-isomers formed can be separated by chromatography over a silica gel column with ethyl acetate/methanol/conc. aqueous ammonia (90:10:1) as eluant:

### 1-(tert.-butyloxycarbonyl)-4-benzylamino-3-hydroxy-piperidine

Yield: 0.68 g (14% of theory)

Rf value: 0.68 (silica gel, ethyl acetate/methanol/conc. aqueous ammonia=90:10:1)

Mass spectrum (ESI⁺): m/z=307 [M+H]⁺

### 1-(tert.-butyloxycarbonyl)-3-benzylamino-4-hydroxy-piperidine

Yield: 1.13 g (24% of theory)

Rf value: 0.56 (silica gel, ethyl acetate/methanol/conc. aqueous ammonia=90:10:1)

Mass spectrum (ESI⁺): m/z=307 [M+H]⁺

### EXAMPLE LXIII

### 1,3-dimethyl-2-thioxo-7-benzyl-8-[3-(tert.-butyloxycarbonylamino)-piperidin-1-yl]-xanthine

Prepared by reacting potassium {3-methyl-7-benzyl-8-[3-(tert.-butyloxycarbonylamino)-piperidin-1-yl]-xanthine}-2-thiolate with dimethylsulphate in a mixture of water and dimethylformamide. The desired product is separated by chromatography from the 2-methylsulphanyl-3-methyl-7-benzyl-8-(3-amino-piperidin-1-yl)-xanthine which is also formed.

Mass spectrum (EI): m/z=484 [M]⁺

### Preparation of the Final Compounds:

### EXAMPLE 1

### 1,3-dimethyl-7-benzyl-8-(3-amino-pyrrolidin-1-yl)-xanthine

A mixture of 200 mg of 1,3-dimethyl-7-benzyl-8-chloro-xanthine, 420 mg of 3-amino-pyrrolidine-dihydrochloride, 0.92 ml of triethylamine and 2 ml of dimethylformamide is stirred for 2 days at 50° C. The reaction mixture is diluted with 20 ml of water and extracted twice with 10 ml of ethyl acetate. The organic phase is washed with saturated saline solution, dried and evaporated down. The residue is crystallised with diethylether/diisopropylether (1:1). The solid is suction filtered and dried.

Yield: 92 mg (40% of theory)

Melting point: 150° C.

Mass spectrum (ESI⁺): m/z=355 [M+H]⁺

R_{f} value: 0.08 (silica gel, ethyl acetate/methanol/conc. aqueous ammonia=9:1:0.1)

The following compounds are obtained analogously to Example 1:

### (1) 1,3-dimethyl-7-(3-methyl-2-buten-1-yl)-8-(3-amino-pyrrolidin-1-yl)-xanthine

Melting point: 119° C.

Mass spectrum (ESI⁺): m/z=333 [M+H]⁺

R₁ value: 0.07 (silica gel, ethyl acetate/methanol/conc. aqueous ammonia=9:1:0.1)

### (2) 1,3-dimethyl-7-benzyl-8-(3-amino-piperidin-1-yl)-xanthine

Mass spectrum (ESI⁺): m/z=369 [M+H]⁺

R_{f} value: 0.06 (silica gel, ethyl acetate/methanol/conc. aqueous ammonia=9:1:0.1)

### (3) 1,3-dimethyl-7-(3-methyl-2-buten-1-yl)-8-[(trans-2-amino-cyclohexyl)amino]-xanthine

Mass spectrum (ESI⁺): m/z=361 [M+H]⁺

### (4) 1,3-dimethyl-7-(3-methyl-2-buten-1-yl)-8-(3-amino-piperidin-1-yl)-xanthine

Mass spectrum (ESI⁺): m/z=347 [M+H]⁺

### (5) 1,3-dimethyl-7-(3-methyl-2-buten-1-yl)-8-(4-amino-piperidin-1-yl)-xanthine

Mass spectrum (ESI⁺): m/z=347 [M+H]⁺

### (6) 1,3-dimethyl-7-(3-methyl-2-buten-1-yl)-8-[(cis-2-amino-cyclohexyl)amino]-xanthine

Mass spectrum (ESI⁺): m/z=361 [M+H]⁺

### (7) 1,3-dimethyl-7-(2-butyn-1-yl)-8-(3-amino-piperidin-1-yl)-xanthine

Mass spectrum (ESI⁺): m/z=331 [M+H]⁺

R_{f} value: 0.08 (silica gel, ethyl acetate/methanol/conc. aqueous ammonia=9:1:0.1)

### (8) 1,3-dimethyl-7-[(1-cyclopenten-1-yl)methyl]-8-(3-amino-piperidin-1-yl)-xanthine

Mass spectrum (ESI⁺): m/z=359 [M+H]⁺

R_{f} value: 0.09 (silica gel, ethyl acetate/methanol/conc. aqueous ammonia=9:1:0.1)

### (9) 1,3-dimethyl-7-(2-thienylmethyl)-8-(3-amino-piperidin-1-yl)-xanthine

Mass spectrum (ESI⁺): m/z=375 [M+H]⁺

R_{f} value: 0.08 (silica gel, ethyl acetate/methanol/conc. aqueous ammonia=9:1:0.1)

### (10) 1,3-dimethyl-7-(3-fluorobenzyl)-8-(3-amino-piperidin-1-yl)-xanthine

Mass spectrum (ESI⁺): m/z=387 [M+H]⁺

R_{f} value: 0.08 (silica gel, ethyl acetate/methanol/conc. aqueous ammonia=9:1:0.1)

### (11) 1,3-dimethyl-7-(2-fluorobenzyl)-8-(3-amino-piperidin-1-yl)-xanthine

Mass spectrum (ESI⁺): m/z=387 [M+H]⁺

R_{f} value: 0.08 (silica gel, ethyl acetate/methanol/conc. aqueous ammonia=9:1:0.1)

### (12) 1,3-dimethyl-7-(4-fluorobenzyl)-8-(3-amino-piperidin-1-yl)-xanthine

Mass spectrum (ESI⁺): m/z=387 [M+H]⁺

### (13) 1,3-dimethyl-7-(2-buten-1-yl)-8-(3-amino-piperidin-1-yl)-xanthine

Mass spectrum (ESI⁺): m/z=333 [M+H]⁺

### (14) 1,3-bis-(cyclopropylmethyl)-7-benzyl-8-(3-amino-piperidin-1-yl)-xanthine

Mass spectrum (ESI⁺): m/z=449 [M+H]⁺

### (15) 3-methyl-7-(3-methyl-2-buten-1-yl)-8-(3-amino-piperidin-1-yl)-xanthine

Mass spectrum (ESI⁺): m/z=333 [M+H]⁺

### (16) 1-ethyl-3-methyl-7-(3-methyl-2-buten-1-yl)-8-(3-amino-piperidin-1-yl)-xanthine

Mass spectrum (ESI⁺): m/z=361 [M+H]⁺

### (17) 1-propyl-3-methyl-7-(3-methyl-2-buten-1-yl)-8-(3-amino-piperidin-1-yl)-xanthine

Mass spectrum (ESI⁺): m/z=375 [M+H]⁺

### (18) 1-butyl-3-methyl-7-(3-methyl-2-buten-1-yl)-8-(3-amino-piperidin-1-yl)-xanthine

Mass spectrum (ESI⁺): m/z=389 [M+H]⁺

### (19) 1-(2-propyl)-3-methyl-7-(3-methyl-2-buten-1-yl)-8-(3-amino-piperidin-1-yl)-xanthine

Mass spectrum (ESI⁺): m/z=375 [M+H]⁺

### (20) 1-(2-methylpropyl)-3-methyl-7-(3-methyl-2-buten-1-yl)-8-(3-amino-piperidin-1-yl)-xanthine

Mass spectrum (ESI⁺): m/z=389 [M+H]⁺

### (21) 1-(2-propen-1-yl)-3-methyl-7-(3-methyl-2-buten-1-yl)-8-(3-amino-piperidin-1-yl)-xanthine

Mass spectrum (ESI⁺): m/z=373 [M+H]⁺

### (22) 1-(2-propyn-1-yl)-3-methyl-7-(3-methyl-2-buten-1-yl)-8-(3-amino-piperidin-1-yl)-xanthine

Mass spectrum (ESI⁺): m/z=371 [M+H]⁺

### (23) 1-(cyclopropylmethyl)-3-methyl-7-(3-methyl-2-buten-1-yl)-8-(3-amino-piperidin-1-yl)-xanthine

Mass spectrum (ESI⁺): m/z=387 [M+H]⁺

### (24) 1-benzyl-3-methyl-7-(3-methyl-2-buten-1-yl)-8-(3-amino-piperidin-1-yl)-xanthine

Mass spectrum (ESI⁺): m/z=423 [M+H]⁺

### (25) 1-(2-phenylethyl)-3-methyl-7-(3-methyl-2-buten-1-yl)-8-(3-amino-piperidin-1-yl)-xanthine

Mass spectrum (ESI⁺): m/z=437 [M+H]⁺

### (26) 1-(3-phenylpropyl)-3-methyl-7-(3-methyl-2-buten-1-yl)-8-(3-amino-piperidin-1-yl)-xanthine

Mass spectrum (ESI⁺): m/z=451 [M+H]⁺

### (27) 1-(2-hydroxyethyl)-3-methyl-7-(3-methyl-2-buten-1-yl)-8-(3-amino-piperidin-1-yl)-xanthine

Mass spectrum (ESI⁺): m/z=377 [M+H]⁺

### (28) 1-(2-methoxyethyl)-3-methyl-7-(3-methyl-2-buten-1-yl)-8-(3-amino-piperidin-1-yl)-xanthine

Mass spectrum (ESI⁺): m/z=391 [M+H]⁺

### (29) 1-(3-hydroxypropyl)-3-methyl-7-(3-methyl-2-buten-1-yl)-8-(3-amino-piperidin-1-yl)-xanthine

Mass spectrum (ESI⁺): m/z=391 [M+H]⁺

### (30) 1-[2-(dimethylamino)ethyl]-3-methyl-7-(3-methyl-2-buten-1-yl)-8-(3-amino-piperidin-1-yl)-xanthine

Mass spectrum (ESI⁺): m/z=404 [M+H]⁺

### (31) 1-[3-(dimethylamino)propyl]-3-methyl-7-(3-methyl-2-buten-1-yl)-8-(3-amino-piperidin-1-yl)-xanthine

Mass spectrum (ESI⁺): m/z=418 [M+H]⁺

### (32) 1-methyl-3-(cyclopropylmethyl)-7-benzyl-8-(3-amino-piperidin-1-yl)-xanthine

Mass spectrum (ESI⁺): m/z=409 [M+H]⁺

### (33) 1,3-diethyl-7-benzyl-8-(3-amino-piperidin-1-yl)-xanthine

Mass spectrum (ESI⁺): m/z=397 [M+H]⁺

### (34) 1-methyl-3-ethyl-7-benzyl-8-(3-amino-piperidin-1-yl)-xanthine

Mass spectrum (ESI⁺): m/z=383 [M+H]⁺

### (35) 1,3-dimethyl-7-(3-methyl-2-buten-1-yl)-8-[N-(2-aminoethyl)-methylamino]-xanthine

Mass spectrum (ESI⁺): m/z=321 [M+H]⁺

### (36) 1-[2-(2,4,6-trimethyl-phenyl)-ethyl]-3-methyl-7-(3-methyl-2-buten-1-yl)-8-(3-amino-piperidin-1-yl)-xanthine

Melting point: 153-154.5° C.

Mass spectrum (ESI⁺): m/z=479 [M+H]⁺

### (37) 1-[2-(2,4-dichloro-phenyl)-ethyl]-3-methyl-7-(3-methyl-2-buten-1-yl)-8-(3-amino-piperidin-1-yl)-xanthine

Melting point: 130-132° C.

Mass spectrum (ESI⁺): m/z=505, 507, 509 [M+H]⁺

### (38) 1-[2-(thiophen-2-yl)-ethyl]-3-methyl-7-(3-methyl-2-buten-1-yl)-8-(3-amino-piperidin-1-yl)-xanthine

R_{f} value: 0.20 (silica gel, ethyl acetate/methanol/conc. aqueous ammonia=5:1:0.1)

Mass spectrum (ESI⁺): m/z=443 [M+H]⁺

### (39) 1-[2-(thiophen-3-yl)-ethyl]-3-methyl-7-(3-methyl-2-buten-1-yl)-8-(3-amino-piperidin-1-yl)-xanthine

R_{f} value: 0.20 (silica gel, ethyl acetate/methanol/conc. aqueous ammonia=5:1:0.1)

Mass spectrum (ESI⁺): m/z=443 [M+H]⁺

### (40) 1-[2-(4-tert.-butyl-phenyl)-ethyl]-3-methyl-7-(3-methyl-2-buten-1-yl)-8-(3-amino-piperidin-1-yl)-xanthine

R_{f} value: 0.25 (silica gel, ethyl acetate/methanol/conc. aqueous ammonia=5:1:0.1)

Mass spectrum (ESI⁺): m/z=493 [M+H]⁺

### (41) 1-[2-(4-fluoro-phenyl)-ethyl]-3-methyl-7-(3-methyl-2-buten-1-yl)-8-(3-amino-piperidin-1-yl)-xanthine

R_{f} value: 0.20 (silica gel, ethyl acetate/methanol/conc. aqueous ammonia=5:1:0.1)

Mass spectrum (ESI⁺): m/z=455 [M+H]⁺

### (42) 1-[2-(4-methoxy-phenyl)-ethyl]-3-methyl-7-(3-methyl-2-buten-1-yl)-8-(3-amino-piperidin-1-yl)-xanthine

R_{f} value: 0.18 (silica gel, ethyl acetate/methanol/conc. aqueous ammonia=5:1:0.1)

Mass spectrum (ESI⁺): m/z=467 [M+H]⁺

### (43) 1-methyl-3,7-dibenzyl-8-(3-amino-piperidin-1-yl)-xanthine

Mass spectrum (ESI⁺): m/z=445 [M+H]⁺

### (44) 1-methyl-3-[(methoxycarbonyl)-methyl]-7-benzyl-8-(3-amino-piperidin-1-yl)-xanthine

R_{f} value: 0.27 (silica gel, methylene chloride/methanol/conc. aqueous ammonia=9:1:0.1)

Mass spectrum (ESI⁺): m/z=427 [M+H]⁺

### (45) 1,3-dimethyl-7-(3-methyl-2-buten-1-yl)-8-[N-(2-methylamino-ethyl)-N-methyl-amino]-xanthine

Mass spectrum (ESI⁺): m/z=335 [M+H]⁺

### (46) 1,3-dimethyl-7-(3-methyl-2-buten-1-yl)-8-[N-(2-dimethylamino-ethyl)-N-methyl-amino]-xanthine

Mass spectrum (ESI⁺): m/z=349 [M+H]⁺

### (47) 1-methyl-3-isopropyl-7-benzyl-8-(3-amino-piperidin-1-yl)-xanthine

R_{f} value: 0.32 (silica gel, methylene chloride/methanol/conc. aqueous ammonia=9:1:0.1)

Mass spectrum (ESI⁺): m/z=397 [M+H]⁺

### (48) 1,3-dimethyl-7-(2-pentyn-1-yl)-8-(3-amino-piperidin-1-yl)-xanthine

Mass spectrum (ESI⁺): m/z=345 [M+H]⁺

### (49) 1-methyl-3-(2-methoxy-ethyl)-7-benzyl-8-(3-amino-piperidin-1-yl)-xanthine

R_{f} value: 0.31 (silica gel, methylene chloride/methanol/conc. aqueous ammonia=9:1:0.1)

Mass spectrum (ESI⁺): m/z=413 [M+H]⁺

### (50) 1-methyl-3-cyanomethyl-7-benzyl-8-(3-amino-piperidin-1-yl)-xanthine

R_{f} value: 0.24 (silica gel, methylene chloride/methanol/conc. aqueous ammonia=9:1:0.1)

Mass spectrum (ESI⁺): m/z=394 [M+H]⁺

### (51) 1-[2-(2-fluoro-phenyl)-ethyl]-3-methyl-7-(3-methyl-2-buten-1-yl)-8-(3-amino-piperidin-1-yl)-xanthine

R_{f} value: 0.30 (silica gel, methylene chloride/methanol/conc. aqueous ammonia=10:1:0.1)

Mass spectrum (ESI⁺): m/z=455 [M+H]⁺

### (52) 1-[2-(2-methyl-phenyl)-ethyl]-3-methyl-7-(3-methyl-2-buten-1-yl)-8-(3-amino-piperidin-1-yl)-xanthine

R_{f} value: 0.34 (silica gel, methylene chloride/methanol/conc. aqueous ammonia=10:1:0.1)

Mass spectrum (ESI⁺): m/z=451 [M+H]⁺

### (53) 1-methyl-3-(2-propyn-1-yl)-7-benzyl-8-(3-amino-piperidin-1-yl)-xanthine

R_{f} value: 0.23 (silica gel, methylene chloride/methanol/conc. aqueous ammonia=9:1:0.1)

Mass spectrum (ESI⁺): m/z=393 [M+H]⁺

### (54) 1-methyl-3-(2-propen-1-yl)-7-benzyl-8-(3-amino-piperidin-1-yl)-xanthine

R_{f} value: 0.31 (silica gel, methylene chloride/methanol/conc. aqueous ammonia=9:1:0.1)

Mass spectrum (ESI⁺): m/z=395 [M+H]⁺

### (55) 1-[2-(3-methyl-phenyl)-ethyl]-3-methyl-7-(3-methyl-2-buten-1-yl)-8-(3-amino-piperidin-1-yl)-xanthine

R_{f} value: 0.20 (silica gel, ethyl acetate/methanol/conc. aqueous ammonia=7:3:0.1)

Mass spectrum (ESI⁺): m/z=451 [M+H]⁺

### (56) 1-[2-(1-naphthyl)-ethyl]-3-methyl-7-(3-methyl-2-buten-1-yl)-8-(3-amino-piperidin-1-yl)-xanthine

R_{f} value: 0.30 (silica gel, methylene chloride/methanol/conc. aqueous ammonia=15:1:0.1)

Mass spectrum (ESI⁺): m/z=487 [M+H]⁺

### (57) 1-[2-(2-naphthyl)-ethyl]-3-methyl-7-(3-methyl-2-buten-1-yl)-8-(3-amino-piperidin-1-yl)-xanthine

R_{f} value: 0.25 (silica gel, ethyl acetate/methanol/conc. aqueous ammonia=7:3:0.1)

Mass spectrum (ESI⁺): m/z=487 [M+H]⁺

### (58) 1-(4-phenyl-butyl)-3-methyl-7-(3-methyl-2-buten-1-yl)-8-(3-amino-piperidin-1-yl)-xanthine

R_{f} value: 0.22 (silica gel, ethyl acetate/methanol/conc. aqueous ammonia=7:3:0.1)

Mass spectrum (ESI⁺): m/z=465 [M+H]⁺

### (59) 1-[2-(3-trifluoromethyl-phenyl)-ethyl]-3-methyl-7-(3-methyl-2-buten-1-yl)-8-(3-amino-piperidin-1-yl)-xanthine

R_{f} value: 0.30 (silica gel, ethyl acetate/methanol/conc. aqueous ammonia=7:3:0.1)

Mass spectrum (ESI⁺): m/z=505 [M+H]⁺

### (60) 1-[2-(pyridin-2-yl)-ethyl]-3-methyl-7-(3-methyl-2-buten-1-yl)-8-(3-amino-piperidin-1-yl)-xanthine

Melting point: 117-120° C.

Mass spectrum (ESI⁺): m/z=438 [M+H]⁺

### (61) 1-[2-(pyrrol-1-yl)-ethyl]-3-methyl-7-(3-methyl-2-buten-1-yl)-8-(3-amino-piperidin-1-yl)-xanthine

Melting point: 136-138.6° C.

Mass spectrum (ESI⁺): m/z=426 [M+H]⁺

### (62) 1,3-dimethyl-7-(3-methyl-phenyl)-8-(3-amino-piperidin-1-yl)-xanthine

Mass spectrum (ESI⁺): m/z=369 [M+H]⁺

### (63) 1-[2-([1,2,3]triazol-1-yl)-ethyl]-3-methyl-7-(3-methyl-2-buten-1-yl)-8-(3-amino-piperidin-1-yl)-xanthine

R_{f} value: 0.15 (silica gel, ethyl acetate/methanol/conc. aqueous ammonia=7:3:0.1)

Mass spectrum (ESI⁺): m/z=428 [M+H]⁺

### (64) 1-[2-(pyridin-4-yl)-ethyl]-3-methyl-7-(3-methyl-2-buten-1-yl)-8-(3-amino-piperidin-1-yl)-xanthine

R_{f} value: 0.12 (silica gel, ethyl acetate/methanol/conc. aqueous ammonia=7:3:0.1)

Mass spectrum (ESI⁺): m/z=438 [M+H]⁺

### (65) 1-(3-butyn-1-yl)-3-methyl-7-(3-methyl-2-buten-1-yl)-8-(3-amino-piperidin-1-yl)-xanthine

Melting point: 150-152° C.

Mass spectrum (ESI⁺): m/z=385 [M+H]⁺

### (66) 1-(3-butene-1-yl)-3-methyl-7-(3-methyl-2-buten-1-yl)-8-(3-amino-piperidin-1-yl)-xanthine

Melting point: 111-112.6° C.

Mass spectrum (ESI⁺): m/z=387 [M+H]⁺

### (67) 1-(4-pentyn-1-yl)-3-methyl-7-(3-methyl-2-buten-1-yl)-8-(3-amino-piperidin-1-yl)-xanthine

R_{f} value: 0.12 (silica gel, ethyl acetate/methanol/conc. aqueous ammonia=8:2:0.1)

Mass spectrum (ESI⁺): m/z=399 [M+H]⁺

### (68) 1-(2-phenyl-ethyl)-3-methyl-7-benzyl-8-(3-amino-piperidin-1-yl)-xanthine

Mass spectrum (ESI⁺): m/z=459 [M+H]⁺

### (69) 1-(2-phenyl-ethyl)-3-methyl-7-cyclopropylmethyl-8-(3-amino-piperidin-1-yl)-xanthine

Mass spectrum (ESI⁺): m/z=423 [M+H]⁺

### (70) 1-methyl-3-(2-phenyl-ethyl)-7-benzyl-8-(3-amino-piperidin-1-yl)-xanthine

R_{f} value: 0.23 (silica gel, methylene chloride/methanol/conc. aqueous ammonia=9:1:0.1)

Mass spectrum (ESI⁺): m/z=459 [M+H]⁺

### (71) 1-(2-phenyl-ethyl)-3-methyl-7-(2-butyn-1-yl)-8-(3-amino-piperidin-1-yl)-xanthine

Mass spectrum (ESI⁺): m/z=421 [M+H]⁺

### (72) 1-(4-penten-1-yl)-3-methyl-7-(3-methyl-2-buten-1-yl)-8-(3-amino-piperidin-1-yl)-xanthine

R_{f} value: 0.18 (silica gel, ethyl acetate/methanol/conc. aqueous ammonia=7:3:0.1)

Mass spectrum (ESI⁺): m/z=401 [M+H]⁺

### (73) 1,3-dimethyl-7-benzyl-8-(homopiperazin-1-yl)-xanthine

R_{f} value: 0.33 (silica gel, methylene chloride/methanol/conc. aqueous ammonia=90:10:0.1)

Mass spectrum (ESI⁺): m/z=369 [M+H]⁺

### (74) 1,3-dimethyl-7-(3-methyl-2-buten-1-yl)-8-{[(piperidin-2-yl)methyl]-amino}-xanthine

R_{f} value: 0.24 (silica gel, methylene chloride/methanol/conc. aqueous ammonia=90:10:1)

Mass spectrum (ESI⁺): m/z=361 [M+H]⁺

### (75) 1,3-dimethyl-7-(3-methyl-2-buten-1-yl)-8-{(R)-[2-(aminomethyl)-pyrrolidin-1-yl]}-xanthine

R_{f} value: 0.27 (silica gel, methylene chloride/methanol/conc. aqueous ammonia=90:10:1)

Mass spectrum (ESI⁺): m/z=347 [M+H]⁺

### (76) 1,3-dimethyl-7-(3-methyl-2-buten-1-yl)-8-{(S)-[2-(aminomethyl)-pyrrolidin-1-yl]}-xanthine

Melting point: 112-115° C.

Mass spectrum (ESI⁺): m/z=347 [M+H]⁺

### (77) 1,3-dimethyl-7-(3-methyl-2-buten-1-yl)-8-[cis-(2-methylamino-cyclohexyl)amino]-xanthine

Melting point: 172.5-175° C.

Mass spectrum (ESI⁺): m/z=375 [M+H]⁺

### (78) 1,3-dimethyl-7-(3-methyl-2-buten-1-yl)-8-(homopiperazin-1-yl)-xanthine

R_{f} value: 0.31 (silica gel, ethyl acetate/methanol/conc. aqueous ammonia=90:10:1)

Mass spectrum (ESI⁺): m/z=347 [M+H]⁺

### (79) 1,3-dimethyl-7-(3-methyl-2-buten-1-yl)-8-[N—((S)-2-amino-propyl)-N-methyl-amino]-xanthine

Carried out with sodium carbonate and Hünig base in dimethylsulphoxide at 150° C. in a Roth bomb

R_{f} value: 0.31 (silica gel, methylene chloride/methanol/conc. aqueous ammonia=90:10:1)

Mass spectrum (ESI⁺): m/z=335 [M+H]⁺

### (80) 1,3-dimethyl-7-(3-methyl-2-buten-1-yl)-8-(piperazin-1-yl)-xanthine

R_{f} value: 0.42 (silica gel, methylene chloride/methanol=9:1)

Mass spectrum (ESI⁺): m/z=333 [M+H]⁺

### (81) 1,3-dimethyl-7-(3-methyl-2-buten-1-yl)-8-[N—((R)-2-amino-propyl)-N-methyl-amino]-xanthine

Carried out with sodium carbonate and Hünig base in dimethylsulphoxide at 150° C. in a Roth bomb

Melting point: 101-104.5° C.

Mass spectrum (ESI⁺): m/z=335 [M+H]⁺

### (82) 1-[2-(pyridin-3-yl)-ethyl]-3-methyl-7-(3-methyl-2-buten-1-yl)-8-(3-amino-piperidin-1-yl)-xanthine

Mass spectrum (ESI⁺): m/z=438 [M+H]⁺

R_{f} value: 0.18 (silica gel, ethyl acetate/methanol/conc. aqueous ammonia=7:3:0.1)

### (83) 1-[2-(4-methyl-thiazol-5-yl)-ethyl]-3-methyl-7-(3-methyl-2-buten-1-yl)-8-(3-amino-piperidin-1-yl)-xanthine

Mass spectrum (ESI⁺): m/z=458 [M+H]⁺

R_{f} value: 0.14 (silica gel, ethyl acetate/methanol/conc. aqueous ammonia=7:3:0.1)

### (84) 1-methyl-3-(2-dimethylamino-ethyl)-7-benzyl-8-(3-amino-piperidin-1-yl)-xanthine

R_{f} value: 0.18 (silica gel, methylene chloride/methanol/conc. aqueous ammonia=9:1:0.1)

Mass spectrum (ESI⁺): m/z=426 [M+H]⁺

### (85) 1-cyanomethyl-3-methyl-7-(3-methyl-2-buten-1-yl)-8-(3-amino-piperidin-1-yl)-xanthine

R_{f} value: 0.33 (silica gel, ethyl acetate/methanol/conc. aqueous ammonia=7:3:0.1)

Mass spectrum (ESI⁺): m/z=372 [M+H]⁺

### (86) 1-[2-(3-methoxy-phenyl)-ethyl]-3-methyl-7-(3-methyl-2-buten-1-yl)-8-(3-amino-piperidin-1-yl)-xanthine

Melting point: 118.5-119.5° C.

Mass spectrum (ESI⁺): m/z=467 [M+H]⁺

### (87) 1-[2-(3-bromo-phenyl)-ethyl]-3-methyl-7-(3-methyl-2-buten-1-yl)-8-(3-amino-piperidin-1-yl)-xanthine

Melting point: 116.5-117.5° C.

Mass spectrum (ESI⁺): m/z=515, 517 [M+H]⁺

### (88) 1-[2-(3-chloro-phenyl)-ethyl]-3-methyl-7-(3-methyl-2-buten-1-yl)-8-(3-amino-piperidin-1-yl)-xanthine

R_{f} value: 0.21 (silica gel, methylene chloride/methanol/conc. aqueous ammonia=9:1:0.1)

Mass spectrum (ESI⁺): m/z=471, 473 [M+H]⁺

### (89) 1,3-dimethyl-7-((E)-1-hexen-1-yl)-8-(3-amino-piperidin-1-yl)-xanthine

Mass spectrum (ESI⁺): m/z=361 [M+H]⁺

### (90) 1-((E)-2-phenyl-vinyl)-3-methyl-7-(3-methyl-2-buten-1-yl)-8-(3-amino-piperidin-1-yl)-xanthine

R_{f} value: 0.11 (silica gel, ethyl acetate/methanol/conc. aqueous ammonia=7:3:0.1)

Mass spectrum (ESI⁺): m/z=435 [M+H]⁺

### (91) 1-[2-(2-chloro-phenyl)-ethyl]-3-methyl-7-(3-methyl-2-buten-1-yl)-8-(3-amino-piperidin-1-yl)-xanthine

R_{f} value: 0.25 (silica gel, ethyl acetate/methanol/conc. aqueous ammonia=7:3:0.1)

Mass spectrum (ESI⁺): m/z=471, 473 [M+H]⁺

### (92) 1,3-dimethyl-7-((E)-2-phenyl-vinyl)-8-(3-amino-piperidin-1-yl)-xanthine

Mass spectrum (ESI⁺): m/z=381 [M+H]⁺

### (93) 1-[2-(2-methoxy-phenyl)-ethyl]-3-methyl-7-(3-methyl-2-buten-1-yl)-8-(3-amino-piperidin-1-yl)-xanthine

R_{f} value: 0.15 (silica gel, ethyl acetate/methanol/conc. aqueous ammonia=7:3:0.1)

Mass spectrum (ESI⁺): m/z=467 [M+H]⁺

### (94) 1-[2-(2-trifluoromethyl-phenyl)-ethyl]-3-methyl-7-(3-methyl-2-buten-1-yl)-8-(3-amino-piperidin-1-yl)-xanthine

R_{f} value: 0.16 (silica gel, ethyl acetate/methanol/conc. aqueous ammonia=7:3:0.1)

Mass spectrum (ESI⁺): m/z=505 [M+H]⁺

### (95) 1-[2-(2-bromo-phenyl)-ethyl]-3-methyl-7-(3-methyl-2-buten-1-yl)-8-(3-amino-piperidin-1-yl)-xanthine

R_{f} value: 0.15 (silica gel, ethyl acetate/methanol/conc. aqueous ammonia=7:3:0.1)

Mass spectrum (ESI⁺): m/z=515, 517 [M+H]⁺

### (96) 1-(2-phenyl-ethyl)-3-methyl-7-(3-methyl-2-buten-1-yl)-8-(piperazin-1-yl)-xanthine

Mass spectrum (ESI⁺): m/z=423 [M+H]⁺

### (97) 1-(2-phenyl-ethyl)-3-methyl-7-(3-methyl-2-buten-1-yl)-8-(homopiperazin-1-yl)-xanthine

Mass spectrum (ESI⁺): m/z=437 [M+H]⁺

### (98) 1-[2-(3-fluoro-phenyl)-ethyl]-3-methyl-7-(3-methyl-2-buten-1-yl)-8-(3-amino-piperidin-1-yl)-xanthine

Melting point: 126.8-127.5° C.

Mass spectrum (ESI⁺): m/z=455 [M+H]⁺

### (99) 1-[2-(3-nitro-phenyl)-ethyl]-3-methyl-7-(3-methyl-2-buten-1-yl)-8-(3-amino-piperidin-1-yl)-xanthine

Melting point: 120.8-122° C.

Mass spectrum (ESI⁺): m/z=482 [M+H]⁺

### (100) 1-[2-(4-methyl-phenyl)-ethyl]-3-methyl-7-(3-methyl-2-buten-1-yl)-8-(3-amino-piperidin-1-yl)-xanthine

Melting point: 129-130.2° C.

Mass spectrum (ESI⁺): m/z=451 [M+H]⁺

### (101) 1,3-dimethyl-7-(3-methyl-2-buten-1-yl)-8-(3-aminomethyl-pyrrolidin-1-yl)-xanthine

R_{f} value: 0.50 (silica gel, methylene chloride/methanol/conc. aqueous ammonia=90:10:1)

Mass spectrum (ESI⁺): m/z=347 [M+H]⁺

### (102) 1,3-dimethyl-7-[(thiophen-3-yl)-methyl]-8-(piperazin-1-yl)-xanthine

(Carried out in tetrahydrofuran at 60° C.)

R_{f} value: 0.14 (silica gel, methylene chloride/methanol=9:1)

Mass spectrum (ESI⁺): m/z=361 [M+H]⁺

### (103) 1,3-dimethyl-7-[(thiophen-2-yl)-methyl]-8-(piperazin-1-yl)-xanthine

(Carried out in tetrahydrofuran at 60° C.)

R_{f} value: 0.19 (silica gel, methylene chloride/methanol=9:1)

Mass spectrum (ESI⁺): m/z=361 [M+H]⁺

### (104) 1,3-dimethyl-7-[(furan-3-yl)-methyl]-8-(piperazin-1-yl)-xanthine

(Carried out in tetrahydrofuran at 60° C.)

R_{f} value: 0.13 (silica gel, methylene chloride/methanol=9:1)

Mass spectrum (ESI⁺): m/z=345 [M+H]⁺

### (105) 1,3-dimethyl-7-[(furan-2-yl)-methyl]-8-(piperazin-1-yl)-xanthine

(Carried out in tetrahydrofuran at 60° C.)

R_{f} value: 0.13 (silica gel, methylene chloride/methanol=9:1)

Mass spectrum (ESI⁺): m/z=345 [M+H]⁺

### (106) 1,3-dimethyl-7-(2-propyn-1-yl)-8-(piperazin-1-yl)-xanthine

(Carried out in tetrahydrofuran at 60° C.)

R_{f} value: 0.16 (silica gel, methylene chloride/methanol=9:1)

Mass spectrum (ESI⁺): m/z=303 [M+H]⁺

### (107) 1,3-dimethyl-7-(2,3-dimethyl-2-buten-1-yl)-8-(piperazin-1-yl)-xanthine

(Carried out in tetrahydrofuran at 60° C.)

R_{f} value: 0.24 (silica gel, methylene chloride/methanol=9:1)

Mass spectrum (ESI⁺): m/z=347 [M+H]⁺

### (108) 1,3-dimethyl-7-((E)-2-methyl-2-buten-1-yl)-8-(piperazin-1-yl)-xanthine

(Carried out in tetrahydrofuran at 60° C.)

R_{f} value: 0.27 (silica gel, methylene chloride/methanol=9:1)

Mass spectrum (ESI⁺): m/z=333 [M+H]⁺

### (109) 1,3-dimethyl-7-[(1-cyclohexen-1-yl)-methyl]-8-(piperazin-1-yl)-xanthine

(Carried out in tetrahydrofuran at 60° C.)

R_{f} value: 0.17 (silica gel, methylene chloride/methanol=9:1)

Mass spectrum (ESI⁺): m/z=359 [M+H]⁺

### (110) 1,3-dimethyl-7-[(1-cyclopenten-1-yl)-methyl]-8-(piperazin-1-yl)-xanthine

(Carried out in tetrahydrofuran at 60° C.)

R_{f} value: 0.19 (silica gel, methylene chloride/methanol=9:1)

Mass spectrum (ESI⁺): m/z=345 [M+H]⁺

### (111) 1,3-dimethyl-7-((Z)-2-methyl-2-buten-1-yl)-8-(piperazin-1-yl)-xanthine (Carried out in tetrahydrofuran at 60° C.)

R_{f} value: 0.23 (silica gel, methylene chloride/methanol=9:1)

Mass spectrum (ESI⁺): m/z=333 [M+H]⁺

### (112) 1,3-dimethyl-7-((E)-2-hexen-1-yl)-8-(3-amino-piperidin-1-yl)-xanthine

Mass spectrum (ESI⁺): m/z=361 [M+H]⁺

### (113) 1,3-dimethyl-7-(3-methyl-2-buten-1-yl)-8-((S)-2-aminomethyl-azetidin-1-yl)-xanthine

R_{f} value: 0.52 (silica gel, methylene chloride/methanol/conc. aqueous ammonia=90:10:1)

Mass spectrum (ESI⁺): m/z=333 [M+H]⁺

### (114) 1,3-dimethyl-7-((E)-1-buten-1-yl)-8-(3-amino-piperidin-1-yl)-xanthine

Mass spectrum (ESI⁺): m/z=333 [M+H]⁺

### (115) 1,3,7-trimethyl-8-(3-amino-piperidin-1-yl)-xanthine Carried out with potassium carbonate in dimethylformamide

Melting point: 147° C.

Mass spectrum (ESI⁺): m/z=293 [M+H]⁺

### (116) 1,3-dimethyl-7-(2-naphthyl)-8-(3-amino-piperidin-1-yl)-xanthine

Carried out with potassium carbonate in dimethylformamide

Mass spectrum (ESI⁺): m/z=405 [M+H]⁺

### (117) 1,3-dimethyl-7-phenyl-8-(3-amino-piperidin-1-yl)-xanthine

Carried out with potassium carbonate in dimethylformamide

Mass spectrum (ESI⁺): m/z=355 [M+H]⁺

### (118) 1,3-dimethyl-7-(3,5-dimethyl-phenyl)-8-(3-amino-piperidin-1-yl)-xanthine

Carried out with potassium carbonate in dimethylformamide

Mass spectrum (ESI⁺): m/z=383 [M+H]⁺

### (119) 1,3-dimethyl-7-[(2-naphthyl)methyl]-8-(3-amino-piperidin-1-yl)-xanthine

Carried out with potassium carbonate in dimethylformamide

Mass spectrum (ESI⁺): m/z=419 [M+H]⁺

### (120) 1,3-dimethyl-7-[(1-naphthyl)methyl]-8-(3-amino-piperidin-1-yl)-xanthine

Carried out with potassium carbonate in dimethylformamide

Mass spectrum (ESI⁺): m/z=419 [M+H]⁺

### (121) 1,3-dimethyl-7-(2-cyano-benzyl)-8-(3-amino-piperidin-1-yl)-xanthine

Carried out with potassium carbonate in dimethylformamide

Mass spectrum (ESI⁺): m/z=394 [M+H]⁺

### (122) 1,3-dimethyl-7-(4-methyl-phenyl)-8-(3-amino-piperidin-1-yl)-xanthine

Carried out with potassium carbonate in dimethylformamide

Mass spectrum (ESI⁺): m/z=369 [M+H]⁺

### (123) 1,3-dimethyl-7-(3-cyano-benzyl)-8-(3-amino-piperidin-1-yl)-xanthine

Carried out with potassium carbonate in dimethylformamide

Mass spectrum (ESI⁺): m/z=394 [M+H]⁺

### (124) 1,3-dimethyl-7-(3,5-difluoro-benzyl)-8-(3-amino-piperidin-1-yl)-xanthine

Carried out with potassium carbonate in dimethylformamide

Mass spectrum (ESI⁺): m/z=405 [M+H]⁺

### (125) 1,3-dimethyl-7-(4-cyano-benzyl)-8-(3-amino-piperidin-1-yl)-xanthine

Carried out with potassium carbonate in dimethylformamide

Mass spectrum (ESI⁺): m/z=394 [M+H]⁺

### (126) 1,3-dimethyl-7-(3-nitro-benzyl)-8-(3-amino-piperidin-1-yl)-xanthine

Carried out with potassium carbonate in dimethylformamide

Mass spectrum (ESI⁺): m/z=414 [M+H]⁺

### (127) 1,3-dimethyl-7-(4-nitro-benzyl)-8-(3-amino-piperidin-1-yl)-xanthine

Carried out with potassium carbonate in dimethylformamide

Mass spectrum (ESI⁺): m/z=414 [M+H]⁺

### (128) 1,3-dimethyl-7-(2-nitro-benzyl)-8-(3-amino-piperidin-1-yl)-xanthine

Carried out with potassium carbonate in dimethylformamide

Mass spectrum (ESI⁺): m/z=414 [M+H]⁺

### (129) 1,3-dimethyl-7-(3-trifluoromethyl-phenyl)-8-(3-amino-piperidin-1-yl)-xanthine

Carried out with potassium carbonate in dimethylformamide

Mass spectrum (ESI⁺): m/z=423 [M+H]⁺

### (130) 1,3-dimethyl-7-(3-cyano-phenyl)-8-(3-amino-piperidin-1-yl)-xanthine

Carried out with potassium carbonate in dimethylformamide

Mass spectrum (ESI⁺): m/z=380 [M+H]⁺

### (131) 1-(2-phenyl-propyl)-3-methyl-7-(3-methyl-2-buten-1-yl)-8-(3-amino-piperidin-1-yl)-xanthine

Carried out with potassium carbonate in dimethylsulphoxide

R_{f} value: 0.50 (silica gel, methylene chloride/methanol/conc. aqueous ammonia=80:20:1)

Mass spectrum (ESI⁺): m/z=451 [M+H]⁺

### (132) 1,3-dimethyl-7-(3-fluoro-phenyl)-8-(3-amino-piperidin-1-yl)-xanthine

Carried out with potassium carbonate in dimethylformamide

R_{f} value: 0.10 (silica gel, methylene chloride/methanol=9:1)

Mass spectrum (ESI⁺): m/z=373 [M+H]⁺

### (133) 1-(2-methoxy-2-phenyl-ethyl)-3-methyl-7-(3-methyl-2-buten-1-yl)-8-(3-amino-piperidin-1-yl)-xanthine

Carried out with potassium carbonate in dimethylsulphoxide

R_{f} value: 0.20 (silica gel, ethyl acetate/methanol=8:2)

Mass spectrum (ESI⁺): m/z=467 [M+H]⁺

### (134) 1,3-dimethyl-7-(3-methyl-2-buten-1-yl)-8-(2-amino-2-methyl-propylamino)-xanthine

Carried out with sodium carbonate in dimethylsulphoxide

Melting point: 140.5-143° C.

Mass spectrum (ESI⁺): m/z=335 [M+H]⁺

### (135) 1,3-dimethyl-7-(3-methyl-2-buten-1-yl)-8-((R)-2-amino-propylamino)-xanthine

Carried out with sodium carbonate in dimethylsulphoxide

Melting point: 141-144° C.

Mass spectrum (ESI⁺): m/z=321 [M+H]⁺

### (136) 1,3-dimethyl-7-(3-methyl-2-buten-1-yl)-8-((S)-2-amino-propylamino)-xanthine

Carried out with potassium tert. butoxide and sodium carbonate in dimethylsulphoxide

Melting point: 142-145° C.

Mass spectrum (ESI⁺): m/z=321 [M+H]⁺

### (137) 1,3-dimethyl-7-(2-cyano-benzyl)-8-(homopiperazin-1-yl)-xanthine

Mass spectrum (ESI⁺): m/z=394 [M+H]⁺

Rf value: 0.10 (silica gel, methylene chloride/methanol=9:1)

### (138) 1,3-dimethyl-7-(2-iod-benzyl)-8-(3-amino-piperidin-1-yl)-xanthine

Mass spectrum (ESI⁺): m/z=495 [M+H]⁺

### (139) 1,3-dimethyl-7-(3-methyl-2-buten-1-yl)-8-[3-amino-3-(pyrrolidin-1-ylcarbonyl)-piperidin-1-yl]-xanthine

Carried out in the presence of sodium carbonate in dimethylsulphoxide.

Melting point: 159-160° C.

Mass spectrum (ESI⁺): m/z=444 [M+H]⁺

### (140) 1,3-dimethyl-7-(3-methyl-2-buten-1-yl)-8-(3-amino-4-hydroxy-piperidin-1-yl)-xanthine

Carried out in the presence of sodium carbonate in dimethylsulphoxide.

Rf value: 0.64 (Reversed Phase ready-made TLC plate (E. Merck), acetonitrile/water/trifluoroacetic acid=50:50:1)

Mass spectrum (ESI⁺): m/z=363 [M+H]⁺

### EXAMPLE 2

### (R)-1,3-dimethyl-7-(3-methyl-2-buten-1-yl)-8-(3-amino-piperidin-1-yl)-xanthine

980 mg of (R)-1,3-dimethyl-7-(3-methyl-2-buten-1-yl)-8-[3-(tert.-butyloxycarbonyl-amino)-piperidin-1-yl]-xanthine in 12 ml methylene chloride are combined with 3 ml of trifluoroacetic acid and stirred for 2 hours at ambient temperature. Then the mixture is diluted with methylene chloride and made alkaline with 1 M sodium hydroxide solution. The organic phase is separated off, dried and evaporated to dryness.

Yield: 680 mg (89% of theory)

Mass spectrum (ESI⁺): m/z=347 [M+H]⁺

R_{f} value: 0.20 (aluminium oxide, ethyl acetate/methanol=9:1)

The following compounds are obtained analogously to Example 2:

### (1) (S)-1,3-dimethyl-7-(3-methyl-2-buten-1-yl)-8-(3-amino-piperidin-1-yl)-xanthine

Mass spectrum (ESI⁺): m/z=347 [M+H]⁺

### (2) 1,3-dimethyl-7-(3-methyl-2-buten-1-yl)-8-(3-amino-hexahydroazepin-1-yl)-xanthine

Mass spectrum (ESI⁺): m/z=361 [M+H]⁺

### (3) 1,3-dimethyl-7-(3-methyl-2-buten-1-yl)-8-(4-amino-hexahydroazepin-1-yl)-xanthine

Mass spectrum (ESI⁺): m/z=361 [M+H]⁺

### (4) 1,3-dimethyl-7-(3-methyl-2-buten-1-yl)-8-(cis-3-amino-cyclohexyl)-xanthine-hydrochloride

The reaction was carried out with hydrochloric acid.

¹H-NMR (400 MHz, 6 mg in 0.5 ml DMSO-d₆, 30° C.): characteristic signals at 3.03 ppm (1H, m, H-1) and 3.15 ppm (1H, m, H-3)

### (5) 1,3-dimethyl-7-(3-methyl-2-buten-1-yl)-8-(3-aminopropyl)-xanthine

The reaction was carried out with hydrochloric acid.

Mass spectrum (ESI⁺): m/z=306 [M+H]⁺

### (6) 1,3-dimethyl-7-(3-methyl-2-buten-1-yl)-8-(3-amino-4-methyl-piperidin-1-yl)-xanthine

Mass spectrum (ESI⁺): m/z=361 [M+H]⁺

### (7) 1-methyl-3-(4-methoxy-benzyl)-7-benzyl-8-((S)-3-amino-piperidin-1-yl)-xanthine

Mass spectrum (ESI⁺): m/z=475 [M+H]⁺

R_{f} value: 0.38 (silica gel, methylene chloride/methanol/conc. aqueous ammonia=9:1:0.1)

### (8) 1,3-dimethyl-7-(3-methyl-2-buten-1-yl)-8-[N-(2-aminoethyl)-N-ethyl-amino]-xanthine

Mass spectrum (ESI⁺): m/z=335 [M+H]⁺

### (9) 1,3-dimethyl-7-(3-methyl-2-buten-1-yl)-8-(piperidin-4-yl)-xanthine

Mass spectrum (ESI⁺): m/z=332 [M+H]⁺

### (10) 1,3-dimethyl-7-(3-methyl-2-buten-1-yl)-8-(trans-2-amino-cyclohexyl)-xanthine

Mass spectrum (ESI⁺): m/z=346 [M+H]⁺

### (11) 1-methyl-3-hexyl-7-benzyl-8-((S)-3-amino-piperidin-1-yl)-xanthine

R_{f} value: 0.18 (silica gel, methylene chloride/methanol/conc. aqueous ammonia=9:1:0.1)

Mass spectrum (ESI⁺): m/z=439 [M+H]⁺

### (12) 1-methyl-3-(2-hydroxy-ethyl)-7-benzyl-8-((S)-3-amino-piperidin-1-yl)-xanthine

R_{f} value: 0.19 (silica gel, methylene chloride/methanol/conc. aqueous ammonia=9:1:0.1)

Mass spectrum (ESI⁺): m/z=399 [M+H]⁺

### (13) 1-(2-phenyl-ethyl)-3-methyl-7-(3-methyl-2-buten-1-yl)-8-((S)-3-amino-piperidin-1-yl)-xanthine

Mass spectrum (ESI⁺): m/z=437 [M+H]⁺

### (14) 1-(2-phenyl-ethyl)-3-methyl-7-(3-methyl-2-buten-1-yl)-8-((R)-3-amino-piperidin-1-yl)-xanthine

Mass spectrum (ESI⁺): m/z=437 [M+H]⁺

### (15) 1,3-dimethyl-7-(3-methyl-2-buten-1-yl)-8-[2-(aminomethyl)-piperidin-1-yl)]-xanthine

R_{f} value: 0.34 (silica gel, methylene chloride/methanol/conc. aqueous ammonia=90:10:1)

Mass spectrum (ESI⁺): m/z=361 [M+H]⁺

### (16) 1,3-dimethyl-7-(3-methyl-2-buten-1-yl)-8-[(pyrrolidin-3-yl)amino]-xanthine

Carried out with hydrochloric acid in dioxan

R_{f} value: 0.15 (silica gel, methylene chloride/methanol/conc. aqueous ammonia=90:10:1)

Mass spectrum (ESI⁺): m/z=333 [M+H]⁺

### (17) 1,3-dimethyl-7-(3-methyl-2-buten-1-yl)-8-[N-(piperidin-3-yl)-N-methyl-amino]-xanthine

R_{f} value: 0.44 (silica gel, methylene chloride/methanol/conc. aqueous ammonia=90:10:1)

Mass spectrum (ESI⁺): m/z=361 [M+H]⁺

### (18) 1-[2-(4-hydroxy-phenyl)-ethyl]-3-methyl-7-(3-methyl-2-buten-1-yl)-8-((S)-3-amino-piperidin-1-yl)-xanthine

Carried out in tetrahydrofuran/water at 50-80° C.

R_{f} value: 0.58 (ready-made reversed phase TLC plate(E. Merck), acetonitrile/water/trifluoroacetic acid=50:50:1)

Mass spectrum (ESI⁺): m/z=453 [M+H]⁺

### (19) 1-[(methoxycarbonyl)-methyl]-3-methyl-7-(3-methyl-2-buten-1-yl)-8-((S)-3-amino-piperidin-1-yl)-xanthine

Melting point: 102-105° C.

Mass spectrum (ESI⁺): m/z=405 [M+H]⁺

### (20) 1-[3-(methoxycarbonyl)-propyl]-3-methyl-7-(3-methyl-2-buten-1-yl)-8-((S)-3-amino-piperidin-1-yl)-xanthine

R_{f} value: 0.15 (silica gel, methylene chloride/methanol=9:1)

Mass spectrum (ESI⁺): m/z=433 [M+H]⁺

### (21) 1-{2-[4-(ethoxycarbonyl)-phenyl]-ethyl}-3-methyl-7-(3-methyl-2-buten-1-yl)-8-((S)-3-amino-piperidin-1-yl)-xanthine

Melting point: 142-144° C.

Mass spectrum (ESI⁺): m/z=509 [M+H]⁺

### (22) 1-[2-(3-hydroxy-phenyl)-ethyl]-3-methyl-7-(3-methyl-2-buten-1-yl)-8-((S)-3-amino-piperidin-1-yl)-xanthine

Carried out in tetrahydrofuran/water at 80° C.

Melting point: 168-170° C.

Mass spectrum (ESI⁺): m/z=453 [M+H]⁺

### (23) 1-[2-(methoxycarbonyl)-ethyl]-3-methyl-7-(3-methyl-2-buten-1-yl)-8-((S)-3-amino-piperidin-1-yl)-xanthine

R_{f} value: 0.26 (silica gel, methylene chloride/methanol=9:1)

Mass spectrum (ESI⁺): m/z=419 [M+H]⁺

### (24) 1,3-dimethyl-7-(3-methyl-2-buten-1-yl)-8-[(piperidin-4-yl)amino]-xanthine

Mass spectrum (ESI⁺): m/z=347 [M+H]⁺

R_{f} value: 0.25 (silica gel, methylene chloride/methanol/conc. aqueous ammonia=9:1:0.1)

### (25) 1,3-dimethyl-7-(3-methyl-2-buten-1-yl)-8-[(piperidin-3-yl)amino]-xanthine

Mass spectrum (ESI⁺): m/z=347 [M+H]⁺

R_{f} value: 0.13 (silica gel, methylene chloride/methanol=9:1)

### (26) 1-phenyl-7-(3-methyl-2-buten-1-yl)-8-(3-amino-piperidin-1-yl)-xanthine

Mass spectrum (ESI⁺): m/z=395 [M+H]⁺

### (27) 1-phenyl-3-methyl-7-(3-methyl-2-buten-1-yl)-8-(3-amino-piperidin-1-yl)-xanthine

R_{f} value: 0.70 (aluminium oxide, methylene chloride/methanol=19:1)

Mass spectrum (ESI⁺): m/z=409 [M+H]⁺

### (28) 1-(2-phenyl-2-oxo-ethyl)-3-methyl-7-(3-methyl-2-buten-1-yl)-8-(3-amino-piperidin-1-yl)-xanthine

R_{f} value: 0.16 (silica gel, ethyl acetate/methanol/conc. aqueous ammonia=7:3:0.1)

Mass spectrum (ESI⁺): m/z=451 [M+H]⁺

### (29) 1,3-dimethyl-7-(3-methyl-2-buten-1-yl)-8-[N-(pyrrolidin-3-yl)-N-methyl-amino]-xanthine

R_{f} value: 0.43 (silica gel, methylene chloride/methanol/conc. aqueous ammonia=90:10:1)

Mass spectrum (ESI⁺): m/z=347 [M+H]⁺

### (30) 1,3-dimethyl-7-(3-methyl-2-buten-1-yl)-8-(3-amino-cyclohexyl)-xanthine

(According to NMR spectrum cis/trans mixture=65:35)

Mass spectrum (ESI⁺): m/z=346 [M+H]⁺

### (31) 1,3-bis(2-phenyl-ethyl)-7-(3-methyl-2-buten-1-yl)-8-(3-amino-piperidin-1-yl)-xanthine

R_{f} value: 0.33 (silica gel, methylene chloride/methanol/conc. aqueous ammonia=90:10:1)

Mass spectrum (ESI⁺): m/z=527 [M+H]⁺

### (32) 1-(2-phenyl-ethyl)-7-(3-methyl-2-buten-1-yl)-8-(3-amino-piperidin-1-yl)-xanthine

Mass spectrum (ESI⁺): m/z=423 [M+H]⁺

### (33) 1-(2-phenyl-ethyl)-3-cyanomethyl-7-(3-methyl-2-buten-1-yl)-8-(3-amino-piperidin-1-yl)-xanthine

R_{f} value: 0.31 (silica gel, methylene chloride/methanol/conc. aqueous ammonia=90:10:1)

Mass spectrum (ESI⁺): m/z=462 [M+H]⁺

### (34) 1-(2-phenyl-ethyl)-3-[(methoxycarbonyl)-methyl]-7-(3-methyl-2-buten-1-yl)-8-(3-amino-piperidin-1-yl)-xanthine

Mass spectrum (ESI⁺): m/z=495 [M+H]⁺

### (35) 1-[2-(2-nitro-phenyl)-ethyl]-3-methyl-7-(3-methyl-2-buten-1-yl)-8-(3-amino-piperidin-1-yl)-xanthine

R_{f} value: 0.25 (silica gel, methylene chloride/methanol/conc. aqueous ammonia=90:10:1)

Mass spectrum (ESI⁺): m/z=482 [M+H]⁺

### (36) 1-[2-(3,5-difluoro-phenyl)-ethyl]-3-methyl-7-(3-methyl-2-buten-1-yl)-8-(3-amino-piperidin-1-yl)-xanthine

Melting point: 162-163.5° C.

Mass spectrum (ESI⁺): m/z=473 [M+H]⁺

### (37) 1-[2-(2-methoxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=481 [M+H]⁺

### (38) 1-[2-(thiophen-3-yl)-2-oxo-ethyl]-3-methyl-7-(3-methyl-2-buten-1-yl)-8-(3-amino-piperidin-1-yl)-xanthine

Mass spectrum (ESI⁺): m/z=457 [M+H]⁺

### (39) 1-[2-(2,6-difluoro-phenyl)-ethyl]-3-methyl-7-(3-methyl-2-buten-1-yl)-8-(3-amino-piperidin-1-yl)-xanthine

R_{f} value: 0.35 (silica gel, methylene chloride/methanol/conc. aqueous ammonia=90:10:1)

Mass spectrum (ESI⁺): m/z=473 [M+H]⁺

### (40) 1-[2-(4-methoxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=481 [M+H]⁺

### (41) 1-(2-phenyl-2-oxo-ethyl)-3-methyl-7-(3-methyl-2-buten-1-yl)-8-((S)-3-amino-piperidin-1-yl)-xanthine

Mass spectrum (ESI⁺): m/z=451 [M+H]⁺

### (42) 1-(2-phenyl-2-oxo-ethyl)-3-methyl-7-(3-methyl-2-buten-1-yl)-8-((R)-3-amino-piperidin-1-yl)-xanthine

Mass spectrum (ESI⁺): m/z=451 [M+H]⁺

### (43) 1-[2-(3,5-dimethyl-phenyl)-ethyl]-3-methyl-7-(3-methyl-2-buten-1-yl)-8-(3-amino-piperidin-1-yl)-xanthine

R_{f} value: 0.15 (silica gel, methylene chloride/methanol/conc. aqueous ammonia=90:10:1)

Mass spectrum (ESI⁺): m/z=465 [M+H]⁺

### (44) 1-(phenylsulphanylmethyl)-3-methyl-7-(3-methyl-2-buten-1-yl)-8-(3-amino-piperidin-1-yl)-xanthine

R_{f} value: 0.40 (silica gel, methylene chloride/methanol/conc. aqueous ammonia=90:10:1)

Mass spectrum (ESI⁺): m/z=455 [M+H]⁺

### (45) 1-(phenylsulphinylmethyl)-3-methyl-7-(3-methyl-2-buten-1-yl)-8-(3-amino-piperidin-1-yl)-xanthine

R_{f} value: 0.42 (silica gel, methylene chloride/methanol/conc. aqueous ammonia=90:10:1)

Mass spectrum (ESI⁺): m/z=471 [M+H]⁺

### (46) 1,3-dimethyl-7-(3-methyl-2-buten-1-yl)-8-(cis-2-amino-cyclopropylamino)-xanthine

Mass spectrum (ESI⁺): m/z=319 [M+H]⁺

R_{f} value: 0.55 (silica gel, methylene chloride/methanol/conc. aqueous ammonia=90:10:0.1)

### (47) 1-[2-(3-methoxy-phenyl)-2-oxo-ethyl]-3-methyl-7-(3-methyl-2-buten-1-yl)-8-(3-amino-piperidin-1-yl)-xanthine

R_{f} value: 0.14 (silica gel, methylene chloride/methanol/conc. aqueous ammonia=90:10:1)

Mass spectrum (ESI⁺): m/z=481 [M+H]⁺

### (48) 1-[2-(4-methyl-phenyl)-2-oxo-ethyl]-3-methyl-7-(3-methyl-2-buten-1-yl)-8-(3-amino-piperidin-1-yl)-xanthine

R_{f} value: 0.35 (silica gel, methylene chloride/methanol/conc. aqueous ammonia=90:10:1)

Mass spectrum (ESI⁺): m/z=465 [M+H]⁺

### (49) 1-(2-methoxycarbonyl-2-propen-1-yl)-3-methyl-7-(3-methyl-2-buten-1-yl)-8-(3-amino-piperidin-1-yl)-xanthine

R_{f} value: 0.30 (silica gel, methylene chloride/methanol/conc. aqueous ammonia=90:10:1)

Mass spectrum (ESI⁺): m/z=431 [M+H]⁺

### (50) 1-(2-phenyl-ethyl)-3-(2-dimethylamino-ethyl)-7-(3-methyl-2-buten-1-yl)-8-(3-amino-piperidin-1-yl)-xanthine

R_{f} value: 0.15 (silica gel, methylene chloride/methanol/conc. aqueous ammonia=90:10:1)

Mass spectrum (ESI⁺): m/z=494 [M+H]⁺

### (51) 1-(2-phenyl-ethyl)-3-(2-propyn-1-yl)-7-(3-methyl-2-buten-1-yl)-8-(3-amino-piperidin-1-yl)-xanthine

R_{f} value: 0.71 (silica gel, methylene chloride/methanol/conc. aqueous ammonia=80:20:1)

Mass spectrum (ESI⁺): m/z=461 [M+H]⁺

### (52) 1-(2-phenyl-ethyl)-3-((E)-2-phenyl-vinyl)-7-(3-methyl-2-buten-1-yl)-8-(3-amino-piperidin-1-yl)-xanthine

R_{f} value: 0.27 (silica gel, methylene chloride/methanol/conc. aqueous ammonia=90:10:1)

Mass spectrum (ESI⁺): m/z=525 [M+H]⁺

### (53) 1,3-dimethyl-7-(3-methyl-2-buten-1-yl)-8-(piperidin-3-yl)-xanthine

Mass spectrum (ESI⁺): m/z=332 [M+H]⁺

### (54) 1-(2-phenyl-ethyl)-3-vinyl-7-(3-methyl-2-buten-1-yl)-8-(3-amino-piperidin-1-yl)-xanthine

R_{f} value: 0.26 (silica gel, methylene chloride/methanol/conc. aqueous ammonia=90:10:1)

Mass spectrum (ESI⁺): m/z=449 [M+H]⁺

### (55) 1-(3-oxo-3-phenyl-propyl)-3-methyl-7-(3-methyl-2-buten-1-yl)-8-(3-amino-piperidin-1-yl)-xanthine

Mass spectrum (ESI⁺): m/z=465 [M+H]⁺

### (56) 1-methyl-3-(2-phenyl-2-oxo-ethyl)-7-(3-methyl-2-buten-1-yl)-8-(3-amino-piperidin-1-yl)-xanthine

R_{f} value: 0.30 (silica gel, methylene chloride/methanol/conc. aqueous ammonia=90:10:1)

Mass spectrum (ESI⁺): m/z=451 [M+H]⁺

### (57) 1-methyl-3-cyanomethyl-7-(3-methyl-2-buten-1-yl)-8-(3-amino-piperidin-1-yl)-xanthine

R_{f} value: 0.23 (silica gel, methylene chloride/methanol/conc. aqueous ammonia=90:10:1)

Mass spectrum (ESI⁺): m/z=372 [M+H]⁺

### (58) 1-methyl-3-(2-phenyl-ethyl)-7-(3-methyl-2-buten-1-yl)-8-(3-amino-piperidin-1-yl)-xanthine

R_{f} value: 0.20 (silica gel, methylene chloride/methanol/conc. aqueous ammonia=90:10:1)

Mass spectrum (ESI⁺): m/z=437 [M+H]⁺

### (59) 1-methyl-3-(2-dimethylamino-ethyl)-7-(3-methyl-2-buten-1-yl)-8-(3-amino-piperidin-1-yl)-xanthine

R_{f} value: 0.14 (silica gel, methylene chloride/methanol/conc. aqueous ammonia=80:20:1)

Mass spectrum (ESI⁺): m/z=404 [M+H]⁺

### (60) 1-methyl-3-isopropyl-7-(3-methyl-2-buten-1-yl)-8-(3-amino-piperidin-1-yl)-xanthine

Melting point: 115-117° C.

Mass spectrum (ESI⁺): m/z=375 [M+H]⁺

### (61) 1-(2-hydroxy-2-phenyl-ethyl)-3-methyl-7-(3-methyl-2-buten-1-yl)-8-(3-amino-piperidin-1-yl)-xanthine

R_{f} value: 0.20 (silica gel, methylene chloride/methanol/conc. aqueous ammonia=90:10:1)

Mass spectrum (ESI⁺): m/z=453 [M+H]⁺

### (62) 1-methyl-3-(2-cyano-ethyl)-7-(3-methyl-2-buten-1-yl)-8-(3-amino-piperidin-1-yl)-xanthine

Melting point: 146-149° C.

Mass spectrum (ESI⁺): m/z=386 [M+H]⁺

### (63) 1-methyl-3-[2-(4-methoxy-phenyl)-ethyl]-7-(3-methyl-2-buten-1-yl)-8-(3-amino-piperidin-1-yl)-xanthine

R_{f} value: 0.34 (silica gel, methylene chloride/methanol/conc. aqueous ammonia=90:10:1)

Mass spectrum (ESI⁺): m/z=467 [M+H]⁺

### (64) 1-methyl-3-phenyl-7-(3-methyl-2-buten-1-yl)-8-(3-amino-piperidin-1-yl)-xanthine

R_{f} value: 0.38 (silica gel, methylene chloride/methanol/conc. aqueous ammonia=90:10:1)

Mass spectrum (ESI⁺): m/z=409 [M+H]⁺

### (65) 1-methyl-3-[2-(3-methoxy-phenyl)-ethyl]-7-(3-methyl-2-buten-1-yl)-8-(3-amino-piperidin-1-yl)-xanthine

R_{f} value: 0.35 (silica gel, methylene chloride/methanol/conc. aqueous ammonia=90:10:1)

Mass spectrum (ESI⁺): m/z=467 [M+H]⁺

### (66) 1-methyl-3-[2-(2-methoxy-phenyl)-ethyl]-7-(3-methyl-2-buten-1-yl)-8-(3-amino-piperidin-1-yl)-xanthine

R_{f} value: 0.31 (silica gel, methylene chloride/methanol/conc. aqueous ammonia=90:10:1)

Mass spectrum (ESI⁺): m/z=467 [M+H]⁺

### (67) 1-methyl-3-[2-(3-methyl-phenyl)-ethyl]-7-(3-methyl-2-buten-1-yl)-8-(3-amino-piperidin-1-yl)-xanthine

R_{f} value: 0.13 (silica gel, methylene chloride/methanol/conc. aqueous ammonia=90:10:1)

Mass spectrum (ESI⁺): m/z=451 [M+H]⁺

### (68) 1-methyl-3-[2-(4-methyl-phenyl)-ethyl]-7-(3-methyl-2-buten-1-yl)-8-(3-amino-piperidin-1-yl)-xanthine

R_{f} value: 0.16 (silica gel, methylene chloride/methanol/conc. aqueous ammonia=95:5:1)

Mass spectrum (ESI⁺): m/z=451 [M+H]⁺

### (69) 1-methyl-3-[2-(2-methyl-phenyl)-ethyl]-7-(3-methyl-2-buten-1-yl)-8-(3-amino-piperidin-1-yl)-xanthine

R_{f} value: 0.16 (silica gel, methylene chloride/methanol/conc. aqueous ammonia=95:5:1)

Mass spectrum (ESI⁺): m/z=451 [M+H]⁺

### (70) 1-methyl-3-[2-(2-fluoro-phenyl)-ethyl]-7-(3-methyl-2-buten-1-yl)-8-(3-amino-piperidin-1-yl)-xanthine

R_{f} value: 0.35 (silica gel, methylene chloride/methanol/conc. aqueous ammonia=90:10:1)

Mass spectrum (ESI⁺): m/z=455 [M+H]⁺

### (71) 1-(2-oxo-propyl)-3-methyl-7-(3-methyl-2-buten-1-yl)-8-(3-amino-piperidin-1-yl)-xanthine x trifluoroacetic acid

(The product is isolated as the trifluoroacetate.)

Mass spectrum (ESI⁺): m/z=389 [M+H]⁺

### (72) 1-methyl-3-(4-phenyl-butyl)-7-(3-methyl-2-buten-1-yl)-8-(3-amino-piperidin-1-yl)-xanthine

R_{f} value: 0.36 (silica gel, methylene chloride/methanol/conc. aqueous ammonia=90:10:1)

Mass spectrum (ESI⁺): m/z=465 [M+H]⁺

### (73) 1-methyl-3-(3-phenyl-propyl)-7-(3-methyl-2-buten-1-yl)-8-(3-amino-piperidin-1-yl)-xanthine

R_{f} value: 0.33 (silica gel, methylene chloride/methanol/conc. aqueous ammonia=90:10:1)

Mass spectrum (ESI⁺): m/z=451 [M+H]⁺

### (74) 1-(2-phenyl-2-oxo-ethyl)-3-methyl-7-(2-cyano-benzyl)-8-(3-amino-piperidin-1-yl)-xanthine

Mass spectrum (ESI⁺): m/z=498 [M+H]⁺

### (75) 1-(2-phenyl-ethyl)-3-methyl-7-(2-cyano-benzyl)-8-(3-amino-piperidin-1-yl)-xanthine

Mass spectrum (ESI⁺): m/z=484 [M+H]⁺

### (76) 1-(3-methoxycarbonyl-2-propen-1-yl)-3-methyl-7-(3-methyl-2-buten-1-yl)-8-(3-amino-piperidin-1-yl)-xanthine

R_{f} value: 0.35 (silica gel, methylene chloride/methanol/conc. aqueous ammonia=80:20:1)

Mass spectrum (ESI⁺): m/z=431 [M+H]⁺

### (77) 1-methyl-3-[2-(4-fluoro-phenyl)-ethyl]-7-(3-methyl-2-buten-1-yl)-8-(3-amino-piperidin-1-yl)-xanthine

R_{f} value: 0.28 (silica gel, methylene chloride/methanol/conc. aqueous ammonia=90:10:1)

Mass spectrum (ESI⁺): m/z=455 [M+H]⁺

### (78) 1-methyl-3-[2-(3-fluoro-phenyl)-ethyl]-7-(3-methyl-2-buten-1-yl)-8-(3-amino-piperidin-1-yl)-xanthine

R_{f} value: 0.35 (silica gel, methylene chloride/methanol/conc. aqueous ammonia=90:10:1)

Mass spectrum (ESI⁺): m/z=455 [M+H]⁺

### (79) 1-[2-(2,5-dimethoxy-phenyl)-2-oxo-ethyl]-3-methyl-7-(3-methyl-2-buten-1-yl)-8-(3-amino-piperidin-1-yl)-xanthine

R_{f} value: 0.29 (silica gel, ethyl acetate/methanol/conc. aqueous ammonia=70:30:1)

Mass spectrum (ESI⁺): m/z=511 [M+H]⁺

### (80) 1-[2-(4-fluoro-phenyl)-2-oxo-ethyl]-3-methyl-7-(3-methyl-2-buten-1-yl)-8-(3-amino-piperidin-1-yl)-xanthine

R_{f} value: 0.35 (silica gel, methylene chloride/methanol/conc. aqueous ammonia=80:20:1)

Mass spectrum (ESI⁺): m/z=469 [M+H]⁺

### (81) 1-phenylcarbonylamino-7-(3-methyl-2-buten-1-yl)-8-(3-amino-piperidin-1-yl)-xanthine

(Contaminated with 1-phenylcarbonylamino-7-(3-methyl-butyl)-8-(3-amino-piperidin-1-yl)-xanthine)

R_{f} value: 0.26 (silica gel, methylene chloride/methanol/conc. aqueous ammonia=80:20:1)

Mass spectrum (ESI⁺): m/z=438 [M+H]⁺

### (82) 1-amino-7-(3-methyl-2-buten-1-yl)-8-(3-amino-piperidin-1-yl)-xanthine

(Contaminated with 1-amino-7-(3-methyl-butyl)-8-(3-amino-piperidin-1-yl)-xanthine)

R_{f} value: 0.22 (silica gel, methylene chloride/methanol/conc. aqueous ammonia=80:20:1)

Mass spectrum (ESI⁺): m/z=334 [M+H]⁺

### (83) 1-[2-(3-methanesulphonyloxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=545 [M+H]⁺

### (84) 1-[2-(3-allyloxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=507 [M+H]⁺

### (85) 1-{2-oxo-2-[3-(2-propyn-1-yloxy)-phenyl]-ethyl}-3-methyl-7-(3-methyl-2-buten-1-yl)-8-(3-amino-piperidin-1-yl)-xanthine

Mass spectrum (ESI⁺): m/z=505 [M+H]⁺

### (86) 1-(3-methoxycarbonyl-2-propen-1-yl)-3-methyl-7-(2-cyano-benzyl)-8-(3-amino-piperidin-1-yl)-xanthine

Mass spectrum (ESI⁺): m/z=478 [M+H]⁺

### (87) 1-(2-{3-[(methoxycarbonyl)methoxy]-phenyl}-2-oxo-ethyl)-3-methyl-7-(3-methyl-2-buten-1-yl)-8-(3-amino-piperidin-1-yl)-xanthine

Mass spectrum (ESI⁺): m/z=539 [M+H]⁺

### (88) 1-[2-(3-cyanomethoxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=506 [M+H]⁺

### (89) 1-[2-(3-benzyloxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=557 [M+H]⁺

### (90) 1-[2-(3-phenylsulphonyloxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=607 [M+H]⁺

### (91) 1-[2-(3-hydroxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=467 [M+H]⁺

### (92) 1-[(pyridin-2-yl)methyl]-3-methyl-7-(2-cyano-benzyl)-8-(3-amino-piperidin-1-yl)-xanthine

R_{f} value: 0.20 (silica gel, methylene chloride/methanol/conc. aqueous ammonia=90:10:1)

Mass spectrum (ESI⁺): m/z=471 [M+H]⁺

### (93) 1-[2-(3-phenyloxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=543 [M+H]⁺

### (94) 1-(2-phenyl-2-oxo-ethyl)-3-[(methoxycarbonyl)methyl]-7-(3-methyl-2-buten-1-yl)-8-(3-amino-piperidin-1-yl)-xanthine

R_{f} value: 0.29 (silica gel, methylene chloride/methanol/conc. aqueous ammonia=90:10:1)

Mass spectrum (ESI⁺): m/z=509 [M+H]⁺

### (95) 1-(2-phenyl-2-oxo-ethyl)-3-methyl-7-(3-methyl-2-buten-1-yl)-8-(piperazin-1-yl)-xanthine

R_{f} value: 0.10 (silica gel, methylene chloride/methanol=90:10)

Mass spectrum (ESI⁺): m/z=437 [M+H]⁺

### (96) 1-[2-(3-amino-phenyl)-2-oxo-ethyl]-3-methyl-7-(3-methyl-2-buten-1-yl)-8-(3-amino-piperidin-1-yl)-xanthine

R_{f} value: 0.25 (silica gel, methylene chloride/methanol/conc. aqueous ammonia=80:20:1)

Mass spectrum (ESI⁺): m/z=466 [M+H]⁺

### (97) 1-(2-{3-[bis(methanesulphonyl)-amino]-phenyl}-2-oxo-ethyl)-3-methyl-7-(3-methyl-2-buten-1-yl)-8-(3-amino-piperidin-1-yl)-xanthine

R_{f} value: 0.45 (silica gel, methylene chloride/methanol/conc. aqueous ammonia=80:20:1)

Mass spectrum (ESI⁺): m/z=622 [M+H]⁺

### (98) 1-[2-(2-bromo-5-dimethylamino-phenyl)-2-oxo-ethyl]-3-methyl-7-(3-methyl-2-buten-1-yl)-8-(3-amino-piperidin-1-yl)-xanthine

Mass spectrum (ESI⁺): m/z=572, 574 [M+H]⁺

### (99) 1-[2-(3-nitro-phenyl)-2-oxo-ethyl]-3-methyl-7-(3-methyl-2-buten-1-yl)-8-(3-amino-piperidin-1-yl)-xanthine

Mass spectrum (ESI⁺): m/z=496 [M+H]⁺

### (100) 1-[2-(3-methoxycarbonylamino-phenyl)-2-oxo-ethyl]-3-methyl-7-(3-methyl-2-buten-1-yl)-8-(3-amino-piperidin-1-yl)-xanthine

Mass spectrum (ESI⁺): m/z=524 [M+H]⁺

### (101) 1-[2-(3-acetylamino-phenyl)-2-oxo-ethyl]-3-methyl-7-(3-methyl-2-buten-1-yl)-8-(3-amino-piperidin-1-yl)-xanthine

Mass spectrum (ESI⁺): m/z=508 [M+H]⁺

### (102) 1-[2-(3-{[(ethoxycarbonylamino)carbonyl]amino}-phenyl)-2-oxo-ethyl]-3-methyl-7-(3-methyl-2-buten-1-yl)-8-(3-amino-piperidin-1-yl)-xanthine

Mass spectrum (ESI⁺): m/z=581 [M+H]⁺

### (103) 1-(2-phenyl-2-oxo-ethyl)-3-methyl-7-(3-methyl-2-buten-1-yl)-8-(homopiperazin-1-yl)-xanthine

R_{f} value: 0.10 (silica gel, methylene chloride/methanol=90:10)

Mass spectrum (ESI⁺): m/z=451 [M+H]⁺

### (104) 1-[2-(3-cyanomethylamino-phenyl)-2-oxo-ethyl]-3-methyl-7-(3-methyl-2-buten-1-yl)-8-(3-amino-piperidin-1-yl)-xanthine

R_{f} value: 0.35 (silica gel, methylene chloride/methanol/conc. aqueous ammonia=80:20:1)

Mass spectrum (ESI⁺): m/z=505 [M+H]⁺

### (105) 1,3-dimethyl-7-(3-methyl-2-buten-1-yl)-8-(4-aminomethyl-piperidin-1-yl)-xanthine

Carried out with isopropanolic hydrochloric acid (5-6M) in methylene chloride

Melting point: 110-112° C.

Mass spectrum (ESI⁺): m/z=361 [M+H]⁺

### (106) 1,3-dimethyl-7-(3-methyl-2-buten-1-yl)-8-(3-aminomethyl-piperidin-1-yl)-xanthine

Carried out with isopropanolic hydrochloric acid (5-6M) in methylene chloride.

R_{f} value: 0.48 (silica gel, methylene chloride/methanol/conc. aqueous ammonia=90:10:0.1)

Mass spectrum (ESI⁺): m/z=361 [M+H]⁺

### (107) 1,3-dimethyl-7-(3-methyl-2-buten-1-yl)-8-(trans-2-amino-cyclobutylamino)-xanthine

Carried out with isopropanolic hydrochloric acid (5-6M) in methylene chloride.

R_{f} value: 0.65 (silica gel, methylene chloride/methanol/conc. aqueous ammonia=90:10:0.1)

Mass spectrum (ESI⁺): m/z=333 [M+H]⁺

### (108) 1,3-dimethyl-7-(3-methyl-2-buten-1-yl)-8-[N—((S)-2-amino-1-methyl-ethyl)-N-methyl-amino]-xanthine

Carried out with isopropanolic hydrochloric acid (5-6M) in methylene chloride.

Melting point: 109.5-113° C.

Mass spectrum (ESI⁺): m/z=335 [M+H]⁺

### (109) 1,3-dimethyl-7-(3-methyl-2-buten-1-yl)-8-[N—((R)-2-amino-1-methyl-ethyl)-N-methyl-amino]-xanthine

Carried out with isopropanolic hydrochloric acid (5-6M) in methylene chloride.

R_{f} value: 0.50 (silica gel, methylene chloride/methanol/conc. aqueous ammonia=90:10:1)

Mass spectrum (ESI⁺): m/z=335 [M+H]⁺

### (110) 1,3-dimethyl-7-(3-methyl-2-buten-1-yl)-8-[cis-N-(2-amino-cyclohexyl)-N-methyl-amino]-xanthine

Carried out with isopropanolic hydrochloric acid (5-6M) in methylene chloride.

R_{f} value: 0.71 (silica gel, methylene chloride/methanol/conc. aqueous ammonia=90:10:1)

Mass spectrum (ESI⁺): m/z=375 [M+H]⁺

### (111) 1,3-dimethyl-7-(3-methyl-2-buten-1-yl)-8-(6-amino-[1,4]diazepan-1-yl)-xanthine

Carried out with isopropanolic hydrochloric acid (5-6M) in methylene chloride.

R_{f} value: 0.41 (silica gel, methylene chloride/methanol/conc. aqueous ammonia=90:10:1)

Mass spectrum (ESI⁺): m/z=362 [M+H]⁺

### (112) 1,3-dimethyl-7-(3-methyl-2-buten-1-yl)-8-[N-(2-amino-2-methyl-propyl)-N-methyl-amino]-xanthine

Carried out with isopropanolic hydrochloric acid (5-6M) in methylene chloride.

Melting point: 156.5-159.5° C.

Mass spectrum (ESI⁺): m/z=349 [M+H]⁺

### (113) 1-[(pyridin-2-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

Melting point: 136-139.5° C.

Mass spectrum (ESI⁺): m/z=424 [M+H]⁺

### (114) 1-[(thiazol-2-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

Melting point: 124-127° C.

Mass spectrum (ESI⁺): m/z=430 [M+H]⁺

### (115) 1,3-dimethyl-7-(3-methyl-2-buten-1-yl)-8-(trans-2-amino-cyclopentylamino)-xanthine

Carried out with isopropanolic hydrochloric acid (5-6M) in methylene chloride.

R_{f} value: 0.25 (silica gel, methylene chloride/methanol/conc. aqueous ammonia=95:5:0.1)

Mass spectrum (ESI⁺): m/z=347 [M+H]⁺

### (116) 1,3-dimethyl-7-(3-methyl-2-buten-1-yl)-8-(trans-3-amino-cyclohexylamino)-xanthine (contaminated with about 25% of cis compound)

Carried out with isopropanolic hydrochloric acid (5-6M) in methylene chloride.

R_{f} value: 0.16 (silica gel, methylene chloride/methanol/conc. aqueous ammonia=90:10:1)

Mass spectrum (ESI⁻): m/z=359 [M−H]⁻

### (117) 1,3-dimethyl-7-(3-methyl-2-buten-1-yl)-8-(cis-3-amino-cyclohexylamino)-xanthine (contaminated with about 21% of trans compound)

Carried out with isopropanolic hydrochloric acid (5-6M) in methylene chloride.

R_{f} value: 0.21 (silica gel, methylene chloride/methanol/conc. aqueous ammonia=90:10:1)

Mass spectrum (ESI⁻): m/z=359 [M−H]⁻

### (118) 1,3-dimethyl-7-(3-methyl-2-buten-1-yl)-8-(cis-2-amino-cyclopentylamino)-xanthine

Carried out with isopropanolic hydrochloric acid (5-6M) in methylene chloride.

R_{f} value: 0.25 (silica gel, methylene chloride/methanol/conc. aqueous ammonia=95:5:0.1)

Mass spectrum (ESI⁺): m/z=347 [M+H]⁺

### (119) 1-[(isoquinolin-1-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

Melting point: 146-149° C.

Mass spectrum (ESI⁺): m/z=474 [M+H]⁺

### (120) 1,3-dimethyl-7-(3-methyl-2-buten-1-yl)-8-(cis-3-amino-cyclopentylamino)-xanthine

Carried out with isopropanolic hydrochloric acid (5-6M) in methylene chloride.

Melting point: 146-148° C.

Mass spectrum (ESI⁺): m/z=347 [M+H]⁺

### (121) 1-[(benzo[d]isothiazol-3-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

Melting point: 129-131° C.

Mass spectrum (ESI⁺): m/z=480 [M+H]⁺

### (122) 1-[(pyridin-3-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

R_{f} value: 0.42 (silica gel, methylene chloride/methanol/conc. aqueous ammonia=90:10:1)

Mass spectrum (ESI⁺): m/z=424 [M+H]⁺

### (123) 1-[(pyridin-4-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

R_{f} value: 0.48 (silica gel, methylene chloride/methanol/conc. aqueous ammonia=90:10:1)

Mass spectrum (ESI⁺): m/z=424 [M+H]⁺

### (124) 1-[(isoxazol-3-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

Melting point: 124-127.5° C.

Mass spectrum (ESI⁺): m/z=414 [M+H]⁺

### (125) 1-[(isoquinolin-1-yl)methyl]-3-methyl-7-(3-methyl-2-buten-1-yl)-8-((R)-3-amino-piperidin-1-yl)-xanthine

Carried out with isopropanolic hydrochloric acid (5-6M) in methylene chloride.

R_{f} value: 0.50 (silica gel, methylene chloride/methanol/conc. aqueous ammonia=90:10:1)

Mass spectrum (ESI⁺): m/z=474 [M+H]⁺

### (126) 1-[(isoquinolin-1-yl)methyl]-3-methyl-7-(3-methyl-2-buten-1-yl)-8-((S)-3-amino-piperidin-1-yl)-xanthine

Carried out with isopropanolic hydrochloric acid (5-6M) in methylene chloride.

Mass spectrum (ESI⁺): m/z=474 [M+H]⁺

### (127) 1-[(1-naphthyl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

R_{f} value: 0.51 (silica gel, methylene chloride/methanol/conc. aqueous ammonia=90:10:1)

Mass spectrum (ESI⁺): m/z=473 [M+H]⁺

### (128) 1-[(benzo[d]isoxazol-3-yl)methyl]-3-methyl-7-(3-methyl-2-buten-1-yl)-8-(3-amino-piperidin-1-yl)-xanthine

R_{f} value: 0.20 (silica gel, methylene chloride/methanol=9:1)

Mass spectrum (ESI⁺): m/z=464 [M+H]⁺

### (129) 1-(2-phenyl-2-oxo-ethyl)-3-methyl-7-(3-methyl-2-buten-1-yl)-8-(3-amino-3-methyl-piperidin-1-yl)-xanthine

R_{f} value: 0.18 (silica gel, ethyl acetate/methanol/conc. aqueous ammonia=90:10:1)

Mass spectrum (ESI⁺): m/z=465 [M+H]⁺

### (130) 1,3-dimethyl-7-(3-methyl-2-buten-1-yl)-8-(3-amino-3-methyl-piperidin-1-yl)-xanthine

R_{f} value: 0.41 (aluminium oxide, methylene chloride/methanol=20:1)

Mass spectrum (ESI⁺): m/z=361 [M+H]⁺

### (131) 1,3-dimethyl-7-(3-methyl-2-buten-1-yl)-8-[N-(2-amino-3-dimethylamino-3-oxo-propyl)-N-methyl-amino]-xanthine x trifluoroacetic acid

R_{f} value: 0.31 (silica gel, methylene chloride/methanol/conc. aqueous ammonia=40:10:1)

Mass spectrum (ESI⁺): m/z=392 [M+H]⁺

### (132) 1,3-dimethyl-7-(3-methyl-2-buten-1-yl)-8-[N-(2,3-diamino-3-oxo-propyl)-N-methyl-amino]-xanthine x trifluoroacetic acid

R_{f} value: 0.28 (silica gel, methylene chloride/methanol/conc. aqueous ammonia=40:10:1)

Mass spectrum (ESI⁺): m/z=364 [M+H]⁺

### (133) 1-[(aminocarbonyl)methyl)]-3-methyl-7-(2-cyano-benzyl)-8-(3-amino-piperidin-1-yl)-xanthine

Prepared from 1-cyanomethyl-3-methyl-7-(2-cyano-benzyl)-8-[3-(tert.-butyloxycarbonylamino)-piperidin-1-yl]-xanthine. During the treatment with trifluoroacetic acid the protecting group is cleaved and the cyano group is hydrolysed to form the amide.

R_{f} value: 0.10 (silica gel, methylene chloride/methanol/conc. aqueous ammonia=90:10:0.1)

Mass spectrum (ESI⁺): m/z=437 [M+H]⁺

### (134) 1-[2-(3-methanesulphonylamino-phenyl)-2-oxo-ethyl]-3-methyl-7-(3-methyl-2-buten-1-yl)-8-(3-amino-piperidin-1-yl)-xanthine

Mass spectrum (ESI⁺): m/z=544 [M+H]⁺

R_{f} value: 0.45 (silica gel, methylene chloride/methanol/triethylamine=90:10:0.1)

### (135) 1-[2-(2-nitro-phenyl)-2-oxo-ethyl]-3-methyl-7-(3-methyl-2-buten-1-yl)-8-(3-amino-piperidin-1-yl)-xanthine

Mass spectrum (ESI⁺): m/z=496 [M+H]⁺

### (136) 1-[2-(2-amino-phenyl)-2-oxo-ethyl]-3-methyl-7-(3-methyl-2-buten-1-yl)-8-(3-amino-piperidin-1-yl)-xanthine

Mass spectrum (ESI⁺): m/z=466 [M+H]⁺

### (137) 1-(2-{3-[(methylamino)thiocarbonylamino]-phenyl}-2-oxo-ethyl)-3-methyl-7-(3-methyl-2-buten-1-yl)-8-(3-amino-piperidin-1-yl)-xanthine

R_{f} value: 0.30 (silica gel, methylene chloride/methanol/conc. aqueous ammonia=80:20:0.1)

Mass spectrum (ESI⁺): m/z=539 [M+H]⁺

### (138) 1-[2-(2-acetylamino-phenyl)-2-oxo-ethyl]-3-methyl-7-(3-methyl-2-buten-1-yl)-8-(3-amino-piperidin-1-yl)-xanthine

Mass spectrum (ESI⁺): m/z=508 [M+H]⁺

### (139) 1-[(6-methyl-pyridin-2-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

Melting point: 127.5-130° C.

Mass spectrum (ESI⁺): m/z=438 [M+H]⁺

### (140) 1-[(isoquinolin-4-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

R_{f} value: 0.40 (silica gel, methylene chloride/methanol/conc. aqueous ammonia=90:10:1)

Mass spectrum (ESI⁺): m/z=474 [M+H]⁺

### (141) 1-[(1-methyl-1H-indazol-3-yl)methyl]-3-methyl-7-(3-methyl-2-buten-1-yl)-8-(3-amino-piperidin-1-yl)-xanthine

Carried out with isopropanolic hydrochloric acid (5-6M) in methylene chloride.

R_{f} value: 0.31 (silica gel, methylene chloride/methanol/conc. aqueous ammonia=90:10:1)

Mass spectrum (ESI⁺): m/z=477 [M+H]⁺

### (142) 1,3-dimethyl-7-(3-methyl-2-buten-1-yl)-8-{N-[2-amino-3-oxo-3-(pyrrolidin-1-yl)-propyl]-N-methyl-amino}-xanthine

Melting point: 138° C.

Mass spectrum (ESI⁺): m/z=418 [M+H]⁺

### (143) 1,3-dimethyl-7-(3-methyl-2-buten-1-yl)-8-[N-(2-amino-3-methylamino-3-oxo-propyl)-N-methyl-amino]-xanthine

R_{f} value: 0.20 (silica gel, methylene chloride/methanol/conc. aqueous ammonia=90:10:1)

Mass spectrum (ESI⁺): m/z=378 [M+H]⁺

### (144) 1-(2-{3-[(methoxycarbonyl)methylamino]-phenyl}-2-oxo-ethyl)-3-methyl-7-(3-methyl-2-buten-1-yl)-8-(3-amino-piperidin-1-yl)-xanthine

Rf value: 0.29 (silica gel, methylene chloride/methanol/conc. aqueous ammonia=80:20:0.1)

Mass spectrum (ESI⁺): m/z=538 [M+H]⁺

### (145) 1-cyanomethyl-3-methyl-7-(2-cyano-benzyl)-8-(3-amino-piperidin-1-yl)-xanthine

Carried out with isopropanolic hydrochloric acid (5-6M) in methylene chloride.

Rf value: 0.60 (silica gel, methylene chloride/methanol=9:2)

Mass spectrum (ESI⁺): m/z=419 [M+H]⁺

### (146) 1-[2-(2-hydroxy-phenyl)-2-oxo-ethyl]-3-methyl-7-(3-methyl-2-buten-1-yl)-8-(3-amino-piperidin-1-yl)-xanthine x trifluoroacetic acid

Mass spectrum (ESI⁺): m/z=467 [M+H]⁺

### (147) 1-[2-(2-methanesulphonyloxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=545 [M+H]⁺

### (148) 1-(2-{2-[(methoxycarbonyl)methoxy]-phenyl}-2-oxo-ethyl)-3-methyl-7-(3-methyl-2-buten-1-yl)-8-(3-amino-piperidin-1-yl)-xanthine

Mass spectrum (ESI⁺): m/z=539 [M+H]⁺

### (149) 1-[2-(2-cyanomethoxy-phenyl)-2-oxo-ethyl]-3-methyl-7-(3-methyl-2-buten-1-yl)-8-(3-amino-piperidin-1-yl)-xanthine

Mass spectrum (ESI⁺): m/z=506 [M+H]⁺

### (150) 1-(2-{3-[(dimethylaminocarbonyl)methoxy]-phenyl}-2-oxo-ethyl)-3-methyl-7-(3-methyl-2-buten-1-yl)-8-(3-amino-piperidin-1-yl)-xanthine

Rf value: 0.45 (silica gel, methylene chloride/methanol/triethylamine=80:20:0.1)

Mass spectrum (ESI⁺): m/z=552 [M+H]⁺

### (151) 1-(2-{3-[(methylaminocarbonyl)methoxy]-phenyl}-2-oxo-ethyl)-3-methyl-7-(3-methyl-2-buten-1-yl)-8-(3-amino-piperidin-1-yl)-xanthine

Rf value: 0.55 (silica gel, methylene chloride/methanol/triethylamine=80:20:0.1)

Mass spectrum (ESI⁺): m/z=538 [M+H]⁺

### (152) 1-(2-{3-[(aminocarbonyl)methoxy]-phenyl}-2-oxo-ethyl)-3-methyl-7-(3-methyl-2-buten-1-yl)-8-(3-amino-piperidin-1-yl)-xanthine

Mass spectrum (ESI⁺): m/z=524 [M+H]⁺

### (153) 1-(2-{2-[bis(methanesulphonyl)-amino]-phenyl}-2-oxo-ethyl)-3-methyl-7-(3-methyl-2-buten-1-yl)-8-(3-amino-piperidin-1-yl)-xanthine

Mass spectrum (ESI⁺): m/z=622 [M+H]⁺

### (154) 1-methyl-3-[2-(4-methoxy-phenyl)-ethyl]-7-(2-cyano-benzyl)-8-(3-amino-piperidin-1-yl)-xanthine

Rf value: 0.35 (silica gel, methylene chloride/methanol=9:1)

Mass spectrum (ESI⁺): m/z=514 [M+H]⁺

### (155) 1-methyl-3-(2-phenyl-ethyl)-7-(2-cyano-benzyl)-8-(3-amino-piperidin-1-yl)-xanthine

Mass spectrum (ESI⁺): m/z=484 [M+H]⁺

### (156) 1-(2-{3-[(aminocarbonyl)amino]-phenyl}-2-oxo-ethyl)-3-methyl-7-(3-methyl-2-buten-1-yl)-8-(3-amino-piperidin-1-yl)-xanthine

Mass spectrum (ESI⁺): m/z=509 [M+H]⁺

### (157) 1-(2-{3-[(dimethylaminocarbonyl)amino]-phenyl}-2-oxo-ethyl)-3-methyl-7-(3-methyl-2-buten-1-yl)-8-(3-amino-piperidin-1-yl)-xanthine

Mass spectrum (ESI⁺): m/z=537 [M+H]⁺

### (158) 1-methyl-3-((E)-2-phenyl-vinyl)-7-(3-methyl-2-buten-1-yl)-8-(3-amino-piperidin-1-yl)-xanthine

Rf value: 0.49 (silica gel, methylene chloride/methanol/conc. aqueous ammonia=90:10:1)

Mass spectrum (ESI⁺): m/z=435 [M+H]⁺

### (159) 1-(4-oxo-4H-chromen-3-yl)-3-methyl-7-(3-methyl-2-buten-1-yl)-8-(3-amino-piperidin-1-yl)-xanthine x trifluoroacetic acid

Mass spectrum (ESI⁺): m/z=477 [M+H]⁺

### (160) 1-[(3-methyl-pyridin-2-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Rf value: 0.54 (silica gel, methylene chloride/methanol/conc. aqueous ammonia=90:10:1)

Mass spectrum (ESI⁺): m/z=438 [M+H]⁺

### (161) 1-[(5-methyl-pyridin-2-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Rf value: 0.35 (silica gel, methylene chloride/methanol/conc. aqueous ammonia=90:10:1)

Mass spectrum (ESI⁺): m/z=438 [M+H]⁺

### (162) 1-[(4-methyl-pyridin-2-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Rf value: 0.39 (silica gel, methylene chloride/methanol/conc. aqueous ammonia=90:10:1)

Mass spectrum (ESI⁺): m/z=438 [M+H]⁺

### (163) 1-[(quinolin-4-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Rf value: 0.53 (silica gel, methylene chloride/methanol/conc. aqueous ammonia=90:10:1)

Mass spectrum (ESI⁺): m/z=474 [M+H]⁺

### (164) 1,3-dimethyl-7-(3-methyl-2-buten-1-yl)-8-(endo-6-amino-2-aza-bicyclo[2.2.2]oct-2-yl)-xanthine

Carried out with isopropanolic hydrochloric acid (5-6 M) in methylene chloride.

Melting point: 174-179° C.

Mass spectrum (ESI⁺): m/z=373 [M+H]⁺

### (165) 1-[(quinolin-8-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Melting point: 175-177° C.

Mass spectrum (ESI⁺): m/z=474 [M+H]⁺

### (166) 1-[(5-nitro-isoquinolin-1-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Rf value: 0.47 (silica gel, methylene chloride/methanol/conc. aqueous ammonia=90:10:1)

Mass spectrum (ESI⁺): m/z=519 [M+H]⁺

### (167) 1,3-dimethyl-7-(3-methyl-2-buten-1-yl)-8-(exo-6-amino-2-aza-bicyclo[2.2.2]oct-2-yl)-xanthine

Carried out with isopropanolic hydrochloric acid (5-6 M) in methylene chloride.

Rf value: 0.23 (silica gel, methylene chloride/methanol/conc. aqueous ammonia=95:5:0.1)

Mass spectrum (ESI⁺): m/z=373 [M+H]⁺

### (168) 1-[(2-oxo-1,2-dihydro-quinolin-4-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Rf value: 0.43 (silica gel, methylene chloride/methanol/conc. aqueous ammonia=90:10:1)

Mass spectrum (ESI⁺): m/z=490 [M+H]⁺

### (169) 1-[(5-amino-isoquinolin-1-yl)methyl]-3-methyl-7-(3-methyl-2-buten-1-yl)-8-(3-amino-piperidin-1-yl)-xanthine

Carried out with isopropanolic hydrochloric acid (5-6 M) in methylene chloride.

Rf value: 0.39 (silica gel, methylene chloride/methanol/conc. aqueous ammonia=90:10:1)

Mass spectrum (ESI⁺): m/z=489 [M+H]⁺

### (170) 1-[2-(3-cyano-phenyl)-2-oxo-ethyl]-3-methyl-7-(3-methyl-2-buten-1-yl)-8-(3-amino-piperidin-1-yl)-xanthine

Rf value: 0.65 (silica gel, methylene chloride/methanol/conc. aqueous ammonia=90:10:1)

Mass spectrum (ESI⁺): m/z=476 [M+H]⁺

### (171) 1-[2-(3-aminosulphonyl-phenyl)-2-oxo-ethyl]-3-methyl-7-(3-methyl-2-buten-1-yl)-8-(3-amino-piperidin-1-yl)-xanthine

Rf value: 0.24 (silica gel, methylene chloride/methanol/conc. aqueous ammonia=90:10:1)

Mass spectrum (ESI⁺): m/z=530 [M+H]⁺

### (172) 1-[2-(3-aminocarbonyl-phenyl)-2-oxo-ethyl]-3-methyl-7-(3-methyl-2-buten-1-yl)-8-(3-amino-piperidin-1-yl)-xanthine

Rf value: 0.10 (silica gel, methylene chloride/methanol/conc. aqueous ammonia=90:10:1)

Mass spectrum (ESI⁺): m/z=494 [M+H]⁺

### (173) 1-(2-phenoxy-ethyl)-3-methyl-7-(3-methyl-2-buten-1-yl)-8-(3-amino-piperidin-1-yl)-xanthine

Mass spectrum (ESI⁺): m/z=453 [M+H]⁺

### (174) 1,3-dimethyl-2-thioxo-7-benzyl-8-(3-amino-piperidin-1-yl)-xanthine x trifluoroacetic acid

Rf value: 0.50 (aluminium oxide, methylene chloride/methanol=20:1)

Mass spectrum (ESI⁺): m/z=385 [M+H]⁺

### EXAMPLE 3

### 1,3-dimethyl-7-(3-methyl-2-buten-1-yl)-8-(3-methylamino-piperidin-1-yl)-xanthine

154 mg of 1,3-dimethyl-7-(3-methyl-2-buten-1-yl)-8-(3-amino-piperidin-1-yl)-xanthine and 0.032 ml of aqueous formaldehyde solution (37% by weight) in 0.5 ml of methanol are combined with 24 mg of sodium borohydride and stirred at ambient temperature.

0.01 ml of formaldehyde solution and 10 mg of sodium borohydride are both added twice more and stirring is continued at ambient temperature. The reaction mixture is combined with 1M sodium hydroxide solution and repeatedly extracted with ethyl acetate. The organic phases are combined, dried and evaporated down. The residue is purified by chromatography over an aluminium oxide column with ethyl acetate/methanol.

Yield: 160 mg (25% of theory)

Mass spectrum (ESI⁺): m/z=361 [M+H]⁺

R_{f} value: 0.80 (aluminium oxide, ethyl acetate/methanol=4:1)

The following compound is obtained analogously to Example 3:

### (1) 1,3-dimethyl-7-(3-methyl-2-buten-1-yl)-8-(3-dimethylamino-piperidin-1-yl)-xanthine

Mass spectrum (ESI⁺): m/z=375 [M+H]⁺

R_{f} value: 0.65 (aluminium oxide, methylene chloride/methanol=100:1)

### EXAMPLE 4

### (S)-1,3-dimethyl-7-(3-methyl-2-buten-1-yl)-8-{3-[(2-cyanpyrrolidin-1-ylcarbonyl-methyl)amino]-piperidin-1-yl}-xanthine

Prepared by reacting the compound of Example 1(4) with (S)-1-(bromoacetyl)-2-cyano-pyrrolidine in tetrahydrofuran in the presence of triethylamine at ambient temperature

Melting point: 67-68° C.

Mass spectrum (ESI⁺): m/z=505 [M+Na]⁺

### EXAMPLE 5

### 1-methyl-7-benzyl-8-(3-amino-piperidin-1-yl)-xanthine

Prepared by treating 1-methyl-3-(2-trimethylsilanyl-ethoxymethyl)-7-benzyl-8-(3-amino-piperidin-1-yl)-xanthine with trifluoroacetic acid in methylene chloride at ambient temperature

Mass spectrum (ESI⁺): m/z=355 [M+H]⁺

### EXAMPLE 6

### 1-methyl-3-carboxymethyl-7-benzyl-8-(3-amino-piperidin-1-yl)-xanthine

Prepared by treating 1-methyl-3-[(methoxycarbonyl)-methyl]-7-benzyl-8-(3-amino-piperidin-1-yl)-xanthine with 1N sodium hydroxide solution in methanol

Melting point: 212-215° C.

Mass spectrum (ESI⁺): m/z=413 [M+H]⁺

The following compounds are obtained analogously to Example 6:

### (1) 1-carboxymethyl-3-methyl-7-(3-methyl-2-buten-1-yl)-8-((S)-3-amino-piperidin-1-yl)-xanthine

R_{f} value: 0.54 (ready-made reversed phase TLC plate(E. Merck), acetonitrile/water/trifluoroacetic acid=50:50:1)

Mass spectrum (ESI⁺): m/z=391 [M+H]⁺

### (2) 1-(3-carboxy-propyl)-3-methyl-7-(3-methyl-2-buten-1-yl)-8-((S)-3-amino-piperidin-1-yl)-xanthine

R_{f} value: 0.42 (ready-made reversed phase TLC plate(E. Merck), acetonitrile/water/trifluoroacetic acid=50:50:1)

Mass spectrum (ESI⁺): m/z=419 [M+H]⁺

### (3) 1-[2-(4-carboxy-phenyl)-ethyl]-3-methyl-7-(3-methyl-2-buten-1-yl)-8-((S)-3-amino-piperidin-1-yl)-xanthine

R_{f} value: 0.42 (ready-made reversed phase TLC plate(E. Merck), acetonitrile/water/trifluoroacetic acid=50:50:1)

Mass spectrum (ESI⁺): m/z=481 [M+H]⁺

### (4) 1-(2-carboxy-ethyl)-3-methyl-7-(3-methyl-2-buten-1-yl)-8-((S)-3-amino-piperidin-1-yl)-xanthine

Melting point: 226-228° C.

Mass spectrum (ESI⁺): m/z=405 [M+H]⁺

### (5) 1-(2-phenyl-ethyl)-3-carboxymethyl-7-(3-methyl-2-buten-1-yl)-8-(3-amino-piperidin-1-yl)-xanthine

Melting point: 228-235° C.

Mass spectrum (ESI⁺): m/z=481 [M+H]⁺

### EXAMPLE 7

### 1-[2-(3-amino-phenyl)-ethyl]-3-methyl-7-(3-methyl-2-buten-1-yl)-8-(3-amino-piperidin-1-yl)-xanthine

Prepared by reduction of 1-[2-(3-nitro-phenyl)-ethyl]-3-methyl-7-(3-methyl-2-buten-1-yl)-8-(3-amino-piperidin-1-yl)-xanthine with iron in a mixture of ethanol, water and glacial acetic acid (10:5:1).

R_{f} value: 0.45 (silica gel, methylene chloride/methanol/conc. aqueous ammonia=9:1:0.1)

Mass spectrum (ESI⁺): m/z=452 [M+H]⁺

The following compounds are obtained analogously to Example 7:

### (1) 1-[2-(2-amino-phenyl)-ethyl]-3-methyl-7-(3-methyl-2-buten-1-yl)-8-(3-amino-piperidin-1-yl)-xanthine

R_{f} value: 0.20 (silica gel, methylene chloride/methanol/conc. aqueous ammonia=9:1:0.1)

Mass spectrum (ESI⁺): m/z=452 [M+H]⁺

### (2) 1,3-dimethyl-7-(3-amino-benzyl)-8-(3-amino-piperidin-1-yl)-xanthine

R_{f} value: 0.20 (silica gel, methylene chloride/methanol/conc. aqueous ammonia=90:10:1)

Mass spectrum (ESI⁺): m/z=384 [M+H]⁺

### (3) 1,3-dimethyl-7-(2-amino-benzyl)-8-(3-amino-piperidin-1-yl)-xanthine

Mass spectrum (ESI⁺): m/z=384 [M+H]⁺

### EXAMPLE 8

### 1,3-dimethyl-7-(3-methyl-2-buten-1-yl)-8-(1-amino-piperidin-4-yl)-xanthine

Prepared by treating 1,3-dimethyl-7-(3-methyl-2-buten-1-yl)-8-(1-nitroso-piperidin-4-yl)-xanthine with zinc in a mixture of acetic acid and water (1:1.5) at 80° C.

Mass spectrum (ESI⁺): m/z=347 [M+H]⁺

The following compounds are obtained analogously to Example 8:

### (1) 1,3-dimethyl-7-(3-methyl-2-buten-1-yl)-8-(1-amino-piperidin-3-yl)-xanthine

Mass spectrum (ESI⁺): m/z=347 [M+H]⁺

### EXAMPLE 9

### 1-(2-hydroxyimino-2-phenyl-ethyl)-3-methyl-7-(3-methyl-2-buten-1-yl)-8-((R)-3-amino-piperidin-1-yl)-xanthine

Prepared by reacting 1-(2-phenyl-2-oxo-ethyl)-3-methyl-7-(3-methyl-2-buten-1-yl)-8-((R)-3-amino-piperidin-1-yl)-xanthine with hydroxylamine-hydrochloride in the presence of potassium carbonate in ethanol at 85° C.

R_{f} value: 0.54 (ready-made reversed phase TLC plate(E. Merck), acetonitrile/water/trifluoroacetic acid=10:10:0.2)

Mass spectrum (ESI⁺): m/z=466 [M+H]⁺

### EXAMPLE 10

### 1-[2-(2-methanesulphonylamino-phenyl)-2-oxo-ethyl]-3-methyl-7-(3-methyl-2-buten-1-yl)-8-(3-amino-piperidin-1-yl)-xanthine

Prepared by treating 1-(2-{2-[bis(methanesulphonyl)-amino]-phenyl}-2-oxo-ethyl)-3-methyl-7-(3-methyl-2-buten-1-yl)-8-(3-amino-piperidin-1-yl)-xanthine with 5 N sodium hydroxide solution in tetrahydrofuran at ambient temperature.

Mass spectrum (ESI⁺): m/z=544 [M+H]⁺

The following compounds may also be obtained analogously to the foregoing Examples and other methods known from the literature:
(1) 7-(3-methyl-2-buten-1-yl)-8-(3-amino-piperidin-1-yl)-xanthine
(2) 1-methyl-7-(3-methyl-2-buten-1-yl)-8-(3-amino-piperidin-1-yl)-xanthine
(3) 3-methyl-7-(3-methyl-2-buten-1-yl)-8-(3-amino-piperidin-1-yl)-xanthine
(4) 1-ethyl-3-methyl-7-(3-methyl-2-buten-1-yl)-8-(3-amino-piperidin-1-yl)-xanthine
(5) 1-propyl-3-methyl-7-(3-methyl-2-buten-1-yl)-8-(3-amino-piperidin-1-yl)-xanthine
(6) 1-(2-propyl)-3-methyl-7-(3-methyl-2-buten-1-yl)-8-(3-amino-piperidin-1-yl)-xanthine
(7) 1-butyl-3-methyl-7-(3-methyl-2-buten-1-yl)-8-(3-amino-piperidin-1-yl)-xanthine
(8) 1-(2-butyl)-3-methyl-7-(3-methyl-2-buten-1-yl)-8-(3-amino-piperidin-1-yl)-xanthine
(9) 1-(2-methylpropyl)-3-methyl-7-(3-methyl-2-buten-1-yl)-8-(3-amino-piperidin-1-yl)-xanthine
(10) 1-(2-propen-1-yl)-3-methyl-7-(3-methyl-2-buten-1-yl)-8-(3-amino-piperidin-1-yl)-xanthine
(11) 1-(2-propyn-1-yl)-3-methyl-7-(3-methyl-2-buten-1-yl)-8-(3-amino-piperidin-1-yl)-xanthine
(12) 1-cyclopropylmethyl-3-methyl-7-(3-methyl-2-buten-1-yl)-8-(3-amino-piperidin-1-yl)-xanthine
(13) 1-benzyl-3-methyl-7-(3-methyl-2-buten-1-yl)-8-(3-amino-piperidin-1-yl)-xanthine
(14) 1-(2-phenylethyl)-3-methyl-7-(3-methyl-2-buten-1-yl)-8-(3-amino-piperidin-1-yl)-xanthine
(15) 1-(2-hydroxyethyl)-3-methyl-7-(3-methyl-2-buten-1-yl)-8-(3-amino-piperidin-1-yl)-xanthine
(16) 1-(2-methoxyethyl)-3-methyl-7-(3-methyl-2-buten-1-yl)-8-(3-amino-piperidin-1-yl)-xanthine
(17) 1-(2-ethoxyethyl)-3-methyl-7-(3-methyl-2-buten-1-yl)-8-(3-amino-piperidin-1-yl)-xanthine
(18) 1-[2-(dimethylamino)ethyl]-3-methyl-7-(3-methyl-2-buten-1-yl)-8-(3-amino-piperidin-1-yl)-xanthine
(19) 1-[2-(diethylamino)ethyl]-3-methyl-7-(3-methyl-2-buten-1-yl)-8-(3-amino-piperidin-1-yl)-xanthine
(20) 1-[2-(pyrrolidin-1-yl)ethyl]-3-methyl-7-(3-methyl-2-buten-1-yl)-8-(3-amino-piperidin-1-yl)-xanthine
(21) 1-[2-(piperidin-1-yl)ethyl]-3-methyl-7-(3-methyl-2-buten-1-yl)-8-(3-amino-piperidin-1-yl)-xanthine
(22) 1-[2-(morpholin-4-yl)ethyl]-3-methyl-7-(3-methyl-2-buten-1-yl)-8-(3-amino-piperidin-1-yl)-xanthine
(23) 1-[2-(piperazin-1-yl)ethyl]-3-methyl-7-(3-methyl-2-buten-1-yl)-8-(3-amino-piperidin-1-yl)-xanthine
(24) 1-[2-(4-methyl-piperazin-1-yl)ethyl]-3-methyl-7-(3-methyl-2-buten-1-yl)-8-(3-amino-piperidin-1-yl)-xanthine
(25) 1-(3-hydroxypropyl)-3-methyl-7-(3-methyl-2-buten-1-yl)-8-(3-amino-piperidin-1-yl)-xanthine
(26) 1-(3-methoxypropyl)-3-methyl-7-(3-methyl-2-buten-1-yl)-8-(3-amino-piperidin-1-yl)-xanthine
(27) 1-(3-ethoxypropyl)-3-methyl-7-(3-methyl-2-buten-1-yl)-8-(3-amino-piperidin-1-yl)-xanthine
(28) 1-[3-(dimethylamino)propyl]-3-methyl-7-(3-methyl-2-buten-1-yl)-8-(3-amino-piperidin-1-yl)-xanthine
(29) 1-[3-(diethylamino)propyl]-3-methyl-7-(3-methyl-2-buten-1-yl)-8-(3-amino-piperidin-1-yl)-xanthine
(30) 1-[3-(pyrrolidin-1-yl)propyl]-3-methyl-7-(3-methyl-2-buten-1-yl)-8-(3-amino-piperidin-1-yl)-xanthine
(31) 1-[3-(piperidin-1-yl)propyl]-3-methyl-7-(3-methyl-2-buten-1-yl)-8-(3-amino-piperidin-1-yl)-xanthine
(32) 1-[3-(morpholin-4-yl)propyl]-3-methyl-7-(3-methyl-2-buten-1-yl)-8-(3-amino-piperidin-1-yl)-xanthine
(33) 1-[3-(piperazin-1-yl)propyl]-3-methyl-7-(3-methyl-2-buten-1-yl)-8-(3-amino-piperidin-1-yl)-xanthine
(34) 1-[3-(4-methyl-piperazin-1-yl)propyl]-3-methyl-7-(3-methyl-2-buten-1-yl)-8-(3-amino-piperidin-1-yl)-xanthine
(35) 1-(carboxymethyl)-3-methyl-7-(3-methyl-2-buten-1-yl)-8-(3-amino-piperidin-1-yl)-xanthine
(36) 1-(methoxycarbonylmethyl)-3-methyl-7-(3-methyl-2-buten-1-yl)-8-(3-amino-piperidin-1-yl)-xanthine
(37) 1-(ethoxycarbonylmethyl)-3-methyl-7-(3-methyl-2-buten-1-yl)-8-(3-amino-piperidin-1-yl)-xanthine
(38) 1-(2-carboxyethyl)-3-methyl-7-(3-methyl-2-buten-1-yl)-8-(3-amino-piperidin-1-yl)-xanthine
(39) 1-[2-(methoxycarbonyl)ethyl]-3-methyl-7-(3-methyl-2-buten-1-yl)-8-(3-amino-piperidin-1-yl)-xanthine
(40) 1-[2-(ethoxycarbonyl)ethyl]-3-methyl-7-(3-methyl-2-buten-1-yl)-8-(3-amino-piperidin-1-yl)-xanthine
(41) 1-(aminocarbonylmethyl)-3-methyl-7-(3-methyl-2-buten-1-yl)-8-(3-amino-piperidin-1-yl)-xanthine
(42) 1-(methylaminocarbonylmethyl)-3-methyl-7-(3-methyl-2-buten-1-yl)-8-(3-amino-piperidin-1-yl)-xanthine
(43) 1-(dimethylaminocarbonylmethyl)-3-methyl-7-(3-methyl-2-buten-1-yl)-8-(3-amino-piperidin-1-yl)-xanthine
(44) 1-(pyrrolidin-1-yl-carbonylmethyl)-3-methyl-7-(3-methyl-2-buten-1-yl)-8-(3-amino-piperidin-1-yl)-xanthine
(45) 1-(piperidin-1-yl-carbonylmethyl)-3-methyl-7-(3-methyl-2-buten-1-yl)-8-(3-amino-piperidin-1-yl)-xanthine
(46) 1-(morpholin-4-yl-carbonylmethyl)-3-methyl-7-(3-methyl-2-buten-1-yl)-8-(3-amino-piperidin-1-yl)-xanthine
(47) 1-(cyanomethyl)-3-methyl-7-(3-methyl-2-buten-1-yl)-8-(3-amino-piperidin-1-yl)-xanthine
(48) 1-(2-cyanoethyl)-3-methyl-7-(3-methyl-2-buten-1-yl)-8-(3-amino-piperidin-1-yl)-xanthine
(49) 1-methyl-3-ethyl-7-(3-methyl-2-buten-1-yl)-8-(3-amino-piperidin-1-yl)-xanthine
(50) 1-methyl-3-propyl-7-(3-methyl-2-buten-1-yl)-8-(3-amino-piperidin-1-yl)-xanthine
(51) 1-methyl-3-(2-propyl)-7-(3-methyl-2-buten-1-yl)-8-(3-amino-piperidin-1-yl)-xanthine
(52) 1-methyl-3-butyl-7-(3-methyl-2-buten-1-yl)-8-(3-amino-piperidin-1-yl)-xanthine
(53) 1-methyl-3-(2-butyl)-7-(3-methyl-2-buten-1-yl)-8-(3-amino-piperidin-1-yl)-xanthine
(54) 1-methyl-3-(2-methylpropyl)-7-(3-methyl-2-buten-1-yl)-8-(3-amino-piperidin-1-yl)-xanthine
(55) 1-methyl-3-(2-propen-1-yl)-7-(3-methyl-2-buten-1-yl)-8-(3-amino-piperidin-1-yl)-xanthine
(56) 1-methyl-3-(2-propyn-1-yl)-7-(3-methyl-2-buten-1-yl)-8-(3-amino-piperidin-1-yl)-xanthine
(57) 1-methyl-3-cyclopropylmethyl-7-(3-methyl-2-buten-1-yl)-8-(3-amino-piperidin-1-yl)-xanthine
(58) 1-methyl-3-benzyl-7-(3-methyl-2-buten-1-yl)-8-(3-amino-piperidin-1-yl)-xanthine
(59) 1-methyl-3-(2-phenylethyl)-7-(3-methyl-2-buten-1-yl)-8-(3-amino-piperidin-1-yl)-xanthine
(60) 1-methyl-3-(2-hydroxyethyl)-7-(3-methyl-2-buten-1-yl)-8-(3-amino-piperidin-1-yl)-xanthine
(61) 1-methyl-3-(2-methoxyethyl)-7-(3-methyl-2-buten-1-yl)-8-(3-amino-piperidin-1-yl)-xanthine
(62) 1-methyl-3-(2-ethoxyethyl)-7-(3-methyl-2-buten-1-yl)-8-(3-amino-piperidin-1-yl)-xanthine
(63) 1-methyl-3-[2-(dimethylamino)ethyl]-7-(3-methyl-2-buten-1-yl)-8-(3-amino-piperidin-1-yl)-xanthine
(64) 1-methyl-3-[2-(diethylamino)ethyl]-7-(3-methyl-2-buten-1-yl)-8-(3-amino-piperidin-1-yl)-xanthine
(65) 1-methyl-3-[2-(pyrrolidin-1-yl)ethyl]-7-(3-methyl-2-buten-1-yl)-8-(3-amino-piperidin-1-yl)-xanthine
(66) 1-methyl-3-[2-(piperidin-1-yl)ethyl]-7-(3-methyl-2-buten-1-yl)-8-(3-amino-piperidin-1-yl)-xanthine
(67) 1-methyl-3-[2-(morpholin-4-yl)ethyl]-7-(3-methyl-2-buten-1-yl)-8-(3-amino-piperidin-1-yl)-xanthine
(68) 1-methyl-3-[2-(piperazin-1-yl)ethyl]-7-(3-methyl-2-buten-1-yl)-8-(3-amino-piperidin-1-yl)-xanthine
(69) 1-methyl-3-[2-(4-methyl-piperazin-1-yl)ethyl]-7-(3-methyl-2-buten-1-yl)-8-(3-amino-piperidin-1-yl)-xanthine
(70) 1-methyl-3-(3-hydroxypropyl)-7-(3-methyl-2-buten-1-yl)-8-(3-amino-piperidin-1-yl)-xanthine
(71) 1-methyl-3-(3-methoxypropyl)-7-(3-methyl-2-buten-1-yl)-8-(3-amino-piperidin-1-yl)-xanthine
(72) 1-methyl-3-(3-ethoxypropyl)-7-(3-methyl-2-buten-1-yl)-8-(3-amino-piperidin-1-yl)-xanthine
(73) 1-methyl-3-[3-(dimethylamino)propyl]-7-(3-methyl-2-buten-1-yl)-8-(3-amino-piperidin-1-yl)-xanthine
(74) 1-methyl-3-[3-(diethylamino)propyl]-7-(3-methyl-2-buten-1-yl)-8-(3-amino-piperidin-1-yl)-xanthine
(75) 1-methyl-3-[3-(pyrrolidin-1-yl)propyl]-7-(3-methyl-2-buten-1-yl)-8-(3-amino-piperidin-1-yl)-xanthine
(76) 1-methyl-3-[3-(piperidin-1-yl)propyl]-7-(3-methyl-2-buten-1-yl)-8-(3-amino-piperidin-1-yl)-xanthine
(77) 1-methyl-3-[3-(morpholin-4-yl)propyl]-7-(3-methyl-2-buten-1-yl)-8-(3-amino-piperidin-1-yl)-xanthine
(78) 1-methyl-3-[3-(piperazin-1-yl)propyl]-7-(3-methyl-2-buten-1-yl)-8-(3-amino-piperidin-1-yl)-xanthine
(79) 1-methyl-3-[3-(4-methyl-piperazin-1-yl)propyl]-7-(3-methyl-2-buten-1-yl)-8-(3-amino-piperidin-1-yl)-xanthine
(80) 1-methyl-3-(carboxymethyl)-7-(3-methyl-2-buten-1-yl)-8-(3-amino-piperidin-1-yl)-xanthine
(81) 1-methyl-3-(methoxycarbonylmethyl)-7-(3-methyl-2-buten-1-yl)-8-(3-amino-piperidin-1-yl)-xanthine
(82) 1-methyl-3-(ethoxycarbonylmethyl)-7-(3-methyl-2-buten-1-yl)-8-(3-amino-piperidin-1-yl)-xanthine
(83) 1-methyl-3-(2-carboxyethyl)-7-(3-methyl-2-buten-1-yl)-8-(3-amino-piperidin-1-yl)-xanthine
(84) 1-methyl-3-[2-(methoxycarbonyl)ethyl]-7-(3-methyl-2-buten-1-yl)-8-(3-amino-piperidin-1-yl)-xanthine
(85) 1-methyl-3-[2-(ethoxycarbonyl)ethyl]-7-(3-methyl-2-buten-1-yl)-8-(3-amino-piperidin-1-yl)-xanthine
(86) 1-methyl-3-(aminocarbonylmethyl)-7-(3-methyl-2-buten-1-yl)-8-(3-amino-piperidin-1-yl)-xanthine
(87) 1-methyl-3-(methylaminocarbonylmethyl)-7-(3-methyl-2-buten-1-yl)-8-(3-amino-piperidin-1-yl)-xanthine
(88) 1-methyl-3-(dimethylaminocarbonylmethyl)-7-(3-methyl-2-buten-1-yl)-8-(3-amino-piperidin-1-yl)-xanthine
(89) 1-methyl-3-(pyrrolidin-1-yl-carbonylmethyl)-7-(3-methyl-2-buten-1-yl)-8-(3-amino-piperidin-1-yl)-xanthine
(90) 1-methyl-3-(piperidin-1-yl-carbonylmethyl)-7-(3-methyl-2-buten-1-yl)-8-(3-amino-piperidin-1-yl)-xanthine
(91) 1-methyl-3-(morpholin-4-yl-carbonylmethyl)-7-(3-methyl-2-buten-1-yl)-8-(3-amino-piperidin-1-yl)-xanthine
(92) 1-methyl-3-(cyanomethyl)-7-(3-methyl-2-buten-1-yl)-8-(3-amino-piperidin-1-yl)-xanthine
(93) 1-methyl-3-(2-cyanoethyl)-7-(3-methyl-2-buten-1-yl)-8-(3-amino-piperidin-1-yl)-xanthine
(94) 1,3,7-trimethyl-8-(3-amino-piperidin-1-yl)-xanthine
(95) 1,3-dimethyl-7-ethyl-8-(3-amino-piperidin-1-yl)-xanthine
(96) 1,3-dimethyl-7-propyl-8-(3-amino-piperidin-1-yl)-xanthine
(97) 1,3-dimethyl-7-(2-propyl)-8-(3-amino-piperidin-1-yl)-xanthine
(98) 1,3-dimethyl-7-butyl-8-(3-amino-piperidin-1-yl)-xanthine
(99) 1,3-dimethyl-7-(2-butyl)-8-(3-amino-piperidin-1-yl)-xanthine
(100) 1,3-dimethyl-7-(2-methylpropyl)-8-(3-amino-piperidin-1-yl)-xanthine
(101) 1,3-dimethyl-7-pentyl-8-(3-amino-piperidin-1-yl)-xanthine
(102) 1,3-dimethyl-7-(2-methylbutyl)-8-(3-amino-piperidin-1-yl)-xanthine
(103) 1,3-dimethyl-7-(3-methylbutyl)-8-(3-amino-piperidin-1-yl)-xanthine
(104) 1,3-dimethyl-7-(2,2-dimethylpropyl)-8-(3-amino-piperidin-1-yl)-xanthine
(105) 1,3-dimethyl-7-cyclopropylmethyl-8-(3-amino-piperidin-1-yl)-xanthine
(106) 1,3-dimethyl-7-[(1-methylcyclopropyl)methyl]-8-(3-amino-piperidin-1-yl)-xanthine
(107) 1,3-dimethyl-7-[(2-methylcyclopropyl)methyl]-8-(3-amino-piperidin-1-yl)-xanthine
(108) 1,3-dimethyl-7-cyclobutylmethyl-8-(3-amino-piperidin-1-yl)-xanthine
(109) 1,3-dimethyl-7-cyclopentylmethyl-8-(3-amino-piperidin-1-yl)-xanthine
(110) 1,3-dimethyl-7-cyclohexylmethyl-8-(3-amino-piperidin-1-yl)-xanthine
(111) 1,3-dimethyl-7-[2-(cyclopropyl)ethyl]-8-(3-amino-piperidin-1-yl)-xanthine
(112) 1,3-dimethyl-7-(2-propen-1-yl)-8-(3-amino-piperidin-1-yl)-xanthine
(113) 1,3-dimethyl-7-(2-methyl-2-propen-1-yl)-8-(3-amino-piperidin-1-yl)-xanthine
(114) 1,3-dimethyl-7-(3-phenyl-2-propen-1-yl)-8-(3-amino-piperidin-1-yl)-xanthine
(115) 1,3-dimethyl-7-(2-buten-1-yl)-8-(3-amino-piperidin-1-yl)-xanthine
(116) 1,3-dimethyl-7-(4,4,4-trifluoro-2-buten-1-yl)-8-(3-amino-piperidin-1-yl)-xanthine
(117) 1,3-dimethyl-7-(3-buten-1-yl)-8-(3-amino-piperidin-1-yl)-xanthine
(118) 1,3-dimethyl-7-(2-chloro-2-buten-1-yl)-8-(3-amino-piperidin-1-yl)-xanthine
(119) 1,3-dimethyl-7-(2-bromo-2-buten-1-yl)-8-(3-amino-piperidin-1-yl)-xanthine
(120) 1,3-dimethyl-7-(3-chloro-2-buten-1-yl)-8-(3-amino-piperidin-1-yl)-xanthine
(121) 1,3-dimethyl-7-(3-bromo-2-buten-1-yl)-8-(3-amino-piperidin-1-yl)-xanthine
(122) 1,3-dimethyl-7-(2-methyl-2-buten-1-yl)-8-(3-amino-piperidin-1-yl)-xanthine
(123) 1,3-dimethyl-7-(2,3-dimethyl-2-buten-1-yl)-8-(3-amino-piperidin-1-yl)-xanthine
(124) 1,3-dimethyl-7-(3-trifluoromethyl-2-buten-1-yl)-8-(3-amino-piperidin-1-yl)-xanthine
(125) 1,3-dimethyl-7-(3-methyl-3-buten-1-yl)-8-(3-amino-piperidin-1-yl)-xanthine
(126) 1,3-dimethyl-7-[(2-methyl-1-cyclopenten-1-yl)methyl]-8-(3-amino-piperidin-1-yl)-xanthine
(127) 1,3-dimethyl-7-(1-cyclohexen-1-yl-methyl)-8-(3-amino-piperidin-1-yl)-xanthine
(128) 1,3-dimethyl-7-[2-(1-cyclopenten-1-yl)ethyl]-8-(3-amino-piperidin-1-yl)-xanthine
(129) 1,3-dimethyl-7-(2-propyn-1-yl)-8-(3-amino-piperidin-1-yl)-xanthine
(130) 1,3-dimethyl-7-(3-butyn-1-yl)-8-(3-amino-piperidin-1-yl)-xanthine
(131) 1,3-dimethyl-7-(4-fluorobenzyl)-8-(3-amino-piperidin-1-yl)-xanthine
(132) 1,3-dimethyl-7-(2-chlorobenzyl)-8-(3-amino-piperidin-1-yl)-xanthine
(133) 1,3-dimethyl-7-(3-chlorobenzyl)-8-(3-amino-piperidin-1-yl)-xanthine
(134) 1,3-dimethyl-7-(4-chlorobenzyl)-8-(3-amino-piperidin-1-yl)-xanthine
(135) 1,3-dimethyl-7-(2-bromobenzyl)-8-(3-amino-piperidin-1-yl)-xanthine
(136) 1,3-dimethyl-7-(3-bromobenzyl)-8-(3-amino-piperidin-1-yl)-xanthine
(137) 1,3-dimethyl-7-(4-bromobenzyl)-8-(3-amino-piperidin-1-yl)-xanthine
(138) 1,3-dimethyl-7-(2-methylbenzyl)-8-(3-amino-piperidin-1-yl)-xanthine
(139) 1,3-dimethyl-7-(3-methylbenzyl)-8-(3-amino-piperidin-1-yl)-xanthine
(140) 1,3-dimethyl-7-(4-methylbenzyl)-8-(3-amino-piperidin-1-yl)-xanthine
(141) 1,3-dimethyl-7-(2-methoxybenzyl)-8-(3-amino-piperidin-1-yl)-xanthine
(142) 1,3-dimethyl-7-(3-methoxybenzyl)-8-(3-amino-piperidin-1-yl)-xanthine
(143) 1,3-dimethyl-7-(4-methoxybenzyl)-8-(3-amino-piperidin-1-yl)-xanthine
(144) 1,3-dimethyl-7-(2-phenylethyl)-8-(3-amino-piperidin-1-yl)-xanthine
(145) 1,3-dimethyl-7-(3-phenylpropyl)-8-(3-amino-piperidin-1-yl)-xanthine
(146) 1,3-dimethyl-7-(2-furanylmethyl)-8-(3-amino-piperidin-1-yl)-xanthine
(147) 1,3-dimethyl-7-(3-furanylmethyl)-8-(3-amino-piperidin-1-yl)-xanthine
(148) 1,3-dimethyl-7-(3-thienylmethyl)-8-(3-amino-piperidin-1-yl)-xanthine
(149) 1,3-dimethyl-7-(3-methyl-2-buten-1-yl)-8-(3-methylamino-piperidin-1-yl)-xanthine
(150) 1,3-dimethyl-7-(3-methyl-2-buten-1-yl)-8-(3-ethylamino-piperidin-1-yl)-xanthine
(151) 1,3-dimethyl-7-(3-methyl-2-buten-1-yl)-8-(3-dimethylamino-piperidin-1-yl)-xanthine
(152) 1,3-dimethyl-7-(3-methyl-2-buten-1-yl)-8-(3-diethylamino-piperidin-1-yl)-xanthine
(153) 1,3-dimethyl-7-(3-methyl-2-buten-1-yl)-8-{3-[(2-hydroxyethyl)amino]-piperidin-1-yl}-xanthine
(154) 1,3-dimethyl-7-(3-methyl-2-buten-1-yl)-8-{3-[N-methyl-N-(2-hydroxyethyl)-amino]-piperidin-1-yl}-xanthine
(155) 1,3-dimethyl-7-(3-methyl-2-buten-1-yl)-8-{3-[(3-hydroxypropyl)amino]-piperidin-1-yl}-xanthine
(156) 1,3-dimethyl-7-(3-methyl-2-buten-1-yl)-8-{3-[N-methyl-N-(3-hydroxypropyl)-amino]-piperidin-1-yl}-xanthine
(157) 1,3-dimethyl-7-(3-methyl-2-buten-1-yl)-8-{3-[(carboxymethyl)amino]-piperidin-1-yl}-xanthine
(158) 1,3-dimethyl-7-(3-methyl-2-buten-1-yl)-8-{3-[(methoxycarbonylmethyl)amino]-piperidin-1-yl}-xanthine
(159) 1,3-dimethyl-7-(3-methyl-2-buten-1-yl)-8-{3-[(ethoxycarbonylmethyl)amino]-piperidin-1-yl}-xanthine
(160) 1,3-dimethyl-7-(3-methyl-2-buten-1-yl)-8-{3-[N-methyl-N-(methoxycarbonyl-methyl)-amino]-piperidin-1-yl}-xanthine
(161) 1,3-dimethyl-7-(3-methyl-2-buten-1-yl)-8-{3-[N-methyl-N-(ethoxycarbonyl-methyl)-amino]-piperidin-1-yl}-xanthine
(162) 1,3-dimethyl-7-(3-methyl-2-buten-1-yl)-8-{3-[(2-carboxyethyl)amino]-piperidin-1-yl}-xanthine
(163) 1,3-dimethyl-7-(3-methyl-2-buten-1-yl)-8-(3-{[2-(methoxycarbonyl)ethyl]amino}-piperidin-1-yl)-xanthine
(164) 1,3-dimethyl-7-(3-methyl-2-buten-1-yl)-8-(3-{[2-(ethoxycarbonyl)ethyl]amino}-piperidin-1-yl)-xanthine
(165) 1,3-dimethyl-7-(3-methyl-2-buten-1-yl)-8-(3-{N-methyl-N-[2-(methoxycarbonyl)-ethyl]amino}-piperidin-1-yl)-xanthine
(166) 1,3-dimethyl-7-(3-methyl-2-buten-1-yl)-8-(3-{N-methyl-N-[2-(ethoxycarbonyl)-ethyl]amino}-piperidin-1-yl)-xanthine
(167) 1,3-dimethyl-7-(3-methyl-2-buten-1-yl)-8-{3-[(aminocarbonylmethyl)amino]-piperidin-1-yl}-xanthine
(168) 1,3-dimethyl-7-(3-methyl-2-buten-1-yl)-8-{3-[(methylaminocarbonylmethyl)-amino]-piperidin-1-yl}-xanthine
(169) 1,3-dimethyl-7-(3-methyl-2-buten-1-yl)-8-{3-[(dimethylaminocarbonylmethyl)-amino]-piperidin-1-yl}-xanthine
(170) 1,3-dimethyl-7-(3-methyl-2-buten-1-yl)-8-{3-[(ethylaminocarbonylmethyl)-amino]-piperidin-1-yl}-xanthine
(171) 1,3-dimethyl-7-(3-methyl-2-buten-1-yl)-8-{3-[(diethylaminocarbonylmethyl)-amino]-piperidin-1-yl}-xanthine
(172) 1,3-dimethyl-7-(3-methyl-2-buten-1-yl)-8-{3-[(pyrrolidin-1-ylcarbonylmethyl)-amino]-piperidin-1-yl}-xanthine
(173) 1,3-dimethyl-7-(3-methyl-2-buten-1-yl)-8-{3-[(2-cyanpyrrolidin-1-ylcarbonyl-methyl)amino]-piperidin-1-yl}-xanthine
(174) 1,3-dimethyl-7-(3-methyl-2-buten-1-yl)-8-{3-[(4-cyanothiazolidin-3-ylcarbonyl-methyl)amino]-piperidin-1-yl}-xanthine
(175) 1,3-dimethyl-7-(3-methyl-2-buten-1-yl)-8-{3-[(2-aminocarbonylpyrrolidin-1-yl-carbonylmethyl)amino]-piperidin-1-yl}-xanthine
(176) 1,3-dimethyl-7-(3-methyl-2-buten-1-yl)-8-{3-[(2-carboxypyrrolidin-1-ylcarbonyl-methyl)amino]-piperidin-1-yl}-xanthine
(177) 1,3-dimethyl-7-(3-methyl-2-buten-1-yl)-8-{3-[(2-methoxycarbonylpyrrolidin-1-ylcarbonylmethyl)amino]-piperidin-1-yl}-xanthine
(178) 1,3-dimethyl-7-(3-methyl-2-buten-1-yl)-8-{3-[(piperidin-1-ylcarbonylmethyl)-amino]-piperidin-1-yl}-xanthine
(179) 1,3-dimethyl-7-(3-methyl-2-buten-1-yl)-8-{3-[(morpholin-4-ylcarbonylmethyl)-amino]-piperidin-1-yl}-xanthine
(180) 1,3-dimethyl-7-(3-methyl-2-buten-1-yl)-8-(2-methyl-3-amino-piperidin-1-yl)-xanthine
(181) 1,3-dimethyl-7-(3-methyl-2-buten-1-yl)-8-(3-methyl-3-amino-piperidin-1-yl)-xanthine
(182) 1,3-dimethyl-7-(3-methyl-2-buten-1-yl)-8-(4-methyl-3-amino-piperidin-1-yl)-xanthine
(183) 1,3-dimethyl-7-(3-methyl-2-buten-1-yl)-8-(5-methyl-3-amino-piperidin-1-yl)-xanthine
(184) 1,3-dimethyl-7-(3-methyl-2-buten-1-yl)-8-(6-methyl-3-amino-piperidin-1-yl)-xanthine
(185) 1,3-dimethyl-7-(3-methyl-2-buten-1-yl)-8-(2-amino-8-aza-bicyclo[3.2.1]oct-8-yl)-xanthine
(186) 1,3-dimethyl-7-(3-methyl-2-buten-1-yl)-8-(6-amino-2-aza-bicyclo[2.2.2]oct-2-yl)-xanthine
(187) 1,3-dimethyl-7-(3-methyl-2-buten-1-yl)-8-(3-amino-cyclopentyl)-xanthine
(188) 1,3-dimethyl-7-(3-methyl-2-buten-1-yl)-8-(3-methylamino-cyclohexyl)-xanthine
(189) 1,3-dimethyl-7-(3-methyl-2-buten-1-yl)-8-(3-ethylamino-cyclohexyl)-xanthine
(190) 1,3-dimethyl-7-(3-methyl-2-buten-1-yl)-8-(3-dimethylamino-cyclohexyl)-xanthine
(191) 1,3-dimethyl-7-(3-methyl-2-buten-1-yl)-8-(3-diethylamino-cyclohexyl)-xanthine
(192) 1,3-dimethyl-7-(3-methyl-2-buten-1-yl)-8-(4-amino-cyclohexyl)-xanthine
(193) 1,3-dimethyl-7-(3-methyl-2-buten-1-yl)-8-[(3-amino-cyclohexyl)amino]-xanthine
(194) 1,3-dimethyl-7-(3-methyl-2-buten-1-yl)-8-[(2-amino-cyclopentyl)amino]-xanthine
(195) 1,3-dimethyl-7-(3-methyl-2-buten-1-yl)-8-[(3-amino-cyclopentyl)amino]-xanthine
(196) 1,3-dimethyl-7-(3-methyl-2-buten-1-yl)-8-[(2-amino-cyclobutyl)amino]-xanthine
(197) 1,3-dimethyl-7-(3-methyl-2-buten-1-yl)-8-[(3-amino-cyclobutyl)amino]-xanthine
(198) 1,3-dimethyl-7-(3-methyl-2-buten-1-yl)-8-[(2-amino-cyclopropyl)amino]-xanthine
(199) 1-[2-(4-hydroxy-phenyl)-ethyl]-3-methyl-7-(3-methyl-2-buten-1-yl)-8-(3-amino-piperidin-1-yl)-xanthine
(200) 1-[2-(3-fluoro-4-hydroxy-phenyl)-ethyl]-3-methyl-7-(3-methyl-2-buten-1-yl)-8-(3-amino-piperidin-1-yl)-xanthine
(201) 1-[2-(4-methoxy-phenyl)-ethyl]-3-methyl-7-(3-methyl-2-buten-1-yl)-8-(3-amino-piperidin-1-yl)-xanthine
(202) 1-[2-(4-ethoxy-phenyl)-ethyl]-3-methyl-7-(3-methyl-2-buten-1-yl)-8-(3-amino-piperidin-1-yl)-xanthine
(203) 1-(2-{4-[(carboxymethyl)oxy]-phenyl}-ethyl)-3-methyl-7-(3-methyl-2-buten-1-yl)-8-(3-amino-piperidin-1-yl)-xanthine
(204) 1-(2-{4-[(methoxycarbonyl)methyloxy]-phenyl}-ethyl)-3-methyl-7-(3-methyl-2-buten-1-yl)-8-(3-amino-piperidin-1-yl)-xanthine
(205) 1-[2-(3-hydroxy-phenyl)-ethyl]-3-methyl-7-(3-methyl-2-buten-1-yl)-8-(3-amino-piperidin-1-yl)-xanthine
(206) 1-[2-(2-fluoro-5-hydroxy-phenyl)-ethyl]-3-methyl-7-(3-methyl-2-buten-1-yl)-8-(3-amino-piperidin-1-yl)-xanthine
(207) 1-[2-(3-methoxy-phenyl)-ethyl]-3-methyl-7-(3-methyl-2-buten-1-yl)-8-(3-amino-piperidin-1-yl)-xanthine
(208) 1-{2-[3-(carboxymethyloxy)-phenyl]-ethyl}-3-methyl-7-(3-methyl-2-buten-1-yl)-8-(3-amino-piperidin-1-yl)-xanthine
(209) 1-(2-{3-[(ethoxycarbonyl)methyloxy]-phenyl}-ethyl)-3-methyl-7-(3-methyl-2-buten-1-yl)-8-(3-amino-piperidin-1-yl)-xanthine
(210) 1-[2-(2-hydroxy-phenyl)-ethyl]-3-methyl-7-(3-methyl-2-buten-1-yl)-8-(3-amino-piperidin-1-yl)-xanthine
(211) 1-[2-(2-methoxy-phenyl)-ethyl]-3-methyl-7-(3-methyl-2-buten-1-yl)-8-(3-amino-piperidin-1-yl)-xanthine
(212) 1-{2-[2-(carboxymethyloxy)-phenyl]-ethyl}-3-methyl-7-(3-methyl-2-buten-1-yl)-8-(3-amino-piperidin-1-yl)-xanthine
(213) 1-(2-{2-[(methoxycarbonyl)methyloxy]-phenyl}-ethyl)-3-methyl-7-(3-methyl-2-buten-1-yl)-8-(3-amino-piperidin-1-yl)-xanthine
(214) 1-[2-(4-methyl-phenyl)-ethyl]-3-methyl-7-(3-methyl-2-buten-1-yl)-8-(3-amino-piperidin-1-yl)-xanthine
(215) 1-[2-(4-hydroxymethyl-phenyl)-ethyl]-3-methyl-7-(3-methyl-2-buten-1-yl)-8-(3-amino-piperidin-1-yl)-xanthine
(216) 1-[2-(4-carboxy-phenyl)-ethyl]-3-methyl-7-(3-methyl-2-buten-1-yl)-8-(3-amino-piperidin-1-yl)-xanthine
(217) 1-{2-[4-(methoxycarbonyl)-phenyl]-ethyl}-3-methyl-7-(3-methyl-2-buten-1-yl)-8-(3-amino-piperidin-1-yl)-xanthine
(218) 1-{2-[4-(carboxymethyl)-phenyl]-ethyl}-3-methyl-7-(3-methyl-2-buten-1-yl)-8-(3-amino-piperidin-1-yl)-xanthine
(219) 1-(2-{4-[(methoxycarbonyl)methyl]-phenyl}-ethyl)-3-methyl-7-(3-methyl-2-buten-1-yl)-8-(3-amino-piperidin-1-yl)-xanthine
(220) 1-{2-[4-(2-carboxy-ethyl)-phenyl]-ethyl}-3-methyl-7-(3-methyl-2-buten-1-yl)-8-(3-amino-piperidin-1-yl)-xanthine
(221) 1-(2-{4-[2-(methoxycarbonyl)-ethyl]-phenyl}-ethyl)-3-methyl-7-(3-methyl-2-buten-1-yl)-8-(3-amino-piperidin-1-yl)-xanthine
(222) 1-[2-(3-methyl-phenyl)-ethyl]-3-methyl-7-(3-methyl-2-buten-1-yl)-8-(3-amino-piperidin-1-yl)-xanthine
(223) 1-[2-(3-carboxy-phenyl)-ethyl]-3-methyl-7-(3-methyl-2-buten-1-yl)-8-(3-amino-piperidin-1-yl)-xanthine
(224) 1-{2-[3-(ethoxycarbonyl)-phenyl]-ethyl}-3-methyl-7-(3-methyl-2-buten-1-yl)-8-(3-amino-piperidin-1-yl)-xanthine
(225) 1-{2-[3-(carboxymethyl)-phenyl]-ethyl}-3-methyl-7-(3-methyl-2-buten-1-yl)-8-(3-amino-piperidin-1-yl)-xanthine
(226) 1-(2-{3-[(methoxycarbonyl)methyl]-phenyl}-ethyl)-3-methyl-7-(3-methyl-2-buten-1-yl)-8-(3-amino-piperidin-1-yl)-xanthine
(227) 1-{2-[3-(2-carboxy-ethyl)-phenyl]-ethyl}-3-methyl-7-(3-methyl-2-buten-1-yl)-8-(3-amino-piperidin-1-yl)-xanthine
(228) 1-(2-{3-[2-(methoxycarbonyl)-ethyl]-phenyl}-ethyl)-3-methyl-7-(3-methyl-2-buten-1-yl)-8-(3-amino-piperidin-1-yl)-xanthine
(229) 1-[2-(2-methyl-phenyl)-ethyl]-3-methyl-7-(3-methyl-2-buten-1-yl)-8-(3-amino-piperidin-1-yl)-xanthine
(230) 1-[2-(2-carboxy-phenyl)-ethyl]-3-methyl-7-(3-methyl-2-buten-1-yl)-8-(3-amino-piperidin-1-yl)-xanthine
(231) 1-{2-[2-(methoxycarbonyl)-phenyl]-ethyl}-3-methyl-7-(3-methyl-2-buten-1-yl)-8-(3-amino-piperidin-1-yl)-xanthine
(232) 1-[2-(4-fluoro-phenyl)-ethyl]-3-methyl-7-(3-methyl-2-buten-1-yl)-8-(3-amino-piperidin-1-yl)-xanthine
(233) 1-[2-(4-chloro-phenyl)-ethyl]-3-methyl-7-(3-methyl-2-buten-1-yl)-8-(3-amino-piperidin-1-yl)-xanthine
(234) 1-[2-(4-bromo-phenyl)-ethyl]-3-methyl-7-(3-methyl-2-buten-1-yl)-8-(3-amino-piperidin-1-yl)-xanthine
(235) 1-[2-(4-cyano-phenyl)-ethyl]-3-methyl-7-(3-methyl-2-buten-1-yl)-8-(3-amino-piperidin-1-yl)-xanthine
(236) 1-[2-(4-trifluoromethoxy-phenyl)-ethyl]-3-methyl-7-(3-methyl-2-buten-1-yl)-8-(3-amino-piperidin-1-yl)-xanthine
(237) 1-[2-(4-methylsulphanyl-phenyl)-ethyl]-3-methyl-7-(3-methyl-2-buten-1-yl)-8-(3-amino-piperidin-1-yl)-xanthine
(238) 1-[2-(4-methylsulphinyl-phenyl)-ethyl]-3-methyl-7-(3-methyl-2-buten-1-yl)-8-(3-amino-piperidin-1-yl)-xanthine
(239) 1-[2-(4-methylsulphonyl-phenyl)-ethyl]-3-methyl-7-(3-methyl-2-buten-1-yl)-8-(3-amino-piperidin-1-yl)-xanthine
(240) 1-[2-(4-trifluoromethyl-phenyl)-ethyl]-3-methyl-7-(3-methyl-2-buten-1-yl)-8-(3-amino-piperidin-1-yl)-xanthine
(241) 1-[2-(4-amino-phenyl)-ethyl]-3-methyl-7-(3-methyl-2-buten-1-yl)-8-(3-amino-piperidin-1-yl)-xanthine
(242) 1-(2-{4-[(methylcarbonyl)amino]-phenyl}-ethyl)-3-methyl-7-(3-methyl-2-buten-1-yl)-8-(3-amino-piperidin-1-yl)-xanthine
(243) 1-(2-{4-[(methylsulphonyl)amino]-phenyl}-ethyl)-3-methyl-7-(3-methyl-2-buten-1-yl)-8-(3-amino-piperidin-1-yl)-xanthine
(244) 1-[2-(3-nitro-phenyl)-ethyl]-3-methyl-7-(3-methyl-2-buten-1-yl)-8-(3-amino-piperidin-1-yl)-xanthine
(245) 1-{2-[4-(aminocarbonyl)-phenyl]-ethyl}-3-methyl-7-(3-methyl-2-buten-1-yl)-8-(3-amino-piperidin-1-yl)-xanthine
(246) 1-{2-[4-(methylaminocarbonyl)-phenyl]-ethyl}-3-methyl-7-(3-methyl-2-buten-1-yl)-8-(3-amino-piperidin-1-yl)-xanthine
(247) 1-{2-[4-(dimethylaminocarbonyl)-phenyl]-ethyl}-3-methyl-7-(3-methyl-2-buten-1-yl)-8-(3-amino-piperidin-1-yl)-xanthine
(248) 1-{2-[4-(aminosulphonyl)-phenyl]-ethyl}-3-methyl-7-(3-methyl-2-buten-1-yl)-8-(3-amino-piperidin-1-yl)-xanthine
(249) 1-{2-[4-(methylaminosulphonyl)-phenyl]-ethyl}-3-methyl-7-(3-methyl-2-buten-1-yl)-8-(3-amino-piperidin-1-yl)-xanthine
(250) 1-{2-[4-(dimethylaminosulphonyl)-phenyl]-ethyl}-3-methyl-7-(3-methyl-2-buten-1-yl)-8-(3-amino-piperidin-1-yl)-xanthine
(251) 1-(3-carboxy-propyl)-3-methyl-7-(3-methyl-2-buten-1-yl)-8-(3-amino-piperidin-1-yl)-xanthine
(252) 1-[3-(methoxycarbonyl)-propyl]-3-methyl-7-(3-methyl-2-buten-1-yl)-8-(3-amino-piperidin-1-yl)-xanthine
(253) 1-[3-(ethoxycarbonyl)-propyl]-3-methyl-7-(3-methyl-2-buten-1-yl)-8-(3-amino-piperidin-1-yl)-xanthine
(254) 1-[2-(3,4-dimethyl-phenyl)-ethyl]-3-methyl-7-(3-methyl-2-buten-1-yl)-8-(3-amino-piperidin-1-yl)-xanthine
(255) 1-[2-(2-fluoro-5-chloro-phenyl)-ethyl]-3-methyl-7-(3-methyl-2-buten-1-yl)-8-(3-amino-piperidin-1-yl)-xanthine
(256) 1-[2-(3,5-dimethoxy-phenyl)-ethyl]-3-methyl-7-(3-methyl-2-buten-1-yl)-8-(3-amino-piperidin-1-yl)-xanthine
(257) 1-[2-(naphthalin-2-yl)-ethyl]-3-methyl-7-(3-methyl-2-buten-1-yl)-8-(3-amino-piperidin-1-yl)-xanthine
(258) 1-[2-(pyridin-3-yl)-ethyl]-3-methyl-7-(3-methyl-2-buten-1-yl)-8-(3-amino-piperidin-1-yl)-xanthine
(259) 1-[4-phenyl-butyl]-3-methyl-7-(3-methyl-2-buten-1-yl)-8-(3-amino-piperidin-1-yl)-xanthine
(260) 1-methyl-3-(3-phenyl-propyl)-7-(3-methyl-2-buten-1-yl)-8-(3-amino-piperidin-1-yl)-xanthine
(261) 1-methyl-3-(3-carboxy-propyl)-7-(3-methyl-2-buten-1-yl)-8-(3-amino-piperidin-1-yl)-xanthine
(262) 1-methyl-3-[3-(methoxycarbonyl)-propyl]-7-(3-methyl-2-buten-1-yl)-8-(3-amino-piperidin-1-yl)-xanthine
(263) 1-methyl-3-[3-(ethoxycarbonyl)-propyl]-7-(3-methyl-2-buten-1-yl)-8-(3-amino-piperidin-1-yl)-xanthine
(264) 1,3-dimethyl-7-(3-methyl-2-buten-1-yl)-8-(3-amino-1-methyl-prop-1-yl)-xanthine
(265) 1,3-dimethyl-7-(3-methyl-2-buten-1-yl)-8-(3-amino-1,1-dimethyl-prop-1-yl)-xanthine
(266) 1,3-dimethyl-7-(3-methyl-2-buten-1-yl)-8-(3-amino-1-methyl-but-1-yl)-xanthine
(267) 1,3-dimethyl-7-(3-methyl-2-buten-1-yl)-8-[1-(2-amino-ethyl)-cyclopropyl]-xanthine
(268) 1,3-dimethyl-7-(3-methyl-2-buten-1-yl)-8-[1-(aminomethyl)-cyclopentylmethyl]-xanthine
(269) 1,3-dimethyl-7-(3-methyl-2-buten-1-yl)-8-[2-(aminomethyl)-cyclopropyl]-xanthine
(270) 1,3-dimethyl-7-(3-methyl-2-buten-1-yl)-8-[2-(aminomethyl)-cyclopentyl]-xanthine
(271) 1,3-dimethyl-7-(3-methyl-2-buten-1-yl)-8-(2-amino-cyclopropylmethyl)-xanthine
(272) 1,3-dimethyl-7-(3-methyl-2-buten-1-yl)-8-[(piperidin-3-yl)methyl]-xanthine
(273) 1,3-dimethyl-7-(3-methyl-2-buten-1-yl)-8-[2-(pyrrolidine-2-yl)-ethyl]-xanthine
(274) 1,3-dimethyl-7-(3-methyl-2-buten-1-yl)-8-[N-(2-amino-ethyl)-N-ethyl-amino]-xanthine
(275) 1,3-dimethyl-7-(3-methyl-2-buten-1-yl)-8-[N-(2-amino-ethyl)-N-isopropyl-amino]-xanthine
(276) 1,3-dimethyl-7-(3-methyl-2-buten-1-yl)-8-[N-(2-amino-ethyl)-N-cyclopropyl-amino]-xanthine
(277) 1,3-dimethyl-7-(3-methyl-2-buten-1-yl)-8-[N-(2-amino-ethyl)-N-cyclopropylmethyl-amino]-xanthine
(278) 1,3-dimethyl-7-(3-methyl-2-buten-1-yl)-8-[N-(2-amino-ethyl)-N-phenyl-amino]-xanthine
(279) 1,3-dimethyl-7-(3-methyl-2-buten-1-yl)-8-[N-(2-amino-ethyl)-N-benzyl-amino]-xanthine
(280) 1,3-dimethyl-7-(3-methyl-2-buten-1-yl)-8-[N-(2-amino-1-methyl-ethyl)-N-methyl-amino]-xanthine
(281) 1,3-dimethyl-7-(3-methyl-2-buten-1-yl)-8-[N-(2-amino-prop-1-yl)-N-methyl-amino]-xanthine
(282) 1,3-dimethyl-7-(3-methyl-2-buten-1-yl)-8-[N-(2-amino-1-methyl-prop-1-yl)-N-methyl-amino]-xanthine
(283) 1,3-dimethyl-7-(3-methyl-2-buten-1-yl)-8-[N-(2-amino-2-methyl-propyl)-N-methyl-amino]-xanthine
(284) 1,3-dimethyl-7-(3-methyl-2-buten-1-yl)-8-[N-(1-amino-cyclopropylmethyl)-N-methyl-amino]-xanthine
(285) 1,3-dimethyl-7-(3-methyl-2-buten-1-yl)-8-[N-(2-amino-cyclopropyl)-N-methyl-amino]-xanthine
(286) 1,3-dimethyl-7-(3-methyl-2-buten-1-yl)-8-[N-(2-amino-cyclobutyl)-N-methyl-amino]-xanthine
(287) 1,3-dimethyl-7-(3-methyl-2-buten-1-yl)-8-[N-(2-amino-cyclopentyl)-N-methyl-amino]-xanthine
(288) 1,3-dimethyl-7-(3-methyl-2-buten-1-yl)-8-[N-(2-amino-cyclohexyl)-N-methyl-amino]-xanthine
(289) 1,3-dimethyl-7-(3-methyl-2-buten-1-yl)-8-{N-[(pyrrolidine-2-yl)methyl]-N-methyl-amino}-xanthine
(290) 1,3-dimethyl-7-(3-methyl-2-buten-1-yl)-8-[N-(pyrrolidin-3-yl)-N-methyl-amino]-xanthine
(291) 1,3-dimethyl-7-(3-methyl-2-buten-1-yl)-8-[N-(piperidin-3-yl)-N-methyl-amino]-xanthine
(292) 1-(2-phenyloxy-ethyl)-3-methyl-7-(3-methyl-2-buten-1-yl)-8-(3-amino-piperidin-1-yl)-xanthine
(293) 1-(2-phenylsulphanyl-ethyl)-3-methyl-7-(3-methyl-2-buten-1-yl)-8-(3-amino-piperidin-1-yl)-xanthine
(294) 1-(2-phenylsulphinyl-ethyl)-3-methyl-7-(3-methyl-2-buten-1-yl)-8-(3-amino-piperidin-1-yl)-xanthine
(295) 1-(2-phenylsulphonyl-ethyl)-3-methyl-7-(3-methyl-2-buten-1-yl)-8-(3-amino-piperidin-1-yl)-xanthine
(296) 1-methyl-3-(2-oxo-2-phenyl-ethyl)-7-(3-methyl-2-buten-1-yl)-8-(3-amino-piperidin-1-yl)-xanthine
(297) 1-methyl-3-(2-oxo-propyl)-7-(3-methyl-2-buten-1-yl)-8-(3-amino-piperidin-1-yl)-xanthine
(298) 1-methyl-3-phenyl-7-(3-methyl-2-buten-1-yl)-8-(3-amino-piperidin-1-yl)-xanthine
(299) 1-methyl-3-cyclopropyl-7-(3-methyl-2-buten-1-yl)-8-(3-amino-piperidin-1-yl)-xanthine
(300) 1-[2-(3-fluoro-phenyl)-2-oxo-ethyl]-3-methyl-7-(3-methyl-2-buten-1-yl)-8-(3-amino-piperidin-1-yl)-xanthine
(301) 1-[2-(3-chloro-phenyl)-2-oxo-ethyl]-3-methyl-7-(3-methyl-2-buten-1-yl)-8-(3-amino-piperidin-1-yl)-xanthine
(302) 1-[2-(3-bromo-phenyl)-2-oxo-ethyl]-3-methyl-7-(3-methyl-2-buten-1-yl)-8-(3-amino-piperidin-1-yl)-xanthine
(303) 1-[2-(3-methyl-phenyl)-2-oxo-ethyl]-3-methyl-7-(3-methyl-2-buten-1-yl)-8-(3-amino-piperidin-1-yl)-xanthine
(304) 1-[2-(3-trifluoromethyl-phenyl)-2-oxo-ethyl]-3-methyl-7-(3-methyl-2-buten-1-yl)-8-(3-amino-piperidin-1-yl)-xanthine
(305) 1-[2-(2-methyl-phenyl)-2-oxo-ethyl]-3-methyl-7-(3-methyl-2-buten-1-yl)-8-(3-amino-piperidin-1-yl)-xanthine
(306) 1-[2-(3-methoxy-phenyl)-2-oxo-ethyl]-3-methyl-7-(3-methyl-2-buten-1-yl)-8-(3-amino-piperidin-1-yl)-xanthine
(307) 1-[2-(3-difluoromethoxy-phenyl)-2-oxo-ethyl]-3-methyl-7-(3-methyl-2-buten-1-yl)-8-(3-amino-piperidin-1-yl)-xanthine
(308) 1-[2-(3-trifluoromethoxy-phenyl)-2-oxo-ethyl]-3-methyl-7-(3-methyl-2-buten-1-yl)-8-(3-amino-piperidin-1-yl)-xanthine
(309) 1-[2-(3-ethoxy-phenyl)-2-oxo-ethyl]-3-methyl-7-(3-methyl-2-buten-1-yl)-8-(3-amino-piperidin-1-yl)-xanthine
(310) 1-[2-(3-isopropyloxy-phenyl)-2-oxo-ethyl]-3-methyl-7-(3-methyl-2-buten-1-yl)-8-(3-amino-piperidin-1-yl)-xanthine
(311) 1-[2-(3-cyclopropyloxy-phenyl)-2-oxo-ethyl]-3-methyl-7-(3-methyl-2-buten-1-yl)-8-(3-amino-piperidin-1-yl)-xanthine
(312) 1-[2-(3-cyclopentyloxy-phenyl)-2-oxo-ethyl]-3-methyl-7-(3-methyl-2-buten-1-yl)-8-(3-amino-piperidin-1-yl)-xanthine
(313) 1-[2-(3-cyclopropylmethoxy-phenyl)-2-oxo-ethyl]-3-methyl-7-(3-methyl-2-buten-1-yl)-8-(3-amino-piperidin-1-yl)-xanthine
(314) 1-{2-[3-(2,2,2-trifluorethoxy)-phenyl]-2-oxo-ethyl}-3-methyl-7-(3-methyl-2-buten-1-yl)-8-(3-amino-piperidin-1-yl)-xanthine
(315) 1-[2-(4-hydroxy-phenyl)-2-oxo-ethyl]-3-methyl-7-(3-methyl-2-buten-1-yl)-8-(3-amino-piperidin-1-yl)-xanthine
(316) 1-[2-(3-nitro-phenyl)-2-oxo-ethyl]-3-methyl-7-(3-methyl-2-buten-1-yl)-8-(3-amino-piperidin-1-yl)-xanthine
(317) 1-[2-(3-amino-phenyl)-2-oxo-ethyl]-3-methyl-7-(3-methyl-2-buten-1-yl)-8-(3-amino-piperidin-1-yl)-xanthine
(318) 1-{2-[3-(methylcarbonylamino)-phenyl]-2-oxo-ethyl}-3-methyl-7-(3-methyl-2-buten-1-yl)-8-(3-amino-piperidin-1-yl)-xanthine
(319) 1-{2-[3-(aminocarbonylamino)-phenyl]-2-oxo-ethyl}-3-methyl-7-(3-methyl-2-buten-1-yl)-8-(3-amino-piperidin-1-yl)-xanthine
(320) 1-{2-[3-(methylaminocarbonylamino)-phenyl]-2-oxo-ethyl}-3-methyl-7-(3-methyl-2-buten-1-yl)-8-(3-amino-piperidin-1-yl)-xanthine
(321) 1-{2-[3-(dimethylaminocarbonylamino)-phenyl]-2-oxo-ethyl}-3-methyl-7-(3-methyl-2-buten-1-yl)-8-(3-amino-piperidin-1-yl)-xanthine
(322) 1-{2-[3-(methylsulphonylamino)-phenyl]-2-oxo-ethyl}-3-methyl-7-(3-methyl-2-buten-1-yl)-8-(3-amino-piperidin-1-yl)-xanthine
(323) 1-{2-[3-(aminosulphonyl)-phenyl]-2-oxo-ethyl}-3-methyl-7-(3-methyl-2-buten-1-yl)-8-(3-amino-piperidin-1-yl)-xanthine
(324) 1-{2-[3-(methylaminosulphonyl)-phenyl]-2-oxo-ethyl}-3-methyl-7-(3-methyl-2-buten-1-yl)-8-(3-amino-piperidin-1-yl)-xanthine
(325) 1-{2-[3-(dimethylaminosulphonyl)-phenyl]-2-oxo-ethyl}-3-methyl-7-(3-methyl-2-buten-1-yl)-8-(3-amino-piperidin-1-yl)-xanthine
(326) 1-[2-(3-ethynyl-phenyl)-2-oxo-ethyl]-3-methyl-7-(3-methyl-2-buten-1-yl)-8-(3-amino-piperidin-1-yl)-xanthine
(327) 1-[2-(3-cyano-phenyl)-2-oxo-ethyl]-3-methyl-7-(3-methyl-2-buten-1-yl)-8-(3-amino-piperidin-1-yl)-xanthine
(328) 1-{2-[3-(aminocarbonyl)-phenyl]-2-oxo-ethyl}-3-methyl-7-(3-methyl-2-buten-1-yl)-8-(3-amino-piperidin-1-yl)-xanthine
(329) 1-{2-[3-(methylaminocarbonyl)-phenyl]-2-oxo-ethyl}-3-methyl-7-(3-methyl-2-buten-1-yl)-8-(3-amino-piperidin-1-yl)-xanthine
(330) 1-{2-[3-(dimethylaminocarbonyl)-phenyl]-2-oxo-ethyl}-3-methyl-7-(3-methyl-2-buten-1-yl)-8-(3-amino-piperidin-1-yl)-xanthine
(331) 1-{2-[3-(methylsulphanyl)-phenyl]-2-oxo-ethyl}-3-methyl-7-(3-methyl-2-buten-1-yl)-8-(3-amino-piperidin-1-yl)-xanthine
(332) 1-{2-[3-(methylsulphinyl)-phenyl]-2-oxo-ethyl}-3-methyl-7-(3-methyl-2-buten-1-yl)-8-(3-amino-piperidin-1-yl)-xanthine
(333) 1-{2-[3-(methylsulphonyl)-phenyl]-2-oxo-ethyl}-3-methyl-7-(3-methyl-2-buten-1-yl)-8-(3-amino-piperidin-1-yl)-xanthine
(334) 1-[2-(3,5-dimethyl-phenyl)-2-oxo-ethyl]-3-methyl-7-(3-methyl-2-buten-1-yl)-8-(3-amino-piperidin-1-yl)-xanthine
(335) 1-[2-(3,5-dimethoxy-phenyl)-2-oxo-ethyl]-3-methyl-7-(3-methyl-2-buten-1-yl)-8-(3-amino-piperidin-1-yl)-xanthine
(336) 1-[2-(3-fluoro-5-methyl-phenyl)-2-oxo-ethyl]-3-methyl-7-(3-methyl-2-buten-1-yl)-8-(3-amino-piperidin-1-yl)-xanthine
(337) 1-[2-(pyridin-3-yl)-2-oxo-ethyl]-3-methyl-7-(3-methyl-2-buten-1-yl)-8-(3-amino-piperidin-1-yl)-xanthine
(338) 1-[2-(furan-2-yl)-2-oxo-ethyl]-3-methyl-7-(3-methyl-2-buten-1-yl)-8-(3-amino-piperidin-1-yl)-xanthine
(339) 1-[2-(thiophen-2-yl)-2-oxo-ethyl]-3-methyl-7-(3-methyl-2-buten-1-yl)-8-(3-amino-piperidin-1-yl)-xanthine
(340) 1-[2-(thiazol-2-yl)-2-oxo-ethyl]-3-methyl-7-(3-methyl-2-buten-1-yl)-8-(3-amino-piperidin-1-yl)-xanthine
(341) 1-[2-(thiazol-5-yl)-2-oxo-ethyl]-3-methyl-7-(3-methyl-2-buten-1-yl)-8-(3-amino-piperidin-1-yl)-xanthine
(342) 1-[2-(thiazol-4-yl)-2-oxo-ethyl]-3-methyl-7-(3-methyl-2-buten-1-yl)-8-(3-amino-piperidin-1-yl)-xanthine
(343) 1-(2-phenyl-2-oxo-ethyl)-7-(3-methyl-2-buten-1-yl)-8-(3-amino-piperidin-1-yl)-xanthine
(344) 1-(2-phenyl-2-oxo-ethyl)-3-methyl-7-[(1-cyclopenten-1-yl)-methyl]-8-(3-amino-piperidin-1-yl)-xanthine
(345) 1-(2-phenyl-2-oxo-ethyl)-3-methyl-7-[(2-methyl-1-cyclopenten-1-yl)-methyl]-8-(3-amino-piperidin-1-yl)-xanthine
(346) 1-(2-phenyl-2-oxo-ethyl)-3-methyl-7-(2-butyn-1-yl)-methyl]-8-(3-amino-piperidin-1-yl)-xanthine
(347) 1-(2-phenyl-2-oxo-ethyl)-3-methyl-7-(3-methyl-2-buten-1-yl)-8-(3-amino-cyclohexyl)-xanthine
(348) 1-(2-phenyl-2-oxo-ethyl)-3-methyl-7-(3-methyl-2-buten-1-yl)-8-[N-(2-amino-ethyl)-N-methyl-amino]-xanthine
(349) 1-(2-phenyl-2-oxo-ethyl)-3-methyl-7-(3-methyl-2-buten-1-yl)-8-(piperazin-1-yl)-xanthine
(350) 1-(2-phenyl-2-oxo-ethyl)-3-methyl-7-(3-methyl-2-buten-1-yl)-8-(homopiperazin-1-yl)-xanthine
(351) 1-(2-phenyl-2-oxo-ethyl)-3-methyl-7-(3-methyl-2-buten-1-yl)-8-(4-aminomethyl-piperidin-1-yl)-xanthine
(352) 1-(2-phenyl-2-oxo-ethyl)-3-methyl-7-(3-methyl-2-buten-1-yl)-8-(3-aminomethyl-piperidin-1-yl)-xanthine
(353) 1-(2-phenyl-2-oxo-ethyl)-3-methyl-7-(3-methyl-2-buten-1-yl)-8-(2-amino-cyclohexylamino)-xanthine
(354) 1-(2-phenyl-2-oxo-ethyl)-3-methyl-7-(3-methyl-2-buten-1-yl)-8-(3-amino-3-methyl-piperidin-1-yl)-xanthine
(355) 1-(2-phenyl-2-hydroxyimino-ethyl)-3-methyl-7-(3-methyl-2-buten-1-yl)-8-(3-amino-piperidin-1-yl)-xanthine
(356) 1-(2-phenyl-2-methoxyimino-ethyl)-3-methyl-7-(3-methyl-2-buten-1-yl)-8-(3-amino-piperidin-1-yl)-xanthine
(357) 1-(2-oxo-propyl)-3-methyl-7-(3-methyl-2-buten-1-yl)-8-(3-amino-piperidin-1-yl)-xanthine
(358) 1-(2-oxo-butyl)-3-methyl-7-(3-methyl-2-buten-1-yl)-8-(3-amino-piperidin-1-yl)-xanthine
(359) 1-(3-methyl-2-oxo-butyl)-3-methyl-7-(3-methyl-2-buten-1-yl)-8-(3-amino-piperidin-1-yl)-xanthine
(360) 1-(2-cyclopropyl-2-oxo-ethyl)-3-methyl-7-(3-methyl-2-buten-1-yl)-8-(3-amino-piperidin-1-yl)-xanthine
(361) 1-(2-cyclohexyl-2-oxo-ethyl)-3-methyl-7-(3-methyl-2-buten-1-yl)-8-(3-amino-piperidin-1-yl)-xanthine
(362) 1-(3-dimethylamino-2,3-dioxo-propyl)-3-methyl-7-(3-methyl-2-buten-1-yl)-8-(3-amino-piperidin-1-yl)-xanthine
(363) 1-[3-(piperidin-1-yl)-2,3-dioxo-propyl]-3-methyl-7-(3-methyl-2-buten-1-yl)-8-(3-amino-piperidin-1-yl)-xanthine
(364) 1-(2-phenyl-2-hydroxy-ethyl)-3-methyl-7-(3-methyl-2-buten-1-yl)-8-(3-amino-piperidin-1-yl)-xanthine
(365) 1-(2-phenyl-2-hydroxy-propyl)-3-methyl-7-(3-methyl-2-buten-1-yl)-8-(3-amino-piperidin-1-yl)-xanthine
(366) 1-(2-phenyl-2-methoxy-ethyl)-3-methyl-7-(3-methyl-2-buten-1-yl)-8-(3-amino-piperidin-1-yl)-xanthine
(367) 1-[(isoquinolin-1-yl)methyl]-3-methyl-7-(3-methyl-2-buten-1-yl)-8-(3-amino-piperidin-1-yl)-xanthine
(368) 1-[(quinazolin-4-yl)methyl]-3-methyl-7-(3-methyl-2-buten-1-yl)-8-(3-amino-piperidin-1-yl)-xanthine
(369) 1-[(pyridin-2-yl)methyl]-3-methyl-7-(3-methyl-2-buten-1-yl)-8-(3-amino-piperidin-1-yl)-xanthine
(370) 1-[(5-methyl-isoxazol-3-yl)methyl]-3-methyl-7-(3-methyl-2-buten-1-yl)-8-(3-amino-piperidin-1-yl)-xanthine
(371) 1-[(oxazol-2-yl)methyl]-3-methyl-7-(3-methyl-2-buten-1-yl)-8-(3-amino-piperidin-1-yl)-xanthine
(372) 1-[(thiazol-2-yl)methyl]-3-methyl-7-(3-methyl-2-buten-1-yl)-8-(3-amino-piperidin-1-yl)-xanthine
(373) 1-[(1H-indazol-3-yl)methyl]-3-methyl-7-(3-methyl-2-buten-1-yl)-8-(3-amino-piperidin-1-yl)-xanthine
(374) 1-[(1-methyl-1H-indazol-3-yl)methyl]-3-methyl-7-(3-methyl-2-buten-1-yl)-8-(3-amino-piperidin-1-yl)-xanthine
(375) 1-[(benzo[d]isoxazol-3-yl)methyl]-3-methyl-7-(3-methyl-2-buten-1-yl)-8-(3-amino-piperidin-1-yl)-xanthine
(376) 1-[(benzo[d]isothiazol-3-yl)methyl]-3-methyl-7-(3-methyl-2-buten-1-yl)-8-(3-amino-piperidin-1-yl)-xanthine
(377) 1-[(5-fluoro-benzo[d]isothiazol-3-yl)methyl]-3-methyl-7-(3-methyl-2-buten-1-yl)-8-(3-amino-piperidin-1-yl)-xanthine
(378) 1-[(5-fluoro-benzo[d]isoxazol-3-yl)methyl]-3-methyl-7-(3-methyl-2-buten-1-yl)-8-(3-amino-piperidin-1-yl)-xanthine
(379) 1-[(5-methyl-benzo[d]isoxazol-3-yl)methyl]-3-methyl-7-(3-methyl-2-buten-1-yl)-8-(3-amino-piperidin-1-yl)-xanthine
(380) 1-[(5-methyl-benzo[d]isothiazol-3-yl)methyl]-3-methyl-7-(3-methyl-2-buten-1-yl)-8-(3-amino-piperidin-1-yl)-xanthine
(381) 1-(2-phenyl-2-oxo-ethyl)-3-methyl-7-(3-methyl-2-buten-1-yl)-8-(3-imino-piperazin-1-yl)-xanthine
(382) 1-(2-phenyl-2-oxo-ethyl)-3-methyl-7-(3-methyl-2-buten-1-yl)-8-(6-amino-[1,4]diazepan-1-yl)-xanthine
(383) 1-(2-cyclohexyl-ethyl)-3-methyl-7-(3-methyl-2-buten-1-yl)-8-(3-amino-piperidin-1-yl)-xanthine
(384) 1-[2-(2-difluoromethoxy-phenyl)-ethyl]-3-methyl-7-(3-methyl-2-buten-1-yl)-8-(3-amino-piperidin-1-yl)-xanthine
(385) 1-[2-(2-difluoromethoxy-phenyl)-2-oxo-ethyl]-3-methyl-7-(3-methyl-2-buten-1-yl)-8-(3-amino-piperidin-1-yl)-xanthine
(386) 1-[2-(2-trifluoromethoxy-phenyl)-2-oxo-ethyl]-3-methyl-7-(3-methyl-2-buten-1-yl)-8-(3-amino-piperidin-1-yl)-xanthine
(387) 1-[2-(indan-4-yl)-2-oxo-ethyl]-3-methyl-7-(3-methyl-2-buten-1-yl)-8-(3-amino-piperidin-1-yl)-xanthine
(388) 1-[2-(benzo[1,3]dioxol-4-yl)-2-oxo-ethyl]-3-methyl-7-(3-methyl-2-buten-1-yl)-8-(3-amino-piperidin-1-yl)-xanthine
(389) 1-[2-(2,2-difluoro-benzo[1,3]dioxol-4-yl)-2-oxo-ethyl]-3-methyl-7-(3-methyl-2-buten-1-yl)-8-(3-amino-piperidin-1-yl)-xanthine
(390) 1-[2-(naphth-1-yl)-2-oxo-ethyl]-3-methyl-7-(3-methyl-2-buten-1-yl)-8-(3-amino-piperidin-1-yl)-xanthine
(391) 1-[2-(2-isopropyl-phenyl)-2-oxo-ethyl]-3-methyl-7-(3-methyl-2-buten-1-yl)-8-(3-amino-piperidin-1-yl)-xanthine
(392) 1-[2-(2-cyclopropyl-phenyl)-2-oxo-ethyl]-3-methyl-7-(3-methyl-2-buten-1-yl)-8-(3-amino-piperidin-1-yl)-xanthine
(393) 1-[2-(2-cyclopentyl-phenyl)-2-oxo-ethyl]-3-methyl-7-(3-methyl-2-buten-1-yl)-8-(3-amino-piperidin-1-yl)-xanthine
(394) 1-[2-(2-phenyl-phenyl)-2-oxo-ethyl]-3-methyl-7-(3-methyl-2-buten-1-yl)-8-(3-amino-piperidin-1-yl)-xanthine
(395) 1-[2-(2-cyclopentylmethoxy-phenyl)-2-oxo-ethyl]-3-methyl-7-(3-methyl-2-buten-1-yl)-8-(3-amino-piperidin-1-yl)-xanthine
(396) 1-(3-phenyl-2-oxo-propyl)-3-methyl-7-(3-methyl-2-buten-1-yl)-8-(3-amino-piperidin-1-yl)-xanthine
(397) 1-(3-phenyl-3-oxo-propyl)-3-methyl-7-(3-methyl-2-buten-1-yl)-8-(3-amino-piperidin-1-yl)-xanthine
(398) 1-methyl-3-cyclopentyl-7-(3-methyl-2-buten-1-yl)-8-(3-amino-piperidin-1-yl)-xanthine
(399) 1-methyl-3-cyclohexyl-7-(3-methyl-2-buten-1-yl)-8-(3-amino-piperidin-1-yl)-xanthine
(400) 1-methyl-3-(2-cyclopropyl-ethyl)-7-(3-methyl-2-buten-1-yl)-8-(3-amino-piperidin-1-yl)-xanthine
(401) 1-methyl-3-(2-cyclohexyl-ethyl)-7-(3-methyl-2-buten-1-yl)-8-(3-amino-piperidin-1-yl)-xanthine
(402) 1-methyl-3-(4-fluoro-phenyl)-7-(3-methyl-2-buten-1-yl)-8-(3-amino-piperidin-1-yl)-xanthine
(403) 1-methyl-3-(4-methyl-phenyl)-7-(3-methyl-2-buten-1-yl)-8-(3-amino-piperidin-1-yl)-xanthine
(404) 1-methyl-3-(4-trifluoromethyl-phenyl)-7-(3-methyl-2-buten-1-yl)-8-(3-amino-piperidin-1-yl)-xanthine
(405) 1-methyl-3-(3-methoxy-phenyl)-7-(3-methyl-2-buten-1-yl)-8-(3-amino-piperidin-1-yl)-xanthine
(406) 1-methyl-3-(3-difluoromethoxy-phenyl)-7-(3-methyl-2-buten-1-yl)-8-(3-amino-piperidin-1-yl)-xanthine
(407) 1-methyl-3-[2-(3-fluoro-phenyl)-ethyl]-7-(3-methyl-2-buten-1-yl)-8-(3-amino-piperidin-1-yl)-xanthine
(408) 1-methyl-3-[2-(3-methyl-phenyl)-ethyl]-7-(3-methyl-2-buten-1-yl)-8-(3-amino-piperidin-1-yl)-xanthine
(409) 1-methyl-3-[2-(4-methoxy-phenyl)-ethyl]-7-(3-methyl-2-buten-1-yl)-8-(3-amino-piperidin-1-yl)-xanthine
(410) 1-methyl-3-[2-(4-trifluoromethoxy-phenyl)-ethyl]-7-(3-methyl-2-buten-1-yl)-8-(3-amino-piperidin-1-yl)-xanthine
(411) 1-methyl-3-[2-(4-trifluoromethoxy-phenyl)-2-oxo-ethyl]-7-(3-methyl-2-buten-1-yl)-8-(3-amino-piperidin-1-yl)-xanthine
(412) 1-methyl-3-[2-(4-methoxy-phenyl)-2-oxo-ethyl]-7-(3-methyl-2-buten-1-yl)-8-(3-amino-piperidin-1-yl)-xanthine
(413) 1-methyl-3-[2-(4-hydroxy-phenyl)-2-oxo-ethyl]-7-(3-methyl-2-buten-1-yl)-8-(3-amino-piperidin-1-yl)-xanthine
(414) 1-methyl-3-[2-(3-chloro-phenyl)-2-oxo-ethyl]-7-(3-methyl-2-buten-1-yl)-8-(3-amino-piperidin-1-yl)-xanthine
(415) 1-methyl-3-[2-(pyridin-3-yl)-2-oxo-ethyl]-7-(3-methyl-2-buten-1-yl)-8-(3-amino-piperidin-1-yl)-xanthine
(416) 1-methyl-3-[2-(thiophen-2-yl)-2-oxo-ethyl]-7-(3-methyl-2-buten-1-yl)-8-(3-amino-piperidin-1-yl)-xanthine
(417) 1-methyl-3-[3-methyl-2-oxo-butyl]-7-(3-methyl-2-buten-1-yl)-8-(3-amino-piperidin-1-yl)-xanthine
(418) 1-methyl-3-(2-cyclopentyl-2-oxo-ethyl)-7-(3-methyl-2-buten-1-yl)-8-(3-amino-piperidin-1-yl)-xanthine
(419) 1-methyl-3-(2-phenyloxy-ethyl)-7-(3-methyl-2-buten-1-yl)-8-(3-amino-piperidin-1-yl)-xanthine
(420) 1-(2-phenyl-2-oxo-ethyl)-3-methyl-7-(4-fluoro-phenyl)-8-(3-amino-piperidin-1-yl)-xanthine
(421) 1-(2-phenyl-2-oxo-ethyl)-3-methyl-7-(3-trifluoromethyl-phenyl)-8-(3-amino-piperidin-1-yl)-xanthine
(422) 1-(2-phenyl-2-oxo-ethyl)-3-methyl-7-(3-methoxy-phenyl)-8-(3-amino-piperidin-1-yl)-xanthine
(423) 1-(2-phenyl-2-oxo-ethyl)-3-methyl-7-(3-difluoromethoxy-phenyl)-8-(3-amino-piperidin-1-yl)-xanthine
(424) 1-(2-phenyl-2-oxo-ethyl)-3-methyl-7-(3-trifluoromethoxy-phenyl)-8-(3-amino-piperidin-1-yl)-xanthine
(425) 1-(2-phenyl-2-oxo-ethyl)-3-methyl-7-(3-methyl-2-buten-1-yl)-8-(4-amino-2-aza-bicyclo[3.2.1]oct-2-yl)-xanthine
(426) 1-[2-(2-methylamino-phenyl)-2-oxo-ethyl]-3-methyl-7-(3-methyl-2-buten-1-yl)-8-(3-amino-piperidin-1-yl)-xanthine
(427) 1-{2-[2-(N-cyanomethyl-N-methyl-amino)-phenyl]-2-oxo-ethyl}-3-methyl-7-(3-methyl-2-buten-1-yl)-8-(3-amino-piperidin-1-yl)-xanthine
(428) 1-[2-(2-cyanomethylamino-phenyl)-2-oxo-ethyl]-3-methyl-7-(3-methyl-2-buten-1-yl)-8-(3-amino-piperidin-1-yl)-xanthine
(429) 1-(2-{2-[(methoxycarbonyl)methylamino]-phenyl}-2-oxo-ethyl)-3-methyl-7-(3-methyl-2-buten-1-yl)-8-(3-amino-piperidin-1-yl)-xanthine
(430) 1-[2-(2-methylsulphonylamino-phenyl)-2-oxo-ethyl]-3-methyl-7-(3-methyl-2-buten-1-yl)-8-(3-amino-piperidin-1-yl)-xanthine
(431) 1-(2-{3-[(methoxycarbonyl)methylamino]-phenyl}-2-oxo-ethyl)-3-methyl-7-(3-methyl-2-buten-1-yl)-8-(3-amino-piperidin-1-yl)-xanthine
(432) 1-[2-(3-methylamino-phenyl)-2-oxo-ethyl]-3-methyl-7-(3-methyl-2-buten-1-yl)-8-(3-amino-piperidin-1-yl)-xanthine
(433) 1-{2-[3-(N-cyanomethyl-N-methyl-amino)-phenyl]-2-oxo-ethyl}-3-methyl-7-(3-methyl-2-buten-1-yl)-8-(3-amino-piperidin-1-yl)-xanthine
(434) 1-(2-{3-[(dimethylamino)sulphonylamino]-phenyl}-2-oxo-ethyl)-3-methyl-7-(3-methyl-2-buten-1-yl)-8-(3-amino-piperidin-1-yl)-xanthine
(435) 1-(2-{3-[(morpholin-4-yl)sulphonylamino]-phenyl}-2-oxo-ethyl)-3-methyl-7-(3-methyl-2-buten-1-yl)-8-(3-amino-piperidin-1-yl)-xanthine
(436) 1-[2-(3-aminosulphonylamino-phenyl)-2-oxo-ethyl]-3-methyl-7-(3-methyl-2-buten-1-yl)-8-(3-amino-piperidin-1-yl)-xanthine
(437) 1-[2-(3-ethylsulphonylamino-phenyl)-2-oxo-ethyl]-3-methyl-7-(3-methyl-2-buten-1-yl)-8-(3-amino-piperidin-1-yl)-xanthine
(438) 1-[2-(3-isopropylsulphonylamino-phenyl)-2-oxo-ethyl]-3-methyl-7-(3-methyl-2-buten-1-yl)-8-(3-amino-piperidin-1-yl)-xanthine
(439) 1-{2-[3-(2-oxo-imidazolidin-1-yl)-phenyl]-2-oxo-ethyl}-3-methyl-7-(3-methyl-2-buten-1-yl)-8-(3-amino-piperidin-1-yl)-xanthine
(440) 1-{2-[3-(3-methyl-2-oxo-imidazolidin-1-yl)-phenyl]-2-oxo-ethyl}-3-methyl-7-(3-methyl-2-buten-1-yl)-8-(3-amino-piperidin-1-yl)-xanthine
(441) 1-{2-[3-(3-methyl-2,5-dioxo-imidazolidin-1-yl)-phenyl]-2-oxo-ethyl}-3-methyl-7-(3-methyl-2-buten-1-yl)-8-(3-amino-piperidin-1-yl)-xanthine
(442) 1-{2-[3-(3-methyl-2,4-dioxo-imidazolidin-1-yl)-phenyl]-2-oxo-ethyl}-3-methyl-7-(3-methyl-2-buten-1-yl)-8-(3-amino-piperidin-1-yl)-xanthine
(443) 1-[(2-oxo-1,2-dihydro-quinolin-4-yl)methyl]-3-methyl-7-(3-methyl-2-buten-1-yl)-8-(3-amino-piperidin-1-yl)-xanthine
(444) 1-[(1-methyl-2-oxo-1,2-dihydro-quinolin-4-yl)methyl]-3-methyl-7-(3-methyl-2-buten-1-yl)-8-(3-amino-piperidin-1-yl)-xanthine
(445) 1-[(2-oxo-1,2-dihydro-quinazolin-4-yl)methyl]-3-methyl-7-(3-methyl-2-buten-1-yl)-8-(3-amino-piperidin-1-yl)-xanthine
(446) 1-[(1-methyl-2-oxo-1,2-dihydro-quinazolin-4-yl)methyl]-3-methyl-7-(3-methyl-2-buten-1-yl)-8-(3-amino-piperidin-1-yl)-xanthine
(447) 1-[(2-cyano-naphthalin-1-yl)methyl]-3-methyl-7-(3-methyl-2-buten-1-yl)-8-(3-amino-piperidin-1-yl)-xanthine
(448) 1-[(6-cyano-naphthalin-1-yl)methyl]-3-methyl-7-(3-methyl-2-buten-1-yl)-8-(3-amino-piperidin-1-yl)-xanthine
(449) 1-[(5-cyano-naphthalin-1-yl)methyl]-3-methyl-7-(3-methyl-2-buten-1-yl)-8-(3-amino-piperidin-1-yl)-xanthine
(450) 1-[(8-methyl-isoquinolin-1-yl)methyl]-3-methyl-7-(3-methyl-2-buten-1-yl)-8-(3-amino-piperidin-1-yl)-xanthine
(451) 1-[(5-cyano-isoquinolin-1-yl)methyl]-3-methyl-7-(3-methyl-2-buten-1-yl)-8-(3-amino-piperidin-1-yl)-xanthine
(452) 1-[(5-aminocarbonyl-isoquinolin-1-yl)methyl]-3-methyl-7-(3-methyl-2-buten-1-yl)-8-(3-amino-piperidin-1-yl)-xanthine
(453) 1-[(5-aminosulphonyl-isoquinolin-1-yl)methyl]-3-methyl-7-(3-methyl-2-buten-1-yl)-8-(3-amino-piperidin-1-yl)-xanthine
(454) 1-[(5-methylsulphonyl-isoquinolin-1-yl)methyl]-3-methyl-7-(3-methyl-2-buten-1-yl)-8-(3-amino-piperidin-1-yl)-xanthine
(455) 1-[(5-methylsulphonylamino-isoquinolin-1-yl)methyl]-3-methyl-7-(3-methyl-2-buten-1-yl)-8-(3-amino-piperidin-1-yl)-xanthine
(456) 1-[(5-methoxy-isoquinolin-1-yl)methyl]-3-methyl-7-(3-methyl-2-buten-1-yl)-8-(3-amino-piperidin-1-yl)-xanthine
(457) 1-[(6-methoxy-isoquinolin-1-yl)methyl]-3-methyl-7-(3-methyl-2-buten-1-yl)-8-(3-amino-piperidin-1-yl)-xanthine
(458) 1-[(7-methylsulphonylamino-isoquinolin-1-yl)methyl]-3-methyl-7-(3-methyl-2-buten-1-yl)-8-(3-amino-piperidin-1-yl)-xanthine
(459) 1-[(7-cyano-isoquinolin-1-yl)methyl]-3-methyl-7-(3-methyl-2-buten-1-yl)-8-(3-amino-piperidin-1-yl)-xanthine
(460) 1-[(7-aminocarbonyl-isoquinolin-1-yl)methyl]-3-methyl-7-(3-methyl-2-buten-1-yl)-8-(3-amino-piperidin-1-yl)-xanthine
(461) 1-[2-(2-hydroxy-phenyl)-2-oxo-ethyl]-3-methyl-7-(3-methyl-2-buten-1-yl)-8-(3-amino-piperidin-1-yl)-xanthine
(462) 1-[2-(2-cyanomethoxy-phenyl)-2-oxo-ethyl]-3-methyl-7-(3-methyl-2-buten-1-yl)-8-(3-amino-piperidin-1-yl)-xanthine
(463) 1-(2-{2-[(methoxycarbonyl)methoxy]-phenyl}-2-oxo-ethyl)-3-methyl-7-(3-methyl-2-buten-1-yl)-8-(3-amino-piperidin-1-yl)-xanthine
(464) 1-[2-(2-allyloxy-phenyl)-2-oxo-ethyl]-3-methyl-7-(3-methyl-2-buten-1-yl)-8-(3-amino-piperidin-1-yl)-xanthine
(465) 1-(2-{3-[(aminocarbonyl)methoxy]-phenyl}-2-oxo-ethyl)-3-methyl-7-(3-methyl-2-buten-1-yl)-8-(3-amino-piperidin-1-yl)-xanthine
(466) 1-(2-{3-[(methylaminocarbonyl)methoxy]-phenyl}-2-oxo-ethyl)-3-methyl-7-(3-methyl-2-buten-1-yl)-8-(3-amino-piperidin-1-yl)-xanthine
(467) 1-(2-{3-[(dimethylaminocarbonyl)methoxy]-phenyl}-2-oxo-ethyl)-3-methyl-7-(3-methyl-2-buten-1-yl)-8-(3-amino-piperidin-1-yl)-xanthine
(468) 1-[2-(3-{[(morpholin-4-yl)carbonyl]methoxy}-phenyl)-2-oxo-ethyl]-3-methyl-7-(3-methyl-2-buten-1-yl)-8-(3-amino-piperidin-1-yl)-xanthine
(469) 1-[2-(3-carboxymethoxy-phenyl)-2-oxo-ethyl]-3-methyl-7-(3-methyl-2-buten-1-yl)-8-(3-amino-piperidin-1-yl)-xanthine
(470) 1-[2-(3-methylsulphanylmethoxy-phenyl)-2-oxo-ethyl]-3-methyl-7-(3-methyl-2-buten-1-yl)-8-(3-amino-piperidin-1-yl)-xanthine
(471) 1-[2-(3-methylsulphinylmethoxy-phenyl)-2-oxo-ethyl]-3-methyl-7-(3-methyl-2-buten-1-yl)-8-(3-amino-piperidin-1-yl)-xanthine
(472) 1-[2-(3-methylsulphoylmethoxy-phenyl)-2-oxo-ethyl]-3-methyl-7-(3-methyl-2-buten-1-yl)-8-(3-amino-piperidin-1-yl)-xanthine
(473) 1-[2-(2-oxo-2,3-dihydro-benzoxazol-4-yl)-2-oxo-ethyl]-3-methyl-7-(3-methyl-2-buten-1-yl)-8-(3-amino-piperidin-1-yl)-xanthine
(474) 1-[2-(2-oxo-2,3-dihydro-1H-benzoimidazol-4-yl)-2-oxo-ethyl]-3-methyl-7-(3-methyl-2-buten-1-yl)-8-(3-amino-piperidin-1-yl)-xanthine
(475) 1-[2-(1-methyl-2-oxo-2,3-dihydro-1H-benzoimidazol-4-yl)-2-oxo-ethyl]-3-methyl-7-(3-methyl-2-buten-1-yl)-8-(3-amino-piperidin-1-yl)-xanthine
(476) 1-[2-(1,3-dimethyl-2-oxo-2,3-dihydro-1H-benzoimidazol-4-yl)-2-oxo-ethyl]-3-methyl-7-(3-methyl-2-buten-1-yl)-8-(3-amino-piperidin-1-yl)-xanthine
(477) 1-[2-(1H-benzoimidazol-4-yl)-2-oxo-ethyl]-3-methyl-7-(3-methyl-2-buten-1-yl)-8-(3-amino-piperidin-1-yl)-xanthine
(478) 1-[2-(2-methyl-1H-benzoimidazol-4-yl)-2-oxo-ethyl]-3-methyl-7-(3-methyl-2-buten-1-yl)-8-(3-amino-piperidin-1-yl)-xanthine
(479) 1-[2-(benzoxazol-4-yl)-2-oxo-ethyl]-3-methyl-7-(3-methyl-2-buten-1-yl)-8-(3-amino-piperidin-1-yl)-xanthine
(480) 1-[2-(2-methyl-benzoxazol-4-yl)-2-oxo-ethyl]-3-methyl-7-(3-methyl-2-buten-1-yl)-8-(3-amino-piperidin-1-yl)-xanthine
(481) 1-[2-(3-oxo-3,4-dihydro-2H-benzo[1,4]oxazin-5-yl)-2-oxo-ethyl]-3-methyl-7-(3-methyl-2-buten-1-yl)-8-(3-amino-piperidin-1-yl)-xanthine
(482) 1-[2-(benzo[1,3]dioxol-4-yl)-2-oxo-ethyl]-3-methyl-7-(3-methyl-2-buten-1-yl)-8-(3-amino-piperidin-1-yl)-xanthine
(483) 1-(2-phenyl-2-oxo-ethyl)-3-methyl-7-(3-methyl-2-buten-1-yl)-8-(3-amino-3-aminocarbonyl-piperidin-1-yl)-xanthine
(484) 1-(2-phenyl-2-oxo-ethyl)-3-methyl-7-(3-methyl-2-buten-1-yl)-8-(3-amino-4-aminocarbonyl-piperidin-1-yl)-xanthine
(485) 1-(2-phenyl-2-oxo-ethyl)-3-methyl-7-(3-methyl-2-buten-1-yl)-8-(3-amino-3-methylaminocarbonyl-piperidin-1-yl)-xanthine
(486) 1-(2-phenyl-2-oxo-ethyl)-3-methyl-7-(3-methyl-2-buten-1-yl)-8-(3-amino-3-dimethylaminocarbonyl-piperidin-1-yl)-xanthine
(487) 1-(2-phenyl-2-oxo-ethyl)-3-methyl-7-(3-methyl-2-buten-1-yl)-8-{3-amino-3-[(pyrrolidin-1-yl)carbonyl]-piperidin-1-yl}-xanthine
(488) 1-(2-phenyl-2-oxo-ethyl)-3-methyl-7-(3-methyl-2-buten-1-yl)-8-{3-amino-3-[(2-cyano-pyrrolidin-1-yl)carbonyl]-piperidin-1-yl}-xanthine
(489) 1-(2-phenyl-2-oxo-ethyl)-3-methyl-7-(3-methyl-2-buten-1-yl)-8-{3-amino-3-[(thiazolidin-3-yl)carbonyl]-piperidin-1-yl}-xanthine
(490) 1-(2-phenyl-2-oxo-ethyl)-3-methyl-7-(3-methyl-2-buten-1-yl)-8-{3-amino-3-[(4-cyano-thiazolidin-3-yl)carbonyl]-piperidin-1-yl}-xanthine
(491) 1-(2-phenyl-2-oxo-ethyl)-3-methyl-7-(3-methyl-2-buten-1-yl)-8-(5-amino-6-oxo-piperidin-3-yl)-xanthine
(492) 1-(2-phenyl-2-oxo-ethyl)-3-methyl-7-(3-methyl-2-buten-1-yl)-8-(5-amino-1-methyl-6-oxo-piperidin-3-yl)-xanthine
(493) 1-(2-phenyl-2-oxo-ethyl)-3-methyl-7-(3-methyl-2-buten-1-yl)-8-(3-amino-4-hydroxy-piperidin-1-yl)-xanthine
(494) 1-(2-phenyl-2-oxo-ethyl)-3-methyl-7-(3-methyl-2-buten-1-yl)-8-(3-amino-4-methoxy-piperidin-1-yl)-xanthine
(495) 1-(2-phenyl-2-oxo-ethyl)-3-methyl-7-(3-methyl-2-buten-1-yl)-8-(3-amino-5-hydroxy-piperidin-1-yl)-xanthine
(496) 1-(2-phenyl-2-oxo-ethyl)-3-methyl-7-(3-methyl-2-buten-1-yl)-8-(5-amino-2-oxo-piperidin-1-yl)-xanthine
(497) 1-(2-phenyl-2-oxo-ethyl)-3-methyl-7-(3-methyl-2-buten-1-yl)-8-(3-amino-2-oxo-piperidin-1-yl)-xanthine
(498) 1-(1-methoxycarbonyl-1-phenyl-methyl)-3-methyl-7-(3-methyl-2-buten-1-yl)-8-(3-amino-piperidin-1-yl)-xanthine
(499) 1-(1-carboxy-1-phenyl-methyl)-3-methyl-7-(3-methyl-2-buten-1-yl)-8-(3-amino-piperidin-1-yl)-xanthine
(500) 1-(1-aminocarbonyl-1-phenyl-methyl)-3-methyl-7-(3-methyl-2-buten-1-yl)-8-(3-amino-piperidin-1-yl)-xanthine
(501) 1-(1-methoxycarbonyl-2-phenyl-ethyl)-3-methyl-7-(3-methyl-2-buten-1-yl)-8-(3-amino-piperidin-1-yl)-xanthine
(502) 1-(1-carboxy-2-phenyl-ethyl)-3-methyl-7-(3-methyl-2-buten-1-yl)-8-(3-amino-piperidin-1-yl)-xanthine
(503) 1-(1-aminocarbonyl-2-phenyl-ethyl)-3-methyl-7-(3-methyl-2-buten-1-yl)-8-(3-amino-piperidin-1-yl)-xanthine
(504) 1-[(benzofuran-2-yl)methyl]-3-methyl-7-(3-methyl-2-buten-1-yl)-8-(3-amino-piperidin-1-yl)-xanthine
(505) 1-[(2,3-dihydro-benzofuran-2-yl)methyl]-3-methyl-7-(3-methyl-2-buten-1-yl)-8-(3-amino-piperidin-1-yl)-xanthine
(506) 1-[2-(2-amino-3-cyano-phenyl)-2-oxo-ethyl]-3-methyl-7-(3-methyl-2-buten-1-yl)-8-(3-amino-piperidin-1-yl)-xanthine
(507) 1-[2-(2-amino-3-fluoro-phenyl)-2-oxo-ethyl]-3-methyl-7-(3-methyl-2-buten-1-yl)-8-(3-amino-piperidin-1-yl)-xanthine
(508) 1-(2-phenyl-2-oxo-ethyl)-3-(tetrahydrofuran-3-yl)-7-(3-methyl-2-buten-1-yl)-8-(3-amino-piperidin-1-yl)-xanthine
(509) 1-(2-phenyl-2-oxo-ethyl)-3-(tetrahydropyran-4-yl)-7-(3-methyl-2-buten-1-yl)-8-(3-amino-piperidin-1-yl)-xanthine
(510) 1-(2-phenyl-2-oxo-ethyl)-3-[(tetrahydrofuran-2-yl)methyl]-7-(3-methyl-2-buten-1-yl)-8-(3-amino-piperidin-1-yl)-xanthine
(511) 1-(2-phenyl-2-oxo-ethyl)-3-[(tetrahydropyran-4-yl)methyl]-7-(3-methyl-2-buten-1-yl)-8-(3-amino-piperidin-1-yl)-xanthine
(512) 1-methyl-3-[2-(4-dimethylamino-phenyl)-ethyl]-7-(2-cyano-benzyl)-8-(3-amino-piperidin-1-yl)-xanthine
(513) 1,3-dimethyl-7-(3-methyl-1-buten-1-yl)-8-(3-amino-piperidin-1-yl)-xanthine
(514) 1-(1,4-dioxo-1,4-dihydro-naphthalen-2-yl)-3-methyl-7-(3-methyl-2-buten-1-yl)-8-(3-amino-piperidin-1-yl)-xanthine
(515) 1-(4-oxo-4H-chromen-3-yl)-3-methyl-7-(3-methyl-2-buten-1-yl)-8-(3-amino-piperidin-1-yl)-xanthine
(516) 1-(1-oxo-indan-2-yl)-3-methyl-7-(3-methyl-2-buten-1-yl)-8-(3-amino-piperidin-1-yl)-xanthine
(517) 1-(1-methyl-2-phenyl-2-oxo-ethyl)-3-methyl-7-(3-methyl-2-buten-1-yl)-8-(3-amino-piperidin-1-yl)-xanthine
(518) 1-[2-oxo-2-(3-oxo-3,4-dihydro-2H-benzo[1,4]oxazin-8-yl)-ethyl]-3-methyl-7-(3-methyl-2-buten-1-yl)-8-(3-amino-piperidin-1-yl)-xanthine
(519) 1-[2-oxo-2-(4-methyl-3-oxo-3,4-dihydro-2H-benzo[1,4]oxazin-8-yl)-ethyl]-3-methyl-7-(3-methyl-2-buten-1-yl)-8-(3-amino-piperidin-1-yl)-xanthine
(520) 1-[(cinnolin-4-yl)methyl]-3-methyl-7-(3-methyl-2-buten-1-yl)-8-(3-amino-piperidin-1-yl)-xanthine
(521) 1-[(2-oxo-2H-chromen-4-yl)methyl]-3-methyl-7-(3-methyl-2-buten-1-yl)-8-(3-amino-piperidin-1-yl)-xanthine
(522) 1-[(1-oxo-1,2-dihydro-isoquinolin-4-yl)methyl]-3-methyl-7-(3-methyl-2-buten-1-yl)-8-(3-amino-piperidin-1-yl)-xanthine
(523) 1-[(2-methyl-1-oxo-1,2-dihydro-isoquinolin-4-yl)methyl]-3-methyl-7-(3-methyl-2-buten-1-yl)-8-(3-amino-piperidin-1-yl)-xanthine
(524) 1-[(4-oxo-3,4-dihydro-phthalazin-1-yl)methyl]-3-methyl-7-(3-methyl-2-buten-1-yl)-8-(3-amino-piperidin-1-yl)-xanthine
(525) 1-[(3-methyl-4-oxo-3,4-dihydro-phthalazin-1-yl)methyl]-3-methyl-7-(3-methyl-2-buten-1-yl)-8-(3-amino-piperidin-1-yl)-xanthine
(526) 1-[([1,5]naphthyridin-4-yl)methyl]-3-methyl-7-(3-methyl-2-buten-1-yl)-8-(3-amino-piperidin-1-yl)-xanthine
(527) 1-[([1,7]naphthyridin-8-yl)methyl]-3-methyl-7-(3-methyl-2-buten-1-yl)-8-(3-amino-piperidin-1-yl)-xanthine
(528) 1-[(quinolin-2-yl)methyl]-3-methyl-7-(3-methyl-2-buten-1-yl)-8-(3-amino-piperidin-1-yl)-xanthine
(529) 1-[(isoquinolin-3-yl)methyl]-3-methyl-7-(3-methyl-2-buten-1-yl)-8-(3-amino-piperidin-1-yl)-xanthine
(530) 1-{2-oxo-2-[3-(2-oxo-tetrahydro-pyrimidin-1-yl)-phenyl]-ethyl}-3-methyl-7-(3-methyl-2-buten-1-yl)-8-(3-amino-piperidin-1-yl)-xanthine
(531) 1-{2-oxo-2-[3-(3-methyl-2-oxo-tetrahydro-pyrimidin-1-yl)-phenyl]-ethyl}-3-methyl-7-(3-methyl-2-buten-1-yl)-8-(3-amino-piperidin-1-yl)-xanthine

### EXAMPLE 11

### Coated Tablets Containing 75 mg of Active Substance

1 tablet core contains:

| | | |
|---|---|---|
| active substance | 75.0 | mg |
| calcium phosphate | 93.0 | mg |
| corn starch | 35.5 | mg |
| polyvinylpyrrolidone | 10.0 | mg |
| hydroxypropylmethylcellulose | 15.0 | mg |
| magnesium stearate | 1.5 | mg |
| | 230.0 | mg |

### Preparation:

The active substance is mixed with calcium phosphate, corn starch, polyvinyl-pyrrolidone, hydroxypropylmethylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape.
Weight of core: 230 mg
die: 9 mm, convex

The tablet cores thus produced are coated with a film consisting essentially of hydroxypropylmethylcellulose. The finished film-coated tablets are polished with beeswax.
Weight of coated tablet: 245 mg.

### EXAMPLE 12

### Tablets Containing 100 Mg of Active Substance

### Composition:

1 tablet contains:

| | | |
|---|---|---|
| active substance | 100.0 | mg |
| lactose | 80.0 | mg |
| maize starch | 34.0 | mg |
| polyvinylpyrrolidone | 4.0 | mg |
| magnesium stearate | 2.0 | mg |
| | 220.0 | mg |

### Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.
Weight of tablet: 220 mg
Diameter: 10 mm, biplanar, facetted on both sides and notched on one side.

### EXAMPLE 13

### Tablets Containing 150 Mg of Active Substance

### Composition:

1 tablet contains:

| | | |
|---|---|---|
| active substance | 150.0 | mg |
| powdered lactose | 89.0 | mg |
| maize starch | 40.0 | mg |
| colloidal silica | 10.0 | mg |
| polyvinylpyrrolidone | 10.0 | mg |
| magnesium stearate | 1.0 | mg |
| | 300.0 | mg |

### Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.
Weight of tablet: 300 mg
die: 10 mm, flat

### EXAMPLE 14

### Hard Gelatine Capsules Containing 150 Mg of Active Substance

1 capsule contains:

| | | |
|---|---|---|
| active substance | 150.0 | mg |
| dried maize starch | approx. 180.0 | mg |
| powdered lactose. | approx. 87.0 | mg |
| magnesium stearate | 3.0 | mg |
| | approx. 420.0 | mg |

### Preparation:

The active substance is mixed with the excipients, passed through a screen with a mesh size of 0.75 mm and homogeneously mixed using a suitable apparatus. The finished mixture is packed into size 1 hard gelatine capsules.
Capsule filling: approx. 320 mg
Capsule shell: size 1 hard gelatine capsule.

### EXAMPLE 15

### Suppositories Containing 150 Mg of Active Substance

1 suppository contains:

| | | |
|---|---|---|
| active substance | 150.0 | mg |
| polyethyleneglycol 1500 | 550.0 | mg |
| polyethyleneglycol 6000 | 460.0 | mg |
| polyoxyethylene sorbitan monostearate | 840.0 | mg |
| | 2000.0 | mg |

### Preparation:

After the suppository mass has been melted the active substance is homogeneously distributed therein and the melt is poured into chilled moulds.

### EXAMPLE 16

### Suspension Containing 50 Mg of Active Substance

100 ml of suspension contain:

| | | |
|---|---|---|
| active substance | 1.00 | g |
| Na salt of carboxymethylcellulose | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavouring | 0.30 | g |
| dist. water | ad 100 | ml |

### Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavouring have been added and dissolved, the suspension is evacuated with stirring to eliminate air.
5 ml of suspension contain 50 mg of active substance.

### EXAMPLE 17

### Ampoules Containing 10 Mg of Active Substance

### Composition:

| active substance | 10.0 | mg |
|---|---|---|
| 0.01N hydrochloric acid | q.s. | |
| twice-distilled water | ad 2.0 | ml |

### Preparation:

The active substance is dissolved in the requisite amount of 0.01 N HCl, made isotonic with saline, sterile filtered and transferred into 2 ml ampoules.

### EXAMPLE 18

### Ampoules Containing 50 Mg of Active Substance

### Composition:

| active substance | 50.0 | mg |
|---|---|---|
| 0.01N hydrochloric acid | q.s. | |
| twice-distilled water | ad 10.0 | ml |

### Preparation:

The active substance is dissolved in the requisite amount of 0.01 N HCl, made isotonic with saline, sterile filtered and transferred into 10 ml ampoules.

What is claimed is:

1. A compound of the formula 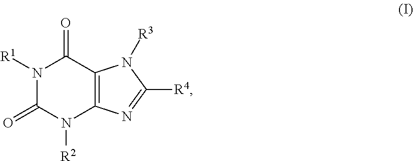
wherein
I. R¹ denotes
A. a hydrogen atom,
B. a C₁₋₈-alkyl group,
C. a C₃₋₈-alkenyl group,
D. a C₃₋₄-alkenyl group which is substituted by a C₁₋₂-alkyloxy-carbonyl, aminocarbonyl, C₁₋₃-alkylamino-carbonyl, di-(C₁₋₃-alkyl)-amino-carbonyl, pyrrolidin-1-ylcarbonyl, piperidin-1-ylcarbonyl- or morpholin-4-ylcarbonyl-group,
E. a C₃₋₈-alkynyl group,
F. a C₁₋₆-alkyl group substituted by a group Rₐ, wherein
i. Rₐ denotes a C₃₋₇-cycloalkyl, heteroaryl, cyano, carboxy, C₁₋₃-alkyloxy-carbonyl, aminocarbonyl, C₁₋₃-alkylamino-carbonyl, di-(C₁₋₃-alkyl)-amino-carbonyl, pyrrolidin-1-ylcarbonyl, piperidin-1-ylcarbonyl, morpholin-4-ylcarbonyl, piperazin-1-ylcarbonyl, 4-methylpiperazin-1-ylcarbonyl or 4-ethylpiperazin-1-ylcarbonyl group,
G. a C₁₋₆-alkyl group substituted by a phenyl group, wherein the phenyl ring is substituted by the groups R¹⁰ to R¹⁴ and
i. R¹⁰ denotes
a. a hydrogen atom,
b. a fluorine, chlorine, bromine or iodine atom,
c. a C₁₋₄-alkyl, hydroxy, or C₁₋₄-alkyloxy group,
d. a nitro, amino, C₁₋₃-alkylamino, di-(C₁₋₃-alkyl)amino, cyano-C₁₋₃-alkylamino, [N-(cyano-C₁₋₃-alkyl)-N—C₁₋₃-alkyl-amino], C₁₋₃-alkyloxy-carbonyl-C₁₋₃-alkylamino, pyrrolidin-1-yl, piperidin-1-yl, morpholin-4-yl, piperazin-1-yl or 4-(C₁₋₃-alkyl)-piperazin-1-yl group,
e. a C₁₋₃-alkyl-carbonylamino, arylcarbonylamino, aryl-C₁₋₃-alkyl-carbonylamino, C₁₋₃-alkyloxy-carbonylamino, aminocarbonylamino, C₁₋₃-alkyl-aminocarbonylamino, di-(C₁₋₃-alkyl)aminocarbonylamino, pyrrolidin-1-yl-carbonyl-amino, piperidin-1-yl-carbonylamino, morpholin-4-yl-carbonylamino, piperazin-1-yl-carbonylamino or 4-(C₁₋₃-alkyl)-piperazin-1-yl-carbonylamino, C₁₋₃-alkyl-sulphonylamino, bis-(C₁₋₃-alkylsulphonyl)-amino, aminosulphonylamino, C₁₋₃-alkylamino-sulphonylamino, di-(C₁₋₃-alkyl)amino-sulphonylamino, pyrrolidin-1-yl-sulphonylamino, piperidin-1-yl-sulphonylamino, morpholin-4-yl-sulphonylamino, piperazin-1-yl-sulphonylamino or 4-(C₁₋₃-alkyl)-piperazin-1-yl-sulphonylamino, (C₁₋₃-alkylamino)thiocarbonylamino, (C₁₋₃-alkyloxy-carbonylamino)carbonylamino, aryl-sulphonylamino or aryl-C₁₋₃-alkyl-sulphonylamino group,
f. an N—(C₁₋₃-alkyl)-C₁₋₃-alkyl-carbonylamino, N—(C₁₋₃-alkyl)-arylcarbonylamino, N—(C₁₋₃-alkyl)-aryl-C₁₋₃-alkyl-carbonylamino, N—(C₁₋₃-alkyl)-C₁₋₃-alkyloxy-carbonyl-amino, N-(aminocarbonyl)-C₁₋₃-alkylamino, N—(C₁₋₃-alkyl-aminocarbonyl)-C₁₋₃-alkylamino, N-[di-(C₁₋₃-alkyl)aminocarbonyl]-C₁₋₃-alkylamino, N—(C₁₋₃-alkyl)-C₁₋₃-alkyl-sulphonylamino, N—(C₁₋₃-alkyl)-arylsulphonylamino or N—(C₁₋₃-alkyl)-aryl-C₁₋₃-alkyl-sulphonylamino group,
g. a 2-oxo-imidazolidin-1-yl, 2,4-dioxo-imidazolidin-1-yl, 2,5-dioxo-imidazolidin-1-yl or 2-oxo-hexahydropyrimidin-1-yl group wherein the nitrogen atom in the 3 position in each case may be substituted by a methyl or ethyl group,
h. a cyano, carboxy, C₁₋₃-alkyloxy-carbonyl, aminocarbonyl, C₁₋₃-alkyl-aminocarbonyl, di-(C₁₋₃-alkyl)-aminocarbonyl, pyrrolidin-1-yl-carbonyl, piperidin-1-yl-carbonyl, morpholin-4-yl-carbonyl, piperazin-1-yl-carbonyl or 4-(C₁₋₃-alkyl)-piperazin-1-yl-carbonyl group,
i. a C₁₋₃-alkyl-carbonyl or an arylcarbonyl group,
j. a carboxy-C₁₋₃-alkyl, C₁₋₃-alkyloxy-carbonyl-C₁₋₃-alkyl, cyano-C₁₋₃-alkyl, aminocarbonyl-C₁₋₃-alkyl, C₁₋₃-alkyl-aminocarbonyl-C₁₋₃-alkyl, di-(C₁₋₃-alkyl)-aminocarbonyl-C₁₋₃-alkyl, pyrrolidin-1-yl-carbonyl-C₁₋₃-alkyl, piperidin-1-yl-carbonyl-C₁₋₃-alkyl, morpholin-4-yl-carbonyl-C₁₋₃-alkyl, piperazin-1-yl-carbonyl-C₁₋₃-alkyl or 4-(C₁₋₃-alkyl)-piperazin-1-yl-carbonyl-C₁₋₃-alkyl group,
k. a carboxy-C₁₋₃-alkyloxy, C₁₋₃-alkyloxy-carbonyl-C₁₋₃-alkyloxy, cyano-C₁₋₃-alkyloxy, aminocarbonyl-C₁₋₃-alkyloxy, C₁₋₃-alkyl-aminocarbonyl-C₁₋₃-alkyloxy, di-(C₁₋₃-alkyl)-aminocarbonyl-C₁₋₃-alkyloxy, pyrrolidin-1-yl-carbonyl-C₁₋₃-alkyl-oxy, piperidin-1-yl-carbonyl-C₁₋₃-alkyloxy, morpholin-4-yl-carbonyl-C₁₋₃-alkyl-oxy, piperazin-1-yl-carbonyl-C₁₋₃-alkyloxy or 4-(C₁₋₃-alkyl)-piperazin-1-yl-carbonyl-C₁₋₃-alkyloxy group,
l. a hydroxy-C₁₋₃-alkyl, C₁₋₃-alkyloxy-C₁₋₃-alkyl, amino-C₁₋₃-alkyl, C₁₋₃-alkylamino-C₁₋₃-alkyl, di-(C₁₋₃-alkyl)-amino-C₁₋₃-alkyl, pyrrolidin-1-yl-C₁₋₃-alkyl, piperidin-1-yl-C₁₋₃-alkyl, morpholin-4-yl-C₁₋₃-alkyl, piperazin-1-yl-C₁₋₃-alkyl, 4-(C₁₋₃-alkyl)-piperazin-1-yl-C₁₋₃-alkyl group,
m. a hydroxy-C₁₋₃-alkyloxy, C₁₋₃-alkyloxy-C₁₋₃-alkyloxy, C₁₋₃-alkylsulphanyl-C₁₋₃-alkyloxy, C₁₋₃-alkylsulphinyl-C₁₋₃-alkyloxy, C₁₋₃-alkylsulphonyl-C₁₋₃-alkyloxy, amino-C₁₋₃-alkyloxy, C₁₋₃-alkylamino-C₁₋₃-alkyloxy, di-(C₁₋₃-alkyl)-amino-C₁₋₃-alkyloxy, pyrrolidin-1-yl-C₁₋₃-alkyloxy, piperidin-1-yl-C₁₋₃-alkyloxy, morpholin-4-yl-C₁₋₃-alkyloxy, piperazin-1-yl-C₁₋₃-alkyloxy, 4-(C₁₋₃-alkyl)-piperazin-1-yl-C₁₋₃-alkyloxy group,
n. a mercapto, C₁₋₃-alkylsulphanyl, C₁₋₃-alkysulphinyl, C₁₋₃-alkylsulphonyl, C₁₋₃-alkylsulphonyloxy, arylsulphonyloxy, trifluoromethylsulphanyl, trifluoromethylsulphinyl or trifluoromethylsulphonyl group,
o. a sulpho, aminosulphonyl, C₁₋₃-alkyl-aminosulphonyl, di-(C₁₋₃-alkyl)-aminosulphonyl, pyrrolidin-1-yl-sulphonyl, piperidin-1-yl-sulphonyl, morpholin-4-yl-sulphonyl, piperazin-1-yl-sulphonyl or 4-(C₁₋₃-alkyl)-piperazin-1-yl-sulphonyl group,
p. a methyl or methoxy group substituted by 1 to 3 fluorine atoms,
q. an ethyl or ethoxy group substituted by 1 to 5 fluorine atoms,
r. a C₂₋₄-alkenyl or C₂₋₄-alkynyl group,
s. a C₃₋₄-alkenyloxy or C₃₋₄-alkynyloxy group,
t. a C₃₋₆-cycloalkyl or C₃₋₆-cycloalkyloxy group,
u. a C₃₋₆-cycloalkyl-C₁₋₃-alkyl or C₃₋₆-cycloalkyl-C₁₋₃-alkyloxy group or
v. an aryl, aryloxy, aryl-C₁₋₃-alkyl or aryl-C₁₋₃-alkyloxy group,
ii. R¹¹ and R¹², which may be identical or different, each denote a hydrogen atom, a fluorine, chlorine, bromine or iodine atom, a C₁₋₃-alkyl, trifluoromethyl, hydroxy or C₁₋₃-alkyloxy group or a cyano group, or R¹¹ together with R¹², if they are bound to adjacent carbon atoms, also denote a methylenedioxy, difluoromethylenedioxy or a straight-chain C₃₋₅-alkylene group, and
iii. R¹³ and R¹⁴, which may be identical or different, each denote a hydrogen atom, a fluorine, chlorine or bromine atom, a trifluoromethyl, C₁₋₃-alkyl or C₁₋₃-alkyloxy group,
H. a phenyl-C₁₋₄-alkyl group wherein the alkyl moiety is substituted by a cyano, carboxy, C₁₋₃-alkyloxy-carbonyl, aminocarbonyl, C₁₋₃-alkyl-aminocarbonyl, di-(C₁₋₃-alkyl)-aminocarbonyl, pyrrolidin-1-yl-carbonyl, piperidin-1-yl-carbonyl, morpholin-4-yl-carbonyl group and the phenyl moiety is substituted by the groups R¹⁰ to R¹⁴, wherein R¹⁰ to R¹⁴ are as hereinbefore defined,
I. a phenyl group substituted by the groups R¹⁰ to R¹⁴, wherein R¹⁰ to R¹⁴ are as hereinbefore defined,
J. a phenyl-C₂₋₃-alkenyl group wherein the phenyl moiety is substituted by the groups R¹⁰ to R¹⁴, wherein R¹⁰ to R¹⁴ are as hereinbefore defined,
K. a phenyl-(CH₂)ₘ-A-(CH₂)ₙ-group wherein the phenyl moiety is substituted by R¹⁰ to R¹⁴, wherein R¹⁰ to R¹⁴ are as hereinbefore defined and
i. A denotes a carbonyl, cyanoiminomethylene, hydroxyiminomethylene or C₁₋₃-alkyloxyiminomethylene group, m denotes the number 0, 1 or 2 and n denotes the number 1, 2 or 3,
L. a phenylcarbonylmethyl group wherein the phenyl moiety is substituted by R¹⁰ to R¹⁴, wherein R¹⁰ to R¹⁴ are as hereinbefore defined and the methyl moiety is substituted by a C₁₋₃-alkyl group,
M. a phenyl-(CH₂)ₘ-B-(CH₂)ₙ group wherein the phenyl moiety is substituted by R¹⁰ to R¹⁴, wherein R¹⁰ to R¹⁴, m and n are as hereinbefore defined and
i. B denotes a methylene group which is substituted by a hydroxy, C₁₋₃-alkyloxy, amino, C₁₋₃-alkylamino, di-(C₁₋₃-alkyl)-amino, mercapto, C₁₋₃-alkylsulphanyl, C₁₋₃-alkylsulphinyl or C₁₋₃-alkylsulphonyl group and is optionally additionally substituted by a methyl or ethyl group,
N. a naphthyl-C₁₋₃-alkyl group wherein the naphthyl moiety is substituted by the groups R¹⁰ to R¹⁴, wherein R¹⁰ to R¹⁴ are as hereinbefore defined,
O. a naphthyl-(CH₂)ₘ-A-(CH₂)ₙ group wherein the naphthyl moiety is substituted by R¹⁰ to R¹⁴, wherein R¹⁰ to R¹⁴, A, m and n are as hereinbefore defined,
P. a naphthyl-(CH₂)ₘ-B-(CH₂)ₙ group wherein the naphthyl moiety is substituted by R¹⁰ to R¹⁴, wherein R¹⁰ to R¹⁴, B, m and n are as hereinbefore defined,
Q. a [1,4]naphthoquinon-2-yl, chromen-4-on-3-yl, 1-oxoindan-2-yl, 1,3-dioxoindan-2-yl- or 2,3-dihydro-3-oxo-benzofuran-2-yl group,
R. a heteroaryl-(CH₂)ₘ-A-(CH₂)ₙ group, wherein A, m and n are as hereinbefore defined,
S. a heteroaryl-(CH₂)ₘ-B-(CH₂)ₙ group, wherein B, m and n are as hereinbefore defined,
T. a C₁₋₆-alkyl-A-(CH₂)ₙ group, wherein A and n are as hereinbefore defined,
U. a C₃₋₇-cycloalkyl-(CH₂)ₘ-A-(CH₂)ₙ group, wherein A, m and n are as hereinbefore defined,
V. a C₃₋₇-cycloalkyl-(CH₂)ₘ-B-(CH₂)ₙ group, wherein B, m and n are as hereinbefore defined,
W. an R²¹-A-(CH₂)ₙ group wherein R²¹ denotes a C₁₋₃-alkyloxycarbonyl, aminocarbonyl, C₁₋₃-alkylaminocarbonyl, di-(C₁₋₃-alkyl)aminocarbonyl, pyrrolidin-1-yl-carbonyl, piperidin-1-yl-carbonyl or morpholin-4-yl-carbonyl, piperazin-1-yl-carbonyl, 4-methylpiperazin-1-yl-carbonyl or 4-ethylpiperazin-1-yl-carbonyl group and A and n are as hereinbefore defined,
X. a phenyl-(CH₂)ₘ-D-C₁₋₃-alkyl group wherein the phenyl moiety is substituted by the groups R¹⁰ to R¹⁴, wherein R¹⁰ to R¹⁴ and m are as hereinbefore defined and D denotes an oxygen or sulphur atom, an imino, C₁₋₃-alkylimino, sulphinyl or sulphonyl group,
Y. a naphthyl-(CH₂)ₘ-D-C₁₋₃-alkyl group wherein the naphthyl moiety is substituted by the groups R¹⁰ to R¹⁴, wherein R¹⁰ to R¹⁴, D and m are as hereinbefore defined,
Z. a C₂₋₆-alkyl group substituted by a group R_{b}, wherein
i. R_{b} is isolated by at least two carbon atoms from the cyclic nitrogen atom in the 1 position of the xanthine skeleton and
ii. R_{b} denotes a hydroxy, C₁₋₃-alkyloxy, mercapto, C₁₋₃-alkylsulphanyl, C₁₋₃-alkylsulphinyl, C₁₋₃-alkylsulphonyl, amino, C₁₋₃-alkylamino, di-(C₁₋₃-alkyl)-amino, pyrrolidin-1-yl, piperidin-1-yl, morpholin-4-yl, piperazin-1-yl or 4-(C₁₋₃-alkyl)-piperazin-1-yl group,
AA. a C₃₋₆-cycloalkyl group, or
BB. an amino or arylcarbonylamino group,
II. R² denotes
A. a hydrogen atom,
B. a C₁₋₈-alkyl group,
C. a C₂₋₆-alkenyl group,
D. a C₃₋₆-alkynyl group,
E. a C₁₋₆-alkyl group substituted by a group Rₐ, wherein Rₐ is as hereinbefore defined,
F. a tetrahydrofuran-3-yl, tetrahydropyran-3-yl, tetrahydropyran-4-yl, tetrahydrofuranyl-C₁₋₃-alkyl or tetrahydropyranyl-C₁₋₃-alkyl group,
G. a C₁₋₆-alkyl group substituted by a phenyl group, wherein the phenyl ring is substituted by the groups R¹⁰ to R¹⁴ and R¹⁰ to R¹⁴ are as hereinbefore defined,
H. a phenyl group substituted by the groups R¹⁰ to R¹⁴, wherein R¹⁰ to R¹⁴ are as hereinbefore defined,
I. a phenyl-C₂₋₃-alkenyl group wherein the phenyl moiety is substituted by the groups R¹⁰ to R¹⁴, wherein R¹⁰ to R¹⁴ are as hereinbefore defined,
J. a phenyl-(CH₂)ₘ-A-(CH₂)ₙ group wherein the phenyl moiety is substituted by R¹⁰ to R¹⁴, wherein R¹⁰ to R¹⁴, A, m and n are as hereinbefore defined,
K. a phenyl-(CH₂)ₘ-B-(CH₂)ₙ group wherein the phenyl moiety is substituted by R¹⁰ to R¹⁴, wherein R¹⁰ to R¹⁴, B, m and n are as hereinbefore defined,
L. a heteroaryl-(CH₂)ₘ-A-(CH₂)ₙ group, wherein A, m and n are as hereinbefore defined,
M. a heteroaryl-(CH₂)ₘ-B-(CH₂)ₙ group, wherein B, m and n are as hereinbefore defined,
N. a C₁₋₆-alkyl-A-(CH₂)ₙ group, wherein A and n are as hereinbefore defined,
O. a C₃₋₇-cycloalkyl-(CH₂)ₘ-A-(CH₂)ₙ group, wherein A, m and n are as hereinbefore defined,
P. a C₃₋₇-cycloalkyl-(CH₂)ₘ-B-(CH₂)ₙ group, wherein B, m and n are as hereinbefore defined,
Q. an R²¹-A-(CH₂)ₙ group wherein R²¹, A and n are as hereinbefore defined,
R. a phenyl-(CH₂)ₘ-D-C₁₋₃-alkyl group wherein the phenyl moiety is substituted by the groups R¹⁰ to R¹⁴, wherein R¹⁰ to R¹⁴, m and D are as hereinbefore defined,
S. a C₂₋₆-alkyl group substituted by a group R_{b}, wherein
i. R_{b} is isolated by at least two carbon atoms from the cyclic nitrogen atom in the 3 position of the xanthine skeleton and is as hereinbefore defined,
T. or a C₃₋₆-cycloalkyl group,
III. R³ denotes
A. a C₁₋₈-alkyl group,
B. a C₁₋₄-alkyl group substituted by the group R_{c}, wherein
i. R_{c} denotes
a. a C₃₋₇-cycloalkyl group optionally substituted by one or two C₁₋₃-alkyl groups,
b. a C₅₋₇-cycloalkenyl group optionally substituted by one or two C₁₋₃-alkyl groups,
c. an aryl group, or
d. a furanyl, thienyl, oxazolyl, isoxazolyl, thiazolyl, isothiazolyl, pyridyl, pyridazinyl, pyrimidyl or pyrazinyl group, wherein the abovementioned heterocyclic groups may each be substituted by one or two C₁₋₃-alkyl groups or by a fluorine, chlorine, bromine or iodine atom or by a trifluoromethyl, cyano or C₁₋₃-alkyloxy group,
C. a C₃₋₈-alkenyl group,
D. a C₃₋₆-alkenyl group substituted by a fluorine, chlorine or bromine atom or a trifluoromethyl group,
E. a C₃₋₈-alkynyl group,
F. an aryl group or
G. an aryl-C₂₋₄-alkenyl group,
and
IV. R⁴ denotes
A. an azetidin-1-yl or pyrrolidin-1-yl group which is substituted in the 3 position by an RₑNR_{d} group and may additionally be substituted by one or two C₁₋₃-alkyl groups, wherein
i. Rₑ denotes a hydrogen atom or a C₁₋₃-alkyl group and
ii. R_{d} denotes a hydrogen atom, a C₁₋₃-alkyl group, an R_{f}—C₁₋₃-alkyl group or an R_{g}—C₂₋₃-alkyl group, wherein
a. R_{f} denotes a carboxy, C₁₋₃-alkyloxy-carbonyl, aminocarbonyl, C₁₋₃-alkylamino-carbonyl, di-(C₁₋₃-alkyl)-aminocarbonyl, pyrrolidin-1-yl-carbonyl, 2-cyanopyrrolidin-1-yl-carbonyl, 2-carboxypyrrolidin-1-yl-carbonyl, 2-methoxycarbonylpyrrolidin-1-yl-carbonyl, 2-ethoxycarbonylpyrrolidin-1-yl-carbonyl, 2-aminocarbonylpyrrolidin-1-yl-carbonyl, 4-cyanothiazolidin-3-yl-carbonyl, 4-carboxythiazolidin-3-yl-carbonyl, 4-methoxycarbonylthiazolidin-3-yl-carbonyl, 4-ethoxy-carbonylthiazolidin-3-yl-carbonyl, 4-aminocarbonylthiazolidin-3-yl-carbonyl, piperidin-1-yl-carbonyl, morpholin-4-yl-carbonyl, piperazin-1-yl-carbonyl, 4-methyl-piperazin-1-yl-carbonyl or 4-ethyl-piperazin-1-yl-carbonyl group and
b. R_{g}, which is separated by two carbon atoms from the nitrogen atom of the RₑNR_{d} group, denotes a hydroxy, methoxy or ethoxy group,
B. a piperidin-1-yl or hexahydroazepin-1-yl group which is substituted in the 3 position or in the 4 position by an RₑNR_{d} group and may additionally be substituted by one or two C₁₋₃-alkyl groups, wherein Rₑ and R_{d} are as hereinbefore defined,
C. a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
D. a 3-amino-piperidin-1-yl group wherein the piperidin-1-yl moiety in the 4 position or in the 5 position is additionally substituted by a hydroxy or methoxy group,
E. a 3-amino-piperidin-1-yl group wherein the methylene group in the 2 position or in the 6 position is replaced by a carbonyl group,
F. a piperidin-1-yl or hexahydroazepin-1-yl-group substituted in the 3 position by an amino, C₁₋₃-alkylamino or di-(C₁₋₃-alkyl)-amino group, wherein in each case two hydrogen atoms at the carbon skeleton of the piperidin-1-yl or hexahydroazepin-1-yl-group are replaced by a straight-chain alkylene bridge, this bridge containing 2 to 5 carbon atoms if the two hydrogen atoms are located on the same carbon atom, or 1 to 4 carbon atoms if the hydrogen atoms are located on adjacent carbon atoms, or 1 to 4 carbon atoms, if the hydrogen atoms are located at carbon atoms separated by one atom, or 1 to 3 carbon atoms if the two hydrogen atoms are located at carbon atoms separated by two atoms,
G. an azetidin-1-yl, pyrrolidin-1-yl, piperidin-1-yl or hexahydroazepin-1-yl group which is substituted by an amino-C₁₋₃-alkyl, C₁₋₃-alkylamino-C₁₋₃-alkyl or a —(C₁₋₃-alkyl)amino-C₁₋₃-alkyl group,
H. a C₃₋₇-cycloalkyl group which is substituted by an amino, C₁₋₃-alkylamino or di-(C₁₋₃-alkyl)-amino group,
I. a C₃₋₇-cycloalkyl group which is substituted by an amino-C₁₋₃-alkyl, C₁₋₃-alkylamino-C₁₋₃-alkyl or a di-(C₁₋₃-alkyl)amino-C₁₋₃-alkyl group,
J. a C₃₋₇-cycloalkyl-C₁₋₂-alkyl group wherein the cycloalkyl moiety is substituted by an amino, C₁₋₃-alkylamino or di-(C₁₋₃-alkyl)-amino group,
K. a C₃₋₇-cycloalkyl-C₁₋₂-alkyl group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
L. a C₃₋₇-cycloalkylamino group wherein the cycloalkyl moiety is substituted by an amino, C₁₋₃-alkylamino or di-(C₁₋₃-alkyl)-amino group, wherein the two nitrogen atoms on the cycloalkyl moiety are separated from one another by at least two carbon atoms,
M. an N—(C₃₋₇-cycloalkyl)-N—(C₁₋₃-alkyl)-amino group wherein the cycloalkyl moiety is substituted by an amino, C₁₋₃-alkylamino or di-(C₁₋₃-alkyl)-amino group, wherein the two nitrogen atoms on the cycloalkyl moiety are separated from one another by at least two carbon atoms,
N. a C₃₋₇-cycloalkylamino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
O. an N—(C₃₋₇-cycloalkyl)-N—(C₁₋₃-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
P. a C₃₋₇-cycloalkyl-C₁₋₂-alkyl-amino group wherein the cycloalkyl moiety is substituted by an amino, C₁₋₃-alkylamino or di-(C₁₋₃-alkyl)-amino group,
Q. an N—(C₃₋₇-cycloalkyl-C₁₋₂-alkyl)-N—(C₁₋₂-alkyl)-amino group wherein the cycloalkyl moiety is substituted by an amino, C₁₋₃-alkylamino or di-(C₁₋₃-alkyl)-amino group,
R. a C₃₋₇-cycloalkyl-C₁₋₂-alkyl-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
S. an N—(C₃₋₇-cycloalkyl-C₁₋₂-alkyl)-N—(C₁₋₂-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
T. an amino group substituted by the groups R¹⁵ and R¹⁶ wherein
i. R¹⁵ denotes a C₁₋₃-alkyl group, and
ii. R¹⁶ denotes an R¹⁷—CH₂—CH₂— group, wherein the —CH₂—CH₂— moiety may be substituted by one to two C₁₋₃-alkyl groups, which may be identical or different, or by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl)carbonyl, thiazolidin-3-yl-carbonyl, (4-cyano-thiazolidin-3-yl)carbonyl, piperidin-1-ylcarbonyl or morpholin-4-ylcarbonyl group and
a. R¹⁷ denotes an amino group,
U. an amino group substituted by R²⁰, wherein
i. R²⁰ denotes an azetidin-3-yl, azetidin-2-ylmethyl, azetidin-3-ylmethyl, pyrrolidin-3-yl, pyrrolidin-2-ylmethyl, pyrrolidin-3-ylmethyl, piperidin-3-yl, piperidin-2-ylmethyl, piperidin-3-ylmethyl or piperidin-4-ylmethyl group, while the groups mentioned for R²⁰ may each be substituted by one or two C₁₋₃-alkyl groups,
V. an amino group substituted by the groups R¹⁵ and R²⁰, wherein
i. R¹⁵ and R²⁰ are as hereinbefore defined, while the groups mentioned for R²⁰ may each be substituted by one or two C₁₋₃-alkyl groups,
W. an R¹⁹—C₃₋₄-alkyl group wherein the C₃₋₄-alkyl moiety is straight-chained and may be substituted by the group R¹⁵ and may additionally be substituted by one or two C₁₋₃-alkyl groups, wherein R¹⁵ is as hereinbefore defined and R¹⁹ denotes an amino, C₁₋₃-alkylamino or di-(C₁₋₃-alkyl)-amino group,
X. a 3-amino-2-oxo-piperidin-5-yl or 3-amino-2-oxo-1-methyl-piperidin-5-yl group,
Y. a pyrrolidin-3-yl, piperidin-3-yl, piperidin-4-yl, hexahydroazepin-3-yl or hexahydroazepin-4-yl group which is substituted in the 1 position by an amino, C₁₋₃-alkylamino or di-(C₁₋₃-alkyl)amino group, or
Z. an azetidin-2-yl-C₁₋₂-alkyl, azetidin-3-yl-C₁₋₂-alkyl, pyrrolidin-2-yl-C₁₋₂-alkyl, pyrrolidin-3-yl, pyrrolidin-3-yl-C₁₋₂-alkyl, piperidin-2-yl-C₁₋₂-alkyl, piperidin-3-yl, piperidin-3-yl-C₁₋₂-alkyl, piperidin-4-yl or piperidin-4-yl-C₁₋₂-alkyl group, wherein the abovementioned groups may each be substituted by one or two C₁₋₃-alkyl groups,
while by the aryl groups mentioned in the definition of the groups mentioned above are meant phenyl or naphthyl groups which may be mono- or disubstituted by Rₕ independently of one another, while the substituents may be identical or different and Rₕ denotes a fluorine, chlorine, bromine or iodine atom, a trifluoromethyl, cyano, nitro, amino, aminocarbonyl, aminosulphonyl, methylsulphonyl, acetylamino, methylsulphonylamino, C₁₋₃-alkyl, cyclopropyl, ethenyl, ethynyl, hydroxy, C₁₋₃-alkyloxy, difluoromethoxy or trifluoromethoxy group,
by the heteroaryl groups mentioned in the definition of the groups mentioned above is meant a pyrrolyl, furanyl, thienyl, pyridyl, indolyl, benzofuranyl, benzothiophenyl, quinolinyl or isoquinolinyl group,
or a pyrrolyl, furanyl, thienyl or pyridyl group wherein one or two methyne groups are replaced by nitrogen atoms,
or an indolyl, benzofuranyl, benzothiophenyl, quinolinyl or isoquinolinyl group wherein one to three methyne groups are replaced by nitrogen atoms,
or a 1,2-dihydro-2-oxo-pyridinyl, 1,4-dihydro-4-oxo-pyridinyl, 2,3-dihydro-3-oxo-pyridazinyl, 1,2,3,6-tetrahydro-3,6-dioxo-pyridazinyl, 1,2-dihydro-2-oxo-pyrimidinyl, 3,4-dihydro-4-oxo-pyrimidinyl, 1,2,3,4-tetrahydro-2,4-dioxo-pyrimidinyl, 1,2-dihydro-2-oxo-pyrazinyl, 1,2,3,4-tetrahydro-2,3-dioxo-pyrazinyl, 2,3-dihydro-2-oxo-indolyl, 2,3-dihydrobenzofuranyl, 2,3-dihydro-2-oxo-1H-benzimidazolyl, 2,3-dihydro-2-oxo-benzoxazolyl, 1,2-dihydro-2-oxo-quinolinyl, 1,4-dihydro-4-oxo-quinolinyl, 1,2-dihydro-1-oxo-isoquinolinyl, 1,4-dihydro-4-oxo-cinnolinyl, 1,2-dihydro-2-oxo-quinazolinyl, 1,4-dihydro-4-oxo-quinazolinyl, 1,2,3,4-tetrahydro-2,4-dioxo-quinazolinyl, 1,2-dihydro-2-oxoquinoxalinyl, 1,2,3,4-tetrahydro-2,3-dioxo-quinoxalinyl, 1,2-dihydro-1-oxo-phthalazinyl, 1,2,3,4-tetrahydro-1,4-dioxo-phthalazinyl, chromanyl, cumarinyl, 2,3-dihydro-benzo[1,4]dioxinyl or 3,4-dihydro-3-oxo-2H-benzo[1,4]oxazinyl group,
wherein the abovementioned heteroaryl groups may be substituted by R¹⁰ to R¹⁴, wherein R¹⁰ to R¹⁴ are as hereinbefore defined,
while, unless otherwise stated, the abovementioned alkyl, alkenyl and alkynyl groups may be straight-chain or branched,
as well as the derivatives wherein the 2-oxo, the 6-oxo- or the 2-oxo- and the 6-oxo group of the xanthine skeleton are replaced by thioxo groups,
with the proviso that the compounds wherein
R¹ denotes a hydrogen atom or a methyl group,
R² denotes a hydrogen atom or a methyl group,
R³ denotes a methyl group
and
R⁴ denotes a 3-aminopropyl, 3-[di-(C₁₋₃-alkyl)amino]-propyl, 1-phenyl-3-[di-(C₁₋₃-alkyl)amino]-propyl, 1-phenyl-3-methyl-3-(dimethylamino)-propyl, 1-(4-chlorophenyl)-3-(dimethylamino)-propyl, 1-phenyl-2-methyl-3-(dimethylamino)-propyl, 1-(3-methoxyphenyl)-3-(dimethylamino)-propyl or a 4-aminobutyl group, are excluded,
and with the proviso that the compound
1,3,7-trimethyl-8-(1-aminocyclohexyl)-xanthine
are excluded,
the tautomers, enantiomers, diastereomers, mixtures thereof and the salts thereof.

2. The compound according to claim 1, wherein
I. R¹ denotes
A. a hydrogen atom,
B. a C₁₋₆-alkyl group,
C. a C₃₋₆-alkenyl group,
D. a C₃₋₄-alkenyl group which is substituted by a C₁₋₂-alkyloxy-carbonyl group,
E. a C₃₋₆-alkynyl group,
F. a C₃₋₆-cycloalkyl-C₁₋₃-alkyl group,
G. a phenyl group which may be substituted by a fluorine, chlorine or bromine atom or by a methyl, trifluoromethyl, hydroxy or methoxy group,
H. a phenyl-C₁₋₄-alkyl group wherein the phenyl moiety is substituted by R¹⁰ to R¹², wherein
i. R¹⁰ denotes
a. a hydrogen atom, a fluorine, chlorine or bromine atom,
b. a C₁₋₄-alkyl, trifluoromethyl, hydroxymethyl, C₃₋₆-cycloalkyl, ethynyl or phenyl group,
c. a hydroxy, C₁₋₄-alkyloxy, difluoromethoxy, trifluoromethoxy, 2,2,2-trifluoroethoxy, phenoxy, benzyloxy, 2-propen-1-yloxy, 2-propyn-1-yloxy, cyano-C₁₋₂-alkyloxy, C₁₋₂-alkylsulphonyloxy, phenylsulphonyloxy, carboxy-C₁₋₃-alkyloxy, C₁₋₃-alkyloxy-carbonyl-C₁₋₃-alkyloxy, aminocarbonyl-C₁₋₃-alkyloxy, C₁₋₂-alkyl-aminocarbonyl-C₁₋₃-alkyloxy, di-(C₁₋₂-alkyl)aminocarbonyl-C₁₋₃-alkyloxy, pyrrolidin-1-yl-carbonyl-C₁₋₃-alkyloxy, piperidin-1-ylcarbonyl-C₁₋₃-alkyloxy, morpholin-4-ylcarbonyl-C₁₋₃-alkyloxy, methylsulphanylmethoxy, methylsulphinylmethoxy, methylsulphonylmethoxy, C₃₋₆-cycloalkyloxy or C₃₋₆-cycloalkyl-C₁₋₂-alkyloxy group,
d. a carboxy, C₁₋₃-alkyloxycarbonyl, carboxy-C₁₋₃-alkyl, C₁₋₃-alkyloxy-carbonyl-C₁₋₃-alkyl, aminocarbonyl, C₁₋₂-alkylaminocarbonyl, di-(C₁₋₂-alkyl)aminocarbonyl, morpholin-4-ylcarbonyl or cyano group,
e. a nitro, amino, C₁₋₂-alkylamino, di-(C₁₋₂-alkyl)amino, cyano-C₁₋₂-alkylamino, [N-(cyano-C₁₋₂-alkyl)-N—C₁₋₂-alkyl-amino], C₁₋₂-alkyloxy-carbonyl-C₁₋₂-alkylamino, C₁₋₂-alkylcarbonylamino, C₁₋₂-alkyloxy-carbonylamino, C₁₋₃-alkylsulphonylamino, bis-(C₁₋₂-alkylsulphonyl)-amino, aminosulphonylamino, C₁₋₂-alkylamino-sulphonylamino, di-(C₁₋₂-alkyl)amino-sulphonylamino, morpholin-4-yl-sulphonylamino, (C₁₋₂-alkylamino)thiocarbonylamino, (C₁₋₂-alkyloxy-carbonylamino)carbonylamino, aminocarbonylamino, C₁₋₂-alkylaminocarbonylamino, di-(C₁₋₂-alkyl)aminocarbonylamino or morpholin-4-ylcarbonylamino group,
f. a 2-oxo-imidazolidin-1-yl, 3-methyl-2-oxo-imidazolidin-1-yl, 2,4-dioxo-imidazolidin-1-yl, 3-methyl-2,4-dioxo-imidazolidin-1-yl, 2,5-dioxo-imidazolidin-1-yl, 3-methyl-2,5-dioxo-imidazolidin-1-yl, 2-oxo-hexahydropyrimidin-1-yl or 3-methyl-2-oxo-hexahydropyrimidin-1-yl group, or
g. a C₁₋₂-alkylsulphanyl, C₁₋₂-alkylsulphinyl, C₁₋₂-alkylsulphonyl, aminosulphonyl, C₁₋₂-alkylaminosulphonyl or di-(C₁₋₂-alkyl)aminosulphonyl group, and
ii. R¹¹ and R¹², which may be identical or different, denote a hydrogen, fluorine, chlorine or bromine atom or a methyl, cyano, trifluoromethyl or methoxy group, or
R¹¹ together with R¹², if they are bound to adjacent carbon atoms, also denote a methylenedioxy, difluoromethylenedioxy, 1,3-propylene or 1,4-butylene group,
I. a phenyl-C₁₋₃-alkyl group wherein the alkyl moiety is substituted by a carboxy, C₁₋₂-alkyloxy-carbonyl, aminocarbonyl, C₁₋₂-alkylaminocarbonyl or di-(C₁₋₂-alkyl)aminocarbonyl group,
J. a phenyl-C₂₋₃-alkenyl group, wherein the phenyl moiety may be substituted by a fluorine, chlorine or bromine atom or by a methyl, trifluoromethyl or methoxy group,
K. a phenyl-(CH₂)ₘ-A-(CH₂)ₙ group wherein the phenyl moiety is substituted by R¹⁰ to R¹², wherein R¹⁰ to R¹² are as hereinbefore defined and
i. A denotes a carbonyl, hydroxyiminomethylene or C₁₋₂-alkyloxyiminomethylene group, m denotes the number 0 or 1 and n denotes the number 1 or 2,
L. a phenylcarbonylmethyl group wherein the phenyl moiety is substituted by R¹⁰ to R¹², wherein R¹⁰ to R¹² are as hereinbefore defined and the methyl moiety is substituted by a methyl or ethyl group,
M. a phenylcarbonylmethyl group wherein two adjacent hydrogen atoms of the phenyl moiety are replaced by a —O—CO—NH, —NH—CO—NH, —N═CH—NH, —N═CH—O or —O—CH₂—CO—NH— bridge, wherein the abovementioned bridges may be substituted by one or two methyl groups,
N. a phenyl-(CH₂)ₘ-B-(CH₂)ₙ group wherein the phenyl moiety is substituted by R¹⁰ to R¹², wherein R¹⁰ to R¹², m and n are as hereinbefore defined and
i. B denotes a methylene group which is substituted by a hydroxy or C₁₋₂-alkyloxy group and is optionally additionally substituted by a methyl group,
O. a naphthylmethyl or naphthylethyl group, wherein the naphthyl moiety is substituted in each case by R¹⁰ to R¹², wherein R¹⁰ to R¹² are as hereinbefore defined,
P. a [1,4]naphthoquinon-2-yl, chromen-4-on-3-yl or 1-oxoindan-2-yl group,
Q. a heteroaryl-C₁₋₃-alkyl group, wherein by the term heteroaryl is meant a pyrrolyl, imidazolyl, triazolyl, furanyl, thienyl, oxazolyl, isoxazolyl, thiazolyl, isothiazolyl, pyridyl, pyridazinyl, pyrimidinyl, pyrazinyl, indolyl, benzimidazolyl, 2,3-dihydro-2-oxo-1H-benzimidazolyl, indazolyl, benzofuranyl, 2,3-dihydrobenzofuranyl, benzoxazolyl, dihydro-2-oxo-benzoxazolyl, benzisoxazolyl, benzothiophenyl, benzothiazolyl, benzoisothiazolyl, quinolinyl, 1,2-dihydro-2-oxo-quinolinyl, isoquinolinyl, 1,2-dihydro-1-oxo-isoquinolinyl, cinnolinyl, quinazolinyl, 1,2-dihydro-2-oxo-quinazolinyl, 1,2-dihydro-1-oxo-phthalazin-4-yl, cumarinyl or 3,4-dihydro-3-oxo-2H-benzo[1,4]oxazinyl group,
wherein the abovementioned heteroaryl groups may be substituted at carbon atoms by a fluorine, chlorine or bromine atom, by a methyl, trifluoromethyl, cyano, aminocarbonyl, aminosulphonyl, methylsulphonyl, nitro, amino, acetylamino, methylsulphonylamino, methoxy, difluoromethoxy or trifluoromethoxy group and the imino groups of the abovementioned heteroaryl groups may be substituted by methyl or ethyl groups,
R. a furanyl-A-CH₂, thienyl-A-CH₂, thiazolyl-A-CH₂ or pyridyl-A-CH₂ group, wherein A is as hereinbefore defined,
S. a furanyl-B-CH₂, thienyl-B-CH₂, thiazolyl-B-CH₂ or pyridyl-B-CH₂ group, wherein B is as hereinbefore defined,
T. a C₁₋₄-alkyl-A-(CH₂)ₙ group, wherein A and n are as hereinbefore defined,
U. a C₃₋₆-cycloalkyl-(CH₂)ₘ-A-(CH₂)ₙ group, wherein A, m and n are as hereinbefore defined,
V. a C₃₋₆-cycloalkyl-(CH₂)ₘ-B-(CH₂)ₙ group, wherein B, m and n are as hereinbefore defined,
W. an R²¹-A-(CH₂)ₙ group wherein R²¹ denotes a C₁₋₂-alkyloxycarbonyl, aminocarbonyl, C₁₋₂-alkylaminocarbonyl, di-(C₁₋₂-alkyl)aminocarbonyl, pyrrolidin-1-yl-carbonyl, piperidin-1-yl-carbonyl or morpholin-4-yl-carbonyl group and A and n are as hereinbefore defined,
X. a phenyl-D-C₁₋₃-alkyl group wherein the phenyl moiety is optionally substituted by a fluorine, chlorine or bromine atom, a methyl, trifluoromethyl or methoxy group and D denotes an oxygen or sulphur atom, a sulphinyl or sulphonyl group,
Y. a C₁₋₄-alkyl group substituted by a group Rₐ, wherein
i. Rₐ denotes a cyano, carboxy, C₁₋₃-alkyloxy-carbonyl, aminocarbonyl, C₁₋₂-alkyl-aminocarbonyl, di-(C₁₋₂-alkyl)aminocarbonyl, pyrrolidin-1-yl-carbonyl, piperidin-1-ylcarbonyl or morpholin-4-ylcarbonyl group,
Z. a C₂₋₄-alkyl group substituted by a group R_{b}, wherein
i. R_{b} denotes a hydroxy, C₁₋₃-alkyloxy, amino, C₁₋₃-alkylamino, di-(C₁₋₃-alkyl)-amino, pyrrolidin-1-yl, piperidin-1-yl, morpholin-4-yl, piperazin-1-yl, 4-methyl-piperazin-1-yl or 4-ethyl-piperazin-1-yl group and is isolated by at least two carbon atoms from the cyclic nitrogen atom in the 1 position of the xanthine skeleton,
AA. or an amino or benzoylamino group,
II. R² denotes
A. a hydrogen atom,
B. a C₁₋₆-alkyl group,
C. a C₂₋₄-alkenyl group,
D. a C₃₋₄-alkynyl group,
E. a C₃₋₆-cycloalkyl group,
F. a C₃₋₆-cycloalkyl-C₁₋₃-alkyl group,
G. a tetrahydrofuran-3-yl, tetrahydropyran-3-yl, tetrahydropyran-4-yl, tetrahydrofuranylmethyl or tetrahydropyranylmethyl group,
H. a phenyl group which is optionally substituted by a fluorine, chlorine or bromine atom or by a methyl, trifluoromethyl, hydroxy, methoxy, difluoromethoxy or trifluoromethoxy group,
I. a phenyl-C₁₋₄-alkyl group wherein the phenyl moiety is optionally substituted by a fluorine, chlorine or bromine atom, a methyl, trifluoromethyl, dimethylamino, hydroxy, methoxy, difluoromethoxy or trifluoromethoxy group,
J. a phenyl-C₂₋₃-alkenyl group, wherein the phenyl moiety may be substituted by a fluorine, chlorine or bromine atom or by a methyl, trifluoromethyl or methoxy group,
K. a phenylcarbonyl-C₁₋₂-alkyl group wherein the phenyl moiety is optionally substituted by a fluorine, chlorine or bromine atom, a methyl, trifluoromethyl, hydroxy, methoxy, difluoromethoxy or trifluoromethoxy group,
L. a heteroaryl-C₁₋₃-alkyl group, wherein the term heteroaryl is as hereinbefore defined,
M. a furanylcarbonylmethyl, thienylcarbonylmethyl, thiazolylcarbonylmethyl or pyridylcarbonylmethyl group,
N. a C₁₋₄-alkyl-carbonyl-C₁₋₂-alkyl group,
O. a C₃₋₆-cycloalkyl-carbonyl-C₁₋₂-alkyl group,
P. a phenyl-D-C₁₋₃-alkyl group wherein the phenyl moiety is optionally substituted by a fluorine, chlorine or bromine atom, a methyl, trifluoromethyl, hydroxy, methoxy, difluoromethoxy or trifluoromethoxy group, and D is as hereinbefore defined, or
Q. a C₁₋₄-alkyl group substituted by a group Rₐ, wherein Rₐ is as hereinbefore defined,
R. a C₂₋₄-alkyl group substituted by a group R_{b}, wherein R_{b} is as hereinbefore defined and is isolated by at least two carbon atoms from the cyclic nitrogen atom in the 3 position of the xanthine skeleton,
III. R³ denotes
A. a C₂₋₆-alkyl group,
B. a C₃₋₇-alkenyl group,
C. a C₃₋₅-alkenyl group which is substituted by a fluorine, chlorine or bromine atom or a trifluoromethyl group,
D. a C₃₋₆-alkynyl group,
E. a C₁₋₃-alkyl group substituted by the group R_{c}, wherein
i. R_{c} denotes
a. a C₃₋₆-cycloalkyl group optionally substituted by one or two methyl groups,
b. a C₅₋₆-cycloalkenyl group optionally substituted by one or two methyl groups,
c. a phenyl group optionally substituted by a fluorine, chlorine, bromine or iodine atom, by a methyl, trifluoromethyl, cyano, nitro, amino, hydroxy, methoxy, difluoromethoxy or trifluoromethoxy group,
d. a phenyl group which is substituted by two fluorine atoms,
e. a naphthyl group or
f. a furanyl, thienyl, oxazolyl, isoxazolyl, thiazolyl, isothiazolyl or pyridyl group optionally substituted by a methyl or trifluoromethyl group,
F. a phenyl group optionally substituted by a fluorine, chlorine or bromine atom, by a methyl, trifluoromethyl, cyano, hydroxy, methoxy, difluoromethoxy or trifluoromethoxy group,
G. a phenyl group which is substituted by two methyl groups,
H. a naphthyl group or
I. a phenyl-C₂₋₃-alkenyl group
and
IV. R⁴ denotes
A. a pyrrolidin-1-yl group which is substituted in the 3 position by an amino, methylamino or dimethylamino group,
B. an azetidin-1-yl group which is substituted by an aminomethyl group,
C. a pyrrolidin-1-yl group which is substituted by an aminomethyl group,
D. a piperidin-1-yl group which is substituted in the 3 position or in the 4 position by an amino, methylamino, dimethylamino or [(2-cyano-pyrrolidin-1-yl-)carbonylmethyl]-amino group, wherein the piperidin-1-yl moiety may additionally be substituted by a methyl or ethyl group,
E. a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
F. a 3-amino-piperidin-1-yl group wherein the piperidin-1-yl moiety in the 4 position or in the 5 position is additionally substituted by a hydroxy or methoxy group,
G. a 3-amino-piperidin-1-yl group wherein the methylene group in the 2 position or in the 6 position is replaced by a carbonyl group,
H. a 3-amino-piperidin-1-yl group wherein a hydrogen atom in the 2 position together with a hydrogen atom in the 5 position is replaced by a —CH₂—CH₂— bridge,
I. a 3-amino-piperidin-1-yl group wherein a hydrogen atom in the 2 position together with a hydrogen atom in the 6 position is replaced by a —CH₂—CH₂— bridge,
J. a 3-amino-piperidin-1-yl group wherein a hydrogen atom in the 4 position together with a hydrogen atom in the 6 position is replaced by a —CH₂—CH₂— bridge,
K. a piperidin-1-yl group which is substituted by an aminomethyl group,
L. a piperidin-3-yl or piperidin-4-yl group,
M. a piperidin-3-yl or piperidin-4-yl group which is substituted in the 1 position by an amino group,
N. a hexahydroazepin-1-yl group which is substituted in the 3 position or in the 4 position by an amino group,
O. a C₃₋₆-cycloalkyl-amino group wherein the cycloalkyl moiety is substituted by an amino, methylamino or dimethylamino group, wherein the two nitrogen atoms are isolated from one another at the cycloalkyl moiety by at least two carbon atoms,
P. an N—(C₃₋₆-cycloalkyl)-N—(C₁₋₂-alkyl)-amino group wherein the cycloalkyl moiety is substituted by an amino, methylamino or dimethylamino group, wherein the two nitrogen atoms are isolated from one another at the cycloalkyl moiety by at least two carbon atoms,
Q. a C₃₋₆-cycloalkyl-amino group wherein the cycloalkyl moiety is substituted by an aminomethyl or aminoethyl group,
R. an N—(C₃₋₆-cycloalkyl)-N—(C₁₋₂-alkyl)-amino group wherein the cycloalkyl moiety is substituted by an aminomethyl or aminoethyl group,
S. a C₃₋₆-cycloalkyl-C₁₋₂-alkyl-amino group wherein the cycloalkyl moiety is substituted by an amino, aminomethyl or aminoethyl group,
T. an N—(C₃₋₆-cycloalkyl-C₁₋₂-alkyl)-N—(C₁₋₂-alkyl)-amino group wherein the cycloalkyl moiety is substituted by an amino, aminomethyl or aminoethyl group,
U. an amino group substituted by the groups R¹⁵ and R¹⁶ wherein
i. R¹⁵ denotes a C₁₋₃-alkyl group and
ii. R¹⁶ denotes a 2-aminoethyl, 2-(methylamino)ethyl, or 2-(dimethylamino)ethyl group, wherein the ethyl moiety may in each case be substituted by one or two methyl or ethyl groups or by an aminocarbonyl, C₁₋₂-alkyl-aminocarbonyl, di-(C₁₋₂-alkyl)aminocarbonyl, pyrrolidin-1-yl-carbonyl, piperidin-1-ylcarbonyl or morpholin-4-ylcarbonyl group,
V. an amino group wherein the nitrogen atom is substituted by a pyrrolidin-3-yl, piperidin-3-yl, pyrrolidin-2-ylmethyl, pyrrolidin-3-ylmethyl, piperidin-2-ylmethyl, piperidin-3-ylmethyl or piperidin-4-ylmethyl group,
W. a C₁₋₂-alkylamino group wherein the nitrogen atom is substituted by a pyrrolidin-3-yl, piperidin-3-yl, piperidin-4-yl, pyrrolidin-2-ylmethyl, pyrrolidin-3-ylmethyl, piperidin-2-ylmethyl, piperidin-3-ylmethyl or piperidin-4-ylmethyl group,
X. a 3-amino-propyl, 3-methylamino-propyl or 3-dimethylamino-propyl group wherein the propyl moiety may be substituted by one or two methyl groups,
Y. a 4-amino-butyl, 4-methylamino-butyl or 4-dimethylamino-butyl group wherein the butyl moiety may be substituted by one or two methyl groups,
Z. a C₁₋₂-alkyl group which is substituted by a 2-pyrrolidinyl, 3-pyrrolidinyl, 2-piperidinyl, 3-piperidinyl or 4-piperidinyl group,
AA. a 3-amino-2-oxo-piperidin-5-yl or 3-amino-2-oxo-1-methyl-piperidin-5-yl group,
BB. a C₃₋₆-cycloalkyl group which is substituted by an amino, aminomethyl or aminoethyl group or
CC. a C₃₋₆-cycloalkyl-C₁₋₂-alkyl group wherein the cycloalkyl moiety is substituted by an amino, aminomethyl or aminoethyl group,
while, unless otherwise stated, the abovementioned alkyl, alkenyl and alkynyl groups may be straight-chain or branched.

3. The compound according to claim 1, wherein
I. R¹ denotes
A. a hydrogen atom,
B. a C₁₋₄-alkyl group,
C. a C₃₋₅-alkenyl group,
D. a 2-propen-1-yl group which is substituted by a methoxycarbonyl group,
E. a C₃₋₅-alkynyl group,
F. a phenyl group,
G. a phenyl-C₁₋₄-alkyl group wherein the phenyl moiety may be substituted by one or two fluorine atoms, one or two chlorine atoms, a bromine atom, one to three methyl groups, a butyl, trifluoromethyl, hydroxy, methoxy, nitro, amino, carboxy or ethoxycarbonyl group,
H. a 2-phenylethyl group wherein the ethyl moiety is substituted in the 2 position by a hydroxy, methoxy or hydroxyimino group,
I. a phenylcarbonylmethyl group wherein the phenyl moiety may be substituted by a fluorine atom or by a methyl, aminocarbonyl, aminosulphonyl, cyano, hydroxy, methoxy, phenoxy, benzyloxy, 2-propen-1-yloxy, 2-propyn-1-yloxy, cyanomethoxy, (methoxycarbonyl)methoxy, (aminocarbonyl)methoxy, (methylaminocarbonyl)methoxy, (dimethylaminocarbonyl)methoxy, methylsulphonyloxy, phenylsulphonyloxy, nitro, amino, (methoxycarbonyl)methylamino, acetylamino, methoxycarbonylamino, methylsulphonylamino, bis-(methylsulphonyl)-amino, aminocarbonylamino, dimethylaminocarbonylamino, (methylamino)thiocarbonylamino, (ethoxycarbonylamino)carbonylamino or cyanomethylamino group,
J. a phenylcarbonylmethyl group wherein the phenyl moiety is substituted by two methoxy groups or by a bromine atom and by a dimethylamino group,
K. a 2-(phenylcarbonyl)ethyl group,
L. a 2-phenylethenyl group,
M. a 2-(phenoxy)ethyl group,
N. a phenylsulphanylmethyl or phenylsulphinylmethyl group,
O. a naphthylmethyl or naphthylethyl group,
P. an isoxazolylmethyl, thiazolylmethyl, pyridylmethyl, benzo[d]isoxazolylmethyl, benzo[d]isothiazolylmethyl, (1H-indazol-3-yl)methyl, quinolinylmethyl or isoquinolinylmethyl group, wherein the heterocyclic moiety may in each case be substituted by a methyl group,
Q. a isoquinolinylmethyl group wherein the isoquinolinyl moiety is substituted by a nitro or amino group,
R. a (1,2-dihydro-2-oxo-quinolin-4-yl)methyl group,
S. a chromen-4-on-3-yl group,
T. a pyrrolylethyl, triazolylethyl, thienylethyl, thiazolylethyl or pyridylethyl group, wherein the heterocyclic moiety may in each case be substituted by a methyl group,
U. a thienylcarbonylmethyl group,
V. a methyl group which is substituted by a cyclopropyl, cyano, carboxy, aminocarbonyl or methoxycarbonyl group,
W. an ethyl group which is substituted in the 2 position by a hydroxy, methoxy, dimethylamino, carboxy or methoxycarbonyl group, or
X. a propyl group which is substituted in the 3 position by a hydroxy, dimethylamino, carboxy or methoxycarbonyl group,
Y. a 2-oxopropyl group or
Z. an amino or benzoylamino group,
II. R² denotes
A. a hydrogen atom,
B. a C₁₋₆-alkyl group,
C. an ethenyl group,
D. a 2-propen-1-yl or 2-propyn-1-yl group,
E. a phenyl group,
F. a phenyl-C₁₋₄-alkyl group, wherein the phenyl moiety may be substituted by a fluorine atom, a methyl or methoxy group,
G. a phenylcarbonylmethyl group,
H. a 2-phenylethenyl group,
I. a methyl group which is substituted by a cyclopropyl, cyano, carboxy or methoxycarbonyl group, or
J. an ethyl group which is substituted in the 2 position by a cyano, hydroxy, methoxy or dimethylamino group,
III. R³ denotes
A. a C₄₋₆-alkenyl group,
B. a 1-cyclopenten-1-ylmethyl or 1-cyclohexen-1-ylmethyl group,
C. a 2-propyn-1-yl, 2-butyn-1-yl or 2-pentyn-1-yl group,
D. a phenyl group which may be substituted by a fluorine atom or a cyano, methyl or trifluoromethyl group,
E. a phenyl group which is substituted by two methyl groups,
F. a benzyl group wherein the phenyl moiety may be substituted by one or two fluorine atoms, an iodine atom or a cyano, nitro or amino group,
G. a furanylmethyl or thienylmethyl group or
H. a cyclopropylmethyl group and
IV. R⁴ denotes
A. a pyrrolidin-1-yl group which is substituted in the 3 position by an amino group,
B. an azetidin-1-yl group which is substituted by an aminomethyl group,
C. a pyrrolidin-1-yl group which is substituted by an aminomethyl group,
D. a piperidin-1-yl group which is substituted in the 3 position or in the 4 position by an amino, methylamino, dimethylamino or [(2-cyano-pyrrolidin-1-yl)carbonylmethyl]-amino group, wherein the piperidin-1-yl moiety may additionally be substituted by a methyl group,
E. a 3-amino-piperidin-1-yl group wherein the piperidin-1-yl moiety is additionally substituted by a pyrrolidin-1-yl-carbonyl group,
F. a 3-amino-piperidin-1-yl group wherein the piperidin-1-yl moiety in the 4 position is additionally substituted by a hydroxy group,
G. a 3-amino-piperidin-1-yl group wherein a hydrogen atom in the 2 position together with a hydrogen atom in the 5 position is replaced by a —CH₂—CH₂-bridge,
H. a piperidin-1-yl group which is substituted by an aminomethyl group,
I. a piperidin-3-yl or piperidin-4-yl group,
J. a hexahydroazepin-1-yl group which is substituted in the 3 position or in the 4 position by an amino group,
K. a 3-aminopropyl group,
L. a cyclohexyl group which is substituted by an amino group,
O. a 2-amino-cyclopentylamino or 3-amino-cyclopentylamino group,
P. a 2-amino-cyclohexylamino, 2-(methylamino)-cyclohexylamino or 3-amino-cyclohexylamino group,
Q. an N-(2-aminocyclohexyl)-methylamino group,
R. an amino group substituted by the groups R¹⁵ and R¹⁶ wherein
i. R¹⁵ denotes a methyl or ethyl group and
ii. R¹⁶ denotes a 2-aminoethyl-group, wherein the ethyl moiety may be substituted by one or two methyl groups or by an aminocarbonyl, methylaminocarbonyl, dimethylaminocarbonyl or pyrrolidin-1-ylcarbonyl group,
while, unless otherwise stated, the abovementioned alkyl and alkenyl groups may be straight-chain or branched.

4. The compound according to claim 1, wherein
I. R¹ denotes
A. a hydrogen atom,
B. a C₁₋₆-alkyl group,
C. a C₃₋₆-alkenyl group,
D. a C₃₋₄-alkenyl group which is substituted by a C₁₋₂-alkyloxy-carbonyl group,
E. a C₃₋₆-alkynyl group,
F. a C₃₋₆-cycloalkyl-C₁₋₃-alkyl group,
G. a phenyl group which may be substituted by a fluorine, chlorine or bromine atom or by a methyl, trifluoromethyl, hydroxy or methoxy group,
H. a phenyl-C₁₋₄-alkyl group wherein the phenyl moiety is substituted by R¹⁰ to R¹², wherein
i. R¹⁰ denotes
a. a hydrogen atom, a fluorine, chlorine or bromine atom,
b. a C₁₋₄-alkyl, trifluoromethyl, hydroxymethyl, C₃₋₆-cycloalkyl, ethynyl or phenyl group,
c. a hydroxy, C₁₋₄-alkyloxy, difluoromethoxy, trifluoromethoxy, 2,2,2-trifluoroethoxy, phenoxy, benzyloxy, 2-propen-1-yloxy, 2-propyn-1-yloxy, cyano-C₁₋₂-alkyloxy, C₁₋₂-alkylsulphonyloxy, phenylsulphonyloxy, carboxy-C₁₋₃-alkyloxy, C₁₋₃-alkyloxy-carbonyl-C₁₋₃-alkyloxy, aminocarbonyl-C₁₋₃-alkyloxy, C₁₋₂-alkyl-aminocarbonyl-C₁₋₃-alkyloxy, di-(C₁₋₂-alkyl)aminocarbonyl-C₁₋₃-alkyloxy, pyrrolidin-1-yl-carbonyl-C₁₋₃-alkyloxy, piperidin-1-ylcarbonyl-C₁₋₃-alkyloxy, morpholin-4-ylcarbonyl-C₁₋₃-alkyloxy, methylsulphanylmethoxy, methylsulphinylmethoxy, methylsulphonylmethoxy, C₃₋₆-cycloalkyloxy or C₃₋₆-cycloalkyl-C₁₋₂-alkyloxy group,
d. a carboxy, C₁₋₃-alkyloxycarbonyl, carboxy-C₁₋₃-alkyl, C₁₋₃-alkyloxy-carbonyl-C₁₋₃-alkyl, aminocarbonyl, C₁₋₂-alkylaminocarbonyl, di-(C₁₋₂-alkyl)aminocarbonyl, morpholin-4-ylcarbonyl or cyano group,
e. a nitro, amino, C₁₋₂-alkylamino, di-(C₁₋₂-alkyl)amino, cyano-C₁₋₂-alkylamino, [N-(cyano-C₁₋₂-alkyl)-N—C₁₋₂-alkyl-amino], C₁₋₂-alkyloxy-carbonyl-C₁₋₂-alkylamino, C₁₋₂-alkyl-carbonylamino, C₁₋₂-alkyloxy-carbonylamino, C₁₋₃-alkylsulphonylamino, bis-(C₁₋₂-alkylsulphonyl)-amino, aminosulphonylamino, C₁₋₂-alkylamino-sulphonylamino, di-(C₁₋₂-alkyl)amino-sulphonylamino, morpholin-4-yl-sulphonylamino, (C₁₋₂-alkylamino)thiocarbonylamino, (C₁₋₂-alkyloxy-carbonylamino)carbonylamino, aminocarbonylamino, C₁₋₂-alkylaminocarbonylamino, di-(C₁₋₂-alkyl)aminocarbonylamino or morpholin-4-ylcarbonylamino group,
f. a 2-oxo-imidazolidin-1-yl, 3-methyl-2-oxo-imidazolidin-1-yl, 2,4-dioxo-imidazolidin-1-yl, 3-methyl-2,4-dioxo-imidazolidin-1-yl, 2,5-dioxo-imidazolidin-1-yl, 3-methyl-2,5-dioxo-imidazolidin-1-yl, 2-oxo-hexahydropyrimidin-1-yl or 3-methyl-2-oxo-hexahydropyrimidin-1-yl group, or
g. a C₁₋₂-alkylsulphanyl, C₁₋₂-alkylsulphinyl, C₁₋₂-alkylsulphonyl, aminosulphonyl, C₁₋₂-alkylaminosulphonyl or di-(C₁₋₂-alkyl)aminosulphonyl group, and
ii. R¹¹ and R¹², which may be identical or different, denote a hydrogen, fluorine, chlorine or bromine atom or a methyl, cyano, trifluoromethyl or methoxy group, or
R¹¹ together with R¹², if they are bound to adjacent carbon atoms, also denote a methylenedioxy, difluoromethylenedioxy, 1,3-propylene or 1,4-butylene group,
I. a phenyl-C₁₋₃-alkyl group wherein the alkyl moiety is substituted by a carboxy, C₁₋₂-alkyloxy-carbonyl, aminocarbonyl, C₁₋₂-alkylaminocarbonyl or di-(C₁₋₂-alkyl)aminocarbonyl group,
J. a phenyl-C₂₋₃-alkenyl group, wherein the phenyl moiety may be substituted by a fluorine, chlorine or bromine atom or by a methyl, trifluoromethyl or methoxy group,
K. a phenyl-(CH₂)ₘ-A-(CH₂)ₙ group wherein the phenyl moiety is substituted by R¹⁰ to R¹², wherein R¹⁰ to R¹² are as hereinbefore defined and
i. A denotes a carbonyl, hydroxyiminomethylene or C₁₋₂-alkyloxyiminomethylene group, m denotes the number 0 or 1 and n denotes the number 1 or 2,
L. a phenylcarbonylmethyl group wherein the phenyl moiety is substituted by R¹⁰ to R¹², wherein R¹⁰ to R¹² are as hereinbefore defined and the methyl moiety is substituted by a methyl or ethyl group,
M. a phenylcarbonylmethyl group wherein two adjacent hydrogen atoms of the phenyl moiety are replaced by a —O—CO—NH, —NH—CO—NH, —N═CH—NH, —N═CH—O or —O—CH₂—CO—NH— bridge, wherein the abovementioned bridges may be substituted by one or two methyl groups,
N. a phenyl-(CH₂)ₘ-B-(CH₂)ₙ group wherein the phenyl moiety is substituted by R¹⁰ to R¹², wherein R¹⁰ to R¹², m and n are as hereinbefore defined and
i. B denotes a methylene group which is substituted by a hydroxy or C₁₋₂-alkyloxy group and is optionally additionally substituted by a methyl group,
O. a naphthylmethyl or naphthylethyl group, wherein the naphthyl moiety is substituted in each case by R¹⁰ to R¹², wherein R¹⁰ to R¹² are as hereinbefore defined,
P. a [1,4]naphthoquinon-2-yl, chromen-4-on-3-yl or 1-oxoindan-2-yl group,
Q. a heteroaryl-C₁₋₃-alkyl group, wherein the term heteroaryl denotes a pyrrolyl, imidazolyl, triazolyl, furanyl, thienyl, oxazolyl, isoxazolyl, thiazolyl, isothiazolyl, pyridyl, pyridazinyl, pyrimidinyl, pyrazinyl, indolyl, benzimidazolyl, 2,3-dihydro-2-oxo-1H-benzimidazolyl, indazolyl, benzofuranyl, 2,3-dihydrobenzofuranyl, benzoxazolyl, dihydro-2-oxo-benzoxazolyl, benzoisoxazolyl, benzothiophenyl, benzothiazolyl, benzoisothiazolyl, quinolinyl, 1,2-dihydro-2-oxo-quinolinyl, isoquinolinyl, 1,2-dihydro-1-oxo-isoquinolinyl, cinnolinyl, quinazolinyl, 1,2-dihydro-2-oxo-quinazolinyl, 1,2-dihydro-1-oxo-phthalazin-4-yl, cumarinyl or 3,4-dihydro-3-oxo-2H-benzo[1,4]oxazinyl group,
wherein the abovementioned heteroaryl groups may be substituted at carbon atoms by a fluorine, chlorine or bromine atom, by a methyl, trifluoromethyl, cyano, aminocarbonyl, aminosulphonyl, methylsulphonyl, nitro, amino, acetylamino, methylsulphonylamino, methoxy, difluoromethoxy or trifluoromethoxy group and the imino groups of the abovementioned heteroaryl groups may be substituted by methyl or ethyl groups,
R. a furanyl-A-CH₂, thienyl-A-CH₂, thiazolyl-A-CH₂ or pyridyl-A-CH₂ group, wherein A is as hereinbefore defined,
S. a furanyl-B—CH₂, thienyl-B—CH₂, thiazolyl-B—CH₂ or pyridyl-B—CH₂ group, wherein B is as hereinbefore defined,
T. a C₁₋₄-alkyl-A-(CH₂)ₙ group, wherein A and n are as hereinbefore defined,
U. a C₃₋₆-cycloalkyl-(CH₂)ₘ-A-(CH₂)ₙ group, wherein A, m and n are as hereinbefore defined,
V. a C₃₋₆-cycloalkyl-(CH₂)ₘ-B-(CH₂)ₙ group, wherein B, m and n are as hereinbefore defined,
W. a R²¹-A-(CH₂)ₙ group wherein R²¹ denotes a C₁₋₂-alkyloxycarbonyl, aminocarbonyl, C₁₋₂-alkylaminocarbonyl, di-(C₁₋₂-alkyl)aminocarbonyl, pyrrolidin-1-yl-carbonyl, piperidin-1-yl-carbonyl or morpholin-4-yl-carbonyl group and A and n are as hereinbefore defined,
X. a phenyl-D-C₁₋₃-alkyl group wherein the phenyl moiety is optionally substituted by a fluorine, chlorine or bromine atom, a methyl, trifluoromethyl or methoxy group and D denotes an oxygen or sulphur atom, a sulphinyl or sulphonyl group,
Y. a C₁₋₄-alkyl group substituted by a group Rₐ, wherein
i. Rₐ denotes a cyano, carboxy, C₁₋₃-alkyloxy-carbonyl, aminocarbonyl, C₁₋₂-alkyl-aminocarbonyl, di-(C₁₋₂-alkyl)aminocarbonyl, pyrrolidin-1-yl-carbonyl, piperidin-1-ylcarbonyl or morpholin-4-ylcarbonyl group,
Z. a C₂₋₄-alkyl group substituted by a group R_{b}, wherein
i. R_{b} denotes a hydroxy, C₁₋₃-alkyloxy, amino, C₁₋₃-alkylamino, di-(C₁₋₃-alkyl)-amino, pyrrolidin-1-yl, piperidin-1-yl, morpholin-4-yl, piperazin-1-yl, 4-methyl-piperazin-1-yl or 4-ethyl-piperazin-1-yl group and is isolated from the cyclic nitrogen atom in the 1 position of the xanthine skeleton by at least two carbon atoms,
AA. or an amino or benzoylamino group,
II. R² denotes
A. a hydrogen atom,
B. a C₁₋₆-alkyl group,
C. a C₂₋₄-alkenyl group,
D. a C₃₋₄-alkynyl group,
E. a C₃₋₆-cycloalkyl group,
F. a C₃₋₆-cycloalkyl-C₁₋₃-alkyl group,
G. a tetrahydrofuran-3-yl, tetrahydropyran-3-yl, tetrahydropyran-4-yl, tetrahydrofuranylmethyl or tetrahydropyranylmethyl group,
H. a phenyl group which is optionally substituted by a fluorine, chlorine or bromine atom or by a methyl, trifluoromethyl, hydroxy, methoxy, difluoromethoxy or trifluoromethoxy group,
I. a phenyl-C₁₋₄-alkyl group wherein the phenyl moiety is optionally substituted by a fluorine, chlorine or bromine atom, a methyl, trifluoromethyl, dimethylamino, hydroxy, methoxy, difluoromethoxy or trifluoromethoxy group,
J. a phenyl-C₂₋₃-alkenyl group, wherein the phenyl moiety may be substituted by a fluorine, chlorine or bromine atom or by a methyl, trifluoromethyl or methoxy group,
K. a phenylcarbonyl-C₁₋₂-alkyl group wherein the phenyl moiety is optionally substituted by a fluorine, chlorine or bromine atom, a methyl, trifluoromethyl, hydroxy, methoxy, difluoromethoxy or trifluoromethoxy group,
L. a heteroaryl-C₁₋₃-alkyl group, wherein the term heteroaryl is as hereinbefore defined,
M. a furanylcarbonylmethyl, thienylcarbonylmethyl, thiazolylcarbonylmethyl or pyridylcarbonylmethyl group,
N. a C₁₋₄-alkyl-carbonyl-C₁₋₂-alkyl group,
O. a C₃₋₆-cycloalkyl-carbonyl-C₁₋₂-alkyl group,
P. a phenyl-D-C₁₋₃-alkyl group wherein the phenyl moiety is optionally substituted by a fluorine, chlorine or bromine atom, a methyl, trifluoromethyl, hydroxy, methoxy, difluoromethoxy or trifluoromethoxy group, and D is as hereinbefore defined, or
Q. a C₁₋₄-alkyl group substituted by a group Rₐ, wherein Rₐ is as hereinbefore defined, or
R. a C₂₋₄-alkyl group substituted by a group R_{b}, wherein R_{b} is as hereinbefore defined and is isolated from the cyclic nitrogen atom in the 3 position of the xanthine skeleton by at least two carbon atoms,
III. R³ denotes
A. a C₁₋₃-alkyl group substituted by the group R_{c}, wherein
i. R_{c} denotes
a. a C₃₋₇-cycloalkyl group optionally substituted by one or two C₁₋₃-alkyl groups,
b. a C₅₋₇-cycloalkenyl group optionally substituted by one or two C₁₋₃-alkyl groups or
c. an aryl group or
d. a furanyl, thienyl, oxazolyl, isoxazolyl, thiazolyl, isothiazolyl, pyridyl, pyridazinyl, pyrimidyl or pyrazinyl group, wherein the abovementioned heterocyclic groups may each be substituted by one or two C₁₋₃-alkyl groups or by a fluorine, chlorine, bromine or iodine atom or by a trifluoromethyl, cyano or C₁₋₃-alkyloxy group,
B. a C₃₋₈-alkenyl group,
C. a C₃₋₆-alkenyl group substituted by a fluorine, chlorine or bromine atom, or a trifluoromethyl group,
E. a C₃₋₈-alkynyl group,
F. an aryl group or
G. an aryl-C₂₋₄-alkenyl group,
and
IV. R⁴ denotes
A. an azetidin-1-yl or pyrrolidin-1-yl group which is substituted in the 3 position by an RₑNR_{d} group and may additionally be substituted by one or two C₁₋₃-alkyl groups, wherein
i. Rₑ denotes a hydrogen atom or a C₁₋₃-alkyl group and
ii. R_{d} denotes a hydrogen atom or a C₁₋₃-alkyl group,
B. a piperidin-1-yl or hexahydroazepin-1-yl group which is substituted in the 3 position or in the 4 position by an RₑNR_{d} group and may additionally be substituted by one or two C₁₋₃-alkyl groups, wherein Rₑ and R_{d} are as hereinbefore defined,
C. a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
D. a 3-amino-piperidin-1-yl group wherein the piperidin-1-yl moiety is additionally substituted in the 4 position or in the 5 position by a hydroxy or methoxy group,
E. a 3-amino-piperidin-1-yl group wherein the methylene group is replaced in the 2 position or in the 6 position by a carbonyl group,
F. a piperidin-1-yl or hexahydroazepin-1-yl group substituted in the 3 position by an amino, C₁₋₃-alkylamino or di-(C₁₋₃-alkyl)-amino group, wherein in each case two hydrogen atoms on the carbon skeleton of the piperidin-1-yl or hexahydroazepin-1-yl group are replaced by a straight-chain alkylene bridge, this bridge containing 2 to 5 carbon atoms if the two hydrogen atoms are located on the same carbon atom, or 1 to 4 carbon atoms if the hydrogen atoms are located on adjacent carbon atoms, or 1 to 4 carbon atoms if the hydrogen atoms are located on carbon atoms which are separated by one atom, or 1 to 3 carbon atoms if the two hydrogen atoms are located on carbon atoms separated by two atoms,
G. an azetidin-1-yl, pyrrolidin-1-yl, piperidin-1-yl or hexahydroazepin-1-yl group which is substituted by an amino-C₁₋₃-alkyl, C₁₋₃-alkylamino-C₁₋₃-alkyl or a di-(C₁₋₃-alkyl)amino-C₁₋₃-alkyl group,
H. a 3-imino-piperazin-1-yl, 3-imino-[1,4]diazepan-1-yl or 5-imino-[1,4]diazepan-1-yl group optionally substituted by one or two C₁₋₃-alkyl groups on the carbon skeleton,
I. a C₃₋₇-cycloalkyl group which is substituted by an amino, C₁₋₃-alkylamino or di-(C₁₋₃-alkyl)-amino group,
J. a C₃₋₇-cycloalkyl group which is substituted by an amino-C₁₋₃-alkyl, C₁₋₃-alkylamino-C₁₋₃-alkyl or a di-(C₁₋₃-alkyl)amino-C₁₋₃-alkyl group,
K. a C₃₋₇-cycloalkyl-C₁₋₂-alkyl group wherein the cycloalkyl moiety is substituted by an amino, C₁₋₃-alkylamino or di-(C₁₋₃-alkyl)-amino group,
L. a C₃₋₇-cycloalkyl-C₁₋₂-alkyl group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
M. a C₃₋₇-cycloalkylamino group wherein the cycloalkyl moiety is substituted by an amino, C₁₋₃-alkylamino or di-(C₁₋₃-alkyl)-amino group, wherein the two nitrogen atoms are separated from one another at the cycloalkyl moiety by at least two carbon atoms,
N. a N—(C₃₋₇-cycloalkyl)-N—(C₁₋₃-alkyl)-amino group wherein the cycloalkyl moiety is substituted by an amino, C₁₋₃-alkylamino or di-(C₁₋₃-alkyl)-amino group, wherein the two nitrogen atoms are separated from one another at the cycloalkyl moiety by at least two carbon atoms,
O. a C₃₋₇-cycloalkylamino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
P. a N—(C₃₋₇-cycloalkyl)-N—(C₁₋₃-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
Q. a C₃₋₇-cycloalkyl-C₁₋₂-alkyl-amino group wherein the cycloalkyl moiety is substituted by an amino, C₁₋₃-alkylamino or di-(C₁₋₃-alkyl)-amino group,
R. a N—(C₃₋₇-cycloalkyl-C₁₋₂-alkyl)-N—(C₁₋₂-alkyl)-amino group wherein the cycloalkyl moiety is substituted by an amino, C₁₋₃-alkylamino or di-(C₁₋₃-alkyl)-amino group,
S. a C₃₋₇-cycloalkyl-C₁₋₂-alkyl-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
T. an N—(C₃₋₇-cycloalkyl-C₁₋₂-alkyl)-N—(C₁₋₂-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
U. an amino group substituted by the groups R¹⁵ and R¹⁶ wherein
i. R¹⁵ denotes a C₁₋₃-alkyl group and
ii. R¹⁶ denotes a R¹⁷—C₂₋₃-alkyl group, wherein the C₂₋₃-alkyl moiety is straight-chained and may be substituted by one to four C₁₋₃-alkyl groups, which may be identical or different, or by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl)carbonyl, thiazolidin-3-yl-carbonyl, (4-cyano-thiazolidin-3-yl)carbonyl, piperidin-1-ylcarbonyl or morpholin-4-ylcarbonyl group and
a. R¹⁷ denotes an amino, C₁₋₃-alkylamino or di-(C₁₋₃-alkyl)-amino group,
V. an amino group substituted by the group R²⁰, wherein
i. R²⁰ denotes an azetidin-3-yl, azetidin-2-ylmethyl, azetidin-3-ylmethyl, pyrrolidin-3-yl, pyrrolidin-2-ylmethyl, pyrrolidin-3-ylmethyl, piperidin-3-yl, piperidin-2-ylmethyl, piperidin-3-ylmethyl or piperidin-4-ylmethyl group, wherein the groups mentioned for R²⁰ may each be substituted by one or two C₁₋₃-alkyl groups,
W. an amino group substituted by the groups R¹⁵ and R²⁰, wherein
i. R¹⁵ and R²⁰ are as hereinbefore defined, wherein the groups mentioned for R²⁰ may each be substituted by one or two C₁₋₃-alkyl groups,
X. a R¹⁹—C₃₋₄-alkyl group wherein the C₃₋₄-alkyl moiety is straight-chained and may be substituted by the group R¹⁵ and may additionally be substituted by one or two C₁₋₃-alkyl groups, wherein R¹⁵ is as hereinbefore defined and R¹⁹ denotes an amino, C₁₋₃-alkylamino or di-(C₁₋₃-alkyl)-amino group,
Y. a 3-amino-2-oxo-piperidin-5-yl or 3-amino-2-oxo-1-methyl-piperidin-5-yl group,
Z. a pyrrolidin-3-yl, piperidin-3-yl, piperidin-4-yl, hexahydroazepin-3-yl or hexahydroazepin-4-yl group, which is substituted in the 1 position by an amino, C₁₋₃-alkylamino or di-(C₁₋₃-alkyl)amino group,
AA. or an azetidin-2-yl-C₁₋₂-alkyl, azetidin-3-yl-C₁₋₂-alkyl, pyrrolidin-2-yl-C₁₋₂-alkyl, pyrrolidin-3-yl, pyrrolidin-3-yl-C₁₋₂-alkyl, piperidin-2-yl-C₁₋₂-alkyl, piperidin-3-yl, piperidin-3-yl-C₁₋₂-alkyl, piperidin-4-yl or piperidin-4-yl-C₁₋₂-alkyl group, wherein the abovementioned groups may each be substituted by one or two C₁₋₃-alkyl groups,
while by the aryl groups mentioned in the definition of the groups mentioned above are meant phenyl or naphthyl groups which may be mono- or disubstituted independently of one another by Rₕ, while the substituents may be identical or different and Rₕ denotes a fluorine, chlorine, bromine or iodine atom, a trifluoromethyl, cyano, nitro, amino, C₁₋₃-alkyl, cyclopropyl, ethenyl, ethynyl, hydroxy, C₁₋₃-alkyloxy, difluoromethoxy or trifluoromethoxy group and
unless otherwise stated, the abovementioned alkyl and alkenyl groups may be straight-chained or branched.

5. The compound according to claim 1, wherein
I. R¹ denotes
A. a hydrogen atom,
B. a C₁₋₄-alkyl group,
C. a C₃₋₅-alkenyl group,
D. a 2-propen-1-yl group which is substituted by a methoxycarbonyl group,
E. a C₃₋₅-alkynyl group,
F. a phenyl-C₁₋₄-alkyl group wherein the phenyl moiety is substituted by R¹⁰ to R¹², wherein
i. R¹⁰ denotes
a. a hydrogen atom, a fluorine, chlorine or bromine atom,
b. a methyl, ethyl, trifluoromethyl or ethynyl group,
c. a hydroxy, methoxy, ethoxy, difluoromethoxy, trifluoromethoxy, 2,2,2-trifluoroethoxy, phenoxy, benzyloxy, 2-propen-1-yloxy, 2-propyn-1-yloxy, cyano-C₁₋₂-alkyloxy, C₁₋₂-alkyl-sulphonyloxy, phenylsulphonyloxy, carboxy-C₁₋₂-alkyloxy, C₁₋₂-alkyloxy-carbonyl-C₁₋₂-alkyloxy, aminocarbonyl-C₁₋₂-alkyloxy, C₁₋₂-alkyl-aminocarbonyl-C₁₋₂-alkyloxy, di-(C₁₋₂-alkyl)aminocarbonyl-C₁₋₂-alkyloxy, pyrrolidin-1-ylcarbonyl-C₁₋₂-alkyloxy, piperidin-1-ylcarbonyl-C₁₋₂-alkyloxy, morpholin-4-ylcarbonyl-C₁₋₂-alkyloxy group,
d. a carboxy, C₁₋₂-alkyloxy-carbonyl, aminocarbonyl, C₁₋₂-alkylaminocarbonyl, di-(C₁₋₂-alkyl)aminocarbonyl, morpholin-4-ylcarbonyl or cyano group,
e. a nitro, amino, C₁₋₂-alkylamino, di-(C₁₋₂-alkyl)amino, cyano-C₁₋₂-alkylamino, [N-(cyano-C₁₋₂-alkyl)-N-methyl-amino], C₁₋₂-alkyloxy-carbonyl-C₁₋₂-alkylamino, C₁₋₂-alkyl-carbonylamino, C₁₋₂-alkyloxy-carbonylamino, C₁₋₂-alkylsulphonylamino, bis-(C₁₋₂-alkylsulphonyl)-amino, aminosulphonylamino, C₁₋₂-alkylamino-sulphonylamino, di-(C₁₋₂-alkyl)amino-sulphonylamino, morpholin-4-yl-sulphonylamino, (C₁₋₂-alkylamino)thiocarbonylamino, (C₁₋₂-alkyloxy-carbonylamino)carbonylamino, aminocarbonylamino, C₁₋₂-alkylaminocarbonylamino, di-(C₁₋₂-alkyl)aminocarbonylamino or morpholin-4-yl-carbonylamino group,
f. a 2-oxo-imidazolidin-1-yl, 3-methyl-2-oxo-imidazolidin-1-yl, 2,4-dioxo-imidazolidin-1-yl, 3-methyl-2,4-dioxo-imidazolidin-1-yl, 2,5-dioxo-imidazolidin-1-yl, 3-methyl-2,5-dioxo-imidazolidin-1-yl, 2-oxo-hexahydropyrimidin-1-yl or 3-methyl-2-oxo-hexahydropyrimidin-1-yl group, or
g. a C₁₋₂-alkylsulphanyl, C₁₋₂-alkylsulphinyl, C₁₋₂-alkylsulphonyl, aminosulphonyl, C₁₋₂-alkylaminosulphonyl or di-(C₁₋₂-alkyl)aminosulphonyl group,
ii. and R¹¹ and R¹², which may be identical or different, denote a hydrogen, fluorine, chlorine or bromine atom or a methyl, cyano or methoxy group, or
R¹¹ together with R¹², if they are bound to adjacent carbon atoms, also denote a methylenedioxy group,
G. a phenylmethyl group wherein the methyl moiety is substituted by a carboxy, methoxycarbonyl or aminocarbonyl group,
H. a 2-phenylethyl group wherein the ethyl moiety is substituted by a carboxy, methoxycarbonyl or aminocarbonyl group,
I. a 2-phenylethyl group wherein the ethyl moiety is substituted in the 2 position by a hydroxy, methoxy, hydroxyimino or methoxyimino group,
J. a 2-phenylethyl group wherein the ethyl moiety is substituted in the 2 position by a hydroxy group and a methyl group,
K. a phenylcarbonylmethyl group wherein the phenyl moiety is substituted by R¹⁰ to R¹², wherein R¹⁰ to R¹² are as hereinbefore defined,
L. a 1-(phenylcarbonyl)ethyl or 2-(phenylcarbonyl)ethyl group,
M. a 2-phenylethenyl group,
N. a phenylsulphanylmethyl or phenylsulphinylmethyl group,
O. a 2-(phenyloxy)ethyl group,
P. a naphthylmethyl or naphthylethyl group, wherein the naphthyl moiety may be substituted in each case by a methyl, nitro, amino, acetylamino, methylsulphonylamino, cyano, aminocarbonyl or aminosulphonyl group,
Q. a [1,4]naphthoquinon-2-yl, chromen-4-on-3-yl or 1-oxoindan-2-yl group
R. an oxazolylmethyl, isoxazolylmethyl, thiazolylmethyl, pyridylmethyl, benzofuranylmethyl, 2,3-dihydrobenzofuranylmethyl, benzo[d]isoxazolylmethyl, benzo[d]isothiazolylmethyl, (1H-indazol-3-yl)methyl, quinolinylmethyl, (1,2-dihydro-2-oxo-quinolin-4-yl)methyl, isoquinolinylmethyl, (1,2-dihydro-1-oxo-isoquinolin-4-yl)methyl, cinnolinylmethyl, quinazolinylmethyl, (1,2-dihydro-2-oxo-quinazolin-4-yl)methyl, (1,2-dihydro-1-oxo-phthalazin-4-yl)methyl or cumarinylmethyl group, wherein the heterocyclic moiety may be substituted by a methyl group in each case,
S. a quinolinylmethyl or isoquinolinylmethyl group, wherein the heterocyclic moiety is substituted in each case by a cyano, nitro, amino, acetylamino, methylsulphonylamino, aminocarbonyl or aminosulphonyl group,
T. a pyrrolylethyl, triazolylethyl, thienylethyl, thiazolylethyl or pyridylethyl group, wherein the heterocyclic moiety may be substituted in each case by a methyl group,
U. a furanylcarbonylmethyl, thienylcarbonylmethyl, thiazolylcarbonylmethyl or pyridylcarbonylmethyl group,
V. a methyl group which is substituted by a cyclopropyl, cyano, carboxy, aminocarbonyl or methoxycarbonyl group,
W. an ethyl group which is substituted in the 2 position by a hydroxy, methoxy, dimethylamino, carboxy or methoxycarbonyl group, or
X. a propyl group which is substituted in the 3 position by a hydroxy, dimethylamino, carboxy or methoxycarbonyl group,
Y. a 2-oxopropyl group or
Z. an amino or benzoylamino group,
II. R² denotes
A. a hydrogen atom,
B. a C₁₋₆-alkyl group,
C. an ethenyl group,
D. a 2-propen-1-yl or 2-propyn-1-yl group,
E. a C₃₋₆-cycloalkyl group,
F. a tetrahydrofuran-3-yl, tetrahydropyran-3-yl, tetrahydropyran-4-yl, tetrahydrofuranylmethyl or tetrahydropyranylmethyl group,
G. a phenyl group,
H. a phenyl-C₁₋₄-alkyl group, wherein the phenyl moiety may be substituted by a fluorine or chlorine atom, a methyl, dimethylamino, hydroxy, methoxy or trifluoromethoxy group,
I. a phenylcarbonylmethyl group, wherein the phenyl moiety may be substituted by a fluorine or chlorine atom, a hydroxy, methoxy or trifluoromethoxy group,
J. a 2-phenylethenyl group,
K. a 2-(phenyloxy)ethyl group,
L. a pyridylmethyl or pyridylethyl group,
M. a methyl group which is substituted by a C₃₋₆-cycloalkyl, cyano, carboxy or methoxycarbonyl group, or
N. an ethyl group which is substituted in the 2 position by a C₃₋₆-cycloalkyl, cyano, carboxy, methoxycarbonyl, hydroxy, methoxy or dimethylamino group,
O. or a propyl group which is substituted in the 3 position by a C₃₋₆-cycloalkyl, cyano, carboxy, methoxycarbonyl, hydroxy, methoxy or dimethylamino group,
III. R³ denotes
A. a C₄₋₆-alkenyl group,
B. a 1-cyclopenten-1-ylmethyl or 1-cyclohexen-1-ylmethyl group,
C. a 1-cyclopenten-1-ylmethyl group wherein the 1-cyclopenten-1-yl moiety is substituted by a methyl group,
D. a 2-propyn-1-yl, 2-butyn-1-yl or 2-pentyn-1-yl group,
E. a phenyl group which may be substituted by a fluorine atom or a cyano, methyl-methoxy or trifluoromethyl group,
F. a phenyl group which is substituted by two methyl groups,
G. a benzyl group wherein the phenyl moiety may be substituted by one or two fluorine atoms, a chlorine, bromine or iodine atom, or a methyl, methoxy, cyano, nitro or amino group,
H. a furanylmethyl or thienylmethyl group,
I. a cyclopropylmethyl group or
J. a cyclopropylmethyl group wherein the cyclopropyl moiety is substituted by a methyl group, and
IV. R⁴ denotes
A. a piperidin-1-yl group which is substituted in the 3 position by an amino group, wherein the piperidin-1-yl moiety may additionally be substituted by a methyl group,
B. a 3-amino-piperidin-1-yl group wherein the piperidin-1-yl moiety is additionally substituted by an aminocarbonyl, methylaminocarbonyl, dimethylaminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
C. a 3-amino-piperidin-1-yl group wherein the piperidin-1-yl moiety in the 4 position or in the 5 position is additionally substituted by a hydroxy or methoxy group,
D. a 3-amino-piperidin-1-yl group wherein a hydrogen atom in the 2 position together with a hydrogen atom in the 5 position is replaced by a —CH₂—CH₂-bridge,
E. a hexahydroazepin-1-yl group which is substituted in the 3 position by an amino group,
F. a 3-amino-2-oxo-piperidin-5-yl or 3-amino-2-oxo-1-methyl-piperidin-5-yl group,
G. a cyclohexyl group which is substituted in the 3 position by an amino group,
H. a 2-amino-cyclohexylamino group,
I. or an amino group substituted by the groups R¹⁵ and R¹⁶ wherein
i. R¹⁵ denotes a methyl or ethyl group and
ii. R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
unless otherwise stated, the abovementioned alkyl and alkenyl groups may be straight-chained or branched.

6. The compound according to claim 1, wherein
I. R¹ denotes
A. a hydrogen atom,
B. a C₁₋₄-alkyl group,
C. a C₃₋₅-alkenyl group,
D. a 2-propen-1-yl group which is substituted by a methoxycarbonyl group,
E. a C₃₋₅-alkynyl group,
F. a phenyl-C₁₋₄-alkyl group wherein the phenyl moiety may be substituted by one or two fluorine atoms, one or two chlorine atoms, a bromine atom, one to three methyl groups, a trifluoromethyl, hydroxy, methoxy, nitro, amino, carboxy or ethoxycarbonyl group,
G. a 2-phenylethyl group wherein the ethyl moiety is substituted in the 2 position by a hydroxy, methoxy or hydroxyimino group,
H. a phenylcarbonylmethyl group wherein the phenyl moiety may be substituted by a fluorine atom or by a methyl, aminocarbonyl, aminosulphonyl, cyano, hydroxy, methoxy, phenoxy, benzyloxy, 2-propen-1-yloxy, 2-propyn-1-yloxy, cyanomethoxy, (methoxycarbonyl)methoxy, (aminocarbonyl)methoxy, (methylaminocarbonyl)methoxy, (dimethylaminocarbonyl)methoxy, methylsulphonyloxy, phenylsulphonyloxy, nitro, amino, (methoxycarbonyl)methylamino, acetylamino, methoxycarbonylamino, methylsulphonylamino, bis-(methylsulphonyl)-amino, aminocarbonylamino, dimethylaminocarbonylamino, (methylamino)thiocarbonylamino, (ethoxycarbonyl-amino)carbonylamino or cyanomethylamino group,
I. a phenylcarbonylmethyl group wherein the phenyl moiety is substituted by two methoxy groups or by a bromine atom and by a dimethylamino group,
J. a 2-(phenylcarbonyl)ethyl group,
K. a 2-phenylethenyl group,
L. a 2-(phenoxy)ethyl group,
M. a phenylsulphanylmethyl or phenylsulphinylmethyl group,
N. a naphthylmethyl or naphthylethyl group,
O. an isoxazolylmethyl, thiazolylmethyl, pyridylmethyl, benzo[d]isoxazolylmethyl, benzo[d]isothiazolylmethyl, (1H-indazol-3-yl)methyl, quinolinylmethyl or isoquinolinylmethyl group, wherein the heterocyclic moiety may be substituted in each case by a methyl group,
P. an isoquinolinylmethyl group wherein the isoquinolinyl moiety is substituted by a nitro or amino group,
Q. a (1,2-dihydro-2-oxo-quinolin-4-yl)methyl group,
R. a pyrrolylethyl, triazolylethyl, thienylethyl, thiazolylethyl or pyridylethyl group, wherein the heterocyclic moiety may be substituted in each case by a methyl group,
S. a thienylcarbonylmethyl group,
T. a methyl group which is substituted by a cyclopropyl, cyano, carboxy, aminocarbonyl or methoxycarbonyl group,
U. an ethyl group which is substituted in the 2 position by a hydroxy, methoxy, dimethylamino, carboxy or methoxycarbonyl group, or
V. a propyl group which is substituted in the 3 position by a hydroxy, dimethylamino, carboxy or methoxycarbonyl group,
W. a 2-oxopropyl group or
X. an amino or benzoylamino group,
II. R² denotes
A. a hydrogen atom,
B. a C₁₋₆-alkyl group,
C. an ethenyl group,
D. a 2-propen-1-yl or 2-propyn-1-yl group,
E. a phenyl group,
F. a phenyl-C₁₋₄-alkyl group wherein the phenyl moiety may be substituted by a fluorine atom, a methyl or methoxy group,
G. a phenylcarbonylmethyl group,
H. a 2-phenylethenyl group,
I. a methyl group which is substituted by a cyclopropyl, cyano, carboxy or methoxycarbonyl group, or
J. an ethyl group which is substituted in the 2 position by a cyano, hydroxy, methoxy or dimethylamino group,
III. R³ denotes
A. a C₄₋₆-alkenyl group,
B. a 1-cyclopenten-1-ylmethyl or 1-cyclohexen-1-ylmethyl group,
C. a 2-propyn-1-yl, 2-butyn-1-yl or 2-pentyn-1-yl group,
D. a phenyl group which may be substituted by a fluorine atom or a cyano, methyl or trifluoromethyl group,
E. a phenyl group which is substituted by two methyl groups,
F. a benzyl group wherein the phenyl moiety may be substituted by one or two fluorine atoms, an iodine atom or a cyano, nitro or amino group,
G. a furanylmethyl or thienylmethyl group or
H. a cyclopropylmethyl group and
IV. R⁴ denotes
A. a piperidin-1-yl group which is substituted in the 3 position by an amino group, wherein the piperidin-1-yl moiety may additionally be substituted by a methyl group,
B. a 3-amino-piperidin-1-yl group wherein the piperidin-1-yl moiety is additionally substituted by a pyrrolidin-1-yl-carbonyl group,
C. a 3-amino-piperidin-1-yl group wherein the piperidin-1-yl moiety is additionally substituted in the 4 position by a hydroxy group,
D. a 3-amino-piperidin-1-yl group wherein a hydrogen atom in the 2 position together with a hydrogen atom in the 5 position is replaced by a —CH₂—CH₂-bridge,
E. a hexahydroazepin-1-yl group which is substituted in the 3 position by an amino group,
F. a cyclohexyl group which is substituted in the 3 position by an amino group,
G. a 2-amino-cyclohexylamino group, or
H. an amino group substituted by the groups R¹⁵ and R¹⁶ wherein
i. R¹⁵ denotes a methyl or ethyl group and
ii. R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
unless otherwise stated, the abovementioned alkyl and alkenyl groups may be straight-chained or branched.

7. The compound according to claim 4, wherein
I. R¹, R² and R³ are defined as in claim 4 and
II. R⁴ denotes
A. an azetidin-1-yl or pyrrolidin-1-yl group which is substituted in the 3 position by a RₑNR_{d} group and may additionally be substituted by one or two C₁₋₃-alkyl groups, wherein
i. Rₑ denotes a hydrogen atom or a C₁₋₃-alkyl group and
ii. R_{d} denotes a hydrogen atom or a C₁₋₃-alkyl group,
B. a piperidin-1-yl or hexahydroazepin-1-yl group which is substituted in the 3 position or in the 4 position by a RₑNR_{d} group and may additionally be substituted by one or two C₁₋₃-alkyl groups, wherein Rₑ and R_{d} are as hereinbefore defined,
C. a 3-amino-piperidin-1-yl group wherein the piperidin-1-yl moiety is additionally substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
D. a 3-amino-piperidin-1-yl group wherein the piperidin-1-yl moiety is additionally substituted in the 4 position or in the 5 position by a hydroxy or methoxy group,
E. a 3-amino-piperidin-1-yl group wherein the methylene group in the 2 position or in the 6 position is replaced by a carbonyl group,
F. a piperidin-1-yl or hexahydroazepin-1-yl group substituted in the 3 position by an amino, C₁₋₃-alkylamino or di-(C₁₋₃-alkyl)-amino group, wherein in each case two hydrogen atoms on the carbon skeleton of the piperidin-1-yl or hexahydroazepin-1-yl group are replaced by a straight-chain alkylene bridge, this bridge containing 2 to 5 carbon atoms if the two hydrogen atoms are located on the same carbon atom, or 1 to 4 carbon atoms, if the hydrogen atoms are located on adjacent carbon atoms, or 1 to 4 carbon atoms, if the hydrogen atoms are located on carbon atoms separated by one atom, or 1 to 3 carbon atoms if the two hydrogen atoms are located on carbon atoms separated by two atoms,
G. an azetidin-1-yl, pyrrolidin-1-yl, piperidin-1-yl or hexahydroazepin-1-yl group which is substituted by an amino-C₁₋₃-alkyl, C₁₋₃-alkylamino-C₁₋₃-alkyl or a di-(C₁₋₃-alkyl)amino-C₁₋₃-alkyl group,
H. a C₃₋₇-cycloalkyl group which is substituted by an amino, C₁₋₃-alkylamino or di-(C₁₋₃-alkyl)-amino group,
I. a C₃₋₇-cycloalkyl group which is substituted by an amino-C₁₋₃-alkyl, C₁₋₃-alkylamino-C₁₋₃-alkyl or a di-(C₁₋₃-alkyl)amino-C₁₋₃-alkyl group,
J. a C₃₋₇-cycloalkyl-C₁₋₂-alkyl group wherein the cycloalkyl moiety is substituted by an amino, C₁₋₃-alkylamino or di-(C₁₋₃-alkyl)-amino group,
K. a C₃₋₇-cycloalkyl-C₁₋₂-alkyl group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
L. a C₃₋₇-cycloalkylamino group wherein the cycloalkyl moiety is substituted by an amino, C₁₋₃-alkylamino or di-(C₁₋₃-alkyl)-amino group, wherein the two nitrogen atoms at the cycloalkyl moiety are separated from one another by at least two carbon atoms,
M. a N—(C₃₋₇-cycloalkyl)-N—(C₁₋₃-alkyl)-amino group wherein the cycloalkyl moiety is substituted by an amino, C₁₋₃-alkylamino or di-(C₁₋₃-alkyl)-amino group, wherein the two nitrogen atoms at the cycloalkyl moiety are separated from one another by at least two carbon atoms,
N. a C₃₋₇-cycloalkylamino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
O. a N—(C₃₋₇-cycloalkyl)-N—(C₁₋₃-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
P. a C₃₋₇-cycloalkyl-C₁₋₂-alkyl-amino group wherein the cycloalkyl moiety is substituted by an amino, C₁₋₃-alkylamino or di-(C₁₋₃-alkyl)-amino group,
Q. a N—(C₃₋₇-cycloalkyl-C₁₋₂-alkyl)-N—(C₁₋₂-alkyl)-amino group wherein the cycloalkyl moiety is substituted by an amino, C₁₋₃-alkylamino or di-(C₁₋₃-alkyl)-amino group,
R. a C₃₋₇-cycloalkyl-C₁₋₂-alkyl-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
S. a N—(C₃₋₇-cycloalkyl-C₁₋₂-alkyl)-N—(C₁₋₂-alkyl)-amino group wherein the cycloalkyl moiety is substituted by an amino-C₁₋₃-alkyl, C₁₋₃-alkylamino-C₁₋₃-alkyl or a di-(C₁₋₃-alkyl)amino-C₁₋₃-alkyl group,
T. an amino group substituted by the groups R¹⁵ and R¹⁶ wherein
i. R¹⁵ denotes a C₁₋₄-alkyl group and
ii. R¹⁶ denotes a R¹⁷—C₂₋₃-alkyl group, wherein the C₂₋₃-alkyl moiety is straight-chained and may be substituted by one to four C₁₋₃-alkyl groups, which may be identical or different, or may be substituted by an aminocarbonyl, C₁₋₂-alkyl-aminocarbonyl, di-(C₁₋₂-alkyl)aminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group and
a. R¹⁷ denotes an amino, C₁₋₃-alkylamino or di-(C₁₋₃-alkyl)-amino group,
U. an amino group substituted by the group R²⁰, wherein
i. R²⁰ denotes an azetidin-3-yl, azetidin-2-ylmethyl, azetidin-3-ylmethyl, pyrrolidin-3-yl, pyrrolidin-2-ylmethyl, pyrrolidin-3-ylmethyl, piperidin-3-yl, piperidin-2-ylmethyl, piperidin-3-ylmethyl or piperidin-4-ylmethyl group, wherein the groups mentioned for R²⁰ may each be substituted by one or two C₁₋₃-alkyl groups,
V. an amino group substituted by the groups R¹⁵ and R²⁰ wherein
i. R¹⁵ and R²⁰ are as hereinbefore defined, wherein the groups mentioned for R²⁰ may each be substituted by one or two C₁₋₃-alkyl groups,
W. an R¹⁹—C₃₋₄-alkyl group wherein the C₃₋₄-alkyl moiety is straight-chained and may be substituted by the group R¹⁵ and may additionally be substituted by one or two C₁₋₃-alkyl groups, wherein R¹⁵ is as hereinbefore defined and R¹⁹ denotes an amino, C₁₋₃-alkylamino or di-(C₁₋₃-alkyl)-amino group,
X. a 3-amino-2-oxo-piperidin-5-yl or 3-amino-2-oxo-1-methyl-piperidin-5-yl group,
Y. a pyrrolidin-3-yl, piperidin-3-yl, piperidin-4-yl, hexahydroazepin-3-yl or hexahydroazepin-4-yl group, which is substituted in the 1 position by an amino, C₁₋₃-alkylamino or di-(C₁₋₃-alkyl)amino group, or
Z. an azetidin-2-yl-C₁₋₂-alkyl, azetidin-3-yl-C₁₋₂-alkyl, pyrrolidin-2-yl-C₁₋₂-alkyl, pyrrolidin-3-yl, pyrrolidin-3-yl-C₁₋₂-alkyl, piperidin-2-yl-C₁₋₂-alkyl, piperidin-3-yl, piperidin-3-yl-C₁₋₂-alkyl, piperidin-4-yl or piperidin-4-yl-C₁₋₂-alkyl group, wherein the abovementioned groups may each be substituted by one or two C₁₋₃-alkyl groups.
while unless otherwise stated, the abovementioned alkyl- and alkenyl groups may be straight-chained or branched.

8. The compound according to claim 5, wherein
I. R¹, R² and R³ are defined as in claim 5 and
II. R⁴ denotes
A. a piperidin-1-yl group which is substituted in the 3 position by an amino group, wherein the piperidin-1-yl moiety may additionally be substituted by a methyl group,
B. a 3-amino-piperidin-1-yl group wherein the piperidin-1-yl moiety is additionally substituted by an aminocarbonyl, methylaminocarbonyl, dimethylaminocarbonyl, pyrrolidin-1-yl-carbonyl, (2-cyano-pyrrolidin-1-yl-)carbonyl, thiazolidin-3-yl-carbonyl, (4-cyano-thiazolidin-3-yl)carbonyl, piperidin-1-ylcarbonyl or morpholin-4-ylcarbonyl group,
C. a 3-amino-piperidin-1-yl group wherein the piperidin-1-yl moiety is additionally substituted in the 4 position or in the 5 position by a hydroxy or methoxy group,
D. a 3-amino-piperidin-1-yl group wherein a hydrogen atom in the 2 position together with a hydrogen atom in the 5 position is replaced by a —CH₂—CH₂-bridge,
E. a hexahydroazepin-1-yl group which is substituted in the 3 position by an amino group,
F. a 3-amino-2-oxo-piperidin-5-yl or 3-amino-2-oxo-1-methyl-piperidin-5-yl group,
G. a cyclohexyl group which is substituted in the 3 position by an amino group,
H. a 2-amino-cyclohexylamino group,
I. or an amino group substituted by the groups R¹⁵ and R¹⁶, wherein
i. R¹⁵ denotes a methyl or ethyl group and
ii. R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
while unless otherwise stated, the abovementioned alkyl- and alkenyl groups may be straight-chained or branched.

9. The compound according to claim 6, wherein
I. R¹, R² and R³ are defined as in claim 6 and
II. R⁴ denotes
A. a piperidin-1-yl group which is substituted in the 3 position by an amino group, wherein the piperidin-1-yl moiety may additionally be substituted by a methyl group,
B. a 3-amino-piperidin-1-yl group wherein the piperidin-1-yl moiety is additionally substituted by a pyrrolidin-1-yl-carbonyl group,
C. a 3-amino-piperidin-1-yl group wherein the piperidin-1-yl moiety in the 4 position is additionally substituted by a hydroxy group,
D. a 3-amino-piperidin-1-yl group wherein a hydrogen atom in the 2 position together with a hydrogen atom in the 5 position is replaced by a —CH₂—CH₂-bridge,
E. a hexahydroazepin-1-yl group which is substituted in the 3 position by an amino group,
F. a cyclohexyl group which is substituted in the 3 position by an amino group,
G. a 2-amino-cyclohexylamino group,
H. or an amino group substituted by the groups R¹⁵ and R¹⁶, wherein
i. R¹⁵ denotes a methyl or ethyl group and
ii. R¹⁶ denotes a 2-aminoethyl group, wherein the ethyl moiety may be substituted by one or two methyl groups or by an aminocarbonyl, methylaminocarbonyl, dimethylaminocarbonyl or pyrrolidin-1-ylcarbonyl group,
while unless otherwise stated the abovementioned alkyl- and alkenyl groups may be straight-chained or branched.

10. A method of treating a condition or disease which can be affected by the inhibition of DPP-4 activity in a patient comprising administering to the patient a compound selected from the group consisting of:
(1) 1,3-dimethyl-7-benzyl-8-(3-amino-pyrrolidin-1-yl)-xanthine,
(2) 1,3-dimethyl-7-(3-methyl-2-buten-1-yl)-8-(3-amino-pyrrolidin-1-yl)-xanthine,
(3) 1,3-dimethyl-7-benzyl-8-(3-amino-piperidin-1-yl)-xanthine,
(4) 1,3-dimethyl-7-(3-methyl-2-buten-1-yl)-8-[(trans-2-amino-cyclohexyl)amino]-xanthine,
(5) 1,3-dimethyl-7-(3-methyl-2-buten-1-yl)-8-(3-amino-piperidin-1-yl)-xanthine,
(6) 1,3-dimethyl-7-(3-methyl-2-buten-1-yl)-8-(4-amino-piperidin-1-yl)-xanthine,
(7) 1,3-dimethyl-7-(3-methyl-2-buten-1-yl)-8-[(cis-2-amino-cyclohexyl)amino]-xanthine,
(8) 1,3-dimethyl-7-(2-butyn-1-yl)-8-(3-amino-piperidin-1-yl)-xanthine,
(9) 1,3-dimethyl-7-[(1-cyclopenten-1-yl)methyl]-8-(3-amino-piperidin-1-yl)-xanthine,
(10) 1,3-dimethyl-7-(2-thienylmethyl)-8-(3-amino-piperidin-1-yl)-xanthine,
(11) 1,3-dimethyl-7-(3-fluorobenzyl)-8-(3-amino-piperidin-1-yl)-xanthine,
(12) 1,3-dimethyl-7-(2-fluorobenzyl)-8-(3-amino-piperidin-1-yl)-xanthine,
(13) 1,3-dimethyl-7-(4-fluorobenzyl)-8-(3-amino-piperidin-1-yl)-xanthine,
(14) 1,3-dimethyl-7-(2-buten-1-yl)-8-(3-amino-piperidin-1-yl)-xanthine,
(15) 1,3-bis-(cyclopropylmethyl)-7-benzyl-8-(3-amino-piperidin-1-yl)-xanthine,
(16) (R)-1,3-dimethyl-7-(3-methyl-2-buten-1-yl)-8-(3-amino-piperidin-1-yl)-xanthine,
(17) (S)-1,3-dimethyl-7-(3-methyl-2-buten-1-yl)-8-(3-amino-piperidin-1-yl)-xanthine,
(18) 1,3-dimethyl-7-(3-methyl-2-buten-1-yl)-8-(3-amino-hexahydroazepin-1-yl)-xanthine,
(19) 1,3-dimethyl-7-(3-methyl-2-buten-1-yl)-8-(4-amino-hexahydroazepin-1-yl)-xanthine,
(20) 1,3-dimethyl-7-(3-methyl-2-buten-1-yl)-8-(cis-3-amino-cyclohexyl)-xanthine-hydrochloride,
(21) 1,3-dimethyl-7-(3-methyl-2-buten-1-yl)-8-(3-methylamino-piperidin-1-yl)-xanthine,
(22) 1-(2-phenylethyl)-3-methyl-7-(3-methyl-2-buten-1-yl)-8-(3-amino-piperidin-1-yl)-xanthine,
(23) 1,3-dimethyl-7-(3-methyl-2-buten-1-yl)-8-[N-(2-aminoethyl)-methylamino]-xanthine,
(24) 1-[2-(thiophen-2-yl)-ethyl]-3-methyl-7-(3-methyl-2-buten-1-yl)-8-(3-amino-piperidin-1-yl)-xanthine,
(25) 1-[2-(thiophen-3-yl)-ethyl]-3-methyl-7-(3-methyl-2-buten-1-yl)-8-(3-amino-piperidin-1-yl)-xanthine,
(26) 1-[2-(2-methyl-phenyl)-ethyl]-3-methyl-7-(3-methyl-2-buten-1-yl)-8-(3-amino-piperidin-1-yl)-xanthine,
(27) 1-[2-(3-methyl-phenyl)-ethyl]-3-methyl-7-(3-methyl-2-buten-1-yl)-8-(3-amino-piperidin-1-yl)-xanthine,
(28) 1-[2-(3-methoxy-phenyl)-ethyl]-3-methyl-7-(3-methyl-2-buten-1-yl)-8-(3-amino-piperidin-1-yl)-xanthine,
(29) 1-((E)-2-phenyl-vinyl)-3-methyl-7-(3-methyl-2-buten-1-yl)-8-(3-amino-piperidin-1-yl)-xanthine,
(30) 1-(2-phenyl-ethyl)-3-methyl-7-(3-methyl-2-buten-1-yl)-8-((S)-3-amino-piperidin-1-yl)-xanthine,
(31) 1-(2-phenyl-ethyl)-3-methyl-7-(3-methyl-2-buten-1-yl)-8-((R)-3-amino-piperidin-1-yl)-xanthine,
(32) 1-[2-(2-methoxy-phenyl)-2-oxo-ethyl]-3-methyl-7-(3-methyl-2-buten-1-yl)-8-(3-amino-piperidin-1-yl)-xanthine,
(33) 1-[2-(thiophen-3-yl)-2-oxo-ethyl]-3-methyl-7-(3-methyl-2-buten-1-yl)-8-(3-amino-piperidin-1-yl)-xanthine,
(34) 1-(2-phenyl-2-oxo-ethyl)-3-methyl-7-(3-methyl-2-buten-1-yl)-8-((S)-3-amino-piperidin-1-yl)-xanthine,
(35) 1-(2-phenyl-2-oxo-ethyl)-3-methyl-7-(3-methyl-2-buten-1-yl)-8-((R)-3-amino-piperidin-1-yl)-xanthine,
(36) 1-[(isoquinolin-1-yl)methyl]-3-methyl-7-(3-methyl-2-buten-1-yl)-8-((R)-3-amino-piperidin-1-yl)-xanthine,
(37) 1-[(isoquinolin-1-yl)methyl]-3-methyl-7-(3-methyl-2-buten-1-yl)-8-((S)-3-amino-piperidin-1-yl)-xanthine and
(38) 1-[(1-naphthyl)methyl]-3-methyl-7-(3-methyl-2-buten-1-yl)-8-(3-amino-piperidin-1-yl)-xanthine
and the salts thereof.

11. A physiologically acceptable salt of the compound according to claim 1.

12. A pharmaceutical composition comprising a pharmaceutically effective amount of the compound according to claim 1 and one or more of a carrier or diluent.

13. A method of treating Type II diabetes mellitus comprising administering to a patient a pharmaceutically effective amount of a compound according to claim 1.

14. A method of treating a condition or disease which can be affected by the inhibition of DPP-IV activity comprising administering to a patient a pharmaceutically effective amount of a compound according to claim 1.

15. The method of claim 10, wherein the condition or disease is selected from the group consisting of type I and type II diabetes mellitus, diabetic complications, metabolic acidosis or ketosis, insulin resistance, dyslipidaemias of various origins, arthritis, atherosclerosis and related diseases, obesity, allograft transplantation and osteoporosis caused by calcitonin.

16. The method of claim 14, wherein the condition or disease is selected from the group consisting of type I and type II diabetes mellitus, diabetic complications, metabolic acidosis or ketosis, insulin resistance, dyslipidaemias of various origins, arthritis, atherosclerosis and related diseases, obesity, allograft transplantation and osteoporosis caused by calcitonin.
